(19) United States
(12) Patent Application Publication
(10) Pub. No.: US 2024/0262922 A1
(43) Pub. Date: Aug. 8, 2024

(54) PROTEINS THAT BIND CD80 AND/OR CD86, AND OX40L

(71) Applicant: Merck Patent GmbH, Darmstadt (DE)

(72) Inventors: Alec Gross, Billerica, MA (US); Chia Chi Sun, Billerica, MA (US); Gang Chen, Billerica, MA (US); Yves Fomekong Nanfack, Billerica, MA (US); Qi An, Billerica, MA (US); Bijan Zakeri, Woburn, MA (US); Qingyong Ji, Billerica, MA (US)

(21) Appl. No.: 18/530,536

(22) Filed: Dec. 6, 2023

Related U.S. Application Data
(63) Continuation of application No. PCT/US22/32482, filed on Jun. 7, 2022.
(60) Provisional application No. 63/208,478, filed on Jun. 8, 2021.
(60) Provisional application No. 63/343,268, filed on May 18, 2022.

(51) Int. Cl. C07K 16/28; A61K 39/00; A61P 37/06; C07K 14/705

(52) U.S. Cl. CPC C07K 16/2875; A61P 37/06; C07K 14/70521; A61K 2039/505; C07K 2317/33; C07K 2317/53; C07K 2317/55; C07K 2317/565; C07K 2317/76; C07K 2317/92

(57) ABSTRACT

This disclosure relates generally to proteins that inhibit T cell costimulatory signaling comprising: a polypeptide or complex of two or more polypeptides that specifically binds CD80 and/or CD86, and a polypeptide or complex of two or more polypeptides that specifically binds OX40L. In some embodiments, the present application provides antibodies that specifically bind OX40L. In some embodiments the application also provides therapeutic methods for using such proteins in the treatment of autoimmune diseases.

### CROSS-REFERENCE TO RELATED APPLICATIONS

This application is a continuation of International Patent Application No. PCT/US2022/032482 filed Jun. 7, 2022, and designating the United States, which application claims the benefit of and priority to U.S. Provisional Patent Application No. 63/208,478, filed Jun. 8, 2021, and to U.S. Provisional Patent Application No. 63/343,268, filed May 18, 2022, the entire disclosures of each of which are incorporated by reference herein for all purposes.

### SEQUENCE LISTING

The instant application contains a Sequence Listing which has been submitted electronically in XML format. The Sequence Listing XML is hereby incorporated by reference in its entirety. Said XML file, created on Feb. 12, 2024, is named EMD-015USWOC1.xml and is 393,522 bytes in size.

### FIELD OF THE DISCLOSURE

The present disclosure relates generally to proteins capable of modulating T cell costimulatory signaling, and uses of the same in the treatment of autoimmune diseases/disorders.

### BACKGROUND

A major therapeutic goal for the treatment of autoimmune diseases is to modulate T cell stimulation by specifically targeting costimulatory pathways. T cells become fully activated when they receive two signals from: 1) T-cell receptor (TCR) activation due to interaction with a peptide/MHC complex on the surface of an antigen presenting cell (APC); and 2) T cell costimulatory receptor activation due to interaction with their cognate ligands. T cell stimulation in the absence of the second costimulatory signal results in the development of anergic or tolerogenic T cell responses.

There are two major groups of costimulatory molecules: 1) the B7-CD28 superfamily, and 2) the tumor necrosis factor receptor (TNFR) superfamily. Costimulatory molecules in the B7-CD28 superfamily include CD28 and ICOS on T cells and their cognate ligands CD80/CD86 (also known as B7.1/B7.2) and ICOS-L, respectively, on the surface of APCs. Costimulatory molecules in the TNFR superfamily include 4-1BB, CD40L, OX40, CD27, GITR, and CD30 on T cells and their cognate ligands 4-1BBL, CD40, OX40L, CD70, GITRL, and CD30L, respectively.

APCs such as B cells, monocytes, macrophages and dendritic cells up-regulate the expression of CD80 and CD86 upon activation by antigenic, inflammatory, or pathogen-associated molecular pattern receptors. These ligands engage CD28 on T cells and provide the necessary second costimulatory signal to activate naïve T cells and promote T cell proliferation.

Since CD80 and CD86 have a higher affinity to CTLA4 than CD28, CTLA4 expression on T cells inhibits CD80/CD86 binding to CD28 by sequestering the costimulatory ligands and attenuating T cell activation. Abatacept is a soluble recombinant CTLA4-Ig fusion protein comprising of the extracellular domain of human CTLA4 and a modified human immunoglobulin 1 (IgG1) Fc domain. Abatacept selectively binds CD80/CD86 on APCs and prevent costimulatory activation of CD28 on T cells, resulting in decreased T cell activation, signaling and proliferation. Abatacept has demonstrated clinical efficacy in treating a number of autoimmune diseases including adult rheumatoid arthritis, psoriatic arthritis, and juvenile idiopathic arthritis. However, in clinical trials for systemic lupus erythematosus (SLE), lupus nephritis (LN), and primary Sjogren's Syndrome (pSS), Abatacept has missed its clinical endpoints.

OX40 ligand (OX40L, also known as CD252, gp34, and TNFSF4) is a 34 kDa type II transmembrane protein of the TNF superfamily. OX40L expression is induced upon activation of APCs and chronically activated T cells. OX40L is also upregulated in non-lymphoid cells like human tonsillar mast cells, bronchial smooth muscle cells, and vascular endothelial cells of inflamed or diseased tissues. OX40L exists in a soluble form due to cleavage by proteolytic enzymes as is typical of most TNF family ligands. Soluble OX40L serum levels have been reported to be elevated in autoimmune disease patients as compared to healthy subjects.

OX40L on APCs binds to OX40 (CD134, TNFRSF4) expressed on activated CD4 and CD8 T cells. OX40 is not expressed on resting naïve or resting memory T cells. OX40L binds as a homotrimeric complex to three OX40 monomers on T cells. Binding of OX40L to OX40 stimulates T cell expansion, differentiation, and survival of T effector subsets including T helper 1 (Th1), T helper 2 (Th2), T follicular helper (Tfh), and T helper 17 (Th17) cells. OX40 signaling is also critical for the generation, maintenance, and optimal re-activation of memory CD4+ T cells.

Dysregulation of the OX40L-OX40 pathway had been implicated in a wide variety of inflammatory diseases such as multiple sclerosis, arthritis, graft versus host disease (GVHD), lupus nephritis and rheumatoid arthritis. TNFSF4, the gene encoding OX40L, is a susceptibility locus for systemic lupus erythematosus (SLE) and systemic sclerosis (SSc). Myeloid cells and B cells expressing OX40L have been demonstrated to support Tfh cell development, contributing to SLE disease pathogenesis. OX40L-OX40 signaling also contributes to the pathogenesis of autoimmune disease by dysregulating T regulatory cells (Tregs or T_{regs}). Foxp3+ Tregs play an important role in establishing immunological tolerance by suppressing over-activated T effector (Teff, T_{eff}, or T_{effector}) cells. In human SLE, OX40L-OX40 signaling inhibits Foxp3 expression in Tregs and negatively impacts their suppressive function.

### SUMMARY OF THE DISCLOSURE

The invention generally relates to proteins capable of modulating T cell costimulatory signaling, and uses of the same in the treatment of autoimmune diseases/disorders.

In one aspect the present disclosure provides a protein comprising a polypeptide or complex of two or more polypeptides that specifically binds CD80 and/or CD86, and a polypeptide or complex of two or more polypeptides that specifically binds OX40L. In some embodiments, the polypeptide or complex of two or more polypeptides that specifically binds CD80 and/or CD86 is a polypeptide comprising a CTLA4 extracellular domain or functional fragment thereof; a polypeptide comprising a CD28 extracellular domain or functional fragment thereof; an antibody; a Fab; a Fab′, a F(ab′)₂, a single-chain variable fragment (scFv); a minibody; or a nanobody (VHH). In some embodiments the polypeptide or complex of two or more polypeptides that specifically binds CD80 and/or CD86 comprises an extracellular domain of CTLA4 or functional fragment thereof consisting of or comprising a sequence having at least 90% sequence identity to SEQ ID NO: 29, SEQ ID NO: 173, or SEQ ID NO: 174. In some embodiments, the polypeptide or complex of two or more polypeptides that specifically binds CD80 and/or CD86 inhibits CD28 costimulatory signaling in T cells.

In another aspect of the present disclosure, the polypeptide or complex of two or more polypeptides that specifically binds OX40L inhibits binding of endogenous OX40L to endogenous OX40. In some embodiments, the polypeptide or complex of two or more polypeptides that specifically binds OX40L comprises an antigen-binding site. In some embodiments, the antigen binding site is an antibody, a Fab, a Fab′, a F(ab′)₂, a single-chain variable fragment (scFv), a minibody, or a nanobody (VHH). In some embodiments, the antigen-binding site is a Fab comprising a polypeptide comprising the sequence of SEQ ID NO: 336 connected to the C-terminus of the Fab heavy chain CH1 domain. In other embodiments, the polypeptide or complex of two or more polypeptides that specifically binds OX40L is an extracellular domain of OX40 or functional fragment thereof.

In some embodiments, proteins of the present disclosure further comprise a bridging moiety. In some embodiments, the bridging moiety is functional or non-functional. In some embodiments, the bridging moiety comprises polypeptide of an immunoglobulin Fc domain or functional fragment thereof, a human serum albumin (HSA) polypeptide or functional fragment thereof, a polypeptide linker, or a polypeptide hinge. In some embodiments, the bridging moiety comprises a polypeptide of a human IgG1, IgG2, IgG3, or IgG4 Fc domain. In some embodiments, the polypeptide of a human IgG1 Fc domain comprises a sequence having at least 90% or 100% sequence identity to an amino acid sequence selected from a group consisting of SEQ ID NO: 178, SEQ ID NO: 237, SEQ ID NO: 238, SEQ ID NO: 214, SEQ ID NO: 216, SEQ ID NO: 179, SEQ ID NO: 180, SEQ ID NO: 239, and SEQ ID NO: 240. In some embodiments, the polypeptide of a human IgG1 Fc domain comprises one or more than one effector function silencing mutation. In certain embodiments, the hinge polypeptide comprises mutations at positions 234 and 235 from leucine to alanine.

In some embodiments, the bridging moiety further comprises a hinge polypeptide comprising an amino acid sequence having at least 90% sequence identity to an amino acid sequence selected from a group consisting of SEQ ID NO: 175, SEQ ID NO: 242, SEQ ID NO: 176, SEQ ID NO: 249, SEQ ID NO: 177, SEQ ID NO: 243, SEQ ID NO: 212, SEQ ID NO: 244, SEQ ID NO: 213, SEQ ID NO: 245, SEQ ID NO: 247, and SEQ ID NO: 248.

In some embodiments of proteins of the present invention, the bridging moiety is connected to the N-terminus or the C-terminus of the polypeptide or complex of two or more polypeptides that specifically binds CD80 and/or CD86. In some embodiments, the polypeptide or complex of two or more polypeptides that specifically binds CD80 and/or CD86 is connected to the N-terminus of the bridging moiety. In some embodiments, the bridging moiety is connected to the N-terminus or the C-terminus of the polypeptide or complex of two or more polypeptides that specifically binds OX40L. In some embodiments, the polypeptide or complex of two or more polypeptides that specifically binds OX40L is connected to the C-terminus of the bridging moiety. In some embodiments, a linker polypeptide comprising a (GGGGS)ₙ (SEQ ID NO: 181) sequence, wherein n is 1 to 12, connects the polypeptide or complex of two or more polypeptides that specifically binds OX40L to the bridging moiety.

In some embodiments, proteins of the present disclosure comprise a second polypeptide or complex of two or more polypeptides that specifically binds OX40L. In some embodiments, the bridging moiety is a polypeptide of a human IgG1 Fc domain and the polypeptide or complex of two or more polypeptides that specifically binds OX40L is connected to the C-terminus of the polypeptide of the human IgG1 Fc domain and the second polypeptide or complex of two or more polypeptides that specifically binds OX40L is connected to the C-terminus of a second polypeptide of a human IgG1 Fc domain. In some embodiments, the first and/or the second polypeptide or complex of two or more polypeptides that specifically binds OX40L comprises an antigen-binding site, for example a Fab, or an extracellular domain of OX40 or functional fragment thereof. In some embodiments, the second polypeptide or complex of two or more polypeptides that specifically binds OX40L is connected to the bridging moiety by a linker polypeptide comprising a (GGGGS)ₙ (SEQ ID NO: 181) sequence, wherein n is 1 to 12.

In some embodiments, proteins of the present disclosure comprise antigen-binding sites comprising an amino acid sequence of (a) ARHRGX₁YX₂FDX₃ (SEQ ID NO: 220), wherein X₁ is S or I, X₂ is F or H, and X₃ is I or Y; or (b) ARERSX₁X₂WYPX₃DY (SEQ ID NO: 221) wherein X₁ is N or S, X₂ is N, D, G or S, and X₃ is I or F.

In some embodiments, proteins of the present disclosure comprise antigen-binding sites comprising (a) a heavy chain variable complementarity-determining region 1 (VHCDR1) comprising an amino acid sequence GX₁SX₂X₃X₄SX₅YY (SEQ ID NO: 222), wherein X₁ is A, G, or V, X₂ is V, or I, X₃ is S or R, X₄ is S, or T, and X₅ is S, or G; (b) a heavy chain variable complementarity-determining region 2 (VHCDR2) comprising an amino acid sequence of: (1) IX₁YX₂GST (SEQ ID NO: 223) wherein X₁ is Y, or N, and X₂ is S, or G, (2) X₁DYSGT (SEQ ID NO: 224) wherein X₁ is I or M, or (3) IGSVDYSGX₁T (SEQ ID NO: 225), wherein X₁ is N, A, or S; and (c) a heavy chain variable complementarity-determining region 3 (VHCDR3) comprising an amino acid sequence of: (1) ARHRGX₁YX₂FDX₃ (SEQ ID NO: 220), wherein X₁ is S or I, X₂ is F or H, and X₃ is I or Y; or (2) ARERSX₁X₂WYPX₃DY (SEQ ID NO: 221), wherein X₁ is N or S, X₂ is N, D, G or S, and X₃ is I or F.

In some embodiments, proteins of the present disclosure comprise antigen-binding sites comprising (a) a light chain variable complementarity-determining region 1 (VLCDR1) comprising an amino acid sequence of: (1) X₁IENKN (SEQ ID NO: 226), wherein X₁ is N or D, or (2) SX₁RX₂X₃X₄, wherein X₁ is V or L, X₂ is R or N, X₃ is F or Y, and X₄ is F or Y; (b) a light chain variable complementarity-determining region 2 (VLCDR2) comprising an amino acid sequence of: RDN, GKD, or RDS; and (c) a light chain variable complementarity-determining region 3 (VLCDR3) comprising an amino acid sequence of: (1) QVX₁DSX₂X₃VV (SEQ ID NO: 231), wherein X₁ is R or W, X₂ is N, T, or A, and X₃ is I, T, or A, or (2) NSRDSSGYX₁VX₂ (SEQ ID NO: 232), wherein X₁ is L or H, and X₂ is L or V.

In some embodiments, proteins of the present disclosure comprise antigen-binding sites comprising (i) (a) a heavy chain variable complementarity-determining region 1 (VHCDR1) comprising an amino acid sequence GX₁SX₂X₃X₄SX₅YY (SEQ ID NO: 222), wherein X₁ is A, G, or V, X₂ is V, or I, X₃ is S or R, X₄ is S, or T, and X₅ is S, or G; and (b) a heavy chain variable complementarity-determining region 2 (VHCDR2) comprising an amino acid sequence IX₁YX₂GST (SEQ ID NO: 223), wherein X₁ is Y, or N, and X₂ is S, or G; and (c) a heavy chain variable complementarity-determining region 3 (VHCDR3) comprising an amino acid sequence ARHRGX₁YX₂FDX₃ (SEQ ID NO: 220), wherein X₁ is S or I, X₂ is F or H, and X₃ is I or Y; and (ii) (a) a light chain variable complementarity-determining region 1 (VLCDR1) comprising an amino acid sequence X₁IENKN (SEQ ID NO: 226), wherein X₁ is N or D; (b) a light chain variable complementarity-determining region 2 (VLCDR2) comprising an amino acid sequence RDN; and (c) a light chain variable complementarity-determining region 3 (VLCDR3) comprising an amino acid sequence QVX₁DSX₂X₃VV (SEQ ID NO: 231), wherein X₁ is R or W, X₂ is N, T, or A, and X₃ is I, T, or A.

In some embodiments, proteins of the present disclosure comprise antigen-binding sites comprising (i) (a) a heavy chain variable complementarity-determining region 1 (VHCDR1) comprising an amino acid sequence GX₁SX₂X₃X₄SX₅YY (SEQ ID NO: 222), wherein X₁ is A, G, or V, X₂ is V, or I, X₃ is S or R, X₄ is S, or T, and X₅ is S, or G; and (b) a heavy chain variable complementarity-determining region 2 (VHCDR2) comprising an amino acid sequence X₁DYSGT (SEQ ID NO: 224), wherein X₁ is I or M; and (c) a heavy chain variable complementarity-determining region 3 (VHCDR3) comprising an amino acid sequence ARERSX₁X₂WYPX₃DY (SEQ ID NO: 221), wherein X₁ is N or S, X₂ is N, D, G or S, and X₃ is I or F; and (ii) (a) a light chain variable complementarity-determining region 1 (VLCDR1) comprising an amino acid sequence SX₁R X₂X₃X₄, wherein X₁ is V or L, X₂ is R or N, X₃ is F or Y, and X₄ is F or Y; (b) a light chain variable complementarity-determining region 2 (VLCDR2) comprising an amino acid sequence GKD; and (c) a light chain variable complementarity-determining region 3 (VLCDR3) comprising an amino acid sequence NSRDSSGYX₁VX₂ (SEQ ID NO: 232) wherein X₁ is L or H, and X₂ is L or V.

In some embodiments, proteins of the present disclosure comprise antigen-binding sites comprising a heavy chain complementarity-determining region 1 (VHCDR1) comprising an amino acid sequence of GGSISTSSYY (SEQ ID NO: 77), a heavy chain complementarity-determining region 2 (VHCDR2) comprising an amino acid sequence of IYYSGST (SEQ ID NO: 78), a heavy chain complementarity-determining region 3 (VHCDR3) comprising an amino acid sequence of ARHRGSYFFDI (SEQ ID NO: 79), a light chain complementarity-determining region 1 (VLCDR1) comprising an amino acid sequence of DIENKN (SEQ ID NO: 81), a light chain complementarity-determining region 2 (VLCDR2) comprising an amino acid sequence of RDN, and a light chain complementarity-determining region 3 (VLCDR3) comprising an amino acid sequence of QVRDSNIVV (SEQ ID NO: 83).

In some embodiments, proteins of the present disclosure comprise antigen-binding sites comprising a heavy chain complementarity-determining region 1 (VHCDR1) comprising an amino acid sequence of GVSIRSNGYY (SEQ ID NO: 93), a heavy chain complementarity-determining region 2 (VHCDR2) comprising an amino acid sequence of MDYSGT (SEQ ID NO: 94), a heavy chain complementarity-determining region 3 (VHCDR3) comprising an amino acid sequence of ARERSNNWYPIDY (SEQ ID NO: 95), a light chain complementarity-determining region 1 (VLCDR1) comprising an amino acid sequence of SVRRFF (SEQ ID NO: 97), a light chain complementarity-determining region 2 (VLCDR2) comprising an amino acid sequence of GKD, and a light chain complementarity-determining region 3 (VLCDR3) comprising an amino acid sequence of NSRDSSGYLVL (SEQ ID NO: 99).

In some embodiments, proteins of the present disclosure comprise antigen-binding sites comprising a heavy chain complementarity-determining region 1 (VHCDR1) comprising an amino acid sequence of GASVSSSSYY (SEQ ID NO: 85), a heavy chain complementarity-determining region 2 (VHCDR2) comprising an amino acid sequence of INYGGST (SEQ ID NO: 86), a heavy chain complementarity-determining region 3 (VHCDR3) comprising an amino acid sequence of ARHRGIYHFDY (SEQ ID NO: 87), a light chain complementarity-determining region 1 (VLCDR1) comprising an amino acid sequence of NIENKN (SEQ ID NO: 89), a light chain complementarity-determining region 2 (VLCDR2) comprising an amino acid sequence of RDS, and a light chain complementarity-determining region 3 (VLCDR3) comprising an amino acid sequence of QVWDSNTVV (SEQ ID NO: 91).

In some embodiments, proteins of the present disclosure comprise antigen-binding sites comprising a heavy chain complementarity-determining region 1 (VHCDR1) comprising an amino acid sequence of GGSISSSSYY (SEQ ID NO: 101), a heavy chain complementarity-determining region 2 (VHCDR2) comprising an amino acid sequence of IGSVDYSGNT (SEQ ID NO: 102), a heavy chain complementarity-determining region 3 (VHCDR3) comprising an amino acid sequence of ARHRGIYFFDY (SEQ ID NO: 103), a light chain complementarity-determining region 1 (VLCDR1) comprising an amino acid sequence of NIENKN (SEQ ID NO: 89), a light chain complementarity-determining region 2 (VLCDR2) comprising an amino acid sequence of RDS, and a light chain complementarity-determining region 3 (VLCDR3) comprising an amino acid sequence of QVWDSNTVV (SEQ ID NO: 91).

In some embodiments, proteins of the present disclosure comprise antigen-binding sites comprising an amino acid sequence having at least 90% sequence identity to SEQ ID NO: 76, SEQ ID NO: 92, SEQ ID NO: 84, or SEQ ID NO: 100, and an amino acid sequence having at least 90% sequence identity to SEQ ID NO: 80, SEQ ID NO: 96, SEQ ID NO: 88, or SEQ ID NO: 104.

In some embodiments, proteins of the present disclosure comprise antigen-binding sites comprising an amino acid sequence having at least 90% sequence identity to SEQ ID NO: 76, and an amino acid sequence having at least 90% sequence identity to SEQ ID NO: 80.

In some embodiments, proteins of the present disclosure comprise antigen-binding sites comprising an amino acid sequence having at least 90% sequence identity to SEQ ID NO: 92, and an amino acid sequence having at least 90% sequence identity to SEQ ID NO: 96.

In some embodiments, proteins of the present disclosure comprise antigen-binding sites comprising an amino acid sequence having at least 90% sequence identity to SEQ ID NO: 8, SEQ ID NO: 16, SEQ ID NO: 14, or SEQ ID NO: 18, and an amino acid sequence having at least 90% sequence identity to SEQ ID NO: 7, SEQ ID NO: 15, SEQ ID NO: 13, or SEQ ID NO: 17.

In some embodiments, proteins of the present disclosure comprise antigen-binding sites comprising an amino acid sequence having at least 90% sequence identity to SEQ ID NO: 8, and an amino acid sequence having at least 90% sequence identity to SEQ ID NO: 7.

In some embodiments, proteins of the present disclosure comprise antigen-binding sites comprising an amino acid sequence having at least 90% sequence identity to SEQ ID NO: 16, and an amino acid sequence having at least 90% sequence identity to SEQ ID NO: 15.

In some embodiments, proteins of the present disclosure comprise antigen-binding sites that bind at position 60 and/or 83 of the extracellular domain of human OX40L. In some embodiments, the antigen-binding site binds at position 17, 18, 19, 20, 21, 23, 26, 28, 60, 83, 110, 111, 112, 113, and 114 of the extracellular domain of human OX40L. In some embodiments, the antigen-binding site binds at position 58, 59, 60, 61, 62, 63, 81, 82, and 83 of the extracellular domain of human OX40L.

In some embodiments, proteins of the present disclosure comprise (i) a polypeptide comprising the extracellular domain of CTLA4 comprising SEQ ID NO: 174; (ii) a bridging moiety comprising a sequence comprising of SEQ ID NO: 177 and a sequence comprising of SEQ ID NO: 179; and (iii) an antigen-binding site comprising, a heavy chain variable complementarity-determining region 1 (VHCDR1) comprising an amino acid sequence of GGSISTSSYY (SEQ ID NO: 77), a heavy chain variable complementarity-determining region 2 (VHCDR2) comprising an amino acid sequence of IYYSGST (SEQ ID NO: 78), a heavy chain variable complementarity-determining region 3 (VHCDR3) comprising an amino acid sequence of ARHRGSYFFDI (SEQ ID NO: 79), a light chain variable complementarity-determining region 1 (VLCDR1) comprising an amino acid sequence of DIENKN (SEQ ID NO: 81), a light chain variable complementarity-determining region 2 (VLCDR2) comprising an amino acid sequence of RDN, and a light chain variable complementarity-determining region 3 (VLCDR3) comprising an amino acid sequence of QVRDSNIVV (SEQ ID NO: 83).

In some embodiments, proteins of the present disclosure comprise a first arm and a second arm, the first arm comprising (i) a first polypeptide comprising the extracellular domain of CTLA4 comprising SEQ ID NO: 174; (ii) a first bridging moiety comprising a sequence comprising of SEQ ID NO: 177 and a sequence comprising of SEQ ID NO: 179; and (iii) a first antigen-binding site comprising, a heavy chain variable complementarity-determining region 1 (VHCDR1) comprising an amino acid sequence of GGSISTSSYY (SEQ ID NO: 77), a heavy chain variable complementarity-determining region 2 (VHCDR2) comprising an amino acid sequence of IYYSGST (SEQ ID NO: 78), a heavy chain variable complementarity-determining region 3 (VHCDR3) comprising an amino acid sequence of ARHRGSYFFDI (SEQ ID NO: 79), a light chain variable complementarity-determining region 1 (VLCDR1) comprising an amino acid sequence of DIENKN (SEQ ID NO: 81), a light chain variable complementarity-determining region 2 (VLCDR2) comprising an amino acid sequence of RDN, and a light chain variable complementarity-determining region 3 (VLCDR3) comprising an amino acid sequence of QVRDSNIVV (SEQ ID NO: 83), and the second arm comprising: (i) a second polypeptide comprising the extracellular domain of CTLA4 comprising SEQ ID NO: 174; (ii) a second bridging moiety comprising a sequence comprising of SEQ ID NO: 177 and a sequence comprising of SEQ ID NO: 179; and (iii) a second antigen-binding site comprising, a heavy chain variable complementarity-determining region 1 (VHCDR1) comprising an amino acid sequence of GGSISTSSYY (SEQ ID NO: 77), a heavy chain variable complementarity-determining region 2 (VHCDR2) comprising an amino acid sequence of IYYSGST (SEQ ID NO: 78), a heavy chain variable complementarity-determining region 3 (VHCDR3) comprising an amino acid sequence of ARHRGSYFFDI (SEQ ID NO: 79), a light chain variable complementarity-determining region 1 (VLCDR1) comprising an amino acid sequence of DIENKN (SEQ ID NO: 81), a light chain variable complementarity-determining region 2 (VLCDR2) comprising an amino acid sequence of RDN, and a light chain variable complementarity-determining region 3 (VLCDR3) comprising an amino acid sequence of QVRDSNIVV (SEQ ID NO: 83), wherein the first arm and the second arm dimerize with each other.

In some embodiments, proteins of the present disclosure comprise (i) a polypeptide comprising the extracellular domain of CTLA4 comprising SEQ ID NO: 174; (ii) a bridging moiety comprising a sequence comprising of SEQ ID NO: 213 and a sequence comprising of SEQ ID NO: 216; and (iii) an antigen-binding site comprising, a heavy chain variable complementarity-determining region 1 (VHCDR1) comprising an amino acid sequence of GVSIRSNGYY (SEQ ID NO: 93), a heavy chain variable complementarity-determining region 2 (VHCDR2) comprising an amino acid sequence of MDYSGT (SEQ ID NO: 94), a heavy chain variable complementarity-determining region 3 (VHCDR3) comprising an amino acid sequence of ARERSNNWYPIDY (SEQ ID NO: 95), a light chain variable complementarity-determining region 1 (VLCDR1) comprising an amino acid sequence of SVRRFF (SEQ ID NO: 97), a light chain variable complementarity-determining region 2 (VLCDR2) comprising an amino acid sequence of GKD, and a light chain variable complementarity-determining region 3 (VLCDR3) comprising an amino acid sequence of NSRDSSGYLVL (SEQ ID NO: 99).

In some embodiments, proteins of the present disclosure comprise a first arm and a second arm, the first arm comprising (i) a first polypeptide comprising the extracellular domain of CTLA4 comprising SEQ ID NO: 174; (ii) a first bridging moiety comprising a sequence comprising of SEQ ID NO: 213 and a sequence comprising of SEQ ID NO: 216; and (iii) a first antigen-binding site comprising, a heavy chain variable complementarity-determining region 1 (VHCDR1) comprising an amino acid sequence of GVSIRSNGYY (SEQ ID NO: 93), a heavy chain variable complementarity-determining region 2 (VHCDR2) comprising an amino acid sequence of MDYSGT (SEQ ID NO: 94), a heavy chain variable complementarity-determining region 3 (VHCDR3) comprising an amino acid sequence of ARERSNNWYPIDY (SEQ ID NO: 95), a light chain variable complementarity-determining region 1 (VLCDR1) comprising an amino acid sequence of SVRRFF (SEQ ID NO: 97), a light chain variable complementarity-determining region 2 (VLCDR2) comprising an amino acid sequence of GKD, and a light chain variable complementarity-determining region 3 (VLCDR3) comprising an amino acid sequence of NSRDSSGYLVL (SEQ ID NO: 99), and the second arm comprising: (i) a second polypeptide comprising the extracellular domain of CTLA4 comprising SEQ ID NO: 174; (ii) a second bridging moiety comprising a sequence comprising of SEQ ID NO: 213 and a sequence comprising of SEQ ID NO: 216; and (iii) a second antigen-binding site comprising, a heavy chain variable complementarity-determining region 1 (VHCDR1) comprising an amino acid sequence of GVSIRSNGYY (SEQ ID NO: 93), a heavy chain variable complementarity-determining region 2 (VHCDR2) comprising an amino acid sequence of MDYSGT (SEQ ID NO: 94), a heavy chain variable complementarity-determining region 3 (VHCDR3) comprising an amino acid sequence of ARERSNNWYPIDY (SEQ ID NO: 95), a light chain variable complementarity-determining region 1 (VLCDR1) comprising an amino acid sequence of SVRRFF (SEQ ID NO: 97), a light chain variable complementarity-determining region 2 (VLCDR2) comprising an amino acid sequence of GKD, and a light chain variable complementarity-determining region 3 (VLCDR3) comprising an amino acid sequence of NSRDSSGYLVL (SEQ ID NO: 99), wherein the first arm and the second arm dimerize with each other.

In some embodiments, proteins of the present disclosure comprise (i) a polypeptide comprising the extracellular domain of CTLA4 comprising SEQ ID NO: 174; (ii) a bridging moiety comprising a sequence comprising of SEQ ID NO: 213 and a sequence comprising of SEQ ID NO: 214; and (iii) an antigen-binding site comprising, a heavy chain variable complementarity-determining region 1 (VHCDR1) comprising an amino acid sequence of GASVSSSSYY (SEQ ID NO: 85), a heavy chain variable complementarity-determining region 2 (VHCDR2) comprising an amino acid sequence of INYGGST (SEQ ID NO: 86), a heavy chain variable complementarity-determining region 3 (VHCDR3) comprising an amino acid sequence of ARHRGIYHFDY (SEQ ID NO: 87), a light chain variable complementarity-determining region 1 (VLCDR1) comprising an amino acid sequence of NIENKN (SEQ ID NO: 89), a light chain variable complementarity-determining region 2 (VLCDR2) comprising an amino acid sequence of RDS, and a light chain variable complementarity-determining region 3 (VLCDR3) comprising an amino acid sequence of QVWDSNTVV (SEQ ID NO: 91).

In some embodiments, proteins of the present disclosure comprise a first arm and a second arm, the first arm comprising (i) a first polypeptide complex the extracellular domain of CTLA4 comprising SEQ ID NO: 174; (ii) a first bridging moiety comprising a sequence comprising of SEQ ID NO: 213 and a sequence comprising of SEQ ID NO: 214; and (iii) a first antigen-binding site comprising, a heavy chain variable complementarity-determining region 1 (VHCDR1) comprising an amino acid sequence of GASVSSSSYY (SEQ ID NO: 85), a heavy chain variable complementarity-determining region 2 (VHCDR2) comprising an amino acid sequence of INYGGST (SEQ ID NO: 86), a heavy chain variable complementarity-determining region 3 (VHCDR3) comprising an amino acid sequence of ARHRGIYHFDY (SEQ ID NO: 87), a light chain variable complementarity-determining region 1 (VLCDR1) comprising an amino acid sequence of NIENKN (SEQ ID NO: 89), a light chain variable complementarity-determining region 2 (VLCDR2) comprising an amino acid sequence of RDS, and a light chain variable complementarity-determining region 3 (VLCDR3) comprising an amino acid sequence of QVWDSNTVV (SEQ ID NO: 91), and the second arm comprising: (i) a second polypeptide comprising the extracellular domain of CTLA4 comprising SEQ ID NO: 174; (ii) a second bridging moiety comprising a sequence comprising of SEQ ID NO: 213 and a sequence comprising of SEQ ID NO: 214; and (iii) a second antigen-binding site comprising, a heavy chain variable complementarity-determining region 1 (VHCDR1) comprising an amino acid sequence of GASVSSSSYY (SEQ ID NO: 85), a heavy chain variable complementarity-determining region 2 (VHCDR2) comprising an amino acid sequence of INYGGST (SEQ ID NO: 86), a heavy chain variable complementarity-determining region 3 (VHCDR3) comprising an amino acid sequence of ARHRGIYHFDY (SEQ ID NO: 87), a light chain variable complementarity-determining region 1 (VLCDR1) comprising an amino acid sequence of NIENKN (SEQ ID NO: 89), a light chain variable complementarity-determining region 2 (VLCDR2) comprising an amino acid sequence of RDS, and a light chain variable complementarity-determining region 3 (VLCDR3) comprising an amino acid sequence of QVWDSNTVV (SEQ ID NO: 91), wherein the first arm and the second arm dimerize with each other.

In some embodiments, proteins of the present disclosure comprise (i) a polypeptide comprising the extracellular domain of CTLA4 comprising SEQ ID NO: 174; (ii) a bridging moiety comprising a sequence comprising of SEQ ID NO: 213 and a sequence comprising of SEQ ID NO: 216; and (iii) an antigen-binding site comprising, a heavy chain variable complementarity-determining region 1 (VHCDR1) comprising an amino acid sequence of GGSISSSSYY (SEQ ID NO: 101), a heavy chain variable complementarity-determining region 2 (VHCDR2) comprising an amino acid sequence of IGSVDYSGNT (SEQ ID NO: 102), a heavy chain variable complementarity-determining region 3 (VHCDR3) comprising an amino acid sequence of ARHRGIYFFDY (SEQ ID NO: 103), a light chain variable complementarity-determining region 1 (VLCDR1) comprising an amino acid sequence of NIENKN (SEQ ID NO: 89), a light chain variable complementarity-determining region 2 (VLCDR2) comprising an amino acid sequence of RDS, and a light chain variable complementarity-determining region 3 (VLCDR3) comprising an amino acid sequence of QVWDSNTVV (SEQ ID NO: 91).

In some embodiments, proteins of the present disclosure comprise a first arm and a second arm, the first arm comprising (i) a first polypeptide comprising the extracellular domain of CTLA4 comprising SEQ ID NO: 174; (ii) a first bridging moiety comprising a sequence comprising of SEQ ID NO: 213 and a sequence comprising of SEQ ID NO: 216; and (iii) a first antigen-binding site comprising, a heavy chain variable complementarity-determining region 1 (VHCDR1) comprising an amino acid sequence of GGSISSSSYY (SEQ ID NO: 101), a heavy chain variable complementarity-determining region 2 (VHCDR2) comprising an amino acid sequence of IGSVDYSGNT (SEQ ID NO: 102), a heavy chain variable complementarity-determining region 3 (VHCDR3) comprising an amino acid sequence of ARHRGIYFFDY (SEQ ID NO: 103), a light chain variable complementarity-determining region 1 (VLCDR1) comprising an amino acid sequence of NIENKN (SEQ ID NO: 89), a light chain variable complementarity-determining region 2 (VLCDR2) comprising an amino acid sequence of RDS, and a light chain variable complementarity-determining region 3 (VLCDR3) comprising an amino acid sequence of QVWDSNTVV (SEQ ID NO: 91), and the second arm comprising: (i) a second polypeptide comprising the extracellular domain of CTLA4 comprising SEQ ID NO: 174; (ii) a second bridging moiety comprising a sequence comprising of SEQ ID NO: 213 and a sequence comprising of SEQ ID NO: 216; and (iii) a second antigen-binding site comprising, a heavy chain variable complementarity-determining region 1 (VHCDR1) comprising an amino acid sequence of GGSISSSSYY (SEQ ID NO: 101), a heavy chain variable complementarity-determining region 2 (VHCDR2) comprising an amino acid sequence of IGSVDYSGNT (SEQ ID NO: 102), a heavy chain variable complementarity-determining region 3 (VHCDR3) comprising an amino acid sequence of ARHRGIYFFDY (SEQ ID NO: 103), a light chain variable complementarity-determining region 1 (VLCDR1) comprising an amino acid sequence of NIENKN (SEQ ID NO: 89), a light chain variable complementarity-determining region 2 (VLCDR2) comprising an amino acid sequence of RDS, and a light chain variable complementarity-determining region 3 (VLCDR3) comprising an amino acid sequence of QVWDSNTVV (SEQ ID NO: 91), wherein the first arm and the second arm dimerize with each other.

In some embodiments, proteins of the present disclosure comprise (i) a polypeptide comprising the extracellular domain of CTLA4 comprising SEQ ID NO: 174; (ii) a bridging moiety comprising a sequence comprising of SEQ ID NO: 213 and a sequence comprising of SEQ ID NO: 216; and (iii) an antigen-binding site comprising, a heavy chain variable complementarity-determining region 1 (VHCDR1) comprising an amino acid sequence of GGSISTSSYY (SEQ ID NO: 77), a heavy chain variable complementarity-determining region 2 (VHCDR2) comprising an amino acid sequence of IYYSGST (SEQ ID NO: 78), a heavy chain variable complementarity-determining region 3 (VHCDR3) comprising an amino acid sequence of ARHRGSYFFDI (SEQ ID NO: 79), a light chain variable complementarity-determining region 1 (VLCDR1) comprising an amino acid sequence of DIENKN (SEQ ID NO: 81), a light chain variable complementarity-determining region 2 (VLCDR2) comprising an amino acid sequence of RDN, and a light chain variable complementarity-determining region 3 (VLCDR3) comprising an amino acid sequence of QVRDSNIVV (SEQ ID NO: 83).

In some embodiments, proteins of the present disclosure comprise a first arm and a second arm, the first arm comprising (i) a first polypeptide comprising the extracellular domain of CTLA4 comprising SEQ ID NO: 174; (ii) a first bridging moiety comprising a sequence comprising of SEQ ID NO: 213 and a sequence comprising of SEQ ID NO: 216; and (iii) a first antigen-binding site comprising, a heavy chain variable complementarity-determining region 1 (VHCDR1) comprising an amino acid sequence of GGSISTSSYY (SEQ ID NO: 77), a heavy chain variable complementarity-determining region 2 (VHCDR2) comprising an amino acid sequence of IYYSGST (SEQ ID NO: 78), a heavy chain variable complementarity-determining region 3 (VHCDR3) comprising an amino acid sequence of ARHRGSYFFDI (SEQ ID NO: 79), a light chain variable complementarity-determining region 1 (VLCDR1) comprising an amino acid sequence of DIENKN (SEQ ID NO: 81), a light chain variable complementarity-determining region 2 (VLCDR2) comprising an amino acid sequence of RDN, and a light chain variable complementarity-determining region 3 (VLCDR3) comprising an amino acid sequence of QVRDSNIVV (SEQ ID NO: 83), and the second arm comprising: (i) a second polypeptide comprising the extracellular domain of CTLA4 comprising SEQ ID NO: 174; (ii) a second bridging moiety comprising a sequence comprising of SEQ ID NO: 213 and a sequence comprising of SEQ ID NO: 216; and (iii) a second antigen-binding site comprising, a heavy chain variable complementarity-determining region 1 (VHCDR1) comprising an amino acid sequence of GGSISTSSYY (SEQ ID NO: 77), a heavy chain variable complementarity-determining region 2 (VHCDR2) comprising an amino acid sequence of IYYSGST (SEQ ID NO: 78), a heavy chain variable complementarity-determining region 3 (VHCDR3) comprising an amino acid sequence of ARHRGSYFFDI (SEQ ID NO: 79), a light chain variable complementarity-determining region 1 (VLCDR1) comprising an amino acid sequence of DIENKN (SEQ ID NO: 81), a light chain variable complementarity-determining region 2 (VLCDR2) comprising an amino acid sequence of RDN, and a light chain variable complementarity-determining region 3 (VLCDR3) comprising an amino acid sequence of QVRDSNIVV (SEQ ID NO: 83), wherein the first arm and the second arm dimerize with each other.

In some embodiments, proteins of the present disclosure comprise (i) a polypeptide comprising the extracellular domain of CTLA4 comprising a sequence selected from a group consisting of SEQ ID NO: 174, SEQ ID NO: 119, SEQ ID NO: 215, SEQ ID NO: 233, SEQ ID NO: 234, and SEQ ID NO: 235; (ii) a bridging moiety comprising a sequence selected from a group consisting of SEQ ID NO: 175, SEQ ID NO: 176, SEQ ID NO: 177, SEQ ID NO: 212, SEQ ID NO: 213, and SEQ ID NO: 248, and a sequence selected from a group consisting of SEQ ID NO: 178, SEQ ID NO: 237, SEQ ID NO: 214; SEQ ID NO: 216, SEQ ID NO: 179, SEQ ID NO: 180, SEQ ID NO: 239, and SEQ ID NO: 240; and (iii) an antigen-binding site comprising a heavy chain variable complementarity-determining region 1 (VHCDR1) comprising an amino acid sequence of GFTFSNYA (SEQ ID NO: 133), a heavy chain variable complementarity-determining region 2 (VHCDR2) comprising an amino acid sequence of ISGSGGAT (SEQ ID NO: 113), a heavy chain variable complementarity-determining region 3 (VHCDR3) comprising an amino acid sequence of TKDRLIMATVRGPYYYGMDV (SEQ ID NO: 114), a light chain variable complementarity-determining region 1 (VLCDR1) comprising an amino acid sequence of QSISSY (SEQ ID NO: 121), a light chain variable complementarity-determining region 2 (VLCDR2) comprising an amino acid sequence of AAS, and a light chain variable complementarity-determining region 3 (VLCDR3) comprising an amino acid sequence of QQSHSVSFT (SEQ ID NO: 154).

In some embodiments, proteins of the present disclosure comprise a first arm and a second arm, the first arm comprising (i) a first polypeptide comprising the extracellular domain of CTLA4 comprising a sequence selected from a group consisting of SEQ ID NO: 174, SEQ ID NO: 119, SEQ ID NO: 215, SEQ ID NO: 233, SEQ ID NO: 234, and SEQ ID NO: 235; (ii) a first bridging moiety comprising a sequence selected from a group consisting of SEQ ID NO: 175, SEQ ID NO: 176, SEQ ID NO: 177, SEQ ID NO: 212, SEQ ID NO: 213, and SEQ ID NO: 248, and a sequence selected from a group consisting of SEQ ID NO: 178, SEQ ID NO: 237, SEQ ID NO: 214; SEQ ID NO: 216, SEQ ID NO: 179, SEQ ID NO: 180, SEQ ID NO: 239, and SEQ ID NO: 240; and (iii) a first antigen-binding site comprising a heavy chain variable complementarity-determining region 1 (VHCDR1) comprising an amino acid sequence of GFTFSNYA (SEQ ID NO: 133), a heavy chain variable complementarity-determining region 2 (VHCDR2) comprising an amino acid sequence of ISGSGGAT (SEQ ID NO: 113), a heavy chain variable complementarity-determining region 3 (VHCDR3) comprising an amino acid sequence of TKDRLIMATVRGPYYYGMDV (SEQ ID NO: 114), a light chain variable complementarity-determining region 1 (VLCDR1) comprising an amino acid sequence of QSISSY (SEQ ID NO: 121), a light chain variable complementarity-determining region 2 (VLCDR2) comprising an amino acid sequence of AAS, and a light chain variable complementarity-determining region 3 (VLCDR3) comprising an amino acid sequence of QQSHSVSFT (SEQ ID NO: 154), and the second arm comprising: (i) a second polypeptide comprising the extracellular domain of CTLA4 comprising a sequence selected from a group consisting of SEQ ID NO: 174, SEQ ID NO: 119, SEQ ID NO: 215, SEQ ID NO: 233, SEQ ID NO: 234, and SEQ ID NO: 235; (ii) a second bridging moiety comprising a sequence selected from a group consisting of SEQ ID NO: 175, SEQ ID NO: 176, SEQ ID NO: 177, SEQ ID NO: 212, SEQ ID NO: 213, and SEQ ID NO: 248, and a sequence selected from a group consisting of SEQ ID NO: 178, SEQ ID NO: 237, SEQ ID NO: 214; SEQ ID NO: 216, SEQ ID NO: 179, SEQ ID NO: 180, SEQ ID NO: 239, and SEQ ID NO: 240; and (iii) a second antigen-binding site comprising a heavy chain variable complementarity-determining region 1 (VHCDR1) comprising an amino acid sequence of GFTFSNYA (SEQ ID NO: 133), a heavy chain variable complementarity-determining region 2 (VHCDR2) comprising an amino acid sequence of ISGSGGAT (SEQ ID NO: 113), a heavy chain variable complementarity-determining region 3 (VHCDR3) comprising an amino acid sequence of TKDRLIMATVRGPYYYGMDV (SEQ ID NO: 114), a light chain variable complementarity-determining region 1 (VLCDR1) comprising an amino acid sequence of QSISSY (SEQ ID NO: 121), a light chain variable complementarity-determining region 2 (VLCDR2) comprising an amino acid sequence of AAS, and a light chain variable complementarity-determining region 3 (VLCDR3) comprising an amino acid sequence of QQSHSVSFT (SEQ ID NO: 154), wherein the first arm and the second arm dimerize with each other.

In some embodiments, proteins of the present disclosure comprise (i) a polypeptide comprising the extracellular domain of CTLA4 comprising a sequence selected from a group consisting of SEQ ID NO: 174, SEQ ID NO: 119, SEQ ID NO: 215. SEQ ID NO: 233, SEQ ID NO: 234, and SEQ ID NO: 235; (ii) a bridging moiety comprising a sequence selected from a group consisting of SEQ ID NO: 175, SEQ ID NO: 176, SEQ ID NO: 177, SEQ ID NO: 212, SEQ ID NO: 213, and SEQ ID NO: 248, and a sequence selected from a group consisting of SEQ ID NO: 178, SEQ ID NO: 237, SEQ ID NO: 214, SEQ ID NO: 216, SEQ ID NO: 179, SEQ ID NO: 180, SEQ ID NO: 239, and SEQ ID NO: 240; and (iii) an antigen-binding site comprising a heavy chain variable complementarity-determining region 1 (VHCDR1) comprising an amino acid sequence of SYAMS (SEQ ID NO: 105), a heavy chain variable complementarity-determining region 2 (VHCDR2) comprising an amino acid sequence of IISGSGGFTYYADSVK (SEQ ID NO: 106), a heavy chain variable complementarity-determining region 3 (VHCDR3) comprising an amino acid sequence of DRLVAPGTFDY (SEQ ID NO: 107), a light chain variable complementarity-determining region 1 (VLCDR1) comprising an amino acid sequence of RASQGISSWLA (SEQ ID NO: 169), a light chain variable complementarity-determining region 2 (VLCDR2) comprising an amino acid sequence of AASSLQS (SEQ ID NO: 170), and a light chain variable complementarity-determining region 3 (VLCDR3) comprising an amino acid sequence of QQYNSYPYT (SEQ ID NO: 171).

In some embodiments, proteins of the present disclosure comprise a first arm and a second arm, the first arm comprising (i) a first polypeptide comprising the extracellular domain of CTLA4 comprising a sequence selected from a group consisting of SEQ ID NO: 174, SEQ ID NO: 119, SEQ ID NO: 215. SEQ ID NO: 233, SEQ ID NO: 234, and SEQ ID NO: 235; (ii) a first bridging moiety comprising a sequence selected from a group consisting of SEQ ID NO: 175, SEQ ID NO: 176, SEQ ID NO: 177, SEQ ID NO: 212, SEQ ID NO: 213, and SEQ ID NO: 248, and a sequence selected from a group consisting of SEQ ID NO: 178, SEQ ID NO: 237, SEQ ID NO: 214, SEQ ID NO: 216, SEQ ID NO: 179, SEQ ID NO: 180, SEQ ID NO: 239, and SEQ ID NO: 240; and (iii) a first antigen-binding site comprising a heavy chain variable complementarity-determining region 1 (VHCDR1) comprising an amino acid sequence of SYAMS (SEQ ID NO: 105), a heavy chain variable complementarity-determining region 2 (VHCDR2) comprising an amino acid sequence of IISGSGGFTYYADSVK (SEQ ID NO: 106), a heavy chain variable complementarity-determining region 3 (VHCDR3) comprising an amino acid sequence of DRLVAPGTFDY (SEQ ID NO: 107), a light chain variable complementarity-determining region 1 (VLCDR1) comprising an amino acid sequence of RASQGISSWLA (SEQ ID NO: 169), a light chain variable complementarity-determining region 2 (VLCDR2) comprising an amino acid sequence of AASSLQS (SEQ ID NO: 170), and a light chain variable complementarity-determining region 3 (VLCDR3) comprising an amino acid sequence of QQYNSYPYT (SEQ ID NO: 171), and the second arm comprises: (i) a second polypeptide comprising the extracellular domain of CTLA4 comprising a sequence selected from a group consisting of SEQ ID NO: 174, SEQ ID NO: 119, SEQ ID NO: 215. SEQ ID NO: 233, SEQ ID NO: 234, and SEQ ID NO: 235; (ii) a second bridging moiety comprising a sequence selected from a group consisting of SEQ ID NO: 175, SEQ ID NO: 176, SEQ ID NO: 177, SEQ ID NO: 212, SEQ ID NO: 213, and SEQ ID NO: 248, and a sequence selected from a group consisting of SEQ ID NO: 178, SEQ ID NO: 237, SEQ ID NO: 214, SEQ ID NO: 216, SEQ ID NO: 179, SEQ ID NO: 180, SEQ ID NO: 239, and SEQ ID NO: 240; and (iii) a second antigen-binding site comprising a heavy chain variable complementarity-determining region 1 (VHCDR1) comprising an amino acid sequence of SYAMS (SEQ ID NO: 105), a heavy chain variable complementarity-determining region 2 (VHCDR2) comprising an amino acid sequence of IISGSGGFTYYADSVK (SEQ ID NO: 106), a heavy chain variable complementarity-determining region 3 (VHCDR3) comprising an amino acid sequence of DRLVAPGTFDY (SEQ ID NO: 107), a light chain variable complementarity-determining region 1 (VLCDR1) comprising an amino acid sequence of RASQGISSWLA (SEQ ID NO: 169), a light chain variable complementarity-determining region 2 (VLCDR2) comprising an amino acid sequence of AASSLQS (SEQ ID NO: 170), and a light chain variable complementarity-determining region 3 (VLCDR3) comprising an amino acid sequence of QQYNSYPYT (SEQ ID NO: 171), wherein the first arm and the second arm dimerize with each other.

In some embodiments, proteins of the present disclosure comprise (i) a polypeptide comprising the extracellular domain of CTLA4 comprising a sequence selected from a group consisting of SEQ ID NO: 174, SEQ ID NO: 119, SEQ ID NO: 215, SEQ ID NO: 233, SEQ ID NO: 234, and SEQ ID NO: 235; (ii) a bridging moiety comprising a sequence selected from a group consisting of SEQ ID NO: 175, SEQ ID NO: 176, SEQ ID NO: 177, SEQ ID NO: 212, SEQ ID NO: 213, and SEQ ID NO: 248, and a sequence selected from a group consisting of SEQ ID NO: 178, SEQ ID NO: 237, SEQ ID NO: 214, SEQ ID NO: 216, SEQ ID NO: 179, SEQ ID NO: 180, SEQ ID NO: 239, and SEQ ID NO: 240; and (iii) an antigen-binding site comprising, according to the IMGT unique numbering scheme, VHCDR1, VHCDR2, VHCDR3, VLCDR1, VLCDR2, and VLCDR3 each comprising an amino acid sequence corresponding to the VHCDR1, VHCDR2, VHCDR3, VLCDR1, VLCDR2, and VLCDR3 sequences of a heavy chain variable domain and a light chain variable domain listed in TABLE 3, respectively.

In some embodiments, proteins of the present disclosure comprise a first arm and a second arm, the first arm comprising (i) a first polypeptide comprising the extracellular domain of CTLA4 comprising a sequence selected from a group consisting of SEQ ID NO: 174, SEQ ID NO: 119, SEQ ID NO: 215, SEQ ID NO: 233, SEQ ID NO: 234, and SEQ ID NO: 235; (ii) a first bridging moiety comprising a sequence selected from a group consisting of SEQ ID NO: 175, SEQ ID NO: 176, SEQ ID NO: 177, SEQ ID NO: 212, SEQ ID NO: 213, and SEQ ID NO: 248, and a sequence selected from a group consisting of SEQ ID NO: 178, SEQ ID NO: 237, SEQ ID NO: 214, SEQ ID NO: 216, SEQ ID NO: 179, SEQ ID NO: 180, SEQ ID NO: 239, and SEQ ID NO: 240; and (iii) a first antigen-binding site comprising, according to the IMGT unique numbering scheme, VHCDR1, VHCDR2, VHCDR3, VLCDR1, VLCDR2, and VLCDR3 each comprising an amino acid sequence corresponding to the VHCDR1, VHCDR2, VHCDR3, VLCDR1, VLCDR2, and VLCDR3 sequences of a heavy chain variable domain and a light chain variable domain listed in TABLE 3, respectively, and the second arm comprising: (i) a second polypeptide comprising the extracellular domain of CTLA4 comprising a sequence selected from a group consisting of SEQ ID NO: 174, SEQ ID NO: 119, SEQ ID NO: 215, SEQ ID NO: 233, SEQ ID NO: 234, and SEQ ID NO: 235; (ii) a second bridging moiety comprising a sequence selected from a group consisting of SEQ ID NO: 175, SEQ ID NO: 176, SEQ ID NO: 177, SEQ ID NO: 212, SEQ ID NO: 213, and SEQ ID NO: 248, and a sequence selected from a group consisting of SEQ ID NO: 178, SEQ ID NO: 237, SEQ ID NO: 214, SEQ ID NO: 216, SEQ ID NO: 179, SEQ ID NO: 180, SEQ ID NO: 239, and SEQ ID NO: 240; and (iii) a second antigen-binding site comprising, according to the IMGT unique numbering scheme, VHCDR1, VHCDR2, VHCDR3, VLCDR1, VLCDR2, and VLCDR3 each comprising an amino acid sequence corresponding to the VHCDR1, VHCDR2, VHCDR3, VLCDR1, VLCDR2, and VLCDR3 sequences of a heavy chain variable domain and a light chain variable domain listed in TABLE 3, respectively, wherein the first arm and the second arm dimerize with each other.

In some embodiments, proteins of the present disclosure comprise (i) a polypeptide comprising the extracellular domain of CTLA4 comprising a sequence selected from a group consisting of SEQ ID NO: 174, SEQ ID NO: 119, SEQ ID NO: 215, SEQ ID NO: 233, SEQ ID NO: 234, and SEQ ID NO: 235; (ii) a bridging moiety comprising a sequence selected from a group consisting of SEQ ID NO: 175, SEQ ID NO: 176, SEQ ID NO: 177, SEQ ID NO: 212, SEQ ID NO: 213, and SEQ ID NO: 248, and a sequence selected from a group consisting of SEQ ID NO: 178, SEQ ID NO: 237, SEQ ID NO: 214, SEQ ID NO: 216, SEQ ID NO: 179, SEQ ID NO: 180, SEQ ID NO: 239, and SEQ ID NO: 240; and (iii) an antigen-binding site comprising a polypeptide that specifically binds to OX40L.

In some embodiments, proteins of the present disclosure comprise a first arm and a second arm, the first arm comprising (i) a first polypeptide comprising the extracellular domain of CTLA4 comprising a sequence selected from a group consisting of SEQ ID NO: 174, SEQ ID NO: 119, SEQ ID NO: 215, SEQ ID NO: 233, SEQ ID NO: 234, and SEQ ID NO: 235; (ii) a first bridging moiety comprising a sequence selected from a group consisting of SEQ ID NO: 175, SEQ ID NO: 176, SEQ ID NO: 177, SEQ ID NO: 212, SEQ ID NO: 213, and SEQ ID NO: 248, and a sequence selected from a group consisting of SEQ ID NO: 178, SEQ ID NO: 237, SEQ ID NO: 214, SEQ ID NO: 216, SEQ ID NO: 179, SEQ ID NO: 180, SEQ ID NO: 239, and SEQ ID NO: 240; and (iii) a first antigen-binding site comprising a polypeptide that specifically binds to OX40L, and the second arm comprising: (i) a second polypeptide comprising the extracellular domain of CTLA4 comprising a sequence selected from a group consisting of SEQ ID NO: 174, SEQ ID NO: 119, SEQ ID NO: 215, SEQ ID NO: 233, SEQ ID NO: 234, and SEQ ID NO: 235; (ii) a second bridging moiety comprising a sequence selected from a group consisting of SEQ ID NO: 175, SEQ ID NO: 176, SEQ ID NO: 177, SEQ ID NO: 212, SEQ ID NO: 213, and SEQ ID NO: 248, and a sequence selected from a group consisting of SEQ ID NO: 178, SEQ ID NO: 237, SEQ ID NO: 214, SEQ ID NO: 216, SEQ ID NO: 179, SEQ ID NO: 180, SEQ ID NO: 239, and SEQ ID NO: 240; and (iii) a second antigen-binding site comprising a polypeptide that specifically binds to OX40L, wherein the first arm and the second arm dimerize with each other.

In some embodiments, proteins of the present disclosure comprise an amino acid sequence at least 90% identical to the amino acid sequence of SEQ ID NO: 40, SEQ ID NO: 62, SEQ ID NO: 64, SEQ ID NO: 182, SEQ ID NO: 183, SEQ ID NO: 184, SEQ ID NO: 185, SEQ ID NO: 186, SEQ ID NO: 48, SEQ ID NO: 68, SEQ ID NO: 187, SEQ ID NO: 188, SEQ ID NO: 189, SEQ ID NO: 190, SEQ ID NO: 191, SEQ ID NO: 46, SEQ ID NO: 66, SEQ ID NO: 192, SEQ ID NO: 193, SEQ ID NO: 194, SEQ ID NO: 195, SEQ ID NO: 196, SEQ ID NO: 50, SEQ ID NO: 70, SEQ ID NO: 197, SEQ ID NO: 198, SEQ ID NO: 199, SEQ ID NO: 200, SEQ ID NO: 201, SEQ ID NO: 38, SEQ ID NO: 73, SEQ ID NO: 42, SEQ ID NO: 44, SEQ ID NO: 52, SEQ ID NO: 54, SEQ ID NO: 56, SEQ ID NO: 58, SEQ ID NO: 60, SEQ ID NO: 37, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308, SEQ ID NO: 309, SEQ ID NO: 310, SEQ ID NO: 311, SEQ ID NO: 312, SEQ ID NO: 313, SEQ ID NO: 314, SEQ ID NO: 315, SEQ ID NO: 316, SEQ ID NO: 317, SEQ ID NO: 318, SEQ ID NO: 319, SEQ ID NO: 320, SEQ ID NO: 321, SEQ ID NO: 322, SEQ ID NO: 323, SEQ ID NO: 324, SEQ ID NO: 325, SEQ ID NO: 326, SEQ ID NO: 327, SEQ ID NO: 328, SEQ ID NO: 329, SEQ ID NO: 330, SEQ ID NO: 331, SEQ ID NO: 332, SEQ ID NO: 333, SEQ ID NO: 334, or SEQ ID NO: 335.

In some embodiments, proteins of the present disclosure comprise polypeptides comprising amino acid sequences at least 90% identical to the amino acid sequences of: SEQ ID NO: 40 and SEQ ID NO: 39; SEQ ID NO: 62 and SEQ ID NO: 61; SEQ ID NO: 64 and SEQ ID NO: 63; SEQ ID NO: 182 and SEQ ID NO: 63; SEQ ID NO: 183 and SEQ ID NO: 63; SEQ ID NO: 184 and SEQ ID NO: 63; SEQ ID NO: 185 and SEQ ID NO: 63; SEQ ID NO: 186 and SEQ ID NO: 63; SEQ ID NO: 48 and SEQ ID NO: 47; SEQ ID NO: 68 and SEQ ID NO: 67; SEQ ID NO: 187 and SEQ ID NO: 67; SEQ ID NO: 188 and SEQ ID NO: 67; SEQ ID NO: 189 and SEQ ID NO: 67; SEQ ID NO: 190 and SEQ ID NO: 67; SEQ ID NO: 191 and SEQ ID NO: 67; SEQ ID NO: 46 and SEQ ID NO: 45; SEQ ID NO: 66 and SEQ ID NO: 65; SEQ ID NO: 192 and SEQ ID NO: 65; SEQ ID NO: 193 and SEQ ID NO: 65; SEQ ID NO: 194 and SEQ ID NO: 65; SEQ ID NO: 195 and SEQ ID NO: 65; SEQ ID NO: 196 and SEQ ID NO: 65; SEQ ID NO: 50 and SEQ ID NO: 49; SEQ ID NO: 70 and SEQ ID NO: 69; SEQ ID NO: 197 and SEQ ID NO: 69; SEQ ID NO: 198 and SEQ ID NO: 69; SEQ ID NO: 199 and SEQ ID NO: 69; SEQ ID NO: 200 and SEQ ID NO: 69; SEQ ID NO: 201 and SEQ ID NO: 69; SEQ ID NO: 38 and SEQ ID NO: 5; SEQ ID NO: 73 and SEQ ID NO: 5; SEQ ID NO: 42 and SEQ ID NO: 41; SEQ ID NO: 44 and SEQ ID NO: 43; SEQ ID NO: 52 and SEQ ID NO: 51; SEQ ID NO: 54 and SEQ ID NO: 53; SEQ ID NO: 56 and SEQ ID NO: 55; SEQ ID NO: 58 and SEQ ID NO: 57; SEQ ID NO: 60 and SEQ ID NO: 59; SEQ ID NO: 37 and SEQ ID NO: 35; SEQ ID NO: 256 and SEQ ID NO: 63; SEQ ID NO: 257 and SEQ ID NO: 63; SEQ ID NO: 258 and SEQ ID NO: 63; SEQ ID NO: 259 and SEQ ID NO: 63; SEQ ID NO: 260 and SEQ ID NO: 63; SEQ ID NO: 261 and SEQ ID NO: 63; SEQ ID NO: 262 and SEQ ID NO: 63; SEQ ID NO: 263 and SEQ ID NO: 63; SEQ ID NO: 264 and SEQ ID NO: 63; SEQ ID NO: 265 and SEQ ID NO: 63; SEQ ID NO: 266 and SEQ ID NO: 63; SEQ ID NO: 267 and SEQ ID NO: 63; SEQ ID NO: 268 and SEQ ID NO: 63; SEQ ID NO: 269 and SEQ ID NO: 63; SEQ ID NO: 270 and SEQ ID NO: 63; SEQ ID NO: 271 and SEQ ID NO: 63; SEQ ID NO: 272 and SEQ ID NO: 63; SEQ ID NO: 273 and SEQ ID NO: 63; SEQ ID NO: 274 and SEQ ID NO: 63; SEQ ID NO: 275 and SEQ ID NO: 63; SEQ ID NO: 276 and SEQ ID NO: 63; SEQ ID NO: 277 and SEQ ID NO: 63; SEQ ID NO: 278 and SEQ ID NO: 63; SEQ ID NO: 279 and SEQ ID NO: 63; SEQ ID NO: 280 and SEQ ID NO: 63; SEQ ID NO: 281 and SEQ ID NO: 63; SEQ ID NO: 282 and SEQ ID NO: 63; SEQ ID NO: 283 and SEQ ID NO: 63; SEQ ID NO: 284 and SEQ ID NO: 63; SEQ ID NO: 285 and SEQ ID NO: 63; SEQ ID NO: 286 and SEQ ID NO: 63; SEQ ID NO: 287 and SEQ ID NO: 63; SEQ ID NO: 288 and SEQ ID NO: 63; SEQ ID NO: 289 and SEQ ID NO: 63; SEQ ID NO: 290 and SEQ ID NO: 63; SEQ ID NO: 291 and SEQ ID NO: 63; SEQ ID NO: 292 and SEQ ID NO: 63; SEQ ID NO: 293 and SEQ ID NO: 63; SEQ ID NO: 294 and SEQ ID NO: 67; SEQ ID NO: 295 and SEQ ID NO: 67; SEQ ID NO: 296 and SEQ ID NO: 67; SEQ ID NO: 297 and SEQ ID NO: 67; SEQ ID NO: 298, and SEQ ID NO: 67; SEQ ID NO: 299 and SEQ ID NO: 67; SEQ ID NO: 300 and SEQ ID NO: 67; SEQ ID NO: 301 and SEQ ID NO: 67; SEQ ID NO: 302 and SEQ ID NO: 67; SEQ ID NO: 303 and SEQ ID NO: 67; SEQ ID NO: 304 and SEQ ID NO: 67; SEQ ID NO: 305 and SEQ ID NO: 67; SEQ ID NO: 306 and SEQ ID NO: 67; SEQ ID NO: 307 and SEQ ID NO: 67; SEQ ID NO: 308 and SEQ ID NO: 67; SEQ ID NO: 309 and SEQ ID NO: 67; SEQ ID NO: 310 and SEQ ID NO: 67; SEQ ID NO: 311 and SEQ ID NO: 67; SEQ ID NO: 312 and SEQ ID NO: 67; SEQ ID NO: 313 and SEQ ID NO: 67; SEQ ID NO: 314 and SEQ ID NO: 67; SEQ ID NO: 315 and SEQ ID NO: 67; SEQ ID NO: 316 and SEQ ID NO: 67; SEQ ID NO: 317 and SEQ ID NO: 67; SEQ ID NO: 318 and SEQ ID NO: 67; SEQ ID NO: 319 and SEQ ID NO: 67; SEQ ID NO: 320 and SEQ ID NO: 67; SEQ ID NO: 321 and SEQ ID NO: 67; SEQ ID NO: 322 and SEQ ID NO: 67; SEQ ID NO: 323 and SEQ ID NO: 67; SEQ ID NO: 324 and SEQ ID NO: 67; SEQ ID NO: 325 and SEQ ID NO: 67; SEQ ID NO: 326 and SEQ ID NO: 67; SEQ ID NO: 327 and SEQ ID NO: 67; SEQ ID NO: 328 and SEQ ID NO: 67; SEQ ID NO: 329 and SEQ ID NO: 67; SEQ ID NO: 330 and SEQ ID NO: 67; SEQ ID NO: 331 and SEQ ID NO: 67; SEQ ID NO: 332 and SEQ ID NO: 253; SEQ ID NO: 333 and SEQ ID NO: 255; SEQ ID NO: 334 and SEQ ID NO: 253; and SEQ ID NO: 335 or SEQ ID NO: 255.

In some embodiments, proteins of the present disclosure comprise two polypeptides comprising amino acid sequences of SEQ ID NO: 63 and two polypeptides comprising amino acid sequences of SEQ ID NO: 64, or two polypeptides comprising amino acid sequences of SEQ ID NO: 68 and two polypeptides comprising amino acid sequences of SEQ ID NO: 67.

In some embodiments, proteins of the present disclosure have an IC₅₀ less than 12 nM, for example 5 nM to 12 nM or 5 nM to 9 nM, as measured in an OX40L neutralization/OX40-HEK reporter assay.

In some embodiments, proteins of the present disclosure have an IC₅₀ of 2 nM to 12 nM, for example 2 nM to 9.5 nM, as measured in a primary T cell activation assay.

In some embodiments, proteins of the present disclosure have a K_{D} for OX40L binding of less than 75 nM, for example 20 nM to 75 nM, as measured in a Biacore assay.

In some embodiments, a protein of the present disclosure significantly inhibits at least one of IL-2, IFNγ, IL-6, and TNFα production by cells in an in vitro mixed lymphocyte reaction assay as compared to cells treated under the same conditions but in the absence of protein, or in the presence of a human CTLA4 extracellular domain fused at the N-terminus of a human IgG1 Fc domain (CTLA4-Ig) and an anti-OX40L antibody, presented individually or in combination of the two proteins presented separately.

In some embodiments, a protein of the present disclosure significantly inhibits alloreactive CD4+ and CD8+ T cell proliferation in an in vitro mixed lymphocyte reaction assay as compared to alloreactive CD4+ and CD8+ T cells treated under the same conditions but in the absence of protein, or in the presence of a CTLA4-Ig and/or an anti-OX40L antibody.

In some embodiments, a protein of the present disclosure preferentially inhibits alloreactive CD4+ and CD8+ T cell proliferation as compared to regulatory T cell (T_{reg}) proliferation in an in vitro mixed lymphocyte reaction assay as compared to alloreactive CD4+ and CD8+ T cells treated under the same conditions but in the absence of protein, or in the presence of a CTLA4-Ig and/or an anti-OX40L antibody.

In some embodiments, a protein of the present disclosure significantly enhances T_{reg} suppressive function in an in vitro mixed lymphocyte reaction assay as compared to T_{reg} cells treated under the same conditions but in the absence of protein, or in the presence of a CTLA4-Ig and/or an anti-OX40L antibody.

In some embodiments, a protein of the present disclosure significantly inhibits alloreactive CD4+ and CD8+ T cell proliferation in an adoptive transfer assay in humanized mice as compared to alloreactive CD4+ and CD8+ T cells in mice treated under the same conditions but in the absence of protein, or in the presence of a CTLA4-Ig and/or an anti-OX40L antibody.

In some embodiments, a protein of the present disclosure significantly inhibits serum IFNγ levels in an adoptive transfer assay in humanized mice as compared to mice treated under the same conditions but in the absence of protein, or in the presence of a CTLA4-Ig and/or an anti-OX40L antibody.

In some embodiments, a protein of the present disclosure is not significantly internalized into myeloid-derived dendritic cells (MDDCs).

In some embodiments, a protein of the present disclosure is cross-reactive with cynomolgus OX40L and/or is not cross-reactive with mouse, rabbit, or rat OX40L. In some embodiments, a protein of the present disclosure is cross reactive with cynomolgus CD80 and CD86.

In another aspect, the present disclosure provides the use of a heavy chain polypeptide as disclosed herein in the preparation of a protein comprising the heavy chain polypeptide and a light chain polypeptide as disclosed herein.

In another aspect, the present disclosure provides a formulation comprising a protein as disclosed herein and a pharmaceutically acceptable carrier.

In another aspect, the present disclosure provides a nucleic acid encoding a protein as disclosed herein.

In yet another aspect, the present disclosure provides a cell comprising one or more nucleic acids encoding a protein as disclosed herein.

In another aspect, the present disclosure provides a method of treating an autoimmune disease in a patient comprising administering to the patient a protein or formulation as disclosed herein. For example, autoimmune diseases that can be treated by proteins of the present disclosure include rheumatoid arthritis, juvenile idiopathic arthritis, psoriatic arthritis, atopic dermatitis, Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Bal6 disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Cutaneous Lupus, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Thyroid eye disease (TED), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, and Vogt-Koyanagi-Harada Disease. In another aspect, the present disclosure provides a method of treating graft versus host disease (GVHD) in a patient, comprising administering to the patient a protein or formulation as disclosed herein.

In still another aspect, the present disclosure provides an antibody or functional fragment thereof comprising, according to the IMGT unique numbering scheme, a heavy chain variable complementarity-determining region 1 (VHCDR1) comprising an amino acid sequence of GGSISTSSYY (SEQ ID NO: 77), a heavy chain variable complementarity-determining region 2 (VHCDR2) comprising an amino acid sequence of IYYSGST (SEQ ID NO: 78), a heavy chain variable complementarity-determining region 3 (VHCDR3) comprising an amino acid sequence of ARHRGSYFFDI (SEQ ID NO: 79), a light chain variable complementarity-determining region 1 (VLCDR1) comprising an amino acid sequence of DIENKN (SEQ ID NO: 81), a light chain variable complementarity-determining region 2 (VLCDR2) comprising an amino acid sequence of RDN, and a light chain variable complementarity-determining region 3 (VLCDR3) comprising an amino acid sequence of QVRDSNIVV (SEQ ID NO: 83).

In some embodiments, an antibody of the present disclosure comprises a heavy chain variable domain comprising an amino acid sequence at least 90% identical to SEQ ID NO: 76, and a light chain comprising an amino acid sequence at least 90% identical to SEQ ID NO: 80.

In some embodiments, an antibody of the present disclosure or functional fragment thereof comprises, according to the IMGT unique numbering scheme, a VHCDR1 comprising an amino acid sequence of GVSIRSNGYY (SEQ ID NO: 93), a VHCDR2 comprising an amino acid sequence of MDYSGT (SEQ ID NO: 94), a VHCDR3 comprising an amino acid sequence of ARERSNNWYPIDY (SEQ ID NO: 95), a VLCDR1 comprising an amino acid sequence of SVRRFF (SEQ ID NO: 97), a VLCDR2 comprising an amino acid sequence of GKD, and a VLCDR3 comprising an amino acid sequence of NSRDSSGYLVL (SEQ ID NO: 99).

In some embodiments, an antibody of the present disclosure comprises a heavy chain variable domain comprises an amino acid sequence at least 90% identical to SEQ ID NO: 92, and a light chain variable domain comprises an amino acid sequence at least 90% identical to SEQ ID NO: 96.

In some embodiments, an antibody of the present disclosure or functional fragment thereof comprises, according to the IMGT unique numbering scheme, a VHCDR1 comprising an amino acid sequence of GASVSSSSYY (SEQ ID NO: 85), a VHCDR2 comprising an amino acid sequence of INYGGST (SEQ ID NO: 86), a VHCDR3 comprising an amino acid sequence of ARHRGIYHFDY (SEQ ID NO: 87), a VLCDR1 comprising an amino acid sequence of NIENKN (SEQ ID NO: 89), a VLCDR2 comprising an amino acid sequence of RDS, and a VLCDR3 comprising an amino acid sequence of QVWDSNTVV (SEQ ID NO: 91).

In some embodiments, an antibody of the present disclosure comprises a heavy chain variable domain comprising an amino acid sequence at least 90% identical to SEQ ID NO: 84, and the light chain variable domain (VL) comprises an amino acid sequence at least 90% identical to SEQ ID NO: 88.

In some embodiments, an antibody of the present disclosure or functional fragment thereof comprises, according to the IMGT unique numbering scheme, a VHCDR1 comprising an amino acid sequence of GGSISSSSYY (SEQ ID NO: 101), a VHCDR2 comprising an amino acid sequence of IGSVDYSGNT (SEQ ID NO: 102), a VHCDR3 comprising an amino acid sequence of ARHRGIYFFDY (SEQ ID NO: 103), a VLCDR1 comprising an amino acid sequence of NIENKN (SEQ ID NO: 89), a VLCDR2 comprising an amino acid sequence of RDS, and a VLCDR3 comprising an amino acid sequence of QVWDSNTVV (SEQ ID NO: 91).

In some embodiments, an antibody of the present disclosure comprises a heavy chain variable domain (VH) comprising an amino acid sequence at least 90% identical to SEQ ID NO: 100, and a light chain variable domain (VL) comprises an amino acid sequence at least 90% identical to SEQ ID NO: 104.

In some embodiments, antibodies of the present disclosure, or functional fragments thereof, are human IgG1 antibodies.

In another aspect, the present disclosure provides the use of an antibody as disclosed herein or functional fragment thereof, in the preparation of a protein as disclosed herein.

Other embodiments and details of the disclosure are presented herein below.

### BRIEF DESCRIPTION OF THE DRAWINGS

FIGS. 1A-1W provide schematic illustrations of various protein designs of fusion proteins with Ig Fc constant domains that bind CD80 and/or CD86, and OX40L. FIG. 1A is a schematic illustration of an exemplary fusion protein where Extracellular Domains (ECDs) of CD28 are fused to the N-terminus of an Fc domain and ECDs of OX40 are fused to the C-terminus of the same Fc domain. FIG. 1B is a schematic illustration of an exemplary fusion protein where ECDs of OX40 are fused to the N-terminus of an Fc domain and ECDs of CD28 are fused to the C-terminus of the same Fc domain. FIG. 1C is a schematic illustration of an exemplary fusion protein where ECDs of CTLA4 are fused to the N-terminus of an Fc domain and ECDs of OX40 are fused to the C-terminus of the same Fc domain. FIG. 1D is a schematic illustration of an exemplary fusion protein where ECDs of OX40 are fused to the N-terminus of an Fc domain and ECDs of CTLA4 are fused to the C-terminus of the same Fc domain. FIG. 1E is a schematic illustration of an exemplary fusion protein where ECDs of CD28 are each fused in tandem to the N-termini of ECDs of OX40 which are then fused to the N-termini of an Fc domain. FIG. 1F is a schematic illustration of an exemplary fusion protein where ECDs of CTLA4 are each fused in tandem to the N-termini of ECDs of OX40 which are then fused to the N-terminus of an Fc domain. FIG. 1G is a schematic illustration of an exemplary fusion protein where ECDs of OX40 are each fused in tandem to the N-termini of ECDs of CD28 which are then fused to the N-terminus of an Fc domain. FIG. 1H is a schematic illustration of an exemplary fusion protein where an ECD of OX40 is fused in tandem to the N-terminus of an ECD of CTLA4 which is then fused to the N-terminus of an Fc domain. FIG. 1I is a schematic illustration of an exemplary fusion protein where any tandem ECD combination of FIGS. 1E-1H (labelled here as X and Y) are fused to the C-terminus of an Fc domain. FIG. 1J is a schematic illustration of an exemplary fusion protein where an ECD of CD28 is fused to the N-terminus of an Fc domain and an anti-OX40L antibody binding domain is fused to the C-terminus of the same Fc domain. FIG. 1K is a schematic illustration of an exemplary fusion protein where ECDs of CTLA4 are fused to the N-terminus of an Fc domain and anti-OX40L antibody binding domains are fused to the C-terminus of the same Fc domain. FIG. 1L is a schematic illustration of an exemplary fusion protein where an ECD of OX40 is fused to the N-terminus of an Fc domain and anti-CD80 and anti-CD86 antibody binding domains are fused to the C-terminus of the Fc domain. FIG. 1M is a schematic illustration of an exemplary fusion protein where anti-OX40L antibody binding domains are fused to the N-terminus of an Fc domain and anti-CD80 and anti-CD86 antibody binding domains are fused to the C-terminus of the Fc domain. FIG. 1N is a schematic illustration of an exemplary fusion protein where anti-CD80 and anti-CD86 antibody binding domains are fused to the N-terminus of an Fc domain and anti-OX40L antibody binding domains are fused to the C-terminus of the Fc domain. FIG. 1O is a schematic illustration of an exemplary fusion protein where anti-CD80 and anti-CD86 antibody binding domains are fused in tandem to the N-termini of anti-OX40L antibody binding domains which are then fused to the N-terminus of an Fc domain. FIG. 1P is a schematic illustration of an exemplary fusion protein where anti-OX40L antibody binding domains are fused in tandem to the N-termini of anti-CD80 and anti-CD86 antibody binding domains which are then fused to the N-terminus of an Fc domain. FIG. 1Q is a schematic illustration of an exemplary fusion protein where anti-CD80 and anti-CD86 antibody binding domains are fused to the C-terminus of a Fc domain and fused in tandem to the N-termini of anti-OX40L antibody binding domains. FIG. 1R is a schematic illustration of an exemplary fusion protein where anti-OX40L antibody binding domains are fused to the C-terminus of an Fc domain and fused in tandem to the N-termini of anti-CD80 and anti-CD86 antibody binding domains. FIG. 1S is a schematic illustration of an exemplary fusion protein where anti-OX40L antibody binding domains are fused to the N-terminus of an Fc domain and anti-CD80 and anti-CD86 antibody binding domains are fused to each other in tandem and to the C-terminus of the Fc domain. FIG. 1T is a schematic illustration of an exemplary fusion protein where anti-CD80 and anti-CD86 antibody binding domains are fused to each other in tandem and to the N-terminus of an Fc domain and anti-OX40L antibody binding domains are fused to the C-terminus of the Fc domain. FIG. 1U is a schematic illustration of an exemplary fusion protein where anti-OX40L antibody binding domains are fused to the N-terminus of an Fc domain and ECDs of CD28 are fused to the C-terminus of the same Fc domain. FIG. 1V is a schematic illustration of an exemplary fusion protein where anti-OX40L antibody binding domains are fused to the N-terminus of an Fc domain and ECDs of CTLA4 are fused to the C-terminus of the same Fc domain. FIG. 1W is a schematic illustration of an exemplary fusion protein where ECDs of CTLA4 are fused to the N-terminus of a variant IgG1 Fc domain having mutations that reduce Fc effector function and anti-OX40L Fabs are fused to the C-terminus of the variant IgG1 Fc domain.

FIG. 2A is a flow chart showing the antibody screening cascade. FIG. 2B is a flow chart showing the candidate characterization cascade.

FIGS. 3A-3C are graphs showing neutralization of OX40L by anti-OX40L in IgG (FIG. 3A), FcFab (FIG. 3B), and fusion protein (FIG. 3C) format using an OX40 Luciferase reporter assay.

FIG. 4 is a graph showing neutralization of OX40L using a CTLA4_anti OX40L fusion protein (95B06, 98C01, 98E10, 84E11, 68F03, 67B06, 97G07, 89B09, and Ref4_CTLA4_013), reference monoclonal anti-OX40L antibody (Ref1_Anti_OX40L, Ref2_Anti-OX40L), or isotype control using an OX40 Luciferase reporter assay.

FIGS. 5A-5B are graphs showing expression of proinflammatory cytokines IL-2 (FIG. 5A) and TNFα (FIG. 5B) in the presence of varying concentrations of CTLA4_anti OX40L fusion proteins (89B09, 67B06, 98E10, and 98C01), reference single agents (Ref2_Anti-OX40L, Ref3_CTLA4Ig), combination of monoclonal anti-OX40L antibody and CTLA4-Ig (combination), or isotype control using an in vitro mixed lymphocyte reaction (MLR) assay.

FIGS. 6A-6B are graphs showing allo-reactive CD4+OX40+(FIG. 6A) and CD8+OX40+(FIG. 6B) T cell proliferation in the presence of CTLA4_anti OX40L fusion proteins (89B09, 67B06, 98E10, and 98C01), reference single agents (Ref2_Anti-OX40L, Ref3_CTLA4Ig), combination of monoclonal anti-OX40L antibody and CTLA4-Ig (combination), or isotype control using a monocyte-derived dendritic cells: T cells (MDDC:T) MLR assay.

FIG. 7 is a graph showing in vitro Treg induction levels in the presence or absence of CTLA4_anti OX40L fusion proteins (89B09, 67B06, and 98C01), reference single agents (Ref1_Anti-OX40L, Ref2_Anti-OX40L, Ref3_CTLA4Ig), or isotype IgG1 control (Iso) using a Treg induction assay.

FIG. 8 is a histogram showing Treg:Teff ratio in the presence of CTLA4_anti OX40L fusion proteins (89B09, 67B06, and 98C01), reference single agents (Ref2_Anti-OX40L, Ref3_CTLA4Ig), or isotype control using a MLR assay.

FIG. 9A is a graph showing Treg:Teff ratio in the presence of varying concentrations of CTLA4_anti OX40L fusion proteins (89B09, 67B06, and 98C01), reference single agents (Ref2_Anti-OX40L, Ref3_CTLA4Ig), combination of monoclonal anti-OX40L antibody and CTLA4-Ig (combination), rapamycin, voclosporin, or isotype control using a MLR Assay. FIG. 9B is a graph showing effector T cell proliferation in the presence of CTLA4_anti OX40L fusion protein (89B09), reference single agents (Ref2_Anti-OX40L, Ref3_CTLA4Ig), combination of monoclonal anti-OX40L antibody and CTLA4-Ig (combination), or isotype control using a MLR assay.

FIGS. 10A-10C are graphs showing IC₅₀ values of IL-2 (FIG. 10A), TNFU (FIG. 10B), and IL-6 (FIG. 10C) in the presence of CTLA4_anti OX40L fusion proteins (67B06 and 89B09), reference single agents (Ref1_Anti-OX40L, Ref3_CTLA4Ig), or combination of monoclonal anti-OX40L antibody and CTLA4-Ig (combination) using a MLR assay.

FIG. 11A is a graph showing IL-2 concentration levels from a CD80 and OX40L receptor occupancy assay. FIG. 11B is a graph showing percent occupancy of human OX40L from a CD80 and OX40L receptor occupancy assay. FIG. 11C is a graph showing percent occupancy of human CD80 from a CD80 and OX40L receptor occupancy assay.

FIG. 12 is a graph showing real-time internalization of CTLA4-Ig (Ref3_CTLA4Ig), anti-OX40L (Ref1_Anti-OX40L), CTLA4_anti OX40L fusion protein (67B06), IgG isotype controls (Isotype Ctl, Isotype IgG1), and non-internalizing anti-CD20 antibody by myeloid-derived dendritic cells (MDDCs).

FIGS. 13A-13D are graphs showing the effect of CTLA4_anti OX40L fusion protein (67B06), reference single agents (Ref1_Anti-OX40L, Ref3_CTLA4Ig), or combination of monoclonal anti-OX40L antibody and CTLA4-Ig (combination) on expression levels of granulocyte-macrophage colony-stimulating factor (GM-CSF) (FIG. 13A), IL13 (FIG. 13B), Granzyme B (GZMB) (FIG. 13C), and IFNγ (FIG. 13D) in PBMCs derived from systemic erythematosus (SLE) patients.

FIG. 14A is a graph showing the effect of CTLA4_anti OX40L fusion proteins (67B06 and 89B09) or isotype control on suppression of IFNγ responses in a Xenogeneic Graft-Versus-Host Disease (xeno-GVHD) murine model. FIG. 14B is a graph showing the effect of CTLA4_anti OX40L fusion protein (67B06), reference single agents 67B06 (IgG format), Ref3_CTLA4Ig, or isotype control on suppression of IFNγ responses in a xeno-GVHD murine model.

FIG. 15A is a graph showing the effect of CTLA4_anti OX40L fusion protein (67B06), reference monoclonal anti-OX40L antibody (Ref1_Anti_OX40L), or isotype control on weight loss in a xeno-GVHD murine model. FIG. 15B is a graph showing the effect of CTLA4_anti OX40L fusion protein (67B06), reference monoclonal anti-OX40L antibody (Ref1_Anti_OX40L), or isotype control on IFNγ production in a xeno-GVHD murine model. Statistical analysis (One-way ANOVA) was performed on Prism Graphpad 8 ****p=<0.0001, *p=0.0175, ***p=0.0002).

FIG. 16A is a graph showing the effect of CTLA4_anti OX40L fusion protein (67B06), reference CTLA4Ig (Ref3_CTLA4Ig), or isotype control on weight loss in a xeno-GVHD murine model. FIG. 16B is a graph showing the effect of CTLA4_anti OX40L fusion protein (67B06), reference CTLA4Ig (Ref3_CTLA4Ig), or isotype control on IFNγ production in a xeno-GVHD murine model. Statistical analysis (One-way ANOVA) was performed on Prism Graphpad 8 *p=0.0455, **p=0.0086).

FIG. 17A is a graph showing the effect of CTLA4_anti OX40L fusion protein (67B06) reference CTLA4Ig (Ref3_CTLA4Ig), Anti-OX40L (Ref1_anti-OX40L), Combination (Ref3 and Ref1), or isotype control on weight loss in a xeno-GVHD murine model on day 41.

FIG. 17B is a graph showing the effect of CTLA4_anti OX40L fusion protein (67B06) reference CTLA4Ig (Ref3_CTLA4Ig), Anti-OX40L (Ref1_anti-OX40L), Combination (Ref3 and Ref1), or isotype control on IFNγ production in a xeno-GVHD murine model on day 41. Statistical analysis (One-way ANOVA) was performed on Prism Graphpad 8 (*p=0.0110, **p=0.0084).

FIG. 17C is a graph showing effect of CTLA4_anti OX40L fusion protein (89B09) or isotype control on IFNγ production in a xeno-GVHD murine model on day 12. Statistical analysis (unpaired t-test) was performed on Prism Graphpad 8 (**p=0.0036).

### DETAILED DESCRIPTION

The present application provides proteins that inhibit T cell costimulatory signaling comprising: a polypeptide that specifically binds CD80 and/or CD86, and a polypeptide that specifically binds OX40L. In some embodiments, the present application provides antibodies that specifically bind OX40L. In some embodiments the application also provides therapeutic methods for using such proteins in the treatment of autoimmune diseases. Various aspects of the proteins described in the present application are set forth below in sections; however, aspects of the proteins described in one particular section are not to be limited to any particular section.

### Definitions

To facilitate an understanding of the present application, a number of terms and phrases are defined below.

The terms “a” and “an” as used herein mean “one or more” and include the plural unless the context is inappropriate.

As used herein, the term “antigen-binding site” refers to the part of the immunoglobulin (Ig) molecule that participates in antigen binding. In human antibodies, the antigen-binding site is formed by amino acid residues of the N-terminal variable (“V”) domains of the heavy (“H”) and light (“L”) chains. Three highly divergent stretches within the V regions of the heavy and light chains are referred to as “hypervariable regions” which are interposed between more conserved flanking stretches known as “framework regions,” or “FR.” Thus, the term “FR” refers to amino acid sequences which are naturally found between and adjacent to hypervariable regions in immunoglobulins. In a human antibody molecule, the three hypervariable regions of a light chain and the three hypervariable regions of a heavy chain are disposed relative to each other in three-dimensional space to form an antigen-binding surface. The antigen-binding surface is complementary to the three-dimensional surface of an antigen to which the antigen-binding site specifically binds, and the three hypervariable regions of each of the heavy and light chains are referred to as “complementarity-determining regions,” or “CDRs.” In certain animals, such as camels and cartilaginous fish, the antigen-binding site is formed by a single antibody chain providing a “single domain antibody.” Antigen-binding sites can exist in an intact antibody, in an antigen-binding fragment of an antibody that retains the antigen-binding surface (for example. Fab, Fab′, F(ab′)₂, or in a recombinant polypeptide such as an scFv, using a peptide linker to connect the heavy chain variable domain to the light chain variable domain in a single polypeptide, a minibody, or a nanobody (VHH).

As used herein, the term “functional fragment thereof” refers to a portion of a protein or polypeptide that maintains the ability to perform a biological function of the whole protein or polypeptide. For example, a functional fragment of a polypeptide or protein of the present application maintains its ability to bind its cognate binding partner or ligand.

As used herein, the terms “subject” and “patient” refer to an organism to be treated by the methods and compositions described herein. Such organisms preferably include, but are not limited to, mammals (e.g., murines, simians, equines, bovines, porcines, canines, felines, and the like), and more preferably include humans.

As used herein, the term “effective amount” refers to the amount of a compound (e.g., a protein of the present application) sufficient to effect beneficial or desired results. An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, CTLA4 (also known as CD152) refers to the protein of SEQ ID NO: 172 and related isoforms and orthologs.

As used herein, OX40L (also known as TNFSF4, or CD252) refers to the protein of SEQ ID NO: 2 and related isoforms and orthologs.

As used herein OX40 (also known as TNFRSF4, or CD134) refers to the protein of SEQ ID NO: 1 and related isoforms and orthologs.

OX40 Human Amino Acid Sequence:
(SEQ ID NO: 1)
LHCVGDTYPSNDRCCHECRPGNGMVSRCSRSQNTVCRPCGPGFYND
VVSSKPCKPCTWCNLRSGSERKQLCTATQDTVCRCRAGTQPLDSYKP
GVDCAPCPPGHFSPGDNQACKPWTNCTLAGKHTLQPASNSSDAICED
RDPPATQPQETQGPPARPITVQPTEAWPRTSQGPSTRPVEVPGGRAVA
AILGLGLVLGLLGPLAILLALYLLRRDQRLPPDAHKPPGGGSFRTPIQE
EQADAHSTLAKI

As used herein CD28 (also known as TP44) refers to the protein of SEQ ID NO: 230 and related isoforms and orthologs.

CD28 Amino Acid Sequence (underlined amino acids
denote signal peptide):
(SEQ ID NO: 230)
MLRLLLALNLFPSIQVTGNKILVKQSPMLVAYDNAVNLSCKYSYNLF
SREFRASLHKGLDSAVEVCVVYGNYSQQLQVYSKTGFNCDGKLGNE
SVTFYLONLYVNQTDIYFCKIEVMYPPPYLDNEKSNGTIIHVKGKHLC
PSPLFPGPSKPFWVLVVVGGVLACYSLLVTVAFIIFWVRSKRSRLLHS
DYMNMTPRRPGPTRKHYQPYAPPRDFAAYRS

As used herein CD80 (also known as B7.1) refers to the protein of SEQ ID NO: 3 and related isoforms and orthologs.

CD80 Human Amino Acid Sequence
(SEQ ID NO: 3)
VIHVTKEVKEVATLSCGHNVSVEELAQTRIYWQKEKKMVLTMMSGD
MNIWPEYKNRTIFDITNNLSIVILALRPSDEGTYECVVLKYEKDAFKRE
HLAEVTLSVKADFPTPSISDFEIPTSNIRRIICSTSGGFPEPHLSWLENG
EELNAINTTVSQDPETELYAVSSKLDFNMTTNHSFMCLIKYGHLRVNQ
TFNWNTTKQEHFPDNLLPSWAITLISVNGIFVICCLTYCFAPRCRERRR
NERLRRESVRPV

As used herein CD86 (also known as B7.2) refers to the protein of SEQ ID NO: 4 and related isoforms and orthologs.

CD86 Human Amino Acid Sequence
(SEQ ID NO: 4)
APLKIQAYFNETADLPCQFANSQNQSLSELVVFWQDQENLVLNEVYL
GKEKFDSVHSKYMGRTSFDSDSWTLRLHNLQIKDKGLYQCIIHHKKP
TGMIRIHQMNSELSVLANFSQPEIVPISNITENVYINLTCSSIHGYPEPK
KMSVLLRTKNSTIEYDGVMQKSQDNVTELYDVSISLSVSFPDVTSNM
TIFCILETDKTRLLSSPFSIELEDPQPPPDHIPWITAVLPTVIICVMVFC
LILWKWKKKKRPRNSYKCGTNTMEREESEQTKKREKIHIPERSDEAQR
VFKSSKTSSCDKSDTCF

Throughout the description, where a protein is described as having, including, or comprising specific components, or where processes and methods are described as having, including, or comprising specific steps, it is contemplated that, additionally, there are proteins described in the present application that consist essentially of or consist of the recited components, and that there are processes and methods according to the present application that consist essentially of or consist of the recited processing steps.

### I. Proteins

The present application provides proteins that specifically bind to CD80 and CD86 and OX40L expressed on antigen presenting cells (APCs). Binding of the proteins to CD80/CD86 and OX40L inhibits T cell activation by blocking costimulatory signaling downstream of CD28 and OX40, respectively. The proteins of the present disclosure include two arms, each arm including two components (e.g., a polypeptide or a complex of two or more polypeptides), one component which specifically binds to CD80 and/or CD86 and another component which specifically binds to OX40L. In some embodiments, the components that bind to either CD80 and/or CD86 or OX40L can comprise a single polypeptide. In some embodiments, the components that bind to either CD80 and/or CD86 or OX40L can comprise a complex of two or more polypeptides. In some embodiments, the complex of two or more polypeptides of the polypeptide complex may be connected by one or more than one covalent linkage (e.g., a disulfide bond) and/or one or more than one non-covalent interaction (e.g., an ionic or hydrophobic interaction). Further description of exemplary proteins is provided below.

A protein of the present disclosure includes the first component that is a polypeptide or complex of two or more polypeptides that specifically binds CD80 and/or CD86, which can include but is not limited to an extracellular domain of CTLA4 or functional fragment thereof. In some embodiments, the first component can be but is not limited to an extracellular domain of CD28 or functional fragment thereof. In some embodiments, the first component can be but is not limited to one or more antigen-binding site, for example, an antibody, a Fab, a Fab′, a F(ab′)₂, a single-chain variable fragment (scFv), a minibody, or a nanobody (VHH).

A protein of the present disclosure includes the second component, which is a polypeptide or complex of two or more polypeptides that specifically binds OX40L. In some embodiments, the second component can include but is not limited to an antigen-binding site, for example, an antibody, a Fab, a Fab′, a F(ab′)₂, a single-chain variable fragment (scFv), a minibody, or a nanobody (VHH). In some embodiments, the second component can be but is not limited to an extracellular domain of OX40 or functional fragment thereof.

In some embodiments, a protein of the present disclosure additionally comprises a bridging moiety, which can include but is not limited to: a polypeptide of an immunoglobulin Fc domain or functional fragment thereof, a human serum albumin (HSA) polypeptide or functional fragment thereof, or a polypeptide linker. In some embodiments, the bridging moiety is a polypeptide of an immunoglobulin Fc domain. In some embodiments, a protein of the present disclosure further comprises a polypeptide hinge immediately N-terminal to the polypeptide of the immunoglobulin Fc domain or functional fragment thereof.

In some embodiments, a protein of the present disclosure further comprises a linker polypeptide, which connects a polypeptide or complex of two or more polypeptides that specifically binds OX40L to the bridging moeity. In some embodiments, a protein of the present disclosure further comprises a linker polypeptide, which connects a polypeptide or complex of two or more polypeptides that specifically binds CD80 and/or CD86 to the bridging moiety.

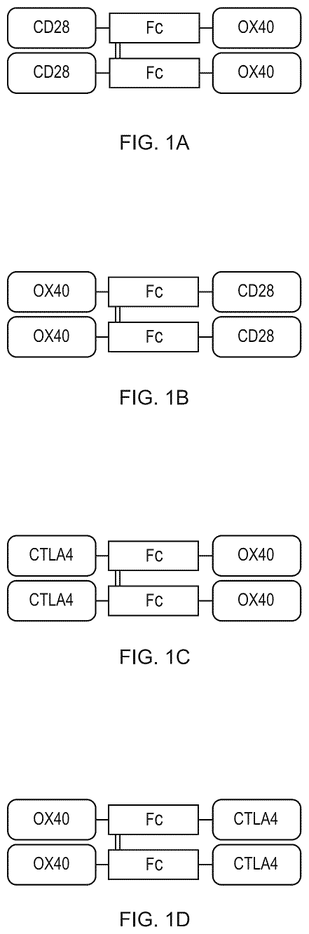

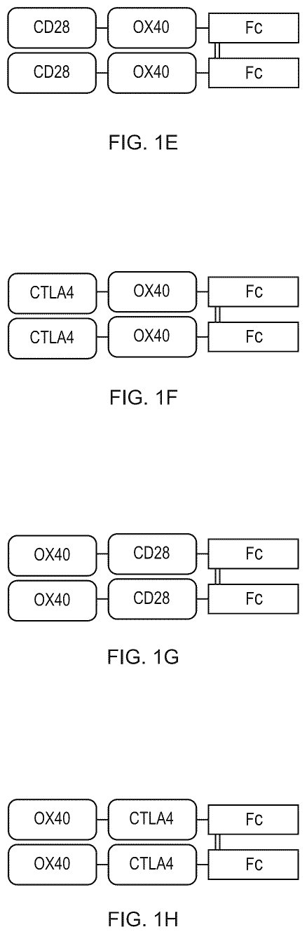

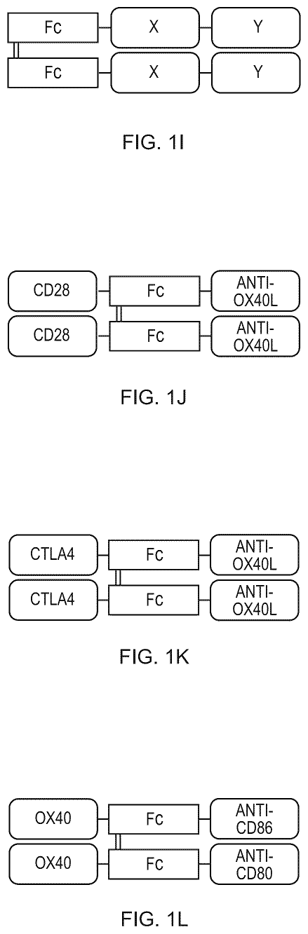

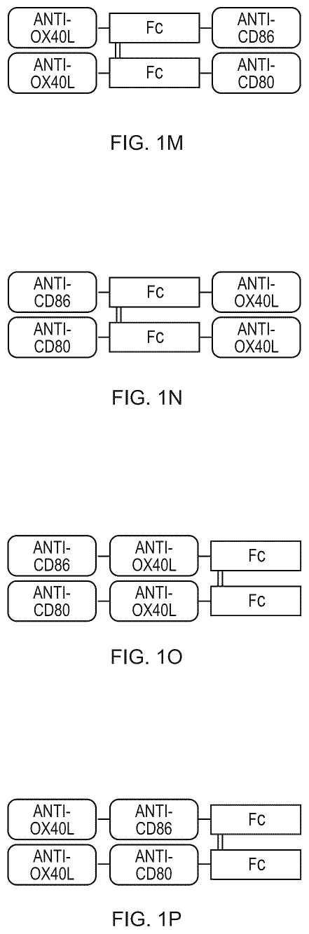

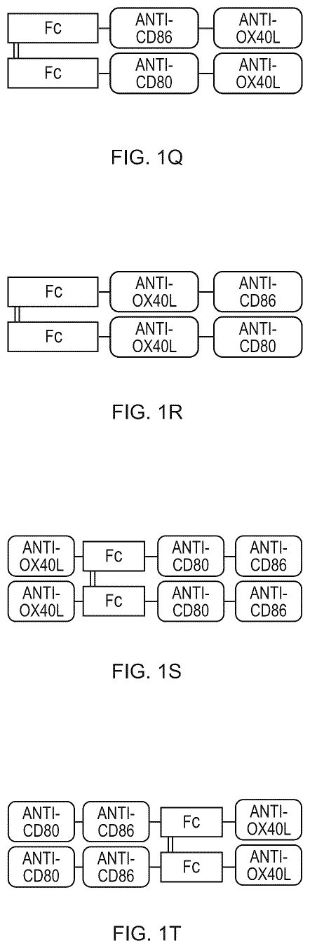

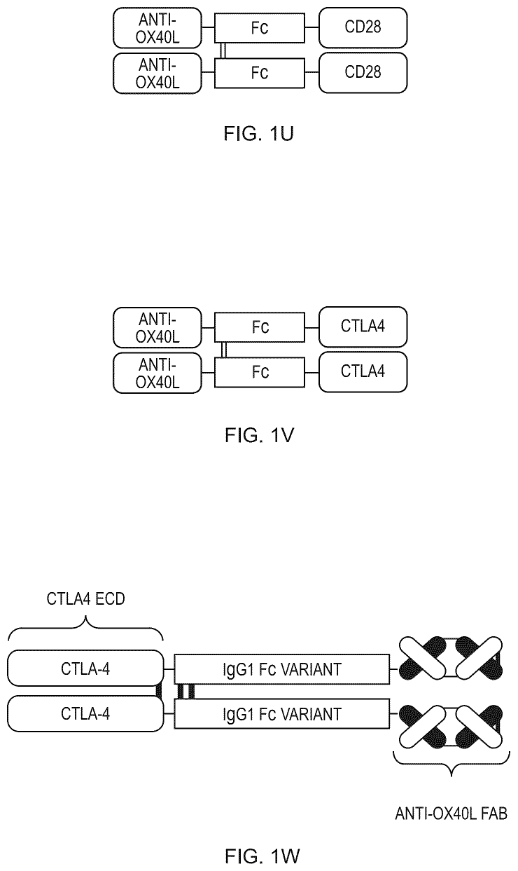

The proteins described herein can take various formats. For example, one protein format comprises: (i) a first arm comprising, from N-terminus to C-terminus, a first polypeptide or complex of two or more polypeptides that specifically binds CD80 and/or CD86, a first polypeptide of an immunoglobulin Fc domain, and a first polypeptide or complex of two or more polypeptides that specifically binds OX40L; and (ii) a second arm comprising, from N-terminus to C-terminus, a second polypeptide or complex of two or more polypeptides that specifically binds CD80 and/or CD86, a second polypeptide of the immunoglobulin Fc domain, and a second polypeptide or complex of two or more polypeptides that specifically binds OX40L, wherein the first and second polypeptides of the immunoglobulin Fc-domain dimerize. In this format, first and second polypeptides or complexes of two or more polypeptides that specifically bind CD80 and/or CD86 include: an extracellular domain of CD28 or functional fragment thereof (FIG. 1A and FIG. 1J); an extracellular domain of CTLA4 or functional fragment thereof (FIG. 1C, FIG. 1K, and FIG. 1W); or wherein the first polypeptide or complex of two or more polypeptides that specifically binds CD80 and/or CD86 is an antigen-binding site (e.g., an antibody, a Fab, a Fab′, a F(ab′)₂, a single-chain variable fragment (scFv), a minibody, or a nanobody (VHH)) that specifically binds CD80, and the second polypeptide or complex of two or more polypeptides that specifically binds CD80 and/or CD86 is an antigen-binding site (e.g., an antibody, a Fab, a Fab′, a F(ab′)₂, a single-chain variable fragment (scFv), a minibody, or a nanobody (VHH)) that specifically binds CD86 (FIG. 1N).

In this format, first and second polypeptides or complexes of two or more polypeptides that specifically bind OX40L include: an extracellular domain of OX40 or functional fragment thereof (FIG. 1A and FIG. 1C); or an antigen-binding site (e.g., an antibody, a Fab, a Fab′, a F(ab′)2, a single-chain variable fragment (scFv), a minibody, or a nanobody (VHH)) (FIG. 1J, FIG. 1K, and FIG. 1N). In some embodiments, the first and second complexes of two or more polypeptides that specifically bind OX40L comprise a Fab, wherein a partial hinge (for example, a polypeptide having the sequence of SEQ ID NO: 336) is connected to the C-terminus of the Fab heavy chain (HC) CH1 domain and forms a disulfide bond with the Fab light chain (LC).

Proteins of this format may optionally comprise a polypeptide hinge in each of the first and second arms connecting the C-terminus of the first or second polypeptide or complex of two or more polypeptides that specifically binds CD80 and/or CD86 to the N-terminus of the first or second polypeptide of the immunoglobulin Fc domain, respectively. Proteins of this format may also optionally comprise a linker polypeptide in one or both of the first and/or second arms connecting the C-terminus of the first and/or second polypeptide of the immunoglobulin Fc domain, to the N-terminus of the first and/or second polypeptide or complex of two or more polypeptides that specifically binds OX40L, respectively.

Another protein format comprises: (i) a first arm comprising, from N-terminus to C-terminus, a first polypeptide or complex of two or more polypeptides that specifically binds OX40L, a first polypeptide of an immunoglobulin Fc domain, and a first polypeptide or complex of two or more polypeptides that specifically binds CD80 and/or CD86; and (ii) a second arm comprising, from N-terminus to C-terminus, a second polypeptide or complex of two or more polypeptides that specifically binds OX40L, a second polypeptide of the immunoglobulin Fc domain, and a second polypeptide or complex of two or more polypeptides that specifically binds CD80 and/or CD86, wherein the first and second polypeptides of the immunoglobulin Fc-domain dimerize. In this format, first and second polypeptides or complexes of two or more polypeptides that specifically bind OX40L include: an extracellular domain of OX40 or functional fragment thereof (FIG. 1B, FIG. 1D, and FIG. 1L); or an antigen-binding site (e.g., an antibody, a Fab, a Fab′, a F(ab′)₂, a single-chain variable fragment (scFv), a minibody, or a nanobody (VHH)) (FIG. 1M, FIG. 1U, and FIG. 1V). In some embodiments, the first and second complexes of two or more polypeptides that specifically bind OX40L comprise a Fab, wherein a partial hinge (for example, a polypeptide having the sequence of SEQ ID NO: 336) is connected to the C-terminus of the Fab heavy chain (HC) CH1 domain and forms a disulfide bond with the Fab light chain (LC). In this format, first and second polypeptides or complexes of two or more polypeptides that specifically bind CD80 and/or CD86 include: an extracellular domain of CD28 or functional fragment thereof (FIG. 1B and FIG. 1U); an extracellular domain of CTLA4 or functional fragment thereof (FIG. 1D and FIG. 1V); or wherein the first polypeptide or complex of two or more polypeptides that specifically binds CD80 and/or CD86 is an antigen-binding site (e.g., an antibody, a Fab, a Fab′, a F(ab′)2, a single-chain variable fragment (scFv), a minibody, or a nanobody (VHH)) that specifically binds CD80, and the second polypeptide or complex of two or more polypeptides that specifically binds CD80 and/or CD86 is an antigen-binding site (e.g., an antibody, a Fab, a Fab′, a F(ab′)₂, a single-chain variable fragment (scFv), a minibody, or a nanobody (VHH)) that specifically binds CD86 (FIG. 1L and FIG. 1M). Proteins of this format may optionally comprise a polypeptide hinge in each of the first and second arms connecting the C-terminus of the first or second polypeptide or complex of two or more polypeptides that specifically binds OX40L to the N-terminus of the first or second polypeptide of the immunoglobulin Fc domain, respectively. Proteins of this format may also optionally comprise a linker polypeptide in one or both of the first and/or second arms connecting the C-terminus of the first and/or second polypeptide of the immunoglobulin Fc domain, to the N-terminus of the first and/or second polypeptide or complex of two or more polypeptides that specifically binds CD80 and/or CD86, respectively.

In another example, a protein format comprises: (i) a first arm comprising, from N-terminus to C-terminus, a first polypeptide or complex of two or more polypeptides that specifically binds CD80 and/or CD86, a first polypeptide or complex of two or more polypeptides that specifically binds OX40L, and a first polypeptide of an immunoglobulin Fc domain; and (ii) a second arm comprising, from N-terminus to C-terminus, a second polypeptide or complex of two or more polypeptides that specifically binds CD80 and/or CD86, a second polypeptide or complex of two or more polypeptides that specifically binds OX40L, and a second polypeptide of the immunoglobulin Fc domain, wherein the first and second polypeptides of the immunoglobulin Fc-domain dimerize. In this format, first and second polypeptides or complexes of two or more polypeptides that specifically bind CD80 and/or CD86 include: an extracellular domain of CD28 or functional fragment thereof (FIG. 1E); an extracellular domain of CTLA4 or functional fragment thereof (FIG. 1F); or wherein the first polypeptide or complex of two or more polypeptides that specifically binds CD80 and/or CD86 is an antigen-binding site (e.g., an antibody, a Fab, a Fab′, a F(ab′)₂, a single-chain variable fragment (scFv), a minibody, or a nanobody (VHH)) that specifically binds CD80, and the second polypeptide or complex of two or more polypeptides that specifically binds CD80 and/or CD86 is an antigen-binding site (e.g., an antibody, a Fab, a Fab′, a F(ab′)₂, a single-chain variable fragment (scFv), a minibody, or a nanobody (VHH)) that specifically binds CD86 (FIG. 1O). In this format, first and second polypeptides or complexes of two or more polypeptides that specifically bind OX40L include: an extracellular domain of OX40 or functional fragment thereof (FIG. 1E and FIG. 1F); or an antigen-binding site (e.g., an antibody, a Fab, a Fab′, a F(ab′)2, a single-chain variable fragment (scFv), a minibody, or a nanobody (VHH)) (FIG. 1O). In some embodiments, the first and second complexes of two or more polypeptides that specifically bind OX40L comprise a Fab, wherein a partial hinge (for example, a polypeptide having the sequence of SEQ ID NO: 336) is connected to the C-terminus of the Fab heavy chain (HC) CH1 domain and forms a disulfide bond with the Fab light chain (LC). Proteins of this format may optionally comprise a polypeptide hinge in each of the first and second arms connecting the C-terminus of the first or second polypeptide or complex of two or more polypeptides that specifically binds OX40L to the N-terminus of the first or second polypeptide of the immunoglobulin Fc domain, respectively. Proteins of this format may also optionally comprise a linker polypeptide in one or both of the first and/or second arms connecting the C-terminus of the first and/or second polypeptide or complex of two or more polypeptides that specifically binds CD80 and/or CD86, to the N-terminus of the first and/or second polypeptide or complex of two or more polypeptides that specifically binds OX40L, respectively.

Another protein format comprises: (i) a first arm comprising, from N-terminus to C-terminus, a first polypeptide or complex of two or more polypeptides that specifically binds OX40L, a first polypeptide or complex of two or more polypeptides that specifically binds CD80 and/or CD86, and a first polypeptide of an immunoglobulin Fc domain; and (ii) a second arm comprising, from N-terminus to C-terminus, a second complex of two or more polypeptides that specifically binds OX40L, a second polypeptide or complex of two or more polypeptides that specifically binds CD80 and/or CD86, and a second polypeptide of the immunoglobulin Fc domain, wherein the first and second polypeptides of the immunoglobulin Fc-domain dimerize. In this format, first and second polypeptides or complexes of two or more polypeptides that specifically bind OX40L include: an extracellular domain of OX40 or functional fragment thereof (FIG. 1G and FIG. 1H); or an antigen-binding site (e.g., an antibody, a Fab, a Fab′, a F(ab′)₂, a single-chain variable fragment (scFv), a minibody, or a nanobody (VHH)) (FIG. 1P). In some embodiments, the first and second complexes of two or more polypeptides that specifically bind OX40L comprise a Fab, wherein a partial hinge (for example, a polypeptide having the sequence of SEQ ID NO: 336) is connected to the C-terminus of the Fab heavy chain (HC) CH1 domain and forms a disulfide bond with the Fab light chain (LC). In this format, first and second polypeptides or complexes of two or more polypeptides that specifically bind CD80 and/or CD86 include: an extracellular domain of CD28 or functional fragment thereof (FIG. 1G); an extracellular domain of CTLA4 or functional fragment thereof (FIG. 1H); or wherein the first polypeptide or complex of two or more polypeptides that specifically binds CD80 and/or CD86 is an antigen-binding site (e.g., an antibody, a Fab, a Fab′, a F(ab′)₂, a single-chain variable fragment (scFv), a minibody, or a nanobody (VHH)) that specifically binds CD80, and the second polypeptide or complex of two or more polypeptides that specifically binds CD80 and/or CD86 is an antigen-binding site (e.g., an antibody, a Fab, a Fab′, a F(ab′)₂, a single-chain variable fragment (scFv), a minibody, or a nanobody (VHH)) that specifically binds CD86 (FIG. 1P). Proteins of this format may optionally comprise a polypeptide hinge in each of the first and second arms connecting the C-terminus of the first or second polypeptide or complex of two or more polypeptides that specifically binds CD80 and/or CD86 to the N-terminus of the first or second polypeptide of the immunoglobulin Fc domain, respectively. Proteins of this format may also optionally comprise a linker polypeptide in one or both of the first and/or second arms connecting the C-terminus of the first and/or second polypeptide or complex of two or more polypeptides that specifically binds OX40L, to the N-terminus of the first and/or second polypeptide or complex of two or more polypeptides that specifically binds CD80 and/or CD86, respectively.

In another example, a protein format comprises: (i) a first arm comprising, from N-terminus to C-terminus, a first polypeptide of an immunoglobulin Fc domain, a first polypeptide or complex of two or more polypeptides that specifically binds CD80 and/or CD86, and a first polypeptide or complex of two or more polypeptides that specifically binds OX40L; and (ii) a second arm comprising, from N-terminus to C-terminus, a second polypeptide of the immunoglobulin Fc domain, a second polypeptide or complex of two or more polypeptides that specifically binds CD80 and/or CD86, and a second polypeptide or complex of two or more polypeptides that specifically binds OX40L, wherein the first and second polypeptides of the immunoglobulin Fc-domain dimerize. In this format, first and second polypeptides or complexes of two or more polypeptides that specifically bind CD80 and/or CD86 include: an extracellular domain of CD28 or functional fragment thereof (FIG. 1I); an extracellular domain of CTLA4 or functional fragment thereof; or wherein the first polypeptide or complex of two or more polypeptides that specifically binds CD80 and/or CD86 is an antigen-binding site (e.g., an antibody, a Fab, a Fab′, a F(ab′)₂, a single-chain variable fragment (scFv), a minibody, or a nanobody (VHH)) that specifically binds CD80, and the second polypeptide or complex of two or more polypeptides that specifically binds CD80 and/or CD86 is an antigen-binding site (e.g., an antibody, a Fab, a Fab′, a F(ab′)₂, a single-chain variable fragment (scFv), a minibody, or a nanobody (VHH)) that specifically binds CD86 (FIG. 1Q). In this format, first and second polypeptides or complexes of two or more polypeptides that specifically bind OX40L include: an extracellular domain of OX40 or functional fragment thereof (FIG. 1I); or an antigen-binding site (e.g., an antibody, a Fab, a Fab′, a F(ab′)2, a single-chain variable fragment (scFv), a minibody, or a nanobody (VHH)) (FIG. 1Q). In some embodiments, the first and second complexes of two or more polypeptides that specifically bind OX40L comprise a Fab, wherein a partial hinge (for example, a polypeptide having the sequence of SEQ ID NO: 336) is connected to the C-terminus of the Fab heavy chain (HC) CH1 domain and forms a disulfide bond with the Fab light chain (LC). Proteins of this format may optionally comprise a polypeptide hinge in each of the first and second arms N-terminal of the first or second polypeptides of the immunoglobulin Fc domain, respectively. Proteins of this format may also optionally comprise a linker polypeptide in one or both of the first and/or second arms connecting the C-terminus of the first and/or second polypeptide of the immunoglobulin Fc domain to the N-terminus of the first and/or second polypeptide or complex of two or more polypeptides that specifically binds CD80 and/or CD86, respectively. Proteins of this format may also optionally comprise a linker polypeptide in one or both of the first and/or second arms connecting the C-terminus of the first and/or second polypeptide or complex of two or more polypeptides that specifically binds CD80 and/or CD86, to the N-terminus of the first and/or second polypeptide or complex of two or more polypeptides that specifically binds OX40L, respectively.

Another protein format comprises: (i) a first arm comprising, from N-terminus to C-terminus, a first polypeptide of an immunoglobulin Fc domain, a first polypeptide or complex of two or more polypeptides that specifically binds OX40L, and a first polypeptide or complex of two or more polypeptides that specifically binds CD80 and/or CD86; and (ii) a second arm comprising, from N-terminus to C-terminus, a second polypeptide of the immunoglobulin Fc domain, a second polypeptide or complex of two or more polypeptides that specifically binds OX40L, and a second polypeptide or complex of two or more polypeptides that specifically binds CD80 and/or CD86, wherein the first and second polypeptides of the immunoglobulin Fc-domain dimerize. In this format, first and second polypeptides or complexes of two or more polypeptides that specifically bind OX40L include: an extracellular domain of OX40 or functional fragment thereof (FIG. 1I); or an antigen-binding site (e.g., an antibody, a Fab, a Fab′, a F(ab′)₂, a single-chain variable fragment (scFv), a minibody, or a nanobody (VHH)) (FIG. 1R). In some embodiments, the first and second complexes of two or more polypeptides that specifically bind OX40L comprise a Fab, wherein a partial hinge (for example, a polypeptide having the sequence of SEQ ID NO: 336) is connected to the C-terminus of the Fab heavy chain (HC) CH1 domain and forms a disulfide bond with the Fab light chain (LC). In this format, first and second polypeptides or complexes of two or more polypeptides that specifically bind CD80 and/or CD86 include: an extracellular domain of CD28 or functional fragment thereof (FIG. 1I); an extracellular domain of CTLA4 or functional fragment thereof; or wherein the first polypeptide or complex of two or more polypeptides that specifically binds CD80 and/or CD86 is an antigen-binding site (e.g., an antibody, a Fab, a Fab′, a F(ab′)₂, a single-chain variable fragment (scFv), a minibody, or a nanobody (VHH)) that specifically binds CD80, and the second polypeptide or complex of two or more polypeptides that specifically binds CD80 and/or CD86 is an antigen-binding site (e.g., an antibody, a Fab, a Fab′, a F(ab′)₂, a single-chain variable fragment (scFv), a minibody, or a nanobody (VHH)) that specifically binds CD86 (FIG. 1R). Proteins of this format may optionally comprise a polypeptide hinge in each of the first and second arms N-terminal of the first or second polypeptide of the immunoglobulin Fc domain, respectively. Proteins of this format may also optionally comprise a linker polypeptide in one or both of the first and/or second arms connecting the C-terminus of the first and/or second polypeptide of the immunoglobulin Fc domain to the N-terminus of the first and/or second polypeptide or complex of two or more polypeptides that specifically binds OX40L, respectively. Proteins of this format may also optionally comprise a linker polypeptide in one or both of the first and/or second arms connecting the C-terminus of the first and/or second polypeptide or complex of two or more polypeptides that specifically binds OX40L, to the N-terminus of the first and/or second polypeptide or complex of two or more polypeptides that specifically binds CD80 and/or CD86, respectively.

Another protein format comprises: (i) a first arm comprising, from N-terminus to C-terminus, a first polypeptide or complex of two or more polypeptides that specifically binds OX40L, a first polypeptide of an immunoglobulin Fc domain, and a first polypeptide or complex of two or more polypeptides that specifically binds CD80 and CD86; and (ii) a second arm comprising, from N-terminus to C-terminus, a second polypeptide or complex of two or more polypeptides that specifically binds OX40L, a second polypeptide of the immunoglobulin Fc domain, and a second polypeptide or complex of two or more polypeptides that specifically binds CD80 and CD86, wherein the first and second polypeptides of the immunoglobulin Fc-domain dimerize. In this format, first and second polypeptides or complexes of two or more polypeptides that specifically bind OX40L include: an extracellular domain of OX40 or functional fragment thereof; or an antigen-binding site (e.g., an antibody, a Fab, a Fab′, a F(ab′)₂, a single-chain variable fragment (scFv), a minibody, or a nanobody (VHH)) (FIG. 1S). In some embodiments, the first and second complexes of two or more polypeptides that specifically bind OX40L comprise a Fab, wherein a partial hinge (for example, a polypeptide having the sequence of SEQ ID NO: 336) is connected to the C-terminus of the Fab heavy chain (HC) CH1 domain and forms a disulfide bond with the Fab light chain (LC). In this format, first and second polypeptides or complexes of two or more polypeptides that specifically bind CD80 and/or CD86 include: an antigen-binding site (e.g., an antibody, a Fab, a Fab′, a F(ab′)₂, a single-chain variable fragment (scFv), a minibody, or a nanobody (VHH)) that specifically binds CD80 connected, with or without a linker polypeptide, to an antigen-binding site (e.g., an antibody, a Fab, a Fab′, a F(ab′)₂, a single-chain variable fragment (scFv), a minibody, or a nanobody (VHH)) that specifically binds CD86 (FIG. 1S), or an antigen-binding site (e.g., an antibody, a Fab, a Fab′, a F(ab′)₂, a single-chain variable fragment (scFv), a minibody, or a nanobody (VHH)) that specifically binds CD86 connected, with or without a linker polypeptide, to an antigen-binding site (e.g., an antibody, a Fab, a Fab′, a F(ab′)₂, a single-chain variable fragment (scFv), a minibody, or a nanobody (VHH)) that specifically binds CD80. Proteins of this format may optionally comprise a polypeptide hinge in each of the first and second arms connecting the C-terminus of the first or second polypeptide or complex of two or more polypeptides that specifically binds OX40L to the N-terminus of the first or second polypeptide of the immunoglobulin Fc domain, respectively. Proteins of this format may also optionally comprise a linker polypeptide in one or both of the first and/or second arms connecting the C-terminus of the first and/or second polypeptide of the immunoglobulin Fc domain, to the N-terminus of the first and/or second polypeptide or complex of two or more polypeptides that specifically binds CD80 and/or CD86, respectively.

Another protein format comprises: (i) a first arm comprising, from N-terminus to C-terminus, a first polypeptide or complex of two or more polypeptides that specifically binds CD80 and CD86, a first polypeptide of an immunoglobulin Fc domain, and a first polypeptide or complex of two or more polypeptides that specifically binds OX40L; and (ii) a second arm comprising, from N-terminus to C-terminus, a second polypeptide or complex of two or more polypeptides that specifically binds CD80 and CD86, a second polypeptide of the immunoglobulin Fc domain, and a second polypeptide or complex of two or more polypeptides that specifically binds OX40L, wherein the first and second polypeptides of the immunoglobulin Fc-domain dimerize. In this format, first and second polypeptides or complexes of two or more polypeptides that specifically bind OX40L include: an extracellular domain of OX40 or functional fragment thereof; or an antigen-binding site (e.g., an antibody, a Fab, a Fab′, a F(ab′)₂, a single-chain variable fragment (scFv), a minibody, or a nanobody (VHH)) (FIG. 1T). In some embodiments, the first and second complexes of two or more polypeptides that specifically bind OX40L comprise a Fab, wherein a partial hinge (for example, a polypeptide having the sequence of SEQ ID NO: 336) is connected to the C-terminus of the Fab heavy chain (HC) CH1 domain and forms a disulfide bond with the Fab light chain (LC). In this format, first and second polypeptides or polypeptide complexes that specifically bind CD80 and/or CD86 include: an antigen-binding site (e.g., an antibody, a Fab, a Fab′, a F(ab′)₂, a single-chain variable fragment (scFv), a minibody, or a nanobody (VHH)) that specifically binds CD80 connected, with or without a linker polypeptide, to an antigen-binding site (e.g., an antibody, a Fab, a Fab′, a F(ab′)₂, a single-chain variable fragment (scFv), a minibody, or a nanobody (VHH)) that specifically binds CD86 (FIG. 1T), or an antigen-binding site (e.g., an antibody, a Fab, a Fab′, a F(ab′)2, a single-chain variable fragment (scFv), a minibody, or a nanobody (VHH)) that specifically binds CD86 connected, with or without a linker polypeptide, to an antigen-binding site (e.g., an antibody, a Fab, a Fab′, a F(ab′)₂, a single-chain variable fragment (scFv), a minibody, or a nanobody (VHH)) that specifically binds CD80. Proteins of this format may optionally comprise a polypeptide hinge in each of the first and second arms connecting the C-terminus of the first or second polypeptide or complex of two or more polypeptides that specifically binds CD80 and/or CD86 to the N-terminus of the first or second polypeptide of the immunoglobulin Fc domain, respectively. Proteins of this format may also optionally comprise a linker polypeptide in one or both of the first and/or second arms connecting the C-terminus of the first and/or second polypeptide of the immunoglobulin Fc domain, to the N-terminus of the first and/or second polypeptide or complex of two or more polypeptides that specifically binds OX40L, respectively. In some embodiments, a first and/or second polypeptide may be a single polypeptide chain. In some embodiments, a first and/or second polypeptide may be a complex of two or more polypeptide chains.

Individual components of the proteins are described in more detail below.

I.A Polypeptides or Complexes of Two or More Polypeptides that Specifically Bind CD80 and/or CD86

Polypeptides of complexes of two or more polypeptides that specifically bind CD80 and/or CD86 on the surface of antigen presenting cells (APCs), such as B cells, monocytes, macrophages and dendritic cells, block binding of endogenous CD80 and/or CD86 to CD28 on the surface of T cells. By preventing the interaction between CD80/CD86 and CD28, polypeptides that specifically bind CD80 and/or CD86 can inhibit T cell costimulatory receptor signaling, resulting in decreased T cell activation, proliferation and induction of an anergic, immunosuppressive, tolerogenic T cell response.

As used herein, a polypeptide or complex of two or more polypeptides that specifically binds CD80 and/or CD86 is a polypeptide or complex of two or more polypeptides that binds to a protein having a sequence of SEQ ID NO: 3 and related isoforms and orthologs and/or a protein having a sequence of SEQ ID NO: 4 and related isoforms and orthologs.

In some embodiments, a polypeptide or complex of two or more polypeptides that specifically binds CD80 and/or CD86 include, but is not limited to, a CTLA4 extracellular domain or functional fragment thereof, an antibody, a Fab, a Fab′, a F(ab′)₂, a single-chain variable fragment (scFv), a minibody, or a nanobody (VHH).

For example, in some embodiments, a polypeptide that specifically binds CD80 and/or CD86 is an extracellular domain of CTLA4 or functional fragment thereof. In some embodiments, the extracellular domain of CTLA4 can comprise a sequence derived from a wildtype sequence of human CTLA4 (e.g., Genbank Accession number NP_005205). Alternatively, the CTLA4 polypeptide sequence may be a variant CTLA4 sequence as described in Larsen et al. Am J Transplant. 2005 March; 5(3):443-53.; Xu et al. J Immunol. 2012 Nov. 1; 189(9):4470-7.; Bernett et al., MAbs. 2013 May-June; 5(3):384-96.; Oshima et al. Protein Eng Des Sel. 2016 May; 29(5):159-67.; Douthwaite et al. J Immunol. 2017 Jan. 1; 198(1):528-537. Erratum in: J Immunol. 2017 Sep. 1; 199(5):1943. For example, a CTLA4 sequence may comprise one or more mutation selected from mutations at positions 16, 24, 25, 27, 28, 29, 30, 32, 49, 50, 51, 53, 54, 55, 56, 58, 61, 64, 65, 70, 80, 85, 93, 96, and 104 (numbering relative to SEQ ID NO: 174).

Polypeptides that specifically bind CD80 and/or CD86 polypeptides of the present invention may comprise an amino acid sequence selected from any wildtype or variant sequence, or functional fragment thereof, listed in TABLE 1.

TABLE 1
Wildtype and Variant CTLA4 Sequences

| DESCRIPTION | SEQUENCE |
|---|---|
| Full unprocessed human | MACLGFORHKAQLNLATRTWPCTLLFFLLFIPVFCKAMH |
| CTLA4 (Genbank | VAQPAVVLASSRGIASFVCEYASPGKATEVRVTVLRQADS |
| Accession number | QVTEVCAATYMMGNELTFLDDSICTGTSSGNQVNLTIQG |
| NP_005205) (underlined | LRAMDTGLYICKVELMYPPPYYLGIGNGTQIYVIDPEPCP |
| amino acids denote signal | DSDFLLWILAAVSSGLFFYSFLLTAVSLSKMLKKRSPLTTG |
| peptide as per RefSeq | VYVKMPPTEPECEKQFQPYFIPIN (SEQ ID NO: 172) |
| annotation) | |
| Human CTLA4 | KAMHVAQPAVVLASSRGIASFVCEYASPGKATEVRVTVL |
| (Extracellular domain | RQADSQVTEVCAATYMMGNELTFLDDSICTGTSSGNQVN |
| amino acid residues 36- | LTIQGLRAMDTGLYICKVELMYPPPYYLGIGNGTQIYVIDP |
| 161) | EPCPDSD (SEQ ID NO: 29) |
| Human CTLA4 | AMHVAQPAVVLASSRGIASFVCEYASPGKATEVRVTVLR |
| (Extracellular domain | QADSQVTEVCAATYMMGNELTFLDDSICTGTSSGNQVNL |
| amino acid residues 37- | TIQGLRAMDTGLYICKVELMYPPPYYLGIGNGTQIYVIDPE |
| 161) | PCPDSD (SEQ ID NO: 173) |
| Human CTLA4 | MHVAQPAVVLASSRGIASFVCEYASPGKATEVRVTVLRQ |
| (Extracellular domain | ADSQVTEVCAATYMMGNELTFLDDSICTGTSSGNQVNLTI |
| amino acid residues 38- | QGLRAMDTGLYICKVELMYPPPYYLGIGNGTQIYVIDPEP |
| 161) | CPDSD (SEQ ID NO: 174) |
| CTLA4 Variant 1 | MHVAQPAVVLASSRGIASFVCEYASPGKYTEVRVTVLRQ |
| Larsen et al. Am J | ADSQVTEVCAATYMMGNELTFLDDSICTGTSSGNQVNLTI |
| Transplant. 2005 | QGLRAMDTGLYICKVELMYPPPYYEGIGNGTQIYVIDPEP |
| March; 5(3):443-53. | CPDSD (SEQ ID NO: 119) |
| [A29Y, L104E] | |
| CTLA4 Variant 2 | MHVAQPAVVLASSRGIASFVCEYASPGKX₁TEVRVTVLRQ |
| Xu et al. J Immunol. | ADSQVTEVCAAX₂YMMGNEGTFLDDSICTGTSSGNQVNL |
| 2012 Nov. 1; | TIQGLRAMDTGLYICX₃VEKMYPPPYYLGIGNGTQIYVIDP |
| 189(9):4470-7. | EPCPDSD |
| [K28H, A29H, A29K, | wherein X₁ is H, K, T, W, or Y; |
| A29T, A29W, A29Y, | X₂ is N or Y; and |
| T51N, T51Y, L58G, | X₃ is M or Q |
| K93M, K93Q, L96K] | (SEQ ID NO: 215) |
| CTLA4 Variant 3 | MHVAQPAVVLASSRGIASFVCEYASPGKX₁TEVRVTVLRQ |
| Bernett et al., MAbs. | ADSQVTEVCFANYYMGNELTFX₂DDSICTGTSSGNQVNLT |
| 2013 May-Jun; 5(3):384- | IQGLRAMDTGLYICQVELMYPPPYYEGIGNGTQIYVIDPEP |
| 96. | CPDSD |
| [A29H, A29K, A29W, | wherein X₁ is H, K, W, or Y; and |
| A29Y, A49F, T51N, | X₂ is D or E |
| M53Y, L61D, L61E, | (SEQ ID NO: 233) |
| K93Q, L104E] | |
| CTLA4 Variant 4 | MHVAQPAVVLASSRGIASFVCEYESPGKANEIRVTVLRQ |
| Oshima et al. Protein | ADSQVTEVCAMTYMKEDELTFLDDPSCTGTFSGNQVNL |
| Eng Des Sel. 2016 | TIQGLRAMDTGLYICKVELMYPPPYYEGIGNGTQIYVIDPE |
| May; 29(5):159-67. | PCPDSD (SEQ ID NO: 234) |
| [A24E, T30N, V32I, | |
| A50M, M54K, G55E, | |
| N56D, S64P, I65S, S70F, | |
| L104E] | |
| CTLA4 Variant 5 | MHVAQPAVVLASSRGX₁ASFVCEYTNPSKATEVRVTVLR |
| Douthwaite et al. J | QADSQVTEVCAATYMKGNEX₂TFLDDSICTGTASGNQVN |
| Immunol. 2017 Jan. 1; | LTIRGLRAX₃DTGLYICQVELMYPPPYYLGIGNGTQIYVID |
| 198(1):528-537. | PEPCPDSD |
| [I16R, I16V, I16S, A24T, | wherein X₁ is R or V; |
| S25N, G27S, M54K, | X₂ is A or G; and |
| L58A, L58G, S70A, | X₃ is Q or S |
| Q80R, M85Q, M85S, | (SEQ ID NO: 235) |
| K93Q] | |

In some embodiments, a polypeptide that specifically binds CD80 and/or CD86 is an extracellular domain of CTLA4 or functional fragment thereof comprising an amino acid sequence at least 90% (e.g., at least 91%, at least 92%, at least 93%, at least 94%, at least 95%, at least 96%, at least 97%, at least 98%, at least 99%, or 100%) identical to a sequence listed in TABLE 1.

In some embodiments, a polypeptide that specifically binds CD80 and/or CD86 comprises an extracellular domain of CD28 (SEQ ID NO: 236) or functional fragment thereof.

CD28 (Underlining denotes the signal peptide
sequence)
(SEQ ID NO: 236)
MLRLLLALNLFPSIQVTGNKILVKQSPMLVAYDNAVNLSWKHLCPSP
LFPGPSKPFWVLVVVGGVLACYSLLVTVAFIIFWVRSKRSRLLHSDY
MNMTPRRPGPTRKHYQPYAPPRDFAAYRS

In some embodiments, a polypeptide or complex of two or more polypeptides that specifically binds CD80 and/or CD86 comprises an antibody, a Fab, a Fab′, a F(ab′)₂, a single-chain variable fragment (scFv), a minibody, and a nanobody (VHH).

In some embodiments of proteins of the present invention, polypeptides or heavy chains of complexes of two or more polypeptides that specifically bind CD80 and/or CD86 can be connected to a polypeptide or heavy chain of a complex of two or more polypeptides that specifically binds OX40L (for example, a polypeptide comprising an amino acid sequence listed in TABLE 3) to form a contiguous polypeptide chain. In some embodiments of proteins of the present invention, polypeptides or complexes of two or more polypeptides that specifically bind CD80 and/or CD86 are connected to a polypeptide or complex of two or more polypeptides that specifically binds OX40L via a bridging moiety (for example, a polypeptide comprising an amino acid sequence listed in TABLE 4), wherein the bridging moiety connects the C-terminus of the polypeptide or heavy chain of a complex of two or more polypeptides that specifically binds CD80 and/or CD86 to the N-terminus of the polypeptide or heavy chain of a complex of two or more polypeptides that specifically binds OX40L to form a contiguous polypeptide chain. In some embodiments, polypeptides or heavy chains of complexes of two or more polypeptides that specifically bind CD80 and/or CD86 are connected to the bridging moiety via a hinge polypeptide (for example, a polypeptide comprising an amino acid sequence listed in TABLE 5), wherein the hinge polypeptide connects the C-terminus of the polypeptide or heavy chain of the complex of two or more polypeptides that specifically binds CD80 and/or CD86 to the N-terminus of the bridging moiety to form a contiguous polypeptide chain.

I.B Polypeptides or Complexes of Two or More Polypeptides that Specifically Bind OX40L

Polypeptides or complexes of two or more poylpeptides that specifically bind OX40L on the surface of antigen presenting cells (APCs), such as B cells, monocytes, macrophages and dendritic cells, block binding to endogenous OX40 on the surface of T cells. By preventing the interaction between OX40L and OX40, polypeptides that specifically bind OX40L can inhibit T cell costimulatory receptor signaling, resulting in decreased T cell activation, proliferation and induction of an anergic, immunosuppressive, tolerogenic T cell response.

As used herein, a polypeptide or complex of two or more polypeptides that specifically binds OX40L is a polypeptide that binds to a protein of Genbank Accession No. NP_003317 (SEQ ID NO: 2) and related isoforms and orthologs.

OX40L (underlining denotes transmembrane region,
bold denotes position 1 of the extracellular
domain)
(SEQ ID NO: 2)
MERVQPLEENVGNAARPRFERNKLLLVASVIQGLGLLLCFTYICLHES
ALQVSHRYPRIQSIKVQFTEYKKEKGFILTSQKEDEIMKVQNNSVIINC
DGFYLISLKGYFSQEVNISLHYQKDEEPLFOLKKVRSVNSLMVASLTY
KDKVYLNVTTDNTSLDDFHVNGGELILIHQNPGEFCVL

In some embodiments, a polypeptide or complex of two or more polypeptides that specifically binds OX40L as described herein binds to position 17, 18, 19, 20, 21, 23, 26, 28, 60, 83, 110, 111, 112, 113 and 114 of the extracellular domain of human OX40L. In other embodiments, a polypeptide or complex of two or more polypeptides that specifically binds OX40L as described herein binds to position 58, 59, 60, 61, 62, 63, 81, 82, and 83 of the extracellular domain of human OX40L. In some embodiments, a polypeptide or complex of two or more polypeptides that specifically binds OX40L binds position 60 and/or 83 of the extracellular domain of human OX40L.

In some embodiments, a polypeptide or complex of two or more polypeptides that specifically binds OX40L include, but is not limited to: an antigen-binding site, for example an antibody, a Fab, a Fab′, a F(ab′)₂, a single-chain variable fragment (scFv), a minibody, or a nanobody (VHH); or an extracellular domain of OX40 or functional fragment thereof.

In some embodiments, a polypeptide or complex of two or more polypeptides that specifically binds OX40L comprises an antigen-binding site. In certain embodiments, the antigen-binding site is a Fab. TABLE 2 lists consensus heavy chain variable domain (VH) and light chain variable domain (VL) complementarity-determining regions (CDRs) that, in combination, can specifically bind to OX40L. In some embodiments, the heavy chain variable domain and the light chain variable domain are arranged in Fab format having VH and VL CDR sequences selected from the consensus VH and VL sequences in TABLE 2. In some embodiments, a partial hinge (for example, a polypeptide having the sequence of SEQ ID NO: 336) is connected to the C-terminus of the Fab heavy chain (HC) CH1 domain and forms a disulfide bond with the Fab light chain (LC).

Unless indicated otherwise, the CDR sequences provided in TABLE 2 are determined under the IMGT unique numbering scheme.

TABLE 2
VHCDR and VLCDR sequences for antigen-binding sites that
specifically bind OX40L
Consensus Sequences

| | |
|---|---|
| Heavy Chain | Light Chain |

CDR1

| | |
|---|---|
| GX₁SX₂X₃X₄SX₅YY | X₁IENKN |
| wherein X₁ is A, G, or V, | wherein X₁ is N or D |
| X₂ is V, or I, | (SEQ ID NO: 226) |
| X₃ is S or R, | |
| X₄ is S, or T, and | |
| X₅ is S, or G; | |
| (SEQ ID NO: 222) | |
| | SX₁RX₂X₃X₄ |
| | wherein X₁ is V or L, |
| | X₂ is R or N, |
| | X₃ is F or Y, and |
| | X₄ is F or Y |

CDR2

| | |
|---|---|
| IX₁YX₂GST | RDN |
| wherein X₁ is Y, or N, and | |
| X₂ is S, or G, | |
| (SEQ ID NO: 223) | |
| X₁DYSGT | GKD |
| wherein X₁ is I or M, and | |
| (SEQ ID NO: 224) | |
| IGSVDYSGX₁T | RDS |
| wherein X₁ is N, A, or S | |
| (SEQ ID NO: 225) | |

CDR3

| | |
|---|---|
| ARHRGX₁YX₂FDX₃ | QVX₁DSX₂X₃VV |
| wherein X₁ is S or I, | wherein X₁ is R or W, |
| X₂ is F or H, and | X₂ is N, T, or A, and |
| X₃ is I or Y | X₃ is I, T, or A, and |
| (SEQ ID NO: 220) | (SEQ ID NO: 231) |
| ARERSX₁X₂WYPX₃DY | NSRDSSGYX₁VX₂ |
| wherein X₁ is N or S, | wherein X₁ is L or H, and |
| X₂ is N, D, G or S, and | X₂ is L or V |
| X₃ is I or F | (SEQ ID NO: 232) |
| (SEQ ID NO: 221) | |

In certain embodiments, the antigen-binding site that specifically binds to OX40L comprises an amino acid sequence selected from the consensus sequence of SEQ ID NO: 220 or SEQ ID NO: 221.

In some embodiments, the antigen-binding site that specifically binds to OX40L comprises: a heavy chain variable complementarity-determining region 1 (VHCDR1) comprising an amino acid sequence of SEQ ID NO: 222; a heavy chain variable complementarity-determining region 2 (VHCDR2) comprising an amino acid sequence of SEQ ID NO: 223, SEQ ID NO: 224, or SEQ ID NO: 225); and a heavy chain variable complementarity-determining region 3 (VHCDR3) comprising an amino acid sequence of: SEQ ID NO: 220 or SEQ ID NO: 221.

In some embodiments, the antigen-binding site that specifically binds to OX40L comprises: a light chain variable complementarity-determining region 1 (VLCDR1) comprising an amino acid sequence of SEQ ID NO: 226 or SX₁RX₂X₃X₄, wherein X₁ is V or L, X₂ is R or N, X₃ is F or Y, and X₄ is F or Y; a light chain variable complementarity-determining region 2 (VLCDR2) comprising an amino acid sequence of RDN, GKD or RDS; and a light chain variable complementarity-determining region 3 (VLCDR3) comprising an amino acid sequence of SEQ ID NO: 231 or SEQ ID NO: 232.

In some embodiments, the antigen-binding site that specifically binds to OX40L comprises: a VHCDR1 comprising an amino acid sequence of SEQ ID NO: 222; a VHCDR2 comprising an amino acid sequence of SEQ ID NO: 223; a VHCDR3 comprising an amino acid sequence of SEQ ID NO: 220; a VLCDR1 comprising an amino acid sequence of SEQ ID NO: 226; a VLCDR2 comprising an amino acid sequence of RDN; and a VLCDR3 comprising an amino acid sequence of SEQ ID NO: 231.

In some embodiments, the antigen-binding site that specifically binds to OX40L comprises: a VHCDR1 comprising an amino acid sequence of SEQ ID NO: 222; a VHCDR2 comprising an amino acid sequence of SEQ ID NO: 224; a VHCDR3 comprising an amino acid sequence of SEQ ID NO: 221; a VLCDR1 comprising an amino acid sequence of SX₁RX₂X₃X₄, wherein X₁ is V or L, X₂ is R or N, X₃ is F or Y, and X₄ is F or Y; a VLCDR2 comprising an amino acid sequence of GKD; and a VLCDR3 comprising an amino acid sequence of SEQ ID NO: 232.

In some embodiments, the antigen-binding site that specifically binds to OX40L comprises, according to the IMGT unique numbering scheme, VHCDR1, VHCDR2, VHCDR3, VLCDR1, VLCDR2, and VLCDR3 each comprising an amino acid sequence corresponding to the VHCDR1, VHCDR2, VHCDR3, VLCDR1, VLCDR2, and VLCDR3 sequences of a VHCDR and VLCDR consensus sequences of TABLE 2, respectively.

In some embodiments, a polypeptide or complex of two or more polypeptides that specifically binds OX40L comprises an antigen-binding site. In certain embodiments, the antigen-binding site is a Fab. TABLE 3 lists polypeptide sequences of heavy chain variable domains (VH) and light chain variable domains (VL) that, in combination, can specifically bind to OX40L. In some embodiments, the heavy chain variable domain and the light chain variable domain are arranged in Fab format having VH and VL sequences selected from the VH and VL sequences in TABLE 3. TABLE 3 additionally lists polypeptide sequences of heavy chains (HC) and light chains (LC) that, in combination, can specifically bind to OX40L. In some embodiments, the heavy chain and the light chains are arranged in Fab format having HC and LC sequences selected from the HC and LC sequences in TABLE 3.

Unless indicated otherwise, the CDR sequences provided in TABLE 3 are determined under the IMGT unique numbering scheme.

TABLE 3
VH/VL and HC/LC sequences for antigen-binding sites that
specifically bind OX40L

| Clone | Amino Acid Sequence | |
|---|---|---|
| 67B06 | Heavy Chain | |
| | QLQLQESGPGLVKPSETLSLTCSVSGGSISTSSYYWGWIRQPPGKGLEW | |
| | SGSIYYSGSTYYRPSLQGRATISVDTSKNQFSLKLTSVTAADTAVYYCA | |
| | RHRGSYFFDIWGLGTLVTVSSASTKGPSVFPLAPSSKSTSGGTAALGCL | |
| | VKDYFPEPVTVSWNSGALTSGVHTFPAVLQSSGLYSLSSVVTVPSSSLG | |
| | TQTYICNVNHKPSNTKVDKRVEPKSCDKTHTCPPCPAPELLGGPSVFLF | |
| | PPKPKDTLMISRTPEVTCVVVDVSHEDPEVKFNWYVDGVEVHNAKTK | |
| | PREEQYNSTYRVVSVLTVLHQDWLNGKEYKCKVSNKALPAPIEKTISK | |
| | AKGQPREPQVYTLPPSREEMTKNQVSLTCLVKGFYPSDIAVEWESNGQ | |
| | PENNYKTTPPVLDSDGSFFLYSKLTVDKSRWQQGNVFSCSVMHEALH | |
| | NHYTQKSLSLSPGK (SEQ ID NO: | |
| | 8) | |
| | Light Chain | |
| | SYELTQPLSVSVALGQTARMTCGGSDIENKNVHWYQQKPGQAPVLVI | |
| | HRDNNRPSGIPERFSGSNSGNTATLTISRAQAGDEADYYCQVRDSNIV | |
| | VFGGGTKLTVLGQPKAAPSVTLFPPSSEELQANKATLVCLISDFYPGAV | |
| | TVAWKADSSPVKAGVETTTPSKQSNNKYAASSYLSLTPEQWKSHKSY | |
| | SCQVTHEGSTVEKTVAPTECS (SEQ ID NO: 7) | |
| | Heavy Chain Variable Domain (VH) | Light Chain Variable Domain (VL) |
| | QLQLQESGPGLVKPSETLSLTCS | SYELTQPLSVSVALGQTARMTCG |
| | VSGGSISTSSYYWGWIRQPPGKG | GSDIENKNVHWYQQKPGQAPVL |
| | LEWSGSIYYSGSTYYRPSLQGRA | VIHRDNNRPSGIPERFSGSNSGNT |
| | TISVDTSKNQFSLKLTSVTAADT | ATLTISRAQAGDEADYYCQVRDS |
| | AVYYCARHRGSYFFDIWGLGTL | NIVVFGGGTKLTVL (SEQ ID NO: |
| | VTVSS (SEQ ID NO: 76) | 80) |
| | CDR1-GGSISTSSYY (SEQ ID | CDR1-DIENKN (SEQ ID NO: 81) |
| | NO: 77) | CDR2-RDN |
| | CDR2-IYYSGST (SEQ ID NO: 78) | CDR3-QVRDSNIVV (SEQ ID NO: |
| | CDR3-ARHRGSYFFDI (SEQ ID | 83) |
| | NO: 79) | |
| 98E10 | Heavy Chain | |
| | QLQLQESGPGLVKPSETLSLTCTVAGASVSSSSYYWGWIRQSPGKGLE | |
| | WIGTINYGGSTYHNPSLKRRVTVSVDTSKNQFSLKLTSVTAADTAVYY | |
| | CARHRGIYHFDYWGQGTLVTVSSASTKGPSVFPLAPSSKSTSGGTAAL | |
| | GCLVKDYFPEPVTVSWNSGALTSGVHTFPAVLQSSGLYSLSSVVTVPS | |
| | SSLGTQTYICNVNHKPSNTKVDKRVEPKSCDKTHTCPPCPAPELLGGPS | |
| | VFLFPPKPKDTLMISRTPEVTCVVVDVSHEDPEVKFNWYVDGVEVHN | |
| | AKTKPREEQYNSTYRVVSVLTVLHQDWLNGKEYKCKVSNKALPAPIE | |
| | KTISKAKGQPREPQVYTLPPSREEMTKNQVSLTCLVKGFYPSDIAVEW | |
| | ESNGQPENNYKTTPPVLDSDGSFFLYSKLTVDKSRWQQGNVFSCSVM | |
| | HEALHNHYTQKSLSLSPGK (SEQ ID NO: 14) | |
| | Light Chain | |
| | SYELTQPLSVSVALGQTARITCGGNNIENKNVHWYQQMPGQAPVLVI | |
| | YRDSNRPSGIPERFSGSNSGNTATLTINRAQAGDEADYYCQVWDSNTV | |
| | VFGGGTKVTVLGQPKAAPSVTLFPPSSEELQANKATLVCLISDFYPGA | |
| | VTVAWKADSSPVKAGVETTTPSKQSNNKYAASSYLSLTPEQWKSHKS | |
| | YSCQVTHEGSTVEKTVAPTECS (SEQ ID NO: 13) | |
| | Heavy Chain Variable Domain (VH) | Light Chain Variable Domain (VL) |
| | QLQLQESGPGLVKPSETLSLTCT | SYELTQPLSVSVALGQTARITCGG |
| | VAGASVSSSSYYWGWIRQSPGK | NNIENKNVHWYQQMPGQAPVLV |
| | GLEWIGTINYGGSTYHNPSLKRR | IYRDSNRPSGIPERFSGSNSGNTAT |
| | VTVSVDTSKNQFSLKLTSVTAAD | LTINRAQAGDEADYYCQVWDSN |
| | TAVYYCARHRGIYHEDYWGQGT | TVVFGGGTKVTVL (SEQ ID NO: |
| | LVTVSS (SEQ ID NO: 84) | 88) |
| | CDR1-GASVSSSSYY (SEQ ID | CDR1-NIENKN (SEQ ID NO: 89) |
| | NO: 85) | CDR2-RDS |
| | CDR2-INYGGST (SEQ ID NO: | CDR3-QVWDSNTVV (SEQ ID |
| | 86) | NO: 91) |
| | CDR3-ARHRGIYHFDY (SEQ ID | |
| | NO: 87) | |
| 89B09 | Heavy Chain | |
| | EVOLVESGPGLVKPSETLSLTCTVSGVSIRSNGYYWGWIRQSPGKGLE | |
| | WIGNMDYSGTYQNPSLTSRVTISGDASKNQFSLKLRSVTAADTAVYFC | |
| | ARERSNNWYPIDYWGQGTLVTVSSASTKGPSVFPLAPSSKSTSGGTAA | |
| | LGCLVKDYFPEPVTVSWNSGALTSGVHTFPAVLQSSGLYSLSSVVTVP | |
| | SSSLGTQTYICNVNHKPSNTKVDKRVEPKSCDKTHTCPPCPAPELLGGP | |
| | SVFLFPPKPKDTLMISRTPEVTCVVVDVSHEDPEVKFNWYVDGVEVH | |
| | NAKTKPREEQYNSTYRVVSVLTVLHQDWLNGKEYKCKVSNKALPAPI | |
| | EKTISKAKGQPREPQVYTLPPSREEMTKNQVSLTCLVKGFYPSDIAVE | |
| | WESNGQPENNYKTTPPVLDSDGSFFLYSKLTVDKSRWQQGNVFSCSV | |
| | MHEALHNHYTQKSLSLSPGK (SEQ ID NO: 16) | |
| | Light Chain | |
| | SSELTQDPAVSVALGQTVRITCQGDSVRRFFAHWYQQKPGQAPILVIS | |
| | GKDTRPSGIPDRFSGSISGNTASLTITGAQAEDEADYYCNSRDSSGYLV | |
| | LFGGGTKLTVLGQPKAAPSVTLFPPSSEELQANKATLVCLISDFYPGAV | |
| | TVAWKADSSPVKAGVETTTPSKQSNNKYAASSYLSLTPEQWKSHKSY | |
| | SCQVTHEGSTVEKTVAPTECS (SEQ ID NO: 15) | |
| | Heavy Chain Variable Domain (VH) | Light Chain Variable Domain (VL) |
| | EVQLVESGPGLVKPSETLSLTCT | SSELTQDPAVSVALGQTVRITCQG |
| | VSGVSIRSNGYYWGWIRQSPGK | DSVRRFFAHWYQQKPGQAPILVIS |
| | GLEWIGNMDYSGTYQNPSLTSR | GKDTRPSGIPDRFSGSISGNTASLT |
| | VTISGDASKNQFSLKLRSVTAAD | ITGAQAEDEADYYCNSRDSSGYL |
| | TAVYFCARERSNNWYPIDYWGQ | VLFGGGTKLTVL (SEQ ID NO: 96) |
| | GTLVTVSS (SEQ ID NO: 92) | CDR1-SVRRFF (SEQ ID NO: 97) |
| | CDR1-GVSIRSNGYY (SEQ ID | CDR2-GKD |
| | NO: 93) | CDR3-NSRDSSGYLVL (SEQ ID |
| | CDR2-MDYSGT (SEQ ID NO: 94) | NO: 99) |
| | CDR3-ARERSNNWYPIDY (SEQ | |
| | ID NO: 95) | |
| 98C01 | Heavy Chain | |
| | QVQLQESGPGLVKPSETLSLTCTVSGGSISSSSYYWGWIRQPPGKGLE | |
| | WIGGIGSVDYSGNTYYKPSLKSRVTISVDTSKNQFSLKLSSVTAADTAV | |
| | YHCARHRGIYFFDYWGQGTLVTVSSASTKGPSVFPLAPSSKSTSGGTA | |
| | ALGCLVKDYFPEPVTVSWNSGALTSGVHTFPAVLQSSGLYSLSSVVTV | |
| | PSSSLGTQTYICNVNHKPSNTKVDKRVEPKSCDKTHTCPPCPAPELLGG | |
| | PSVFLFPPKPKDTLMISRTPEVTCVVVDVSHEDPEVKFNWYVDGVEVH | |
| | NAKTKPREEQYNSTYRVVSVLTVLHQDWLNGKEYKCKVSNKALPAPI | |
| | EKTISKAKGQPREPQVYTLPPSREEMTKNQVSLTCLVKGFYPSDIAVE | |
| | WESNGQPENNYKTTPPVLDSDGSFFLYSKLTVDKSRWQQGNVFSCSV | |
| | MHEALHNHYTQKSLSLSPGK (SEQ ID NO: 18) | |
| | Light Chain | |
| | SYELTQPPSVSVALGQTARITCGGNNIENKNVHWYQQKPGQAPVLVIY | |
| | RDSNRPSGIPERFSGSNSGNTATLSINRAQAGDEADYYCQVWDSNTVV | |
| | FGGGTKLTVLGQPKAAPSVTLFPPSSEELQANKATLVCLISDFYPGAVT | |
| | VAWKADSSPVKAGVETTTPSKQSNNKYAASSYLSLTPEQWKSHKSYS | |
| | CQVTHEGSTVEKTVAPTECS (SEQ ID NO: 17) | |
| | Heavy Chain Variable Domain (VH) | Light Chain Variable Domain (VL) |
| | QVQLQESGPGLVKPSETLSLTCT | SYELTQPPSVSVALGQTARITCGG |
| | VSGGSISSSSYYWGWIRQPPGKG | NNIENKNVHWYQQKPGQAPVLVI |
| | LEWIGGIGSVDYSGNTYYKPSLK | YRDSNRPSGIPERFSGSNSGNTAT |
| | SRVTISVDTSKNQFSLKLSSVTAA | LSINRAQAGDEADYYCQVWDSN |
| | DTAVYHCARHRGIYFFDYWGQG | TVVFGGGTKLTVL (SEQ ID NO: |
| | TLVTVSS (SEQ ID NO: 100) | 104) |
| | CDR1-GGSISSSSYY (SEQ ID | CDR1-NIENKN (SEQ ID NO: 89) |
| | NO: 101) | CDR2-RDS |
| | CDR2-IGSVDYSGNT (SEQ ID | CDR3-QVWDSNTVV (SEQ ID |
| | NO: 102) | NO: 91) |
| | CDR3-ARHRGIYFFDY (SEQ ID | |
| | NO: 103) | |
| 97G07 | Heavy Chain | |
| | QLQLQESGPGLVKPSETLSLTCTVSGGSISSSSYYWGWIRQPPGKGLEW | |
| | IGGIGSVDYSGNTYYKPSLKSRVTISVDTSKNQFSLKLSTVTAADTAVY | |
| | HCARHRGIYFFDYWGQGTLVTVSSASTKGPSVFPLAPSSKSTSGGTAA | |
| | LGCLVKDYFPEPVTVSWNSGALTSGVHTFPAVLQSSGLYSLSSVVTVP | |
| | SSSLGTQTYICNVNHKPSNTKVDKRVEPKSCDKTHTCPPCPAPELLGGP | |
| | SVFLFPPKPKDTLMISRTPEVTCVVVDVSHEDPEVKFNWYVDGVEVH | |
| | NAKTKPREEQYNSTYRVVSVLTVLHQDWLNGKEYKCKVSNKALPAPI | |
| | EKTISKAKGQPREPQVYTLPPSREEMTKNQVSLTCLVKGFYPSDIAVE | |
| | WESNGQPENNYKTTPPVLDSDGSFFLYSKLTVDKSRWQQGNVFSCSV | |
| | MHEALHNHYTQKSLSLSPGK (SEQ ID NO: 6) | |
| | Light Chain | |
| | YYELTQPLSVSVALGQTARIPCGGNNIENKNVHWYQQKPGQAPVLVIY | |
| | RDSNRPSGIPERFSGSNSGNTATLSINRAQAGDEADYYCQVWDSNTVIF | |
| | GGGTKVTVLGQPKAAPSVTLFPPSSEELQANKATLVCLISDFYPGAVT | |
| | VAWKADSSPVKAGVETTTPSKQSNNKYAASSYLSLTPEQWKSHKSYS | |
| | CQVTHEGSTVEKTVAPTECS (SEQ ID NO: 5) | |
| | Heavy Chain Variable Domain (VH) | Light Chain Variable Domain (VL) |
| | QLQLQESGPGLVKPSETLSLTCT | YYELTQPLSVSVALGQTARIPCGG |
| | VSGGSISSSSYYWGWIRQPPGKG | NNIENKNVHWYQQKPGQAPVLVI |
| | LEWIGGIGSVDYSGNTYYKPSLK | YRDSNRPSGIPERFSGSNSGNTAT |
| | SRVTISVDTSKNQFSLKLSTVTAA | LSINRAQAGDEADYYCQVWDSN |
| | DTAVYHCARHRGIYFFDYWGQG | TVIFGGGTKVTVL (SEQ ID NO: |
| | TLVTVSS (SEQ ID NO: 108) | 112) |
| | CDR1-GGSISSSSYY (SEQ ID | CDR1-NIENKN (SEQ ID NO: 89) |
| | NO: 109) | CDR2-RDS |
| | CDR2-IGSVDYSGNT (SEQ ID | CDR3-QVWDSNTVI (SEQ ID NO: |
| | NO: 110) | 115) |
| | CDR3-ARHRGIYFFDY (SEQ ID | |
| | NO: 103) | |
| 68F03 | Heavy Chain | |
| | QLQLQESGPGLVKPSETLSLTCTVSGGSIDTSSQYWAWIRQPPGKGLE | |
| | WIGGIYFSGSTYQNPSLKSRVTTSVDTSKNQFSLRLSSMTAADTAVYY | |
| | CARHRGIYFFDYWGQGTLVTVSSASTKGPSVFPLAPSSKSTSGGTAAL | |
| | GCLVKDYFPEPVTVSWNSGALTSGVHTFPAVLQSSGLYSLSSVVTVPS | |
| | SSLGTQTYICNVNHKPSNTKVDKRVEPKSCDKTHTCPPCPAPELLGGPS | |
| | VFLFPPKPKDTLMISRTPEVTCVVVDVSHEDPEVKFNWYVDGVEVHN | |
| | AKTKPREEQYNSTYRVVSVLTVLHQDWLNGKEYKCKVSNKALPAPIE | |
| | KTISKAKGQPREPQVYTLPPSREEMTKNQVSLTCLVKGFYPSDIAVEW | |
| | ESNGQPENNYKTTPPVLDSDGSFFLYSKLTVDKSRWQQGNVFSCSVM | |
| | HEALHNHYTQKSLSLSPGK (SEQ ID NO: 10) | |
| | Light Chain | |
| | SFELTQPLSVSVALGQTARITCGGINIENKNVHYYQQKPGQAPVLVIYR | |
| | DTNRPSGIPERFSGSNSGNTATLTISRAQAGDEADYYCQVWDSNTVIFG | |
| | GGTKVTVLGQPKAAPSVTLFPPSSEELQANKATLVCLISDFYPGAVTV | |
| | AWKADSSPVKAGVETTTPSKQSNNKYAASSYLSLTPEQWKSHKSYSC | |
| | QVTHEGSTVEKTVAPTECS (SEQ ID NO: 9) | |
| | Heavy Chain Variable Domain (VH) | Light Chain Variable Domain (VL) |
| | QLQLQESGPGLVKPSETLSLTCT | SFELTQPLSVSVALGQTARITCGGI |
| | VSGGSIDTSSQYWAWIRQPPGKG | NIENKNVHYYQQKPGQAPVLVIY |
| | LEWIGGIYFSGSTYQNPSLKSRVT | RDTNRPSGIPERFSGSNSGNTATL |
| | TSVDTSKNQFSLRLSSMTAADTA | TISRAQAGDEADYYCQVWDSNT |
| | VYYCARHRGIYFFDYWGQGTLV | VIFGGGTKVTVL (SEQ ID NO: |
| | TVSS (SEQ ID NO: 116) | 120) |
| | CDR1-GGSIDTSSQY (SEQ ID | CDR1-NIENKN (SEQ ID NO: 89) |
| | NO: 117) | CDR2-RDT |
| | CDR2-IYFSGST (SEQ ID NO: | CDR3-QVWDSNTVI (SEQ ID NO: |
| | 118) | 123) |
| | CDR3-ARHRGIYFFDY (SEQ ID | |
| | NO: 103) | |
| 84E11 | Heavy Chain | |
| | QLQLQESGPGLVKPSETLSLTCTVSGGSISSGSFYWGWIRQPPGKGLEW | |
| | IGTIYYSGSTYYNPSLKSRVAISVDTSKNQFYLNLNFVTAADTAVYHCA | |
| | RERSSSWYPFDYWGQGTLVTVSSASTKGPSVFPLAPSSKSTSGGTAAL | |
| | GCLVKDYFPEPVTVSWNSGALTSGVHTFPAVLQSSGLYSLSSVVTVPS | |
| | SSLGTQTYICNVNHKPSNTKVDKRVEPKSCDKTHTCPPCPAPELLGGPS | |
| | VFLFPPKPKDTLMISRTPEVTCVVVDVSHEDPEVKFNWYVDGVEVHN | |
| | AKTKPREEQYNSTYRVVSVLTVLHQDWLNGKEYKCKVSNKALPAPIE | |
| | KTISKAKGQPREPQVYTLPPSREEMTKNQVSLTCLVKGFYPSDIAVEW | |
| | ESNGQPENNYKTTPPVLDSDGSFFLYSKLTVDKSRWQQGNVFSCSVM | |
| | HEALHNHYTQKSLSLSPGK (SEQ ID NO: 12) | |
| | Light Chain | |
| | SSELTQDPAVSVALGQTVRITCQGDSLRNYFANWYQQKPGQAPVLVIY | |
| | GKNNRPSGIPDRFSGSSSGNTASLTITGAQAEDEADYYCNSRDSSGYH | |
| | VVFGGGTKLTVLGQPKAAPSVTLFPPSSEELQANKATLVCLISDFYPGA | |
| | VTVAWKADSSPVKAGVETTTPSKQSNNKYAASSYLSLTPEQWKSHKS | |
| | YSCQVTHEGSTVEKTVAPTECS (SEQ ID NO: 11) | |
| | Heavy Chain Variable Domain (VH) | Light Chain Variable Domain (VL) |
| | QLQLQESGPGLVKPSETLSLTCT | SSELTQDPAVSVALGQTVRITCQG |
| | VSGGSISSGSFYWGWIRQPPGKG | DSLRNYFANWYQQKPGQAPVLVI |
| | LEWIGTIYYSGSTYYNPSLKSRV | YGKNNRPSGIPDRFSGSSSGNTAS |
| | AISVDTSKNQFYLNLNFVTAADT | LTITGAQAEDEADYYCNSRDSSG |
| | AVYHCARERSSSWYPEDYWGQG | YHVVFGGGTKLTVL (SEQ ID NO: |
| | TLVTVSS (SEQ ID NO: 124) | 128) |
| | CDR1-GGSISSGSFY (SEQ ID | CDR1-SLRNYF (SEQ ID NO: 129) |
| | NO: 125) | CDR2-GKN |
| | CDR2-IYYSGST (SEQ ID NO: | CDR3-NSRDSSGYHVV (SEQ ID |
| | 126) | NO: 131) |
| | CDR3-ARERSSSWYPFDY (SEQ | |
| | ID NO: 127) | |
| 88B06 | Heavy Chain | |
| | EVQLLESGGGLVQPGGSLRLSCAASGFTFSNYAMSWVRQAPGKGLEW | |
| | VSVISGSGDSTYYAESVKGRFTISRDNSKNTLYLQMTSLRAEDTAEYY | |
| | CAKDRTPVYGLDVWGQGTTVTVSSASTKGPSVFPLAPSSKSTSGGTAA | |
| | LGCLVKDYFPEPVTVSWNSGALTSGVHTFPAVLQSSGLYSLSSVVTVP | |
| | SSSLGTQTYICNVNHKPSNTKVDKRVEPKSCDKTHTCPPCPAPELLGGP | |
| | SVFLFPPKPKDTLMISRTPEVTCVVVDVSHEDPEVKFNWYVDGVEVH | |
| | NAKTKPREEQYNSTYRVVSVLTVLHQDWLNGKEYKCKVSNKALPAPI | |
| | EKTISKAKGQPREPQVYTLPPSREEMTKNQVSLTCLVKGFYPSDIAVE | |
| | WESNGQPENNYKTTPPVLDSDGSFFLYSKLTVDKSRWQQGNVFSCSV | |
| | MHEALHNHYTQKSLSLSPGK (SEQ ID NO: 20) | |
| | Light Chain | |
| | SYELTQPLSVSVALGQTARISCGGNNIGRKNVHWYQQKPGQAPVLVIY | |
| | GDSNRPSGIPERFSGSNSGNTATLTIGRAQAGDEADYYCQVWDSSTVF | |
| | GGGTKLTVLGQPKAAPSVTLFPPSSEELQANKATLVCLISDFYPGAVTV | |
| | AWKADSSPVKAGVETTTPSKQSNNKYAASSYLSLTPEQWKSHKSYSC | |
| | QVTHEGSTVEKTVAPTECS (SEQ ID NO: 19) | |
| | Heavy Chain Variable Domain (VH) | Light Chain Variable Domain (VL) |
| | EVQLLESGGGLVQPGGSLRLSCA | SYELTQPLSVSVALGQTARISCGG |
| | ASGFTFSNYAMSWVRQAPGKGL | NNIGRKNVHWYQQKPGQAPVLVI |
| | EWVSVISGSGDSTYYAESVKGRF | YGDSNRPSGIPERFSGSNSGNTAT |
| | TISRDNSKNTLYLQMTSLRAEDT | LTIGRAQAGDEADYYCQVWDSST |
| | AEYYCAKDRTPVYGLDVWGQG | VFGGGTKLTVL (SEQ ID NO: 136) |
| | TTVTVSS (SEQ ID NO: 132) | CDR1-NIGRKN (SEQ ID NO: 137) |
| | CDR1-GFTFSNYA (SEQ ID NO: | CDR2-GDS |
| | 133) | CDR3-QVWDSSTV (SEQ ID NO: |
| | CDR2-ISGSGDST (SEQ ID NO: | 139) |
| | 134) | |
| | CDR3-AKDRTPVYGLDV (SEQ | |
| | ID NO: 135) | |
| 95B06 | Heavy Chain | |
| | EVQLLESGGGLVQPGGSLRLSCAASGFTFSSYAMTWVRQAPGKGLEW | |
| | VSDISGSGGSTKYADSVKGRFTISRDNPKNTLYLQMNSLRAEDTAVYY | |
| | CAKDLGFYSTWDTDYWGQGTLVTVSSASTKGPSVFPLAPSSKSTSGGT | |
| | AALGCLVKDYFPEPVTVSWNSGALTSGVHTFPAVLQSSGLYSLSSVVT | |
| | VPSSSLGTQTYICNVNHKPSNTKVDKRVEPKSCDKTHTCPPCPAPELLG | |
| | GPSVFLFPPKPKDTLMISRTPEVTCVVVDVSHEDPEVKFNWYVDGVEV | |
| | HNAKTKPREEQYNSTYRVVSVLTVLHQDWLNGKEYKCKVSNKALPA | |
| | PIEKTISKAKGQPREPQVYTLPPSREEMTKNQVSLTCLVKGFYPSDIAV | |
| | EWESNGQPENNYKTTPPVLDSDGSFFLYSKLTVDKSRWQQGNVFSCS | |
| | VMHEALHNHYTQKSLSLSPGK (SEQ ID NO: 22) | |
| | Light Chain | |
| | DMQMTQSPSSLSASVGDRVTITCRASQGIRNDLDWYQQKPGKVPKRLI | |
| | HAASSLQSGVPSRFSGSGSGTEFTLTISSLQPEDFVTYYCLQHNTYPWT | |
| | FGQGTKLEIKRTVAAPSVFIFPPSDEQLKSGTASVVCLLNNFYPREAKV | |
| | QWKVDNALQSGNSQESVTEQDSKDSTYSLSSTLTLSKADYEKHKVYA | |
| | CEVTHQGLSSPVTKSFNRGEC (SEQ ID NO: 21) | |
| | Heavy Chain Variable Domain (VH) | Light Chain Variable Domain (VL) |
| | EVQLLESGGGLVQPGGSLRLSCA | DMQMTQSPSSLSASVGDRVTITC |
| | ASGFTFSSYAMTWVRQAPGKGL | RASQGIRNDLDWYQQKPGKVPK |
| | EWVSDISGSGGSTKYADSVKGRF | RLIHAASSLQSGVPSRFSGSGSGT |
| | TISRDNPKNTLYLQMNSLRAEDT | EFTLTISSLQPEDFVTYYCLQHNT |
| | AVYYCAKDLGFYSTWDTDYWG | YPWTFGQGTKLEIK (SEQ ID NO: |
| | QGTLVTVSS (SEQ ID NO: 140) | 144) |
| | CDR1-GFTFSSYA (SEQ ID NO: | CDR1-QGIRND (SEQ ID NO: 145) |
| | 141) | CDR2-AAS |
| | CDR2-ISGSGGST (SEQ ID NO: | CDR3-LQHNTYPWT (SEQ ID NO: |
| | 142) | 147) |
| | CDR3-AKDLGFYSTWDTDY | |
| | (SEQ ID NO: 143) | |
| 30F02 | Heavy Chain | |
| | QLQLQESGPGLVKPSETLSLTCTVSGASISSSNHYWGWGWIRQPPGKG | |
| | LEWIGSIYYSGNTYYNPSLKSRVTISVDTSKNQFSLKLSSVTAADTAVY | |
| | YCARHRGSYFFDHWGQGTLVTVSSASTKGPSVFPLAPSSKSTSGGTAA | |
| | LGCLVKDYFPEPVTVSWNSGALTSGVHTFPAVLQSSGLYSLSSVVTVP | |
| | SSSLGTQTYICNVNHKPSNTKVDKRVEPKSCDKTHTCPPCPAPELLGGP | |
| | SVFLFPPKPKDTLMISRTPEVTCVVVDVSHEDPEVKFNWYVDGVEVH | |
| | NAKTKPREEQYNSTYRVVSVLTVLHQDWLNGKEYKCKVSNKALPAPI | |
| | EKTISKAKGQPREPQVYTLPPSREEMTKNQVSLTCLVKGFYPSDIAVE | |
| | WESNGQPENNYKTTPPVLDSDGSFFLYSKLTVDKSRWQQGNVFSCSV | |
| | MHEALHNHYTQKSLSLSPGK (SEQ ID NO: 24) | |
| | Light Chain | |
| | SYVLTQPLSVSVALGQTARITCGGNNIGDKNVHWYQQKPGQAPVLVIS | |
| | RDSNRPSGIPERFSGSNSGNTATLTISRAQAGDESDYYCQVWDSSTVVF | |
| | GGGTKVTVLGQPKAAPSVTLFPPSSEELQANKATLVCLISDFYPGAVT | |
| | VAWKADSSPVKAGVETTTPSKQSNNKYAASSYLSLTPEQWKSHKSYS | |
| | CQVTHEGSTVEKTVAPTECS (SEQ ID NO: 23) | |
| | Heavy Chain Variable Domain (VH) | Light Chain Variable Domain (VL) |
| | QLQLQESGPGLVKPSETLSLTCT | SYVLTQPLSVSVALGQTARITCGG |
| | VSGASISSSNHYWGWGWIRQPP | NNIGDKNVHWYQQKPGQAPVLV |
| | GKGLEWIGSIYYSGNTYYNPSLK | ISRDSNRPSGIPERFSGSNSGNTAT |
| | SRVTISVDTSKNQFSLKLSSVTAA | LTISRAQAGDESDYYCQVWDSST |
| | DTAVYYCARHRGSYFFDHWGQ | VVFGGGTKVTVL (SEQ ID NO: |
| | GTLVTVSS (SEQ ID NO: 148) | 152) |
| | CDR1-GASISSSNHYWG (SEQ ID | CDR1-NIGDKN (SEQ ID NO: 153) |
| | NO: 149) | CDR2-RDS |
| | CDR2-IYYSGNT (SEQ ID NO: | CDR3-QVWDSSTVV (SEQ ID NO: |
| | 150) | 155) |
| | CDR3-ARHRGSYFFDH (SEQ ID | |
| | NO: 151) | |
| 85F12 | Heavy Chain | |
| | QVQLQQSGPGLVKPSQTLSLTCAISGDSVSSNSAVWNWIRQSPSRGLE | |
| | WLGRTDYRSKWNNDYAVSVKSRITINPDTSKNQFSLQLNSVTPEDTAV | |
| | YYCARGDAGMASFDYWGQGTLVTVSSASTKGPSVFPLAPSSKSTSGG | |
| | TAALGCLVKDYFPEPVTVSWNSGALTSGVHTFPAVLQSSGLYSLSSVV | |
| | TVPSSSLGTQTYICNVNHKPSNTKVDKRVEPKSCDKTHTCPPCPAPELL | |
| | GGPSVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKALP | |
| | APIEKTISKAKGQPREPQVYTLPPSREEMTKNQVSLTCLVKGFYPSDIA | |
| | VEWESNGQPENNYKTTPPVLDSDGSFFLYSKLTVDKSRWQQGNVFSC | |
| | SVMHEALHNHYTQKSLSLSPGK (SEQ ID NO: 26) | |
| | Light Chain | |
| | SSELTQPPSVSVSPGQTASITCSGDKLGDKYACWYQQKAGQSPVVVIY | |
| | QDRKRPSGIPERFSGSNSGNTATLTISGTQAMDEADYYCQTWDRRTAV | |
| | FGGGTKLTVLGQPKAAPSVTLFPPSSEELQANKATLVCLISDFYPGAVT | |
| | VAWKADSSPVKAGVETTTPSKQSNNKYAASSYLSLTPEQWKSHKSYS | |
| | CQVTHEGSTVEKTVAPTECS (SEQ ID NO: 25) | |
| | Heavy Chain Variable Domain (VH) | Light Chain Variable Domain (VL) |
| | QVQLQQSGPGLVKPSQTLSLTCA | SSELTQPPSVSVSPGQTASITCSGD |
| | ISGDSVSSNSAVWNWIRQSPSRG | KLGDKYACWYQQKAGQSPVVVI |
| | LEWLGRTDYRSKWNNDYAVSV | YQDRKRPSGIPERFSGSNSGNTAT |
| | KSRITINPDTSKNQFSLQLNSVTP | LTISGTQAMDEADYYCQTWDRR |
| | EDTAVYYCARGDAGMASFDYW | TAVFGGGTKLTVL (SEQ ID NO: |
| | GQGTLVTVSS (SEQ ID NO: 156) | 160) |
| | CDR1-GDSVSSNSAV (SEQ ID | CDR1-KLGDKY (SEQ ID NO: |
| | NO: 157) | 161) |
| | CDR2-TDYRSKWNN (SEQ ID | CDR2-QDR |
| | NO: 158) | CDR3-QTWDRRTAV (SEQ ID |
| | CDR3-ARGDAGMASFDY (SEQ | NO: 163) |
| | ID NO: 159) | |
| 85F10 | Heavy Chain | |
| | EVQLLESGAGLLKPSETLSLTCAVYGGSFSDYYWSWIRQPPGKGLEWI | |
| | GEINHSGSTNYNPSLKSRVTISVDTSKNQFSLKLSSVTAADTAVYYCAR | |
| | KRGANFFDDWGQGTLVTVSSASTKGPSVFPLAPSSKSTSGGTAALGCL | |
| | VKDYFPEPVTVSWNSGALTSGVHTFPAVLQSSGLYSLSSVVTVPSSSLG | |
| | TQTYICNVNHKPSNTKVDKRVEPKSCDKTHTCPPCPAPELLGGPSVFLF | |
| | PPKPKDTLMISRTPEVTCVVVDVSHEDPEVKFNWYVDGVEVHNAKTK | |
| | PREEQYNSTYRVVSVLTVLHQDWLNGKEYKCKVSNKALPAPIEKTISK | |
| | AKGQPREPQVYTLPPSREEMTKNQVSLTCLVKGFYPSDIAVEWESNGQ | |
| | PENNYKTTPPVLDSDGSFFLYSKLTVDKSRWQQGNVFSCSVMHEALH | |
| | NHYTQKSLSLSPGK (SEQ ID NO: 28) | |
| | Light Chain | |
| | SYELTQPLSVSVALGQTARITCGGNNIENKNVHWYQQKPGQAPVLVIK | |
| | RDSNRPSGIPERLSGSNSGNTATLTISRAQAGDEADYYCQVWDSSTVV | |
| | FGGGTKLTVLGQPKAAPSVTLFPPSSEELQANKATLVCLISDFYPGAVT | |
| | VAWKADSSPVKAGVETTTPSKQSNNKYAASSYLSLTPEQWKSHKSYS | |
| | CQVTHEGSTVEKTVAPTECS (SEQ ID NO: 27) | |
| | Heavy Chain Variable Domain (VH) | Light Chain Variable Domain (VL) |
| | EVQLLESGAGLLKPSETLSLTCA | SYELTQPLSVSVALGQTARITCGG |
| | VYGGSFSDYYWSWIRQPPGKGL | NNIENKNVHWYQQKPGQAPVLVI |
| | EWIGEINHSGSTNYNPSLKSRVTI | KRDSNRPSGIPERLSGSNSGNTAT |
| | SVDTSKNQFSLKLSSVTAADTAV | LTISRAQAGDEADYYCQVWDSST |
| | YYCARKRGANFFDDWGQGTLV | VVFGGGTKLTVL (SEQ ID NO: |
| | TVSS (SEQ ID NO: 164) | 168) |
| | CDR1-GGSFSDYY (SEQ ID NO: | CDR1-NIENKN (SEQ ID NO: 89) |
| | 165) | CDR2-RDS |
| | CDR2-INHSGST (SEQ ID NO: | CDR3-QVWDSSTVV (SEQ ID NO: |
| | 166) | 155) |
| | CDR3-ARKRGANFFDD (SEQ ID | |
| | NO: 167) | |
| Anti-OX40L | Heavy Chain | |
| (U.S. Pat. | EVQLVESGGGLVQPGGSLRLSCAASGFTFSNYAMNWVRQAPGKGLE | |
| No. | WVSTISGSGGATRYADSVKGRFTISRDNSRNTVYLQMNSLRVEDTAVF | |
| 9,139,653) | YCTKDRLIMATVRGPYYYGMDVWGQGTTVTVSSASTKGPSVFPLAPC | |
| | SRSTSESTAALGCLVKDYFPEPVTVSWNSGALTSGVHTFPAVLQSSGL | |
| | YSLSSVVTVPSSSLGTKTYTCNVDHKPSNTKVDKRVESKYGPPCPPCP | |
| | APEFEGGPSVFLFPPKPKDTLMISRTPEVTCVVVDVSQEDPEVQFNWY | |
| | VDGVEVHNAKTKPREEQFNSTYRVVSVLTVLHQDWLNGKEYKCKVS | |
| | NKGLPSSIEKTISKAKGQPREPQVYTLPPSQEEMTKNQVSLTCLVKGFY | |
| | PSDIAVEWESNGQPENNYKTTPPVLDSDGSFFLYSRLTVDKSRWQEGN | |
| | VFSCSVMHEALHNHYTQKSLSLSLGK (SEQ ID NO: 252) | |
| | Light Chain | |
| | DIQMTQSPSSLSASVGDRVTITCRASQSISSYLNWYQQKPGKAPNLLIY | |
| | AASSLQSGVPSRFSGSGSETDFTLTISSLQPEDFATYYCQQSHSVSFTFG | |
| | PGTKVDIKRTVAAPSVFIFPPSDEQLKSGTASVVCLLNNFYPREAKVQ | |
| | WKVDNALQSGNSQESVTEQDSKDSTYSLSSTLTLSKADYEKHKVYAC | |
| | EVTHQGLSSPVTKSFNRGEC (SEQ ID NO: 253) | |
| | Heavy Chain Variable Domain (VH) | Light Chain Variable Domain (VL) |
| | EVQLVESGGGLVQPGGSLRLSCA | DIQMTQSPSSLSASVGDRVTITCR |
| | ASGFTFSNYAMNWVRQAPGKGL | ASQSISSYLNWYQQKPGKAPNLLI |
| | EWVSTISGSGGATRYADSVKGRF | YAASSLQSGVPSRFSGSGSETDFT |
| | TISRDNSRNTVYLQMNSLRVEDT | LTISSLQPEDFATYYCQQSHSVSF |
| | AVFYCTKDRLIMATVRGPYYYG | TFGPGTKVDIK (SEQ ID NO: 30) |
| | MDVWGQGTTVTVSS (SEQ ID | CDR1-QSISSY (SEQ ID NO: 121) |
| | NO: 31) | CDR2-AAS |
| | CDR1-GFTFSNYA (SEQ ID NO: | CDR3-QQSHSVSFT (SEQ ID NO: |
| | 133) | 154) |
| | CDR2-ISGSGGAT (SEQ ID NO: | |
| | 113) | |
| | CDR3- | |
| | TKDRLIMATVRGPYYYGMDV | |
| | (SEQ ID NO: 114) | |
| Anti-OX40L | Heavy Chain | |
| (U.S. Pat. | EVQLLESGGGLVQPGGSLRLSCAASGFTFNSYAMSWVRQAPGKGLEW | |
| No. | VSIISGSGGFTYYADSVKGRFTISRDNSRTTLYLQMNSLRAEDTAVYYC | |
| 7,501,496) | AKDRLVAPGTFDYWGQGALVTVSSASTKGPSVFPLAPSSKSTSGGTAA | |
| *Based on | LGCLVKDYFPEPVTVSWNSGALTSGVHTFPAVLQSSGLYSLSSVVTVP | |
| Kabat | SSSLGTQTYICNVNHKPSNTKVDKKVEPKSCDKTHTCPPCPAPELLGGP | |
| Numbering | SVFLFPPKPKDTLMISRTPEVTCVVVDVSHEDPEVKFNWYVDGVEVH | |
| | NAKTKPREEQYNSTYRVVSVLTVLHQDWLNGKEYKCKVSNKALPAPI | |
| | EKTISKAKGQPREPQVYTLPPSRDELTKNQVSLTCLVKGFYPSDIAVEW | |
| | ESNGQPENNYKTTPPVLDSDGSFFLYSKLTVDKSRWQQGNVFSCSVM | |
| | HEALHNHYTQKSLSLSPG (SEQ ID NO: 254) | |
| | Light Chain | |
| | DIQMTQSPSSLSASVGDRVTITCRASQGISSWLAWYQQKPEKAPKSLIY | |
| | AASSLQSGVPSRFSGSGSGTDFTLTISSLQPEDFATYYCQQYNSYPYTF | |
| | GQGTKLEIKRTVAAPSVFIFPPSDEQLKSGTASVVCLLNNFYPREAKVQ | |
| | WKVDNALQSGNSQESVTEQDSKDSTYSLSSTLTLSKADYEKHKVYAC | |
| | EVTHQGLSSPVTKSFNRGEC (SEQ ID NO: 255) | |
| | Heavy Chain Variable Domain (VH) | Light Chain Variable Domain (VL) |
| | EVQLLESGGGLVQPGGSLRLSCA | DIQMTQSPSSLSASVGDRVTITCR |
| | ASGFTFNSYAMSWVRQAPGKGL | ASQGISSWLAWYQQKPEKAPKSL |
| | EWVSIISGSGGFTYYADSVKGRF | IYAASSLQSGVPSRFSGSGSGTDF |
| | TISRDNSRTTLYLQMNSLRAEDT | TLTISSLQPEDFATYYCQQYNSYP |
| | AVYYCAKDRLVAPGTFDYWGQ | YTFGQGTKLEIK (SEQ ID NO: 32) |
| | GALVTVSS (SEQ ID NO: 33) | CDR1-RASQGISSWLA (SEQ ID |
| | CDR1-SYAMS (SEQ ID NO: 105) | NO: 169) |
| | CDR2-IISGSGGFTYYADSVK | CDR2-AASSLQS (SEQ ID NO: |
| | (SEQ ID NO: 106) | 170) |
| | CDR3-DRLVAPGTFDY (SEQ ID | CDR3-QQYNSYPYT (SEQ ID NO: |
| | NO: 107) | 171) |

In certain embodiments, the antigen-binding site that specifically binds OX40L comprises an antibody heavy chain (HC) that comprises an amino acid sequence at least 90% (e.g., at least 91%, at least 92%, at least 93%, at least 94%, at least 95%, at least 96%, at least 97%, at least 98%, at least 99%, or 100%) identical to the HC of an antibody disclosed in TABLE 3, and an antibody light chain (LC) that comprises an amino acid sequence at least 90% (e.g., at least 91%, at least 92%, at least 93%, at least 94%, at least 95%, at least 96%, at least 97%, at least 98%, at least 99%, or 100%) identical to the LC of the same antibody disclosed in TABLE 3. In certain embodiments, the antigen-binding site that specifically binds OX40L comprises an antibody heavy chain variable domain (VH) that comprises an amino acid sequence at least 90% (e.g., at least 91%, at least 92%, at least 93%, at least 94%, at least 95%, at least 96%, at least 97%, at least 98%, at least 99%, or 100%) identical to the VH of an antibody disclosed in TABLE 3, and an antibody light chain variable domain (VL) that comprises an amino acid sequence at least 90% (e.g., at least 91%, at least 92%, at least 93%, at least 94%, at least 95%, at least 96%, at least 97%, at least 98%, at least 99%, or 100%) identical to the VL of the same antibody disclosed in TABLE 3. In certain embodiments, the antigen-binding site comprises the heavy chain CDR1, CDR2, and CDR3 and the light chain CDR1, CDR2, and CDR3, determined under IMGT unique numbering scheme, Kabat (see Kabat et al., (1991) Sequences of Proteins of Immunological Interest, NIH Publication No. 91-3242, Bethesda), Chothia (see, e.g., Chothia C & Lesk A M, (1987), J. Mol. Biol. 196: 901-917), MacCallum (see MacCallum R M et al., (1996) J. Mol. Biol. 262: 732-745), or any other CDR determination method known in the art, of the VH and VL sequences disclosed in TABLE 3.

In certain embodiments, the antigen-binding site that specifically binds to OX40L comprises a heavy chain complementarity-determining region 1 (VHCDR1) comprising an amino acid sequence of GGSISTSSYY (SEQ ID NO: 77), a heavy chain complementarity-determining region 2 (VHCDR2) comprising an amino acid sequence of IYYSGST (SEQ ID NO: 78), a heavy chain complementarity-determining region 3 (VHCDR3) comprising an amino acid sequence of ARHRGSYFFDI (SEQ ID NO: 79), a light chain complementarity-determining region 1 (VLCDR1) comprising an amino acid sequence of DIENKN (SEQ ID NO: 81), a light chain complementarity-determining region 2 (VLCDR2) comprising an amino acid sequence of RDN, and a light chain complementarity-determining region 3 (VLCDR3) comprising an amino acid sequence of QVRDSNIVV (SEQ ID NO: 83).

In some embodiments, the antigen-binding site that specifically binds to OX40L comprises a VHCDR1 comprising an amino acid sequence of GVSIRSNGYY (SEQ ID NO: 93), a VHCDR2 comprising an amino acid sequence of MDYSGT (SEQ ID NO: 94), a VHCDR3 comprising an amino acid sequence of ARERSNNWYPIDY (SEQ ID NO: 95), a VLCDR1 comprising an amino acid sequence of SVRRFF (SEQ ID NO: 97), a VLCDR2 comprising an amino acid sequence of GKD, and a VLCDR3 comprising an amino acid sequence of NSRDSSGYLVL (SEQ ID NO: 99).

In some embodiments, the antigen-binding site that specifically binds to OX40L comprises a VHCDR1 comprising an amino acid sequence of GASVSSSSYY (SEQ ID NO: 85), a VHCDR2 comprising an amino acid sequence of INYGGST (SEQ ID NO: 86), a VHCDR3 comprising an amino acid sequence of ARHRGIYHFDY (SEQ ID NO: 87), a VLCDR1 comprising an amino acid sequence of NIENKN (SEQ ID NO: 89), a VLCDR2 comprising an amino acid sequence of RDS, and a VLCDR3 comprising an amino acid sequence of QVWDSNTVV (SEQ ID NO: 91).

In some embodiments, the antigen-binding site that specifically binds to OX40L comprises a VHCDR1 comprising an amino acid sequence of GGSISSSSYY (SEQ ID NO: 101), a VHCDR2 comprising an amino acid sequence of IGSVDYSGNT (SEQ ID NO: 102), a VHCDR3 comprising an amino acid sequence of ARHRGIYFFDY (SEQ ID NO: 103), a VLCDR1 comprising an amino acid sequence of NIENKN (SEQ ID NO: 89), a VLCDR2 comprising an amino acid sequence of RDS, and a VLCDR3 comprising an amino acid sequence of QVWDSNTVV (SEQ ID NO: 91).

In some embodiments, the antigen-binding site that specifically binds to OX40L comprises a VHCDR1 comprising an amino acid sequence of GFTFSNYA (SEQ ID NO: 133), a VHCDR2 comprising an amino acid sequence of ISGSGGAT (SEQ ID NO: 113), a VHCDR3 comprising an amino acid sequence of TKDRLIMATVRGPYYYGMDV (SEQ ID NO: 114), a VLCDR1 comprising an amino acid sequence of QSISSY (SEQ ID NO: 121), a VLCDR2 comprising an amino acid sequence of AAS, and a VLCDR3 comprising an amino acid sequence of QQSHSVSFT (SEQ ID NO: 154).

In some embodiments, the antigen-binding site that specifically binds to OX40L comprises a VHCDR1 comprising an amino acid sequence of SYAMS (SEQ ID NO: 105), a VHCDR2 comprising an amino acid sequence of IISGSGGFTYYADSVK (SEQ ID NO: 106), a VHCDR3 comprising an amino acid sequence of DRLVAPGTFDY (SEQ ID NO: 107), a VLCDR1 comprising an amino acid sequence of RASQGISSWLA (SEQ ID NO: 169), a VLCDR2 comprising an amino acid sequence of AASSLQS (SEQ ID NO: 170), and a VLCDR3 comprising an amino acid sequence of QQYNSYPYT (SEQ ID NO: 171).

In certain embodiments, the antigen-binding site that specifically binds OX40L comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 76, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 80.

In certain embodiments, the antigen-binding site that specifically binds OX40L comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 92, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 96.

In certain embodiments, the antigen-binding site that specifically binds OX40L comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 84, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 88.

In certain embodiments, the antigen-binding site that specifically binds OX40L comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 100, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 104.

In certain embodiments, the antigen-binding site that specifically binds OX40L comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 31, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 30.

In certain embodiments, the antigen-binding site that specifically binds OX40L comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 33, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 32.

In certain embodiments, the antigen-binding site that specifically binds OX40L comprises a HC that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 8, and a LC that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 7.

In certain embodiments, the antigen-binding site that specifically binds OX40L comprises a HC that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 16, and a LC that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 15.

In certain embodiments, the antigen-binding site that specifically binds OX40L comprises a HC that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 14, and a LC that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 13.

In certain embodiments, the antigen-binding site that specifically binds OX40L comprises a HC that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 18, and a LC that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 17.

In certain embodiments, the antigen-binding site that specifically binds OX40L comprises a HC that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 252, and a LC that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 253.

In certain embodiments, the antigen-binding site that specifically binds OX40L comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 254, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 255.

### I.C Bridging Moieties

In some embodiments, proteins of the present disclosure further comprise a bridging moiety. In certain embodiments, the bridging moiety may be non-functional, i.e. merely serves as a structural connection and/or appendage and does not perform a biological function or have a biological purpose. In other embodiments, the bridging moiety is functional and has a biological function in the context of the protein.

In some embodiments, the N-terminus of the bridging moiety is connected to the C-terminus of a polypeptide or complex of two or more polypeptides that specifically binds CD80 and/or CD86 and the C-terminus of the bridging moiety is connected to the N-terminus of a polypeptide or complex of two or more polypeptides that specifically binds OX40L. In some embodiments, the bridging moiety connects the polypeptide or heavy chain of a complex of two or more polypeptides that specifically binds CD80 and/or CD86 and the polypeptide or heavy chain of a complex of two or more polypeptides that specifically binds OX40L to form a contiguous polypeptide chain.

In some embodiments, the bridging moiety is connected to the C-terminus of a polypeptide or complex of two or more polypeptides that specifically binds OX40L. In other embodiments, the bridging moiety is connected to the C-terminus of a polypeptide or complex of two or more polypeptides that specifically binds CD80 and/or CD86. In some embodiments, the bridging moiety is connected to the N-terminus of a polypeptide or complex of two or more polypeptides that specifically binds OX40L. In other embodiments, the bridging moiety is connected to the N-terminus of a polypeptide or complex of two or more polypeptides that specifically binds CD80 and/or CD86.

In some embodiments, the bridging moiety comprises, but is not limited to, a polypeptide of an immunoglobulin Fc domain or functional fragment thereof, a human serum albumin (HSA) polypeptide or functional fragment thereof, a polypeptide linker, or a polypeptide hinge. In certain embodiments, the bridging moiety comprises a polypeptide of an immunoglobulin Fc domain. For example, in some embodiments, the bridging moiety is a polypeptide of an IgG, IgM, IgA, IgD, or IgE Fc domain. In certain embodiments, the bridging moiety is a polypeptide of an IgG1, IgG2, IgG3, or IgG4 Fc domain. In certain embodiments, the bridging moiety is a polypeptide of an IgG1 Fc domain. In certain embodiments, the bridging moiety is a polypeptide of a human IgG1 Fc domain. In certain embodiments, proteins of the present disclosure comprise a first polypeptide of an immunoglobulin Fc domain and a second polypeptide of an immunoglobulin Fc domain which dimerize with each other.

### Human IgG Fc

Within the polypeptide of the Fc domain, CD16 binding is mediated by the hinge region and the CH2 domain. For example, within human IgG1, the interaction with CD16 is primarily focused on amino acid residues Asp 265-Glu 269, Asn 297-Thr 299, Ala 327-Ile 332, Leu 234-Ser 239, and carbohydrate residue N-acetyl-D-glucosamine in the CH2 domain (see, Sondermann et al., Nature, 406 (6793):267-273), numbered according to the EU index as in Kabat. Based on the known domains, mutations can be selected to enhance or reduce the binding affinity to CD16, such as by using phage-displayed libraries or yeast surface-displayed cDNA libraries, or can be designed based on the known three-dimensional structure of the interaction. Accordingly, in certain embodiments, a polypeptide of the immunoglobulin Fc domain or the portion thereof comprises a hinge polypeptide and a CH2 domain.

In certain embodiments, the bridging moiety is a polypeptide of a human IgG1 Fc domain comprising one or more mutation(s) to reduce binding to an Fcγ receptor (e.g., FcγRI, FcγRIIA, FcγRIIB, FcγRIIIA, or FcγRIIIB) or a complement component (e.g., C1q) in the first and/or second polypeptides of the human IgG1 Fc domain. Such mutations are useful for reducing effector functions. For example, a protein of the present disclosure includes LALA (L234A and L235A) mutations, LALAPA (L234A, L235A, and P329A) mutations, LALAPG (L234A, L235A, and P329G) mutations, or LALEGAASPS (L234A, L235E, G237A, A330S, and P331S) mutations. In some embodiments the terminal lysine residue of human IgG1 Fc domain is mutated (K447A) or deleted (K447Δ). In some embodiments, amino acids at any one or more of positions 322, 330, 331, 355, and 358 may be mutated. The positions of the amino acid substitutions are all numbered according to the EU index as in Kabat, unless otherwise stated.

TABLE 4 lists exemplary wildtype and mutant polypeptides of human immunoglobulin Fc domains. In some embodiments, the bridging moiety comprises an amino acid sequence selected from any one of the human immunoglobulin Fc sequences listed in TABLE 4.

TABLE 4
Exemplary human IgG Fc domain sequences

| Fc domain | Amino Acid Sequence |
|---|---|
| IgG1Fc wildtype | PSVFLFPPKPKDTLMISRTPEVTCVVVDVSHEDPEVKFNWYVDG |
| | VEVHNAKTKPREEQYNSTYRVVSVLTVLHQDWLNGKEYKCKV |
| | SNKALPAPIEKTISKAKGQPREPQVYTLPPSRDELTKNQVSLTCL |
| | VKGFYPSDIAVEWESNGQPENNYKTTPPVLDSDGSFFLYSKLTV |
| | DKSRWQQGNVFSCSVMHEALHNHYTQKSLSLSPGK (SEQ ID |
| | NO: 178) |
| IgG1 Fc PA | PSVFLFPPKPKDTLMISRTPEVTCVVVDVSHEDPEVKFNWYVDG |
| | VEVHNAKTKPREEQYNSTYRVVSVLTVLHQDWLNGKEYKCKV |
| | SNKALAAPIEKTISKAKGQPREPQVYTLPPSRDELTKNQVSLTCL |
| | VKGFYPSDIAVEWESNGQPENNYKTTPPVLDSDGSFFLYSKLTV |
| | DKSRWQQGNVFSCSVMHEALHNHYTQKSLSLSPGK (SEQ ID |
| | NO: 237) |
| IgG1 Fc PG | PSVFLFPPKPKDTLMISRTPEVTCVVVDVSHEDPEVKFNWYVDG |
| | VEVHNAKTKPREEQYNSTYRVVSVLTVLHQDWLNGKEYKCKV |
| | SNKALGAPIEKTISKAKGQPREPQVYTLPPSRDELTKNQVSLTCL |
| | VKGFYPSDIAVEWESNGQPENNYKTTPPVLDSDGSFFLYSKLTV |
| | DKSRWQQGNVFSCSVMHEALHNHYTQKSLSLSPGK (SEQ ID |
| | NO: 238) |
| IgG1.4 Fc | PSVFLFPPKPKDTLMISRTPEVTCVVVDVSHEDPEVKFNWYVDG |
| | VEVHNAKTKPREEQYNSTYRVVSVLTVLHQDWLNGKEYKCAV |
| | SNKALPSSIEKTISKAKGQPREPQVYTLPPSREEMTKNQVSLTCL |
| | VKGFYPSDIAVEWESNGQPENNYKTTPPVLDSDGSFFLYSKLTV |
| | DKSRWQQGNVFSCSVMHEALHNHYTQKSLSLSPGA (SEQ ID |
| | NO: 214) |
| IgG1.6 Fc | PSVFLFPPKPKDTLMISRTPEVTCVVVDVSHEDPEVKFNWYVDG |
| | VEVHNAKTKPREEQYNSTYRVVSVLTVLHQDWLNGKEYKCAV |
| | SNKALAAPIEKTISKAKGQPREPQVYTLPPSREEMTKNQVSLTCL |
| | VKGFYPSDIAVEWESNGQPENNYKTTPPVLDSDGSFFLYSKLTV |
| | DKSRWQQGNVFSCSVMHEALHNHYTQKSLSLSPGA (SEQ ID |
| | NO: 216) |
| IgG1 Fc K447A | PSVFLFPPKPKDTLMISRTPEVTCVVVDVSHEDPEVKFNWYVDG |
| | VEVHNAKTKPREEQYNSTYRVVSVLTVLHQDWLNGKEYKCKV |
| | SNKALPAPIEKTISKAKGQPREPQVYTLPPSREEMTKNQVSLTCL |
| | VKGFYPSDIAVEWESNGQPENNYKTTPPVLDSDGSFFLYSKLTV |
| | DKSRWQQGNVFSCSVMHEALHNHYTQKSLSLSPGA (SEQ ID |
| | NO: 179) |
| IgG1 Fc K4474 | PSVFLFPPKPKDTLMISRTPEVTCVVVDVSHEDPEVKFNWYVDG |
| | VEVHNAKTKPREEQYNSTYRVVSVLTVLHQDWLNGKEYKCKV |
| | SNKALPAPIEKTISKAKGQPREPQVYTLPPSREEMTKNQVSLTCL |
| | VKGFYPSDIAVEWESNGQPENNYKTTPPVLDSDGSFFLYSKLTV |
| | DKSRWQQGNVFSCSVMHEALHNHYTQKSLSLSPG (SEQ ID NO: |
| | 180) |
| Orencia IgG | PSPAPELLGGSSVFLFPPKPKDTLMISRTPEVTCVVVDVSHEDPE |
| | VKFNWYVDGVEVHNAKTKPREEQYNSTYRVVSVLTVLHQDWL |
| | NGKEYKCKVSNKALPAPIEKTISKAKGQPREPQVYTLPPSRDELT |
| | KNQVSLTCLVKGFYPSDIAVEWESNGQPENNYKTTPPVLDSDGS |
| | FFLYSKLTVDKSRWQQGNVFSCSVMHEALHNHYTQKSLSLSPG |
| | K (SEQ ID NO: 239) |
| CTLA4Ig Variant Fc | PSPAPELLGGSSVFLFPPKPKDTLMISRTPEVTCVVVDVSHEDPE |
| | VKFNWYVDGVEVHNAKTKPREEQYNSTYRVVSVLTVLHQDWL |
| | NGKEYKCKVSNKALPAPIEKTISKAKGQPREPQVYTLPPSREEM |
| | TKNQVSLTCLVKGFYPSDIAVEWESNGQPENNYKTTPPVLDSDG |
| | SFFLYSKLTVDKSRWQQGNVFSCSVMHEALHNHYTQKSLSLSP |
| | G (SEQ ID NO: 240) |
| IgG2 Fc wildtype | PSVFLFPPKPKDTLMISRTPEVTCVVVDVSHEDPEVQFNWYVDG |
| | VEVHNAKTKPREEQFNSTFRVVSVLTVVHQDWLNGKEYKCKV |
| | SNKGLPAPIEKTISKTKGQPREPQVYTLPPSREEMTKNQVSLTCL |
| | VKGFYPSDISVEWESNGQPENNYKTTPPMLDSDGSFFLYSKLTV |
| | DKSRWQQGNVFSCSVMHEALHNHYTQKSLSLSPGK (SEQ ID |
| | NO: 217) |
| IgG3 Fc wildtype | PSVFLFPPKPKDTLMISRTPEVTCVVVDVSHEDPEVQFKWYVDG |
| | VEVHNAKTKPREEQYNSTFRVVSVLTVLHQDWLNGKEYKCKV |
| | SNKALPAPIEKTISKTKGQPREPQVYTLPPSREEMTKNQVSLTCL |
| | VKGFYPSDIAVEWESSGQPENNYNTTPPMLDSDGSFFLYSKLTV |
| | DKSRWQQGNIFSCSVMHEALHNRFTQKSLSLSPG (SEQ ID NO: |
| | 241) |
| IgG4 Fc wildtype | PSVFLFPPKPKDTLMISRTPEVTCVVVDVSQEDPEVQFNWYVDG |
| | VEVHNAKTKPREEQFNSTYRVVSVLTVLHQDWLNGKEYKCKV |
| | SNKGLPSSIEKTISKAKGQPREPQVYTLPPSQEEMTKNQVSLTCV |
| | KGFYPSDIAVEWESNGQPENNYKTTPPVLDSDGSFFLYSKLTVD |
| | KSRWQEGNVFSCSVMHEALHNHYTQKSLSLSLG (SEQ ID NO: |
| | 205) |

In certain embodiments, proteins of the present invention comprise a polypeptide of a human IgG Fc domain bridging moiety that comprises an amino acid sequence at least 90% (e.g., at least 91%, at least 92%, at least 93%, at least 94%, at least 95%, at least 96%, at least 97%, at least 98%, at least 99%, or 100%) identical to a wildtype or variant human IgG Fc domain sequence listed in TABLE 4, numbered according to the EU index as in Kabat.

In certain embodiments, proteins of the present invention comprise a polypeptide of a human IgG1 Fc domain bridging moiety that comprises an amino acid at least 90% (e.g., at least 91%, at least 92%, at least 93%, at least 94%, at least 95%, at least 96%, at least 97%, at least 98%, at least 99%, or 100%) identical to SEQ ID NO: 178.

In certain embodiments, proteins of the present invention comprise a polypeptide of a human IgG1 Fc domain bridging moiety that comprises an amino acid at least 90% (e.g., at least 91%, at least 92%, at least 93%, at least 94%, at least 95%, at least 96%, at least 97%, at least 98%, at least 99%, or 100%) identical to SEQ ID NO: 237.

In certain embodiments, proteins of the present invention comprise a polypeptide of a human IgG1 Fc domain bridging moiety that comprises an amino acid at least 90% (e.g., at least 91%, at least 92%, at least 93%, at least 94%, at least 95%, at least 96%, at least 97%, at least 98%, at least 99%, or 100%) identical to SEQ ID NO: 238.

In certain embodiments, proteins of the present invention comprise a polypeptide of a human IgG1 Fc domain bridging moiety that comprises an amino acid at least 90% (e.g., at least 91%, at least 92%, at least 93%, at least 94%, at least 95%, at least 96%, at least 97%, at least 98%, at least 99%, or 100%) identical to SEQ ID NO: 214.

In certain embodiments, proteins of the present invention comprise a polypeptide of a human IgG1 Fc domain bridging moiety that comprises an amino acid at least 90% (e.g., at least 91%, at least 92%, at least 93%, at least 94%, at least 95%, at least 96%, at least 97%, at least 98%, at least 99%, or 100%) identical to SEQ ID NO: 216.

In certain embodiments, proteins of the present invention comprise a polypeptide of a human IgG1 Fc domain bridging moiety that comprises an amino acid at least 90% (e.g., at least 91%, at least 92%, at least 93%, at least 94%, at least 95%, at least 96%, at least 97%, at least 98%, at least 99%, or 100%) identical to SEQ ID NO: 179.

In certain embodiments, proteins of the present invention comprise a polypeptide of a human IgG1 Fc domain bridging moiety that comprises an amino acid at least 90% (e.g., at least 91%, at least 92%, at least 93%, at least 94%, at least 95%, at least 96%, at least 97%, at least 98%, at least 99%, or 100%) identical to SEQ ID NO: 180.

In certain embodiments, proteins of the present invention comprise a polypeptide of a human IgG1 Fc domain bridging moiety that comprises an amino acid at least 90% (e.g., at least 91%, at least 92%, at least 93%, at least 94%, at least 95%, at least 96%, at least 97%, at least 98%, at least 99%, or 100%) identical to SEQ ID NO: 239.

In certain embodiments, proteins of the present invention comprise a polypeptide of a human IgG1 Fc domain bridging moiety that comprises an amino acid at least 90% (e.g., at least 91%, at least 92%, at least 93%, at least 94%, at least 95%, at least 96%, at least 97%, at least 98%, at least 99%, or 100%) identical to SEQ ID NO: 240.

### Hinge Polypeptide

In some embodiments, the bridging moiety comprises at least a portion of a hinge polypeptide. The hinge polypeptide can be derived from an immunoglobulin heavy chain, e.g., IgG1, IgG2, IgG3, IgG4, or other classes. Preferably, the hinge region is derived from human IgG1, IgG2, IgG3, or IgG4. More preferably the hinge region is derived from a human IgG1 heavy chain.

In some embodiments, at least a portion of hinge polypeptide is connected to the N-terminus of the heavy chain constant domain 2 (CH2) of a polypeptide of a human immunoglobulin Fc domain. In some embodiments, the hinge polypeptide connects the C-terminus of a polypeptide or complex of two or more polypeptides that specifically binds CD80 and/or CD86 to the N-terminus of the CH2 domain of a polypeptide of a human immunoglobulin Fc domain. In other embodiments, the hinge polypeptide connects the C-terminus of a polypeptide or complex of two or more polypeptides that specifically binds OX40L to the N-terminus of the CH2 domain of a polypeptide of a human immunoglobulin Fc domain. In some embodiments, a partial hinge (for example, a polypeptide having the sequence of SEQ ID NO: 336) is connected to the C-terminus of a CH1 domain of a Fab heavy chain (HC) that specifically binds OX40L and forms a disulfide bond with the Fab light chain (LC) that specifically binds OX40L. In some embodiments, a partial hinge (for example, a polypeptide having the sequence of SEQ ID NO: 336) is connected to the C-terminus of a CH1 domain of a Fab heavy chain (HC) that specifically binds CD80 or CD86 and forms a disulfide bond with the Fab light chain (LC) that specifically binds CD80 or CD86, respectively.

In some embodiments, amino acids at any one or more of positions C220, E233, L234 or L235 may be mutated in the hinge region of human IgG1. The positions of the amino acid substitutions are all numbered according to the EU index as in Kabat, unless otherwise stated.

TABLE 5 lists exemplary wildtype and mutant human immunoglobulin hinge polypeptides. In some embodiments, the bridging moiety comprises an amino acid sequence selected from any one of the human immunoglobulin Fc hinge polypeptide sequences listed in TABLE 5.

TABLE 5
Hinge Polypeptides of Human Immunoglobulin

| Hinge | Amino Acid Sequence |
|---|---|
| IgG1 wildtype hinge | EPKSCDKTHTCPPCPAPELLGG (SEQ ID NO: 175) |
| IgG1 wildtype Q | QEPKSCDKTHTCPPCPAPELLGG (SEQ ID NO: 242) |
| hinge | |
| IgG1 hinge C220S | EPKSSDKTHTCPPCPAPELLGG (SEQ ID NO: 176) |
| IgG1 hinge C220S Q | QEPKSSDKTHTCPPCPAPELLGG (SEQ ID NO: 249) |
| IgG1 hinge C220S/ | EPKSSDKTHTCPPCPAPEAAGG (SEQ ID NO: 177) |
| L234A/L235A | |
| IgG1 hinge Q C220S/ | QEPKSSDKTHTCPPCPAPEAAGG (SEQ ID NO: 243) |
| L234A/L235A | |
| IgG1 hinge L234A/ | EPKSCDKTHTCPPCPAPEAAGG (SEQ ID NO: 212) |
| L235A (and LALAPA) | |
| IgG1 hinge Q L234A/ | QEPKSCDKTHTCPPCPAPEAAGG (SEQ ID NO: 244) |
| L235A (and LALAPA) | |
| IgG1.4 hinge | EPKSSDKTHTCPPCPAPPVAG (SEQ ID NO: 213) |
| IgG1.4 hinge Q | QEPKSSDKTHTCPPCPAPPVAG (SEQ ID NO: 245) |
| IgG2 wildtype hinge | ERKCCVECPPCPAPPVAG (SEQ ID NO: 246) |
| IgG4 wildtype hinge | ESKYGPPCPSCPAPEFLGG (SEQ ID NO: 218) |
| IgG4 hinge consensus | RVESKYGPPCPPCPAPEFXGG |
| | wherein X is L or E (SEQ ID NO: 202) |
| IgG4 hinge S228P | RVESKYGPPCPPCPAPEFLGG (SEQ ID NO: 203) |
| IgG4 hinge S228P/ | RVESKYGPPCPPCPAPEFEGG (SEQ ID NO: 204) |
| L235E | |
| Orencia IgG hinge | QEPKSSDKTHTSP (SEQ ID NO: 247) |
| Orencia Hinge variant | EPKSSDKTHTSP (SEQ ID NO: 248) |
| IgG1 Partial Hinge | EPKSC (SEQ ID NO: 336) |

In some embodiments, bridging moieties of the present invention comprise a hinge polypeptide comprising an amino acid sequence at least 90% (e.g., at least 91%, at least 92%, at least 93%, at least 94%, at least 95%, at least 96%, at least 97%, at least 98%, at least 99%, or 100%) identical to a wildtype or variant human immunoglobulin Fc hinge sequence listed in TABLE 5, numbered according to the EU index as in Kabat.

In certain embodiments, bridging moieties of the present invention comprise a hinge polypeptide comprising an amino acid at least 90% (e.g., at least 91%, at least 92%, at least 93%, at least 94%, at least 95%, at least 96%, at least 97%, at least 98%, at least 99%, or 100%) identical to SEQ ID NO: 175.

In certain embodiments, bridging moieties of the present invention comprise a hinge polypeptide comprising an amino acid at least 90% (e.g., at least 91%, at least 92%, at least 93%, at least 94%, at least 95%, at least 96%, at least 97%, at least 98%, at least 99%, or 100%) identical to SEQ ID NO: 242.

In certain embodiments, bridging moieties of the present invention comprise a hinge polypeptide comprising an amino acid at least 90% (e.g., at least 91%, at least 92%, at least 93%, at least 94%, at least 95%, at least 96%, at least 97%, at least 98%, at least 99%, or 100%) identical to SEQ ID NO: 176.

In certain embodiments, bridging moieties of the present invention comprise a hinge polypeptide comprising an amino acid at least 90% (e.g., at least 91%, at least 92%, at least 93%, at least 94%, at least 95%, at least 96%, at least 97%, at least 98%, at least 99%, or 100%) identical to SEQ ID NO: 249.

In certain embodiments, bridging moieties of the present invention comprise a hinge polypeptide comprising an amino acid at least 90% (e.g., at least 91%, at least 92%, at least 93%, at least 94%, at least 95%, at least 96%, at least 97%, at least 98%, at least 99%, or 100%) identical to SEQ ID NO: 177.

In certain embodiments, bridging moieties of the present invention comprise a hinge polypeptide comprising an amino acid at least 90% (e.g., at least 91%, at least 92%, at least 93%, at least 94%, at least 95%, at least 96%, at least 97%, at least 98%, at least 99%, or 100%) identical to SEQ ID NO: 243.

In certain embodiments, bridging moieties of the present invention comprise a hinge polypeptide comprising an amino acid at least 90% (e.g., at least 91%, at least 92%, at least 93%, at least 94%, at least 95%, at least 96%, at least 97%, at least 98%, at least 99%, or 100%) identical to SEQ ID NO: 212.

In certain embodiments, bridging moieties of the present invention comprise a hinge polypeptide comprising an amino acid at least 90% (e.g., at least 91%, at least 92%, at least 93%, at least 94%, at least 95%, at least 96%, at least 97%, at least 98%, at least 99%, or 100%) identical to SEQ ID NO: 244.

In certain embodiments, bridging moieties of the present invention comprise a hinge polypeptide comprising an amino acid at least 90% (e.g., at least 91%, at least 92%, at least 93%, at least 94%, at least 95%, at least 96%, at least 97%, at least 98%, at least 99%, or 100%) identical to SEQ ID NO: 213.

In certain embodiments, bridging moieties of the present invention comprise a hinge polypeptide comprising an amino acid at least 90% (e.g., at least 91%, at least 92%, at least 93%, at least 94%, at least 95%, at least 96%, at least 97%, at least 98%, at least 99%, or 100%) identical to SEQ ID NO: 245.

In certain embodiments, bridging moieties of the present invention comprise a hinge polypeptide comprising an amino acid at least 90% (e.g., at least 91%, at least 92%, at least 93%, at least 94%, at least 95%, at least 96%, at least 97%, at least 98%, at least 99%, or 100%) identical to SEQ ID NO: 247.

In certain embodiments, bridging moieties of the present invention comprise a hinge polypeptide comprising an amino acid at least 90% (e.g., at least 91%, at least 92%, at least 93%, at least 94%, at least 95%, at least 96%, at least 97%, at least 98%, at least 99%, or 100%) identical to SEQ ID NO: 248.

### Human Serum Albumin

In some embodiments, bridging moieties of the present invention comprise a human serum albumin (HSA) polypeptide of functional fragment thereof. For example, a bridging moiety of the present may comprise an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 219.

(SEQ ID NO: 219)
DAHKSEVAHRFKDLGEENFKALVLIAFAQYLQQCPFEDHVKLVNEVT
EFAKTCVADESAENCDKSLHTLFGDKLCTVATLRETYGEMADCCAK
QEPERNECFLQHKDDNPNLPRLVRPEVDVMCTAFHDNEETFLKKYLY
EIARRHPYFYAPELLFFAKRYKAAFTECCQAADKAACLLPKLDELRD
EGKASSAKQRLKCASLQKFGERAFKAWAVARLSQRFPKAEFAEVSK
LVTDLTKVHTECCHGDLLECADDRADLAKYICENQDSISSKLKECCE
KPLLEKSHCIAEVENDEMPADLPSLAADFVESKDVCKNYAEAKDVFL
GMFLYEYARRHPDYSVVLLLRLAKTYETTLEKCCAAADPHECYAKV
FDEFKPLVEEPQNLIKQNCELFEQLGEYKFQNALLVRYTKKVPQVSTP
TLVEVSRNLGKVGSKCCKHPEAKRMPCAEDYLSVVLNQLCVLHEKT
PVSDRVTKCCTESLVNRRPCFSALEVDETYVPKEFNAETFTFHADICT
LSEKERQIKKQTALVELVKHKPKATKEQLKAVMDDFAAFVEKCCKA
DDKETCFAEEGKKLVAASQAALGL

### I.D Linkers

In some embodiments, the N-terminus of a polypeptide or complex of two or more polypeptides that specifically binds OX40L is connected to the C-terminus of a bridging moiety via a linker polypeptide. In other embodiments, the N-terminus of a polypeptide or complex of two or more polypeptides that specifically binds CD80 and/or CD86 is connected to the C-terminus of a bridging moiety via a linker polypeptide. In some embodiments, the N-terminus of a polypeptide or complex of two or more polypeptides that specifically binds CD80 and/or CD86 is connected to the C-terminus of a polypeptide or complex of two or more polypeptides that specifically binds OX40L via a linker polypeptide. In other embodiments, the N-terminus of a polypeptide or complex of two or more polypeptides that specifically binds OX40L is connected to the C-terminus of a polypeptide or complex of two or more polypeptides that specifically binds CD80 and/or CD86 via a linker polypeptide.

Regarding the amino acid composition of the linker polypeptide, linker polypeptide sequences are selected with properties that confer flexibility, and for minimal interference with the structure and function of the other domains and/or polypeptides of the proteins described in the present application. Linker polypeptide sequences are also selected to be resistant to proteolytic cleavage. For example, glycine and serine residues generally provide protease resistance.

In certain embodiments, proteins described herein comprise a (GlyGlyGlyGlySer)₄ ((G₄S)₄) linker (SEQ ID NO: 111). The length of the linker (e.g., flexible linker) can be “short,” e.g., 0, 1, 2, 3, 4, 5, 6, 7, 8, 9, 10, 11 or 12 amino acid residues, or “long,” e.g., at least 13 amino acid residues. In certain embodiments, the linker is 10-50, 10-40, 10-30, 10-25, 10-20, 15-50, 15-40, 15-30, 15-25, 15-20, 20-50, 20-40, 20-30, or 20-25 amino acid residues in length.

In certain embodiments, proteins of the present invention comprise one or more polypeptide linker comprising or consisting of an amino acid sequence listed in Table 6.

TABLE 6
Exemplary Linker Polypeptides

| SEQ ID | Amino Acid Sequence |
|---|---|
| SEQ ID NO: | (GGGGS)ₙ, wherein n is 1 to 12 |
| 181 | |
| SEQ ID NO: | GSGSGSGSGSGSGSGSGSGS |
| 206 | |
| SEQ ID NO: | GGSGGSGGSGGSGGSGGSGGSGGSGGSGGS |
| 207 | |
| SEQ ID NO: | GGGSGGGSGGGSGGGSGGGSGGGSGGGSGGGSGGGSGG |
| 208 | GS |
| SEQ ID NO: | GGSGGGSGGGSGGGSGGGSGGGSGGGSGGGSGGGSGGG |
| 209 | SG |
| SEQ ID NO: | GGSGGGGSGGGGSGGGGSGGGGSGGGGSGGGGSGGGGS |
| 210 | GGGGSGGGGSGG |
| SEQ ID NO: | GGSGGGGSGGGGSGGGGSGGGGSGGGGSGGGGSGGGGS |
| 211 | GGGGSGGGGSGGGGSGGGGSGGGGSGGGGSGGGGSGGG |
| | GSGGGGSGGGGSGGGGSGGGGSGG |

### I.E Exemplary Proteins

Listed below are examples of proteins of the present invention comprising a polypeptide or complex of two or more polypeptides that specifically binds CD80 and/or CD86 and a polypeptide or complex of two or more polypeptides that specifically binds OX40L.

For example, the polypeptide that specifically binds CD80 and/or CD86 can comprise a wildtype or variant extracellular domain of CTLA4 selected from any one of the sequences listed in TABLE 1. Alternatively, the polypeptide or complex of two or more polypeptides that specifically binds CD80 and/or CD86 can comprise a wildtype or variant extracellular domain of CD28 or functional fragment thereof, or an antigen binding site (e.g. an antibody, a Fab; a Fab′, a F(ab′)2, a single-chain variable fragment (scFv); a minibody; or a nanobody (VHH)).

In some embodiments, the polypeptide or complex of two or more polypeptides that specifically binds OX40L is an antigen binding site (e.g. an antibody, a Fab; a Fab′, a F(ab′)2, a single-chain variable fragment (scFv); a minibody; or a nanobody (VHH)). For example, antigen binding sites that specifically bind OX40L can comprise a Fab comprising the VHCDR1, VHCDR2, VHCDR3, VLCDR1, VLCDR2, and VLCDR3 sequences selected from any of the VHCDR1, VHCDR2, VHCDR3, VLCDR1, VLCDR2, and VLCDR3 sequences listed in TABLE 2, the VH and VL sequences listed in TABLE 3, and/or the HC and LC sequences listed in TABLE 3. In other embodiments, the polypeptide that binds OX40L can comprise an extracellular domain of OX40, or functional fragment thereof.

As described above, proteins of the present invention may comprise a bridging moiety. In some embodiments, the polypeptide or complex of two or more polypeptides that specifically binds CD80 and/or CD86 is connected at its C-terminus to the N-terminus of a bridging moiety and the polypeptide or complex of two or more polypeptides that specifically binds OX40L is connected at its N-terminus to the C-terminus of the bridging moiety. As described above, the bridging moiety can be a polypeptide of a wildtype human IgG1 Fc domain optionally comprising one or more mutation and comprising a sequence selected from any one of the sequences listed in TABLE 4. The bridging moiety can also comprise a hinge polypeptide at its N-terminus optionally comprising one or more mutation such as a sequence selected from any one of the sequences listed in TABLE 5.

As also described above, the polypeptide or complex of two or more polypeptides that specifically binds OX40L can be connected to the C-terminus of the bridging moiety via a linker polypeptide. For example, the linker polypeptide can comprise a polypeptide comprising a sequence selected from any one of the sequences listed in TABLE 6.

Proteins of the present invention can comprise a heavy chain and a light chain. For example, a heavy chain of the present invention can comprise, from N-terminus to C-terminus: an extracellular domain of CTLA4 or functional fragment thereof, a bridging moiety comprising at least a portion of a hinge polypeptide at its N-terminus and the CH2 and CH3 domains of polypeptide of a human IgG1 Fc; a linker polypeptide; and a heavy chain of an anti-OX40L Fab comprising a VH domain and CH1 domain. The heavy chain can additionally comprise at the C-terminus of the CH1 domain, a partial IgG1 hinge sequence (for example, a polypeptide comprising the amino acid sequence of SEQ ID NO: 336) comprising a cysteine residue capable of forming a disulfide bond with a cysteine residue in a light chain constant domain (CL). TABLE 7 lists exemplary heavy chain sequences of the present invention.

A light chain of the present invention can comprise, from N-terminus to C-terminus: a VL domain and CL domain of the anti-OX40L Fab. TABLE 7 lists exemplary light chain sequences of the present invention.

In combination, heavy chains and light chains as listed in TABLE 7 associate, for example linked by a disulfide bond, and can specifically bind to CD80 and CD86, and OX40L. As contemplated in proteins of the present invention, the hinge polypeptide and CH3 domain of the polypeptide of the human IgG1 Fc can facilitate dimerization of two heavy chains resulting in a protein comprising of two heavy chains and two light chains.

TABLE 7
Exemplary Heavy Chain and Light Chain Sequences

| | Heavy Chain | Light Chain |
|---|---|---|
| 67B06(1) | AMHVAQPAVVLASSRGIASFVCE | SYELTQPLSVSVALGQTARMTCG |
| | YASPGKATEVRVTVLRQADSQV | GSDIENKNVHWYQQKPGQAPVL |
| | TEVCAATYMMGNELTFLDDSICT | VIHRDNNRPSGIPERFSGSNSGNT |
| | GTSSGNQVNLTIQGLRAMDTGL | ATLTISRAQAGDEADYYCQVRDS |
| | YICKVELMYPPPYYLGIGNGTQI | NIVVFGGGTKLTVLGQPKAAPSV |
| | YVIDPEPCPDSDQEPKSSDKTHT | TLFPPSSEELQANKATLVCLISDFY |
| | CPPCPAPPVAGPSVFLFPPKPKDT | PGAVTVAWKADSSPVKAGVETT |
| | LMISRTPEVTCVVVDVSHEDPEV | TPSKQSNNKYAASSYLSLTPEQW |
| | KFNWYVDGVEVHNAKTKPREE | KSHKSYSCQVTHEGSTVEKTVAP |
| | QYNSTYRVVSVLTVLHQDWLNG | TECS (SEQ ID NO: 39) |
| | KEYKCAVSNKALPSSIEKTISKAK | |
| | GQPREPQVYTLPPSREEMTKNQV | |
| | SLTCLVKGFYPSDIAVEWESNGQ | |
| | PENNYKTTPPVLDSDGSFFLYSK | |
| | LTVDKSRWQQGNVFSCSVMHEA | |
| | LHNHYTQKSLSLSPGAGGGGSG | |
| | GGGSGGGGSQLQLQESGPGLVK | |
| | PSETLSLTCSVSGGSISTSSYYWG | |
| | WIRQPPGKGLEWSGSIYYSGSTY | |
| | YRPSLQGRATISVDTSKNQFSLK | |
| | LTSVTAADTAVYYCARHRGSYF | |
| | FDIWGLGTLVTVSSASTKGPSVF | |
| | PLAPSSKSTSGGTAALGCLVKDY | |
| | FPEPVTVSWNSGALTSGVHTFPA | |
| | VLQSSGLYSLSSVVTVPSSSLGTQ | |
| | TYICNVNHKPSNTKVDKRVEPKS | |
| | C (SEQ ID NO: 40) | |
| 67B06(2) | AMHVAQPAVVLASSRGIASFVCE | SYELTQPLSVSVALGQTARITCGG |
| | YASPGKATEVRVTVLRQADSQV | SDIENKNVHWYQQKPGQAPVLVI |
| | TEVCAATYMMGNELTFLDDSICT | HRDNNRPSGIPERFSGSNSGNTAT |
| | GTSSGNQVNLTIQGLRAMDTGL | LTISRAQAGDEADYYCQVRDSNI |
| | YICKVELMYPPPYYLGIGNGTQI | VVFGGGTKLTVLGQPKAAPSVTL |
| | YVIDPEPCPDSDQEPKSSDKTHT | FPPSSEELQANKATLVCLISDFYP |
| | CPPCPAPPVAGPSVFLFPPKPKDT | GAVTVAWKADSSPVKAGVETTT |
| | LMISRTPEVTCVVVDVSHEDPEV | PSKQSNNKYAASSYLSLTPEQWK |
| | KFNWYVDGVEVHNAKTKPREE | SHKSYSCQVTHEGSTVEKTVAPT |
| | QYNSTYRVVSVLTVLHQDWLNG | ECS (SEQ ID NO: 61) |
| | KEYKCAVSNKALAAPIEKTISKA | |
| | KGQPREPQVYTLPPSREEMTKNQ | |
| | VSLTCLVKGFYPSDIAVEWESNG | |
| | QPENNYKTTPPVLDSDGSFFLYS | |
| | KLTVDKSRWQQGNVFSCSVMHE | |
| | ALHNHYTQKSLSLSPGAGGGGS | |
| | GGGGSGGGGSQLQLQESGPGLV | |
| | KPSETLSLTCSVSGGSISTSSYYW | |
| | GWIRQPPGKGLEWSGSIYYSGST | |
| | YYRPSLQGRATISVDTSKNQFSL | |
| | KLTSVTAADTAVYYCARHRGSY | |
| | FFDIWGLGTLVTVSSASTKGPSV | |
| | FPLAPSSKSTSGGTAALGCLVKD | |
| | YFPEPVTVSWNSGALTSGVHTFP | |
| | AVLQSSGLYSLSSVVTVPSSSLGT | |
| | QTYICNVNHKPSNTKVDKRVEP | |
| | KSC (SEQ ID NO: 62) | |
| 67B06(3) | AMHVAQPAVVLASSRGIASFVCE | SSELTQPLSVSVALGQTARMTCG |
| | YASPGKATEVRVTVLRQADSQV | GSDIENKNVHWYQQKPGQAPVL |
| | TEVCAATYMMGNELTFLDDSICT | VIHRDNNRPSGIPERFSGSNSGNT |
| | GTSSGNQVNLTIQGLRAMDTGL | ATLTISRAQAGDEADYYCQVRDS |
| | YICKVELMYPPPYYLGIGNGTQI | NIVVFGGGTKLTVLGQPKAAPSV |
| | YVIDPEPCPDSDQEPKSSDKTHT | TLFPPSSEELQANKATLVCLISDFY |
| | CPPCPAPEAAGGPSVFLFPPKPKD | PGAVTVAWKADSSPVKAGVETT |
| | TLMISRTPEVTCVVVDVSHEDPE | TPSKQSNNKYAASSYLSLTPEQW |
| | VKFNWYVDGVEVHNAKTKPRE | KSHKSYSCQVTHEGSTVEKTVAP |
| | EQYNSTYRVVSVLTVLHQDWLN | TECS (SEQ ID NO: 63) |
| | GKEYKCKVSNKALPAPIEKTISK | |
| | AKGQPREPQVYTLPPSREEMTKN | |
| | QVSLTCLVKGFYPSDIAVEWESN | |
| | GQPENNYKTTPPVLDSDGSFFLY | |
| | SKLTVDKSRWQQGNVFSCSVMH | |
| | EALHNHYTQKSLSLSPGAGGGGS | |
| | GGGGSGGGGSQLQLQESGPGLV | |
| | KPSETLSLTCSVSGGSISTSSYYW | |
| | GWIRQPPGKGLEWSGSIYYSGST | |
| | YYRPSLQGRATISVDTSKNQFSL | |
| | KLTSVTAADTAVYYCARHRGSY | |
| | FFDIWGLGTLVTVSSASTKGPSV | |
| | FPLAPSSKSTSGGTAALGCLVKD | |
| | YFPEPVTVSWNSGALTSGVHTFP | |
| | AVLQSSGLYSLSSVVTVPSSSLGT | |
| | QTYICNVNHKPSNTKVDKRVEP | |
| | KSC (SEQ ID NO: 64) | |
| 67B06(4) | KAMHVAQPAVVLASSRGIASFV | SSELTQPLSVSVALGQTARMTCG |
| | CEYASPGKATEVRVTVLRQADS | GSDIENKNVHWYQQKPGQAPVL |
| | QVTEVCAATYMMGNELTFLDDS | VIHRDNNRPSGIPERFSGSNSGNT |
| | ICTGTSSGNQVNLTIQGLRAMDT | ATLTISRAQAGDEADYYCQVRDS |
| | GLYICKVELMYPPPYYLGIGNGT | NIVVFGGGTKLTVLGQPKAAPSV |
| | QIYVIDPEPCPDSDQEPKSSDKTH | TLFPPSSEELQANKATLVCLISDFY |
| | TCPPCPAPEAAGGPSVFLFPPKPK | PGAVTVAWKADSSPVKAGVETT |
| | DTLMISRTPEVTCVVVDVSHEDP | TPSKQSNNKYAASSYLSLTPEQW |
| | EVKFNWYVDGVEVHNAKTKPR | KSHKSYSCQVTHEGSTVEKTVAP |
| | EEQYNSTYRVVSVLTVLHQDWL | TECS (SEQ ID NO: 63) |
| | NGKEYKCKVSNKALPAPIEKTIS | |
| | KAKGQPREPQVYTLPPSREEMTK | |
| | NQVSLTCLVKGFYPSDIAVEWES | |
| | NGQPENNYKTTPPVLDSDGSFFL | |
| | YSKLTVDKSRWQQGNVFSCSVM | |
| | HEALHNHYTQKSLSLSPGAGGG | |
| | GSGGGGSGGGGSQLQLQESGPG | |
| | LVKPSETLSLTCSVSGGSISTSSY | |
| | YWGWIRQPPGKGLEWSGSIYYS | |
| | GSTYYRPSLQGRATISVDTSKNQ | |
| | FSLKLTSVTAADTAVYYCARHR | |
| | GSYFFDIWGLGTLVTVSSASTKG | |
| | PSVFPLAPSSKSTSGGTAALGCL | |
| | VKDYFPEPVTVSWNSGALTSGV | |
| | HTFPAVLQSSGLYSLSSVVTVPSS | |
| | SLGTQTYICNVNHKPSNTKVDKR | |
| | VEPKSC (SEQ ID NO: 182) | |
| 67B06(5) | MHVAQPAVVLASSRGIASFVCEY | SSELTQPLSVSVALGQTARMTCG |
| | ASPGKATEVRVTVLRQADSQVT | GSDIENKNVHWYQQKPGQAPVL |
| | EVCAATYMMGNELTFLDDSICT | VIHRDNNRPSGIPERFSGSNSGNT |
| | GTSSGNQVNLTIQGLRAMDTGL | ATLTISRAQAGDEADYYCQVRDS |
| | YICKVELMYPPPYYLGIGNGTQI | NIVVFGGGTKLTVLGQPKAAPSV |
| | YVIDPEPCPDSDQEPKSSDKTHT | TLFPPSSEELQANKATLVCLISDFY |
| | CPPCPAPEAAGGPSVFLFPPKPKD | PGAVTVAWKADSSPVKAGVETT |
| | TLMISRTPEVTCVVVDVSHEDPE | TPSKQSNNKYAASSYLSLTPEQW |
| | VKFNWYVDGVEVHNAKTKPRE | KSHKSYSCQVTHEGSTVEKTVAP |
| | EQYNSTYRVVSVLTVLHQDWLN | TECS (SEQ ID NO: 63) |
| | GKEYKCKVSNKALPAPIEKTISK | |
| | AKGQPREPQVYTLPPSREEMTKN | |
| | QVSLTCLVKGFYPSDIAVEWESN | |
| | GQPENNYKTTPPVLDSDGSFFLY | |
| | SKLTVDKSRWQQGNVFSCSVMH | |
| | EALHNHYTQKSLSLSPGAGGGGS | |
| | GGGGSGGGGSQLQLQESGPGLV | |
| | KPSETLSLTCSVSGGSISTSSYYW | |
| | GWIRQPPGKGLEWSGSIYYSGST | |
| | YYRPSLQGRATISVDTSKNQFSL | |
| | KLTSVTAADTAVYYCARHRGSY | |
| | FFDIWGLGTLVTVSSASTKGPSV | |
| | FPLAPSSKSTSGGTAALGCLVKD | |
| | YFPEPVTVSWNSGALTSGVHTFP | |
| | AVLQSSGLYSLSSVVTVPSSSLGT | |
| | QTYICNVNHKPSNTKVDKRVEP | |
| | KSC (SEQ ID NO: 183) | |
| 67B06(6) | KAMHVAQPAVVLASSRGIASFV | SSELTQPLSVSVALGQTARMTCG |
| | CEYASPGKATEVRVTVLRQADS | GSDIENKNVHWYQQKPGQAPVL |
| | QVTEVCAATYMMGNELTFLDDS | VIHRDNNRPSGIPERFSGSNSGNT |
| | ICTGTSSGNQVNLTIQGLRAMDT | ATLTISRAQAGDEADYYCQVRDS |
| | GLYICKVELMYPPPYYLGIGNGT | NIVVFGGGTKLTVLGQPKAAPSV |
| | QIYVIDPEPCPDSDEPKSSDKTHT | TLFPPSSEELQANKATLVCLISDFY |
| | CPPCPAPEAAGGPSVFLFPPKPKD | PGAVTVAWKADSSPVKAGVETT |
| | TLMISRTPEVTCVVVDVSHEDPE | TPSKQSNNKYAASSYLSLTPEQW |
| | VKFNWYVDGVEVHNAKTKPRE | KSHKSYSCQVTHEGSTVEKTVAP |
| | EQYNSTYRVVSVLTVLHQDWLN | TECS (SEQ ID NO: 63) |
| | GKEYKCKVSNKALPAPIEKTISK | |
| | AKGQPREPQVYTLPPSREEMTKN | |
| | QVSLTCLVKGFYPSDIAVEWESN | |
| | GQPENNYKTTPPVLDSDGSFFLY | |
| | SKLTVDKSRWQQGNVFSCSVMH | |
| | EALHNHYTQKSLSLSPGAGGGGS | |
| | GGGGSGGGGSQLQLQESGPGLV | |
| | KPSETLSLTCSVSGGSISTSSYYW | |
| | GWIRQPPGKGLEWSGSIYYSGST | |
| | YYRPSLQGRATISVDTSKNQFSL | |
| | KLTSVTAADTAVYYCARHRGSY | |
| | FFDIWGLGTLVTVSSASTKGPSV | |
| | FPLAPSSKSTSGGTAALGCLVKD | |
| | YFPEPVTVSWNSGALTSGVHTFP | |
| | AVLQSSGLYSLSSVVTVPSSSLGT | |
| | QTYICNVNHKPSNTKVDKRVEP | |
| | KSC (SEQ ID NO: 184) | |
| 67B06(7) | MHVAQPAVVLASSRGIASFVCEY | SSELTQPLSVSVALGQTARMTCG |
| | ASPGKATEVRVTVLRQADSQVT | GSDIENKNVHWYQQKPGQAPVL |
| | EVCAATYMMGNELTFLDDSICT | VIHRDNNRPSGIPERFSGSNSGNT |
| | GTSSGNQVNLTIQGLRAMDTGL | ATLTISRAQAGDEADYYCQVRDS |
| | YICKVELMYPPPYYLGIGNGTQI | NIVVFGGGTKLTVLGQPKAAPSV |
| | YVIDPEPCPDSDEPKSSDKTHTCP | TLFPPSSEELQANKATLVCLISDFY |
| | PCPAPEAAGGPSVFLFPPKPKDTL | PGAVTVAWKADSSPVKAGVETT |
| | MISRTPEVTCVVVDVSHEDPEVK | TPSKQSNNKYAASSYLSLTPEQW |
| | FNWYVDGVEVHNAKTKPREEQ | KSHKSYSCQVTHEGSTVEKTVAP |
| | YNSTYRVVSVLTVLHQDWLNGK | TECS (SEQ ID NO: 63) |
| | EYKCKVSNKALPAPIEKTISKAK | |
| | GQPREPQVYTLPPSREEMTKNQV | |
| | SLTCLVKGFYPSDIAVEWESNGQ | |
| | PENNYKTTPPVLDSDGSFFLYSK | |
| | LTVDKSRWQQGNVFSCSVMHEA | |
| | LHNHYTQKSLSLSPGAGGGGSG | |
| | GGGSGGGGSQLQLQESGPGLVK | |
| | PSETLSLTCSVSGGSISTSSYYWG | |
| | WIRQPPGKGLEWSGSIYYSGSTY | |
| | YRPSLQGRATISVDTSKNQFSLK | |
| | LTSVTAADTAVYYCARHRGSYF | |
| | FDIWGLGTLVTVSSASTKGPSVF | |
| | PLAPSSKSTSGGTAALGCLVKDY | |
| | FPEPVTVSWNSGALTSGVHTFPA | |
| | VLQSSGLYSLSSVVTVPSSSLGTQ | |
| | TYICNVNHKPSNTKVDKRVEPKS | |
| | C (SEQ ID NO: 185) | |
| 67B06(8) | AMHVAQPAVVLASSRGIASFVCE | SSELTQPLSVSVALGQTARMTCG |
| | YASPGKATEVRVTVLRQADSQV | GSDIENKNVHWYQQKPGQAPVL |
| | TEVCAATYMMGNELTFLDDSICT | VIHRDNNRPSGIPERFSGSNSGNT |
| | GTSSGNQVNLTIQGLRAMDTGL | ATLTISRAQAGDEADYYCQVRDS |
| | YICKVELMYPPPYYLGIGNGTQI | NIVVFGGGTKLTVLGQPKAAPSV |
| | YVIDPEPCPDSDEPKSSDKTHTCP | TLFPPSSEELQANKATLVCLISDFY |
| | PCPAPEAAGGPSVFLFPPKPKDTL | PGAVTVAWKADSSPVKAGVETT |
| | MISRTPEVTCVVVDVSHEDPEVK | TPSKQSNNKYAASSYLSLTPEQW |
| | FNWYVDGVEVHNAKTKPREEQ | KSHKSYSCQVTHEGSTVEKTVAP |
| | YNSTYRVVSVLTVLHQDWLNGK | TECS (SEQ ID NO: 63) |
| | EYKCKVSNKALPAPIEKTISKAK | |
| | GQPREPQVYTLPPSREEMTKNQV | |
| | SLTCLVKGFYPSDIAVEWESNGQ | |
| | PENNYKTTPPVLDSDGSFFLYSK | |
| | LTVDKSRWQQGNVFSCSVMHEA | |
| | LHNHYTQKSLSLSPGAGGGGSG | |
| | GGGSGGGGSQLQLQESGPGLVK | |
| | PSETLSLTCSVSGGSISTSSYYWG | |
| | WIRQPPGKGLEWSGSIYYSGSTY | |
| | YRPSLQGRATISVDTSKNQFSLK | |
| | LTSVTAADTAVYYCARHRGSYF | |
| | FDIWGLGTLVTVSSASTKGPSVF | |
| | PLAPSSKSTSGGTAALGCLVKDY | |
| | FPEPVTVSWNSGALTSGVHTFPA | |
| | VLQSSGLYSLSSVVTVPSSSLGTQ | |
| | TYICNVNHKPSNTKVDKRVEPKS | |
| | C (SEQ ID NO: 186) | |
| 89B09(1) | AMHVAQPAVVLASSRGIASFVCE | SSELTQDPAVSVALGQTVRITCQG |
| | YASPGKATEVRVTVLRQADSQV | DSVRRFFAHWYQQKPGQAPILVIS |
| | TEVCAATYMMGNELTFLDDSICT | GKDTRPSGIPDRFSGSISGNTASLT |
| | GTSSGNQVNLTIQGLRAMDTGL | ITGAQAEDEADYYCNSRDSSGYL |
| | YICKVELMYPPPYYLGIGNGTQI | VLFGGGTKLTVLGQPKAAPSVTL |
| | YVIDPEPCPDSDQEPKSSDKTHT | FPPSSEELQANKATLVCLISDFYP |
| | CPPCPAPPVAGPSVFLFPPKPKDT | GAVTVAWKADSSPVKAGVETTT |
| | LMISRTPEVTCVVVDVSHEDPEV | PSKQSNNKYAASSYLSLTPEQWK |
| | KFNWYVDGVEVHNAKTKPREE | SHKSYSCQVTHEGSTVEKTVAPT |
| | QYNSTYRVVSVLTVLHQDWLNG | ECS (SEQ ID NO: 47) |
| | KEYKCAVSNKALPSSIEKTISKAK | |
| | GQPREPQVYTLPPSREEMTKNQV | |
| | SLTCLVKGFYPSDIAVEWESNGQ | |
| | PENNYKTTPPVLDSDGSFFLYSK | |
| | LTVDKSRWQQGNVFSCSVMHEA | |
| | LHNHYTQKSLSLSPGAGGGGSG | |
| | GGGSGGGGSEVQLVESGPGLVK | |
| | PSETLSLTCTVSGVSIRSNGYYW | |
| | GWIRQSPGKGLEWIGNMDYSGT | |
| | YQNPSLTSRVTISGDASKNQFSL | |
| | KLRSVTAADTAVYFCARERSNN | |
| | WYPIDYWGQGTLVTVSSASTKG | |
| | PSVFPLAPSSKSTSGGTAALGCL | |
| | VKDYFPEPVTVSWNSGALTSGV | |
| | HTFPAVLQSSGLYSLSSVVTVPSS | |
| | SLGTQTYICNVNHKPSNTKVDKR | |
| | VEPKSC (SEQ ID NO: 48) | |
| 89B09(2) | AMHVAQPAVVLASSRGIASFVCE | SSELTQDPAVSVALGQTVRITCQG |
| | YASPGKATEVRVTVLRQADSQV | DSVRRFFAHWYQQKPGQAPVLVI |
| | TEVCAATYMMGNELTFLDDSICT | SGKDTRPSGIPDRFSGSSSGNTASL |
| | GTSSGNQVNLTIQGLRAMDTGL | TITGAQAEDEADYYCNSRDSSGY |
| | YICKVELMYPPPYYLGIGNGTQI | LVLFGGGTKLTVLGQPKAAPSVT |
| | YVIDPEPCPDSDQEPKSSDKTHT | LFPPSSEELQANKATLVCLISDFYP |
| | CPPCPAPPVAGPSVFLFPPKPKDT | GAVTVAWKADSSPVKAGVETTT |
| | LMISRTPEVTCVVVDVSHEDPEV | PSKQSNNKYAASSYLSLTPEQWK |
| | KFNWYVDGVEVHNAKTKPREE | SHKSYSCQVTHEGSTVEKTVAPT |
| | QYNSTYRVVSVLTVLHQDWLNG | ECS (SEQ ID NO: 67) |
| | KEYKCAVSNKALAAPIEKTISKA | |
| | KGQPREPQVYTLPPSREEMTKNQ | |
| | VSLTCLVKGFYPSDIAVEWESNG | |
| | QPENNYKTTPPVLDSDGSFFLYS | |
| | KLTVDKSRWQQGNVFSCSVMHE | |
| | ALHNHYTQKSLSLSPGAGGGGS | |
| | GGGGSGGGGSQLQLQESGPGLV | |
| | KPSETLSLTCTVSGVSIRSSGYYW | |
| | GWIRQPPGKGLEWIGNIDYSGTY | |
| | QNPSLTSRVTISVDTSKNQFSLKL | |
| | SSVTAADTAVYYCARERSNNWY | |
| | PIDYWGQGTLVTVSSASTKGPSV | |
| | FPLAPSSKSTSGGTAALGCLVKD | |
| | YFPEPVTVSWNSGALTSGVHTFP | |
| | AVLQSSGLYSLSSVVTVPSSSLGT | |
| | QTYICNVNHKPSNTKVDKRVEP | |
| | KSC (SEQ ID NO: 68) | |
| 89B09(3) | KAMHVAQPAVVLASSRGIASFV | SSELTQDPAVSVALGQTVRITCQG |
| | CEYASPGKATEVRVTVLRQADS | DSVRRFFAHWYQQKPGQAPVLVI |
| | QVTEVCAATYMMGNELTFLDDS | SGKDTRPSGIPDRFSGSSSGNTASL |
| | ICTGTSSGNQVNLTIQGLRAMDT | TITGAQAEDEADYYCNSRDSSGY |
| | GLYICKVELMYPPPYYLGIGNGT | LVLFGGGTKLTVLGQPKAAPSVT |
| | QIYVIDPEPCPDSDQEPKSSDKTH | LFPPSSEELQANKATLVCLISDFYP |
| | TCPPCPAPEAAGGPSVFLFPPKPK | GAVTVAWKADSSPVKAGVETTT |
| | DTLMISRTPEVTCVVVDVSHEDP | PSKQSNNKYAASSYLSLTPEQWK |
| | EVKFNWYVDGVEVHNAKTKPR | SHKSYSCQVTHEGSTVEKTVAPT |
| | EEQYNSTYRVVSVLTVLHQDWL | ECS (SEQ ID NO: 67) |
| | NGKEYKCKVSNKALPAPIEKTIS | |
| | KAKGQPREPQVYTLPPSREEMTK | |
| | NQVSLTCLVKGFYPSDIAVEWES | |
| | NGQPENNYKTTPPVLDSDGSFFL | |
| | YSKLTVDKSRWQQGNVFSCSVM | |
| | HEALHNHYTQKSLSLSPGAGGG | |
| | GSGGGGSGGGGSEVQLVESGPG | |
| | LVKPSETLSLTCTVSGVSIRSNGY | |
| | YWGWIRQSPGKGLEWIGNMDYS | |
| | GTYQNPSLTSRVTISGDASKNQF | |
| | SLKLRSVTAADTAVYFCARERSN | |
| | NWYPIDYWGQGTLVTVSSASTK | |
| | GPSVFPLAPSSKSTSGGTAALGC | |
| | LVKDYFPEPVTVSWNSGALTSG | |
| | VHTFPAVLQSSGLYSLSSVVTVP | |
| | SSSLGTQTYICNVNHKPSNTKVD | |
| | KRVEPKSC (SEQ ID NO: 187) | |
| 89B09(4) | MHVAQPAVVLASSRGIASFVCEY | SSELTQDPAVSVALGQTVRITCQG |
| | ASPGKATEVRVTVLRQADSQVT | DSVRRFFAHWYQQKPGQAPVLVI |
| | EVCAATYMMGNELTFLDDSICT | SGKDTRPSGIPDRFSGSSSGNTASL |
| | GTSSGNQVNLTIQGLRAMDTGL | TITGAQAEDEADYYCNSRDSSGY |
| | YICKVELMYPPPYYLGIGNGTQI | LVLFGGGTKLTVLGQPKAAPSVT |
| | YVIDPEPCPDSDQEPKSSDKTHT | LFPPSSEELQANKATLVCLISDFYP |
| | CPPCPAPEAAGGPSVFLFPPKPKD | GAVTVAWKADSSPVKAGVETTT |
| | TLMISRTPEVTCVVVDVSHEDPE | PSKQSNNKYAASSYLSLTPEQWK |
| | VKFNWYVDGVEVHNAKTKPRE | SHKSYSCQVTHEGSTVEKTVAPT |
| | EQYNSTYRVVSVLTVLHQDWLN | ECS (SEQ ID NO: 67) |
| | GKEYKCKVSNKALPAPIEKTISK | |
| | AKGQPREPQVYTLPPSREEMTKN | |
| | QVSLTCLVKGFYPSDIAVEWESN | |
| | GQPENNYKTTPPVLDSDGSFFLY | |
| | SKLTVDKSRWQQGNVFSCSVMH | |
| | EALHNHYTQKSLSLSPGAGGGGS | |
| | GGGGSGGGGSEVQLVESGPGLV | |
| | KPSETLSLTCTVSGVSIRSNGYY | |
| | WGWIRQSPGKGLEWIGNMDYSG | |
| | TYQNPSLTSRVTISGDASKNQFSL | |
| | KLRSVTAADTAVYFCARERSNN | |
| | WYPIDYWGQGTLVTVSSASTKG | |
| | PSVFPLAPSSKSTSGGTAALGCL | |
| | VKDYFPEPVTVSWNSGALTSGV | |
| | HTFPAVLQSSGLYSLSSVVTVPSS | |
| | SLGTQTYICNVNHKPSNTKVDKR | |
| | VEPKSC (SEQ ID NO: 188) | |
| 89B09(5) | KAMHVAQPAVVLASSRGIASFV | SSELTQDPAVSVALGQTVRITCQG |
| | CEYASPGKATEVRVTVLRQADS | DSVRRFFAHWYQQKPGQAPVLVI |
| | QVTEVCAATYMMGNELTFLDDS | SGKDTRPSGIPDRFSGSSSGNTASL |
| | ICTGTSSGNQVNLTIQGLRAMDT | TITGAQAEDEADYYCNSRDSSGY |
| | GLYICKVELMYPPPYYLGIGNGT | LVLFGGGTKLTVLGQPKAAPSVT |
| | QIYVIDPEPCPDSDEPKSSDKTHT | LFPPSSEELQANKATLVCLISDFYP |
| | CPPCPAPEAAGGPSVFLFPPKPKD | GAVTVAWKADSSPVKAGVETTT |
| | TLMISRTPEVTCVVVDVSHEDPE | PSKQSNNKYAASSYLSLTPEQWK |
| | VKFNWYVDGVEVHNAKTKPRE | SHKSYSCQVTHEGSTVEKTVAPT |
| | EQYNSTYRVVSVLTVLHQDWLN | ECS (SEQ ID NO: 67) |
| | GKEYKCKVSNKALPAPIEKTISK | |
| | AKGQPREPQVYTLPPSREEMTKN | |
| | QVSLTCLVKGFYPSDIAVEWESN | |
| | GQPENNYKTTPPVLDSDGSFFLY | |
| | SKLTVDKSRWQQGNVFSCSVMH | |
| | EALHNHYTQKSLSLSPGAGGGGS | |
| | GGGGSGGGGSEVQLVESGPGLV | |
| | KPSETLSLTCTVSGVSIRSNGYY | |
| | WGWIRQSPGKGLEWIGNMDYSG | |
| | TYQNPSLTSRVTISGDASKNQFSL | |
| | KLRSVTAADTAVYFCARERSNN | |
| | WYPIDYWGQGTLVTVSSASTKG | |
| | PSVFPLAPSSKSTSGGTAALGCL | |
| | VKDYFPEPVTVSWNSGALTSGV | |
| | HTFPAVLQSSGLYSLSSVVTVPSS | |
| | SLGTQTYICNVNHKPSNTKVDKR | |
| | VEPKSC (SEQ ID NO: 189) | |
| 89B09(6) | MHVAQPAVVLASSRGIASFVCEY | SSELTQDPAVSVALGQTVRITCQG |
| | ASPGKATEVRVTVLRQADSQVT | DSVRRFFAHWYQQKPGQAPVLVI |
| | EVCAATYMMGNELTFLDDSICT | SGKDTRPSGIPDRFSGSSSGNTASL |
| | GTSSGNQVNLTIQGLRAMDTGL | TITGAQAEDEADYYCNSRDSSGY |
| | YICKVELMYPPPYYLGIGNGTQI | LVLFGGGTKLTVLGQPKAAPSVT |
| | YVIDPEPCPDSDEPKSSDKTHTCP | LFPPSSEELQANKATLVCLISDFYP |
| | PCPAPEAAGGPSVFLFPPKPKDTL | GAVTVAWKADSSPVKAGVETTT |
| | MISRTPEVTCVVVDVSHEDPEVK | PSKQSNNKYAASSYLSLTPEQWK |
| | FNWYVDGVEVHNAKTKPREEQ | SHKSYSCQVTHEGSTVEKTVAPT |
| | YNSTYRVVSVLTVLHQDWLNGK | ECS (SEQ ID NO: 67) |
| | EYKCKVSNKALPAPIEKTISKAK | |
| | GQPREPQVYTLPPSREEMTKNQV | |
| | SLTCLVKGFYPSDIAVEWESNGQ | |
| | PENNYKTTPPVLDSDGSFFLYSK | |
| | LTVDKSRWQQGNVFSCSVMHEA | |
| | LHNHYTQKSLSLSPGAGGGGSG | |
| | GGGSGGGGSEVQLVESGPGLVK | |
| | PSETLSLTCTVSGVSIRSNGYYW | |
| | GWIRQSPGKGLEWIGNMDYSGT | |
| | YQNPSLTSRVTISGDASKNQFSL | |
| | KLRSVTAADTAVYFCARERSNN | |
| | WYPIDYWGQGTLVTVSSASTKG | |
| | PSVFPLAPSSKSTSGGTAALGCL | |
| | VKDYFPEPVTVSWNSGALTSGV | |
| | HTFPAVLQSSGLYSLSSVVTVPSS | |
| | SLGTQTYICNVNHKPSNTKVDKR | |
| | VEPKSC (SEQ ID NO: 190) | |
| 89B09(7) | AMHVAQPAVVLASSRGIASFVCE | SSELTQDPAVSVALGQTVRITCQG |
| | YASPGKATEVRVTVLRQADSQV | DSVRRFFAHWYQQKPGQAPVLVI |
| | TEVCAATYMMGNELTFLDDSICT | SGKDTRPSGIPDRFSGSSSGNTASL |
| | GTSSGNQVNLTIQGLRAMDTGL | TITGAQAEDEADYYCNSRDSSGY |
| | YICKVELMYPPPYYLGIGNGTQI | LVLFGGGTKLTVLGQPKAAPSVT |
| | YVIDPEPCPDSDEPKSSDKTHTCP | LFPPSSEELQANKATLVCLISDFYP |
| | PCPAPEAAGGPSVFLFPPKPKDTL | GAVTVAWKADSSPVKAGVETTT |
| | MISRTPEVTCVVVDVSHEDPEVK | PSKQSNNKYAASSYLSLTPEQWK |
| | FNWYVDGVEVHNAKTKPREEQ | SHKSYSCQVTHEGSTVEKTVAPT |
| | YNSTYRVVSVLTVLHQDWLNGK | ECS (SEQ ID NO: 67) |
| | EYKCKVSNKALPAPIEKTISKAK | |
| | GQPREPQVYTLPPSREEMTKNQV | |
| | SLTCLVKGFYPSDIAVEWESNGQ | |
| | PENNYKTTPPVLDSDGSFFLYSK | |
| | LTVDKSRWQQGNVFSCSVMHEA | |
| | LHNHYTQKSLSLSPGAGGGGSG | |
| | GGGSGGGGSEVQLVESGPGLVK | |
| | PSETLSLTCTVSGVSIRSNGYYW | |
| | GWIRQSPGKGLEWIGNMDYSGT | |
| | YQNPSLTSRVTISGDASKNQFSL | |
| | KLRSVTAADTAVYFCARERSNN | |
| | WYPIDYWGQGTLVTVSSASTKG | |
| | PSVFPLAPSSKSTSGGTAALGCL | |
| | VKDYFPEPVTVSWNSGALTSGV | |
| | HTFPAVLQSSGLYSLSSVVTVPSS | |
| | SLGTQTYICNVNHKPSNTKVDKR | |
| | VEPKSC (SEQ ID NO: 191) | |
| 98E10(1) | AMHVAQPAVVLASSRGIASFVCE | SYELTQPLSVSVALGQTARITCGG |
| | YASPGKATEVRVTVLRQADSQV | NNIENKNVHWYQQMPGQAPVLV |
| | TEVCAATYMMGNELTFLDDSICT | IYRDSNRPSGIPERFSGSNSGNTAT |
| | GTSSGNQVNLTIQGLRAMDTGL | LTINRAQAGDEADYYCQVWDSN |
| | YICKVELMYPPPYYLGIGNGTQI | TVVFGGGTKVTVLGQPKAAPSVT |
| | YVIDPEPCPDSDQEPKSSDKTHT | LFPPSSEELQANKATLVCLISDFYP |
| | CPPCPAPPVAGPSVFLFPPKPKDT | GAVTVAWKADSSPVKAGVETTT |
| | LMISRTPEVTCVVVDVSHEDPEV | PSKQSNNKYAASSYLSLTPEQWK |
| | KFNWYVDGVEVHNAKTKPREE | SHKSYSCQVTHEGSTVEKTVAPT |
| | QYNSTYRVVSVLTVLHQDWLNG | ECS (SEQ ID NO: 45) |
| | KEYKCAVSNKALPSSIEKTISKAK | |
| | GQPREPQVYTLPPSREEMTKNQV | |
| | SLTCLVKGFYPSDIAVEWESNGQ | |
| | PENNYKTTPPVLDSDGSFFLYSK | |
| | LTVDKSRWQQGNVFSCSVMHEA | |
| | LHNHYTQKSLSLSPGAGGGGSG | |
| | GGGSGGGGSQLQLQESGPGLVK | |
| | PSETLSLTCTVAGASVSSSSYYW | |
| | GWIRQSPGKGLEWIGTINYGGST | |
| | YHNPSLKRRVTVSVDTSKNQFSL | |
| | KLTSVTAADTAVYYCARHRGIY | |
| | HFDYWGQGTLVTVSSASTKGPS | |
| | VFPLAPSSKSTSGGTAALGCLVK | |
| | DYFPEPVTVSWNSGALTSGVHTF | |
| | PAVLQSSGLYSLSSVVTVPSSSLG | |
| | TQTYICNVNHKPSNTKVDKRVEP | |
| | KSC (SEQ ID NO: 46) | |
| 98E10(2) | AMHVAQPAVVLASSRGIASFVCE | SYELTQPLSVSVALGQTARITCGG |
| | YASPGKATEVRVTVLRQADSQV | NNIENKNVHWYQQKPGQAPVLVI |
| | TEVCAATYMMGNELTFLDDSICT | YRDSNRPSGIPERFSGSNSGNTAT |
| | GTSSGNQVNLTIQGLRAMDTGL | LTISRAQAGDEADYYCQVWDSTT |
| | YICKVELMYPPPYYLGIGNGTQI | VVFGGGTKVTVLGQPKAAPSVTL |
| | YVIDPEPCPDSDQEPKSSDKTHT | FPPSSEELQANKATLVCLISDFYP |
| | CPPCPAPPVAGPSVFLFPPKPKDT | GAVTVAWKADSSPVKAGVETTT |
| | LMISRTPEVTCVVVDVSHEDPEV | PSKQSNNKYAASSYLSLTPEQWK |
| | KFNWYVDGVEVHNAKTKPREE | SHKSYSCQVTHEGSTVEKTVAPT |
| | QYNSTYRVVSVLTVLHQDWLNG | ECS (SEQ ID NO: 65) |
| | KEYKCAVSNKALAAPIEKTISKA | |
| | KGQPREPQVYTLPPSREEMTKNQ | |
| | VSLTCLVKGFYPSDIAVEWESNG | |
| | QPENNYKTTPPVLDSDGSFFLYS | |
| | KLTVDKSRWQQGNVFSCSVMHE | |
| | ALHNHYTQKSLSLSPGAGGGGS | |
| | GGGGSGGGGSQLQLQESGPGLV | |
| | KPSETLSLTCTVSGASVSSSSYY | |
| | WGWIRQPPGKGLEWIGTINYGGS | |
| | TYHNPSLKRRVTISVDTSKNQFS | |
| | LKLSSVTAADTAVYYCARHRGI | |
| | YHFDYWGQGTLVTVSSASTKGP | |
| | SVFPLAPSSKSTSGGTAALGCLV | |
| | KDYFPEPVTVSWNSGALTSGVH | |
| | TFPAVLQSSGLYSLSSVVTVPSSS | |
| | LGTQTYICNVNHKPSNTKVDKR | |
| | VEPKSC (SEQ ID NO: 66) | |
| 98E10(3) | KAMHVAQPAVVLASSRGIASFV | SYELTQPLSVSVALGQTARITCGG |
| | CEYASPGKATEVRVTVLRQADS | NNIENKNVHWYQQKPGQAPVLVI |
| | QVTEVCAATYMMGNELTFLDDS | YRDSNRPSGIPERFSGSNSGNTAT |
| | ICTGTSSGNQVNLTIQGLRAMDT | LTISRAQAGDEADYYCQVWDSTT |
| | GLYICKVELMYPPPYYLGIGNGT | VVFGGGTKVTVLGQPKAAPSVTL |
| | QIYVIDPEPCPDSDQEPKSSDKTH | FPPSSEELQANKATLVCLISDFYP |
| | TCPPCPAPEAAGGPSVFLFPPKPK | GAVTVAWKADSSPVKAGVETTT |
| | DTLMISRTPEVTCVVVDVSHEDP | PSKQSNNKYAASSYLSLTPEQWK |
| | EVKFNWYVDGVEVHNAKTKPR | SHKSYSCQVTHEGSTVEKTVAPT |
| | EEQYNSTYRVVSVLTVLHQDWL | ECS (SEQ ID NO: 65) |
| | NGKEYKCKVSNKALPAPIEKTIS | |
| | KAKGQPREPQVYTLPPSREEMTK | |
| | NQVSLTCLVKGFYPSDIAVEWES | |
| | NGQPENNYKTTPPVLDSDGSFFL | |
| | YSKLTVDKSRWQQGNVFSCSVM | |
| | HEALHNHYTQKSLSLSPGAGGG | |
| | GSGGGGSGGGGSQLQLQESGPG | |
| | LVKPSETLSLTCTVAGASVSSSSY | |
| | YWGWIRQSPGKGLEWIGTINYG | |
| | GSTYHNPSLKRRVTVSVDTSKNQ | |
| | FSLKLTSVTAADTAVYYCARHR | |
| | GIYHFDYWGQGTLVTVSSASTK | |
| | GPSVFPLAPSSKSTSGGTAALGC | |
| | LVKDYFPEPVTVSWNSGALTSG | |
| | VHTFPAVLQSSGLYSLSSVVTVP | |
| | SSSLGTQTYICNVNHKPSNTKVD | |
| | KRVEPKSC (SEQ ID NO: 192) | |
| 98E10(4) | MHVAQPAVVLASSRGIASFVCEY | SYELTQPLSVSVALGQTARITCGG |
| | ASPGKATEVRVTVLRQADSQVT | NNIENKNVHWYQQKPGQAPVLVI |
| | EVCAATYMMGNELTFLDDSICT | YRDSNRPSGIPERFSGSNSGNTAT |
| | GTSSGNQVNLTIQGLRAMDTGL | LTISRAQAGDEADYYCQVWDSTT |
| | YICKVELMYPPPYYLGIGNGTQI | VVFGGGTKVTVLGQPKAAPSVTL |
| | YVIDPEPCPDSDQEPKSSDKTHT | FPPSSEELQANKATLVCLISDFYP |
| | CPPCPAPEAAGGPSVFLFPPKPKD | GAVTVAWKADSSPVKAGVETTT |
| | TLMISRTPEVTCVVVDVSHEDPE | PSKQSNNKYAASSYLSLTPEQWK |
| | VKFNWYVDGVEVHNAKTKPRE | SHKSYSCQVTHEGSTVEKTVAPT |
| | EQYNSTYRVVSVLTVLHQDWLN | ECS (SEQ ID NO: 65) |
| | GKEYKCKVSNKALPAPIEKTISK | |
| | AKGQPREPQVYTLPPSREEMTKN | |
| | QVSLTCLVKGFYPSDIAVEWESN | |
| | GQPENNYKTTPPVLDSDGSFFLY | |
| | SKLTVDKSRWQQGNVFSCSVMH | |
| | EALHNHYTQKSLSLSPGAGGGGS | |
| | GGGGSGGGGSQLQLQESGPGLV | |
| | KPSETLSLTCTVAGASVSSSSYY | |
| | WGWIRQSPGKGLEWIGTINYGGS | |
| | TYHNPSLKRRVTVSVDTSKNQFS | |
| | LKLTSVTAADTAVYYCARHRGI | |
| | YHFDYWGQGTLVTVSSASTKGP | |
| | SVFPLAPSSKSTSGGTAALGCLV | |
| | KDYFPEPVTVSWNSGALTSGVH | |
| | TFPAVLQSSGLYSLSSVVTVPSSS | |
| | LGTQTYICNVNHKPSNTKVDKR | |
| | VEPKSC (SEQ ID NO: 193) | |
| 98E10(5) | KAMHVAQPAVVLASSRGIASFV | SYELTQPLSVSVALGQTARITCGG |
| | CEYASPGKATEVRVTVLRQADS | NNIENKNVHWYQQKPGQAPVLVI |
| | QVTEVCAATYMMGNELTFLDDS | YRDSNRPSGIPERFSGSNSGNTAT |
| | ICTGTSSGNQVNLTIQGLRAMDT | LTISRAQAGDEADYYCQVWDSTT |
| | GLYICKVELMYPPPYYLGIGNGT | VVFGGGTKVTVLGQPKAAPSVTL |
| | QIYVIDPEPCPDSDEPKSSDKTHT | FPPSSEELQANKATLVCLISDFYP |
| | CPPCPAPEAAGGPSVFLFPPKPKD | GAVTVAWKADSSPVKAGVETTT |
| | TLMISRTPEVTCVVVDVSHEDPE | PSKQSNNKYAASSYLSLTPEQWK |
| | VKFNWYVDGVEVHNAKTKPRE | SHKSYSCQVTHEGSTVEKTVAPT |
| | EQYNSTYRVVSVLTVLHQDWLN | ECS (SEQ ID NO: 65) |
| | GKEYKCKVSNKALPAPIEKTISK | |
| | AKGQPREPQVYTLPPSREEMTKN | |
| | QVSLTCLVKGFYPSDIAVEWESN | |
| | GQPENNYKTTPPVLDSDGSFFLY | |
| | SKLTVDKSRWQQGNVFSCSVMH | |
| | EALHNHYTQKSLSLSPGAGGGGS | |
| | GGGGSGGGGSQLQLQESGPGLV | |
| | KPSETLSLTCTVAGASVSSSSYY | |
| | WGWIRQSPGKGLEWIGTINYGGS | |
| | TYHNPSLKRRVTVSVDTSKNQFS | |
| | LKLTSVTAADTAVYYCARHRGI | |
| | YHFDYWGQGTLVTVSSASTKGP | |
| | SVFPLAPSSKSTSGGTAALGCLV | |
| | KDYFPEPVTVSWNSGALTSGVH | |
| | TFPAVLQSSGLYSLSSVVTVPSSS | |
| | LGTQTYICNVNHKPSNTKVDKR | |
| | VEPKSC (SEQ ID NO: 194) | |
| 98E10(6) | MHVAQPAVVLASSRGIASFVCEY | SYELTQPLSVSVALGQTARITCGG |
| | ASPGKATEVRVTVLRQADSQVT | NNIENKNVHWYQQKPGQAPVLVI |
| | EVCAATYMMGNELTFLDDSICT | YRDSNRPSGIPERFSGSNSGNTAT |
| | GTSSGNQVNLTIQGLRAMDTGL | LTISRAQAGDEADYYCQVWDSTT |
| | YICKVELMYPPPYYLGIGNGTQI | VVFGGGTKVTVLGQPKAAPSVTL |
| | YVIDPEPCPDSDEPKSSDKTHTCP | FPPSSEELQANKATLVCLISDFYP |
| | PCPAPEAAGGPSVFLFPPKPKDTL | GAVTVAWKADSSPVKAGVETTT |
| | MISRTPEVTCVVVDVSHEDPEVK | PSKQSNNKYAASSYLSLTPEQWK |
| | FNWYVDGVEVHNAKTKPREEQ | SHKSYSCQVTHEGSTVEKTVAPT |
| | YNSTYRVVSVLTVLHQDWLNGK | ECS (SEQ ID NO: 65) |
| | EYKCKVSNKALPAPIEKTISKAK | |
| | GQPREPQVYTLPPSREEMTKNQV | |
| | SLTCLVKGFYPSDIAVEWESNGQ | |
| | PENNYKTTPPVLDSDGSFFLYSK | |
| | LTVDKSRWQQGNVFSCSVMHEA | |
| | LHNHYTQKSLSLSPGAGGGGSG | |
| | GGGSGGGGSQLQLQESGPGLVK | |
| | PSETLSLTCTVAGASVSSSSYYW | |
| | GWIRQSPGKGLEWIGTINYGGST | |
| | YHNPSLKRRVTVSVDTSKNQFSL | |
| | KLTSVTAADTAVYYCARHRGIY | |
| | HFDYWGQGTLVTVSSASTKGPS | |
| | VFPLAPSSKSTSGGTAALGCLVK | |
| | DYFPEPVTVSWNSGALTSGVHTF | |
| | PAVLQSSGLYSLSSVVTVPSSSLG | |
| | TQTYICNVNHKPSNTKVDKRVEP | |
| | KSC (SEQ ID NO: 195) | |
| 98E10(7) | AMHVAQPAVVLASSRGIASFVCE | SYELTQPLSVSVALGQTARITCGG |
| | YASPGKATEVRVTVLRQADSQV | NNIENKNVHWYQQKPGQAPVLVI |
| | TEVCAATYMMGNELTFLDDSICT | YRDSNRPSGIPERFSGSNSGNTAT |
| | GTSSGNQVNLTIQGLRAMDTGL | LTISRAQAGDEADYYCQVWDSTT |
| | YICKVELMYPPPYYLGIGNGTQI | VVFGGGTKVTVLGQPKAAPSVTL |
| | YVIDPEPCPDSDEPKSSDKTHTCP | FPPSSEELQANKATLVCLISDFYP |
| | PCPAPEAAGGPSVFLFPPKPKDTL | GAVTVAWKADSSPVKAGVETTT |
| | MISRTPEVTCVVVDVSHEDPEVK | PSKQSNNKYAASSYLSLTPEQWK |
| | FNWYVDGVEVHNAKTKPREEQ | SHKSYSCQVTHEGSTVEKTVAPT |
| | YNSTYRVVSVLTVLHQDWLNGK | ECS (SEQ ID NO: 65) |
| | EYKCKVSNKALPAPIEKTISKAK | |
| | GQPREPQVYTLPPSREEMTKNQV | |
| | SLTCLVKGFYPSDIAVEWESNGQ | |
| | PENNYKTTPPVLDSDGSFFLYSK | |
| | LTVDKSRWQQGNVFSCSVMHEA | |
| | LHNHYTQKSLSLSPGAGGGGSG | |
| | GGGSGGGGSQLQLQESGPGLVK | |
| | PSETLSLTCTVAGASVSSSSYYW | |
| | GWIRQSPGKGLEWIGTINYGGST | |
| | YHNPSLKRRVTVSVDTSKNQFSL | |
| | KLTSVTAADTAVYYCARHRGIY | |
| | HFDYWGQGTLVTVSSASTKGPS | |
| | VFPLAPSSKSTSGGTAALGCLVK | |
| | DYFPEPVTVSWNSGALTSGVHTF | |
| | PAVLQSSGLYSLSSVVTVPSSSLG | |
| | TQTYICNVNHKPSNTKVDKRVEP | |
| | KSC (SEQ ID NO: 196) | |
| 98C01(1) | AMHVAQPAVVLASSRGIASFVCE | SYELTQPPSVSVALGQTARITCGG |
| | YASPGKATEVRVTVLRQADSQV | NNIENKNVHWYQQKPGQAPVLVI |
| | TEVCAATYMMGNELTFLDDSICT | YRDSNRPSGIPERFSGSNSGNTAT |
| | GTSSGNQVNLTIQGLRAMDTGL | LSINRAQAGDEADYYCQVWDSN |
| | YICKVELMYPPPYYLGIGNGTQI | TVVFGGGTKLTVLGQPKAAPSVT |
| | YVIDPEPCPDSDQEPKSSDKTHT | LFPPSSEELQANKATLVCLISDFYP |
| | CPPCPAPPVAGPSVFLFPPKPKDT | GAVTVAWKADSSPVKAGVETTT |
| | LMISRTPEVTCVVVDVSHEDPEV | PSKQSNNKYAASSYLSLTPEQWK |
| | KFNWYVDGVEVHNAKTKPREE | SHKSYSCQVTHEGSTVEKTVAPT |
| | QYNSTYRVVSVLTVLHQDWLNG | ECS (SEQ ID NO: 49) |
| | KEYKCAVSNKALPSSIEKTISKAK | |
| | GQPREPQVYTLPPSREEMTKNQV | |
| | SLTCLVKGFYPSDIAVEWESNGQ | |
| | PENNYKTTPPVLDSDGSFFLYSK | |
| | LTVDKSRWQQGNVFSCSVMHEA | |
| | LHNHYTQKSLSLSPGAGGGGSG | |
| | GGGSGGGGSQVQLQESGPGLVK | |
| | PSETLSLTCTVSGGSISSSSYYWG | |
| | WIRQPPGKGLEWIGGIGSVDYSG | |
| | NTYYKPSLKSRVTISVDTSKNQF | |
| | SLKLSSVTAADTAVYHCARHRGI | |
| | YFFDYWGQGTLVTVSSASTKGPS | |
| | VFPLAPSSKSTSGGTAALGCLVK | |
| | DYFPEPVTVSWNSGALTSGVHTF | |
| | PAVLQSSGLYSLSSVVTVPSSSLG | |
| | TQTYICNVNHKPSNTKVDKRVEP | |
| | KSC (SEQ ID NO: 50) | |
| 98C01(2) | AMHVAQPAVVLASSRGIASFVCE | SYELTQPPSMSVAPGQTARITCGG |
| | YASPGKATEVRVTVLRQADSQV | SNIEDKNVHWYQQKPGQAPVLVI |
| | TEVCAATYMMGNELTFLDDSICT | KRDSDRPSGIPERFSGSNSGNTAT |
| | GTSSGNQVNLTIQGLRAMDTGL | LTISRAQAGDEAEYYCQVWDSST |
| | YICKVELMYPPPYYLGIGNGTQI | VVFGGGTKLTVLGQPKAAPSVTL |
| | YVIDPEPCPDSDQEPKSSDKTHT | FPPSSEELQANKATLVCLISDFYP |
| | CPPCPAPPVAGPSVFLFPPKPKDT | GAVTVAWKADSSPVKAGVETTT |
| | LMISRTPEVTCVVVDVSHEDPEV | PSKQSNNKYAASSYLSLTPEQWK |
| | KFNWYVDGVEVHNAKTKPREE | SHKSYSCQVTHEGSTVEKTVAPT |
| | QYNSTYRVVSVLTVLHQDWLNG | ECS (SEQ ID NO: 69) |
| | KEYKCAVSNKALAAPIEKTISKA | |
| | KGQPREPQVYTLPPSREEMTKNQ | |
| | VSLTCLVKGFYPSDIAVEWESNG | |
| | QPENNYKTTPPVLDSDGSFFLYS | |
| | KLTVDKSRWQQGNVFSCSVMHE | |
| | ALHNHYTQKSLSLSPGAGGGGS | |
| | GGGGSGGGGSQVQLQESGPGLV | |
| | KPSETLSLTCTVSGGSISSSSYYW | |
| | GWIRQPPGKGLEWIGGIGSVDYS | |
| | GNTYYKPSLKSRVTISVDTSKNQ | |
| | FSLKLSSVTAADTAVYHCARHR | |
| | GIYFFDYWGQGTLVTVSSASTKG | |
| | PSVFPLAPSSKSTSGGTAALGCL | |
| | VKDYFPEPVTVSWNSGALTSGV | |
| | HTFPAVLQSSGLYSLSSVVTVPSS | |
| | SLGTQTYICNVNHKPSNTKVDKR | |
| | VEPKSC (SEQ ID NO: 70) | |
| 98C01(3) | KAMHVAQPAVVLASSRGIASFV | SYELTQPPSMSVAPGQTARITCGG |
| | CEYASPGKATEVRVTVLRQADS | SNIEDKNVHWYQQKPGQAPVLVI |
| | QVTEVCAATYMMGNELTFLDDS | KRDSDRPSGIPERFSGSNSGNTAT |
| | ICTGTSSGNQVNLTIQGLRAMDT | LTISRAQAGDEAEYYCQVWDSST |
| | GLYICKVELMYPPPYYLGIGNGT | VVFGGGTKLTVLGQPKAAPSVTL |
| | QIYVIDPEPCPDSDQEPKSSDKTH | FPPSSEELQANKATLVCLISDFYP |
| | TCPPCPAPEAAGGPSVFLFPPKPK | GAVTVAWKADSSPVKAGVETTT |
| | DTLMISRTPEVTCVVVDVSHEDP | PSKQSNNKYAASSYLSLTPEQWK |
| | EVKFNWYVDGVEVHNAKTKPR | SHKSYSCQVTHEGSTVEKTVAPT |
| | EEQYNSTYRVVSVLTVLHQDWL | ECS (SEQ ID NO: 69) |
| | NGKEYKCKVSNKALPAPIEKTIS | |
| | KAKGQPREPQVYTLPPSREEMTK | |
| | NQVSLTCLVKGFYPSDIAVEWES | |
| | NGQPENNYKTTPPVLDSDGSFFL | |
| | YSKLTVDKSRWQQGNVFSCSVM | |
| | HEALHNHYTQKSLSLSPGAGGG | |
| | GSGGGGSGGGGSQVQLQESGPG | |
| | LVKPSETLSLTCTVSGGSISSSSY | |
| | YWGWIRQPPGKGLEWIGGIGSV | |
| | DYSGNTYYKPSLKSRVTISVDTS | |
| | KNQFSLKLSSVTAADTAVYHCA | |
| | RHRGIYFFDYWGQGTLVTVSSAS | |
| | TKGPSVFPLAPSSKSTSGGTAAL | |
| | GCLVKDYFPEPVTVSWNSGALTS | |
| | GVHTFPAVLQSSGLYSLSSVVTV | |
| | PSSSLGTQTYICNVNHKPSNTKV | |
| | DKRVEPKSC (SEQ ID NO: 197) | |
| 98C01(4) | MHVAQPAVVLASSRGIASFVCEY | SYELTQPPSMSVAPGQTARITCGG |
| | ASPGKATEVRVTVLRQADSQVT | SNIEDKNVHWYQQKPGQAPVLVI |
| | EVCAATYMMGNELTFLDDSICT | KRDSDRPSGIPERFSGSNSGNTAT |
| | GTSSGNQVNLTIQGLRAMDTGL | LTISRAQAGDEAEYYCQVWDSST |
| | YICKVELMYPPPYYLGIGNGTQI | VVFGGGTKLTVLGQPKAAPSVTL |
| | YVIDPEPCPDSDQEPKSSDKTHT | FPPSSEELQANKATLVCLISDFYP |
| | CPPCPAPEAAGGPSVFLFPPKPKD | GAVTVAWKADSSPVKAGVETTT |
| | TLMISRTPEVTCVVVDVSHEDPE | PSKQSNNKYAASSYLSLTPEQWK |
| | VKFNWYVDGVEVHNAKTKPRE | SHKSYSCQVTHEGSTVEKTVAPT |
| | EQYNSTYRVVSVLTVLHQDWLN | ECS (SEQ ID NO: 69) |
| | GKEYKCKVSNKALPAPIEKTISK | |
| | AKGQPREPQVYTLPPSREEMTKN | |
| | QVSLTCLVKGFYPSDIAVEWESN | |
| | GQPENNYKTTPPVLDSDGSFFLY | |
| | SKLTVDKSRWQQGNVFSCSVMH | |
| | EALHNHYTQKSLSLSPGAGGGGS | |
| | GGGGSGGGGSQVQLQESGPGLV | |
| | KPSETLSLTCTVSGGSISSSSYYW | |
| | GWIRQPPGKGLEWIGGIGSVDYS | |
| | GNTYYKPSLKSRVTISVDTSKNQ | |
| | FSLKLSSVTAADTAVYHCARHR | |
| | GIYFFDYWGQGTLVTVSSASTKG | |
| | PSVFPLAPSSKSTSGGTAALGCL | |
| | VKDYFPEPVTVSWNSGALTSGV | |
| | HTFPAVLQSSGLYSLSSVVTVPSS | |
| | SLGTQTYICNVNHKPSNTKVDKR | |
| | VEPKSC (SEQ ID NO: 198) | |
| 98C01(5) | KAMHVAQPAVVLASSRGIASFV | SYELTQPPSMSVAPGQTARITCGG |
| | CEYASPGKATEVRVTVLRQADS | SNIEDKNVHWYQQKPGQAPVLVI |
| | QVTEVCAATYMMGNELTFLDDS | KRDSDRPSGIPERFSGSNSGNTAT |
| | ICTGTSSGNQVNLTIQGLRAMDT | LTISRAQAGDEAEYYCQVWDSST |
| | GLYICKVELMYPPPYYLGIGNGT | VVFGGGTKLTVLGQPKAAPSVTL |
| | QIYVIDPEPCPDSDEPKSSDKTHT | FPPSSEELQANKATLVCLISDFYP |
| | CPPCPAPEAAGGPSVFLFPPKPKD | GAVTVAWKADSSPVKAGVETTT |
| | TLMISRTPEVTCVVVDVSHEDPE | PSKQSNNKYAASSYLSLTPEQWK |
| | VKFNWYVDGVEVHNAKTKPRE | SHKSYSCQVTHEGSTVEKTVAPT |
| | EQYNSTYRVVSVLTVLHQDWLN | ECS (SEQ ID NO: 69) |
| | GKEYKCKVSNKALPAPIEKTISK | |
| | AKGQPREPQVYTLPPSREEMTKN | |
| | QVSLTCLVKGFYPSDIAVEWESN | |
| | GQPENNYKTTPPVLDSDGSFFLY | |
| | SKLTVDKSRWQQGNVFSCSVMH | |
| | EALHNHYTQKSLSLSPGAGGGGS | |
| | GGGGSGGGGSQVQLQESGPGLV | |
| | KPSETLSLTCTVSGGSISSSSYYW | |
| | GWIRQPPGKGLEWIGGIGSVDYS | |
| | GNTYYKPSLKSRVTISVDTSKNQ | |
| | FSLKLSSVTAADTAVYHCARHR | |
| | GIYFFDYWGQGTLVTVSSASTKG | |
| | PSVFPLAPSSKSTSGGTAALGCL | |
| | VKDYFPEPVTVSWNSGALTSGV | |
| | HTFPAVLQSSGLYSLSSVVTVPSS | |
| | SLGTQTYICNVNHKPSNTKVDKR | |
| | VEPKSC (SEQ ID NO: 199) | |
| 98C01(6) | MHVAQPAVVLASSRGIASFVCEY | SYELTQPPSMSVAPGQTARITCGG |
| | ASPGKATEVRVTVLRQADSQVT | SNIEDKNVHWYQQKPGQAPVLVI |
| | EVCAATYMMGNELTFLDDSICT | KRDSDRPSGIPERFSGSNSGNTAT |
| | GTSSGNQVNLTIQGLRAMDTGL | LTISRAQAGDEAEYYCQVWDSST |
| | YICKVELMYPPPYYLGIGNGTQI | VVFGGGTKLTVLGQPKAAPSVTL |
| | YVIDPEPCPDSDEPKSSDKTHTCP | FPPSSEELQANKATLVCLISDFYP |
| | PCPAPEAAGGPSVFLFPPKPKDTL | GAVTVAWKADSSPVKAGVETTT |
| | MISRTPEVTCVVVDVSHEDPEVK | PSKQSNNKYAASSYLSLTPEQWK |
| | FNWYVDGVEVHNAKTKPREEQ | SHKSYSCQVTHEGSTVEKTVAPT |
| | YNSTYRVVSVLTVLHQDWLNGK | ECS (SEQ ID NO: 69) |
| | EYKCKVSNKALPAPIEKTISKAK | |
| | GQPREPQVYTLPPSREEMTKNQV | |
| | SLTCLVKGFYPSDIAVEWESNGQ | |
| | PENNYKTTPPVLDSDGSFFLYSK | |
| | LTVDKSRWQQGNVFSCSVMHEA | |
| | LHNHYTQKSLSLSPGAGGGGSG | |
| | GGGSGGGGSQVQLQESGPGLVK | |
| | PSETLSLTCTVSGGSISSSSYYWG | |
| | WIRQPPGKGLEWIGGIGSVDYSG | |
| | NTYYKPSLKSRVTISVDTSKNQF | |
| | SLKLSSVTAADTAVYHCARHRGI | |
| | YFFDYWGQGTLVTVSSASTKGPS | |
| | VFPLAPSSKSTSGGTAALGCLVK | |
| | DYFPEPVTVSWNSGALTSGVHTF | |
| | PAVLQSSGLYSLSSVVTVPSSSLG | |
| | TQTYICNVNHKPSNTKVDKRVEP | |
| | KSC (SEQ ID NO: 200) | |
| 98C01(7) | AMHVAQPAVVLASSRGIASFVCE | SYELTQPPSMSVAPGQTARITCGG |
| | YASPGKATEVRVTVLRQADSQV | SNIEDKNVHWYQQKPGQAPVLVI |
| | TEVCAATYMMGNELTFLDDSICT | KRDSDRPSGIPERFSGSNSGNTAT |
| | GTSSGNQVNLTIQGLRAMDTGL | LTISRAQAGDEAEYYCQVWDSST |
| | YICKVELMYPPPYYLGIGNGTQI | VVFGGGTKLTVLGQPKAAPSVTL |
| | YVIDPEPCPDSDEPKSSDKTHTCP | FPPSSEELQANKATLVCLISDFYP |
| | PCPAPEAAGGPSVFLFPPKPKDTL | GAVTVAWKADSSPVKAGVETTT |
| | MISRTPEVTCVVVDVSHEDPEVK | PSKQSNNKYAASSYLSLTPEQWK |
| | FNWYVDGVEVHNAKTKPREEQ | SHKSYSCQVTHEGSTVEKTVAPT |
| | YNSTYRVVSVLTVLHQDWLNGK | ECS (SEQ ID NO: 69) |
| | EYKCKVSNKALPAPIEKTISKAK | |
| | GQPREPQVYTLPPSREEMTKNQV | |
| | SLTCLVKGFYPSDIAVEWESNGQ | |
| | PENNYKTTPPVLDSDGSFFLYSK | |
| | LTVDKSRWQQGNVFSCSVMHEA | |
| | LHNHYTQKSLSLSPGAGGGGSG | |
| | GGGSGGGGSQVQLQESGPGLVK | |
| | PSETLSLTCTVSGGSISSSSYYWG | |
| | WIRQPPGKGLEWIGGIGSVDYSG | |
| | NTYYKPSLKSRVTISVDTSKNQF | |
| | SLKLSSVTAADTAVYHCARHRGI | |
| | YFFDYWGQGTLVTVSSASTKGPS | |
| | VFPLAPSSKSTSGGTAALGCLVK | |
| | DYFPEPVTVSWNSGALTSGVHTF | |
| | PAVLQSSGLYSLSSVVTVPSSSLG | |
| | TQTYICNVNHKPSNTKVDKRVEP | |
| | KSC (SEQ ID NO: 201) | |
| 97G07 | AMHVAQPAVVLASSRGIASFVCE | YYELTQPLSVSVALGQTARIPCGG |
| | YASPGKATEVRVTVLRQADSQV | NNIENKNVHWYQQKPGQAPVLVI |
| | TEVCAATYMMGNELTFLDDSICT | YRDSNRPSGIPERFSGSNSGNTAT |
| | GTSSGNQVNLTIQGLRAMDTGL | LSINRAQAGDEADYYCQVWDSN |
| | YICKVELMYPPPYYLGIGNGTQI | TVIFGGGTKVTVLGQPKAAPSVT |
| | YVIDPEPCPDSDQEPKSSDKTHT | LFPPSSEELQANKATLVCLISDFYP |
| | CPPCPAPPVAGPSVFLFPPKPKDT | GAVTVAWKADSSPVKAGVETTT |
| | LMISRTPEVTCVVVDVSHEDPEV | PSKQSNNKYAASSYLSLTPEQWK |
| | KFNWYVDGVEVHNAKTKPREE | SHKSYSCQVTHEGSTVEKTVAPT |
| | QYNSTYRVVSVLTVLHQDWLNG | ECS (SEQ ID NO: 5) |
| | KEYKCAVSNKALPSSIEKTISKAK | |
| | GQPREPQVYTLPPSREEMTKNQV | |
| | SLTCLVKGFYPSDIAVEWESNGQ | |
| | PENNYKTTPPVLDSDGSFFLYSK | |
| | LTVDKSRWQQGNVFSCSVMHEA | |
| | LHNHYTQKSLSLSPGAGGGGSG | |
| | GGGSGGGGSQLQLQESGPGLVK | |
| | PSETLSLTCTVSGGSISSSSYYWG | |
| | WIRQPPGKGLEWIGGIGSVDYSG | |
| | NTYYKPSLKSRVTISVDTSKNQF | |
| | SLKLSTVTAADTAVYHCARHRGI | |
| | YFFDYWGQGTLVTVSSASTKGPS | |
| | VFPLAPSSKSTSGGTAALGCLVK | |
| | DYFPEPVTVSWNSGALTSGVHTF | |
| | PAVLQSSGLYSLSSVVTVPSSSLG | |
| | TQTYICNVNHKPSNTKVDKRVEP | |
| | KSC (SEQ ID NO: 38) | |
| 97G07(2) | EPKSSDKTHTCPPCPAPPVAGPS | YYELTQPLSVSVALGQTARIPCGG |
| | VFLFPPKPKDTLMISRTPEVTCVV | NNIENKNVHWYQQKPGQAPVLVI |
| | VDVSHEDPEVKFNWYVDGVEV | YRDSNRPSGIPERFSGSNSGNTAT |
| | HNAKTKPREEQYNSTYRVVSVL | LSINRAQAGDEADYYCQVWDSN |
| | TVLHQDWLNGKEYKCAVSNKA | TVIFGGGTKVTVLGQPKAAPSVT |
| | LPSSIEKTISKAKGQPREPQVYTL | LFPPSSEELQANKATLVCLISDFYP |
| | PPSREEMTKNQVSLTCLVKGFYP | GAVTVAWKADSSPVKAGVETTT |
| | SDIAVEWESNGQPENNYKTTPPV | PSKQSNNKYAASSYLSLTPEQWK |
| | LDSDGSFFLYSKLTVDKSRWQQ | SHKSYSCQVTHEGSTVEKTVAPT |
| | GNVFSCSVMHEALHNHYTQKSL | ECS (SEQ ID NO: 5) |
| | SLSPGAGGGGSGGGGSGGGGSQ | |
| | LQLQESGPGLVKPSETLSLTCTVS | |
| | GGSISSSSYYWGWIRQPPGKGLE | |
| | WIGGIGSVDYSGNTYYKPSLKSR | |
| | VTISVDTSKNQFSLKLSTVTAAD | |
| | TAVYHCARHRGIYFFDYWGQGT | |
| | LVTVSSASTKGPSVFPLAPSSKST | |
| | SGGTAALGCLVKDYFPEPVTVS | |
| | WNSGALTSGVHTFPAVLQSSGL | |
| | YSLSSVVTVPSSSLGTQTYICNVN | |
| | HKPSNTKVDKRVEPKSC (SEQ ID | |
| | NO: 73) | |
| 68F03 | AMHVAQPAVVLASSRGIASFVCE | SFELTQPLSVSVALGQTARITCGGI |
| | YASPGKATEVRVTVLRQADSQV | NIENKNVHYYQQKPGQAPVLVIY |
| | TEVCAATYMMGNELTFLDDSICT | RDTNRPSGIPERFSGSNSGNTATL |
| | GTSSGNQVNLTIQGLRAMDTGL | TISRAQAGDEADYYCQVWDSNT |
| | YICKVELMYPPPYYLGIGNGTQI | VIFGGGTKVTVLGQPKAAPSVTLF |
| | YVIDPEPCPDSDQEPKSSDKTHT | PPSSEELQANKATLVCLISDFYPG |
| | CPPCPAPPVAGPSVFLFPPKPKDT | AVTVAWKADSSPVKAGVETTTPS |
| | LMISRTPEVTCVVVDVSHEDPEV | KQSNNKYAASSYLSLTPEQWKSH |
| | KFNWYVDGVEVHNAKTKPREE | KSYSCQVTHEGSTVEKTVAPTEC |
| | QYNSTYRVVSVLTVLHQDWLNG | S (SEQ ID NO: 41) |
| | KEYKCAVSNKALPSSIEKTISKAK | |
| | GQPREPQVYTLPPSREEMTKNQV | |
| | SLTCLVKGFYPSDIAVEWESNGQ | |
| | PENNYKTTPPVLDSDGSFFLYSK | |
| | LTVDKSRWQQGNVFSCSVMHEA | |
| | LHNHYTQKSLSLSPGAGGGGSG | |
| | GGGSGGGGSQLQLQESGPGLVK | |
| | PSETLSLTCTVSGGSIDTSSQYWA | |
| | WIRQPPGKGLEWIGGIYFSGSTY | |
| | QNPSLKSRVTTSVDTSKNQFSLR | |
| | LSSMTAADTAVYYCARHRGIYFF | |
| | DYWGQGTLVTVSSASTKGPSVFP | |
| | LAPSSKSTSGGTAALGCLVKDYF | |
| | PEPVTVSWNSGALTSGVHTFPAV | |
| | LQSSGLYSLSSVVTVPSSSLGTQT | |
| | YICNVNHKPSNTKVDKRVEPKSC | |
| | (SEQ ID NO: 42) | |
| 84E11 | AMHVAQPAVVLASSRGIASFVCE | SSELTQDPAVSVALGQTVRITCQG |
| | YASPGKATEVRVTVLRQADSQV | DSLRNYFANWYQQKPGQAPVLVI |
| | TEVCAATYMMGNELTFLDDSICT | YGKNNRPSGIPDRFSGSSSGNTAS |
| | GTSSGNQVNLTIQGLRAMDTGL | LTITGAQAEDEADYYCNSRDSSG |
| | YICKVELMYPPPYYLGIGNGTQI | YHVVFGGGTKLTVLGQPKAAPSV |
| | YVIDPEPCPDSDQEPKSSDKTHT | TLFPPSSEELQANKATLVCLISDFY |
| | CPPCPAPPVAGPSVFLFPPKPKDT | PGAVTVAWKADSSPVKAGVETT |
| | LMISRTPEVTCVVVDVSHEDPEV | TPSKQSNNKYAASSYLSLTPEQW |
| | KFNWYVDGVEVHNAKTKPREE | KSHKSYSCQVTHEGSTVEKTVAP |
| | QYNSTYRVVSVLTVLHQDWLNG | TECS (SEQ ID NO: 43) |
| | KEYKCAVSNKALPSSIEKTISKAK | |
| | GQPREPQVYTLPPSREEMTKNQV | |
| | SLTCLVKGFYPSDIAVEWESNGQ | |
| | PENNYKTTPPVLDSDGSFFLYSK | |
| | LTVDKSRWQQGNVFSCSVMHEA | |
| | LHNHYTQKSLSLSPGAGGGGSG | |
| | GGGSGGGGSQLQLQESGPGLVK | |
| | PSETLSLTCTVSGGSISSGSFYWG | |
| | WIRQPPGKGLEWIGTIYYSGSTY | |
| | YNPSLKSRVAISVDTSKNQFYLN | |
| | LNFVTAADTAVYHCARERSSSW | |
| | YPFDYWGQGTLVTVSSASTKGPS | |
| | VFPLAPSSKSTSGGTAALGCLVK | |
| | DYFPEPVTVSWNSGALTSGVHTF | |
| | PAVLQSSGLYSLSSVVTVPSSSLG | |
| | TQTYICNVNHKPSNTKVDKRVEP | |
| | KSC (SEQ ID NO: 44) | |
| 88B06 | AMHVAQPAVVLASSRGIASFVCE | SYELTQPLSVSVALGQTARISCGG |
| | YASPGKATEVRVTVLRQADSQV | NNIGRKNVHWYQQKPGQAPVLVI |
| | TEVCAATYMMGNELTFLDDSICT | YGDSNRPSGIPERFSGSNSGNTAT |
| | GTSSGNQVNLTIQGLRAMDTGL | LTIGRAQAGDEADYYCQVWDSST |
| | YICKVELMYPPPYYLGIGNGTQI | VFGGGTKLTVLGQPKAAPSVTLF |
| | YVIDPEPCPDSDQEPKSSDKTHT | PPSSEELQANKATLVCLISDFYPG |
| | CPPCPAPPVAGPSVFLFPPKPKDT | AVTVAWKADSSPVKAGVETTTPS |
| | LMISRTPEVTCVVVDVSHEDPEV | KQSNNKYAASSYLSLTPEQWKSH |
| | KFNWYVDGVEVHNAKTKPREE | KSYSCQVTHEGSTVEKTVAPTEC |
| | QYNSTYRVVSVLTVLHQDWLNG | S (SEQ ID NO: 51) |
| | KEYKCAVSNKALPSSIEKTISKAK | |
| | GQPREPQVYTLPPSREEMTKNQV | |
| | SLTCLVKGFYPSDIAVEWESNGQ | |
| | PENNYKTTPPVLDSDGSFFLYSK | |
| | LTVDKSRWQQGNVFSCSVMHEA | |
| | LHNHYTQKSLSLSPGAGGGGSG | |
| | GGGSGGGGSEVQLLESGGGLVQ | |
| | PGGSLRLSCAASGFTFSNYAMS | |
| | WVRQAPGKGLEWVSVISGSGDS | |
| | TYYAESVKGRFTISRDNSKNTLY | |
| | LQMTSLRAEDTAEYYCAKDRTP | |
| | VYGLDVWGQGTTVTVSSASTKG | |
| | PSVFPLAPSSKSTSGGTAALGCL | |
| | VKDYFPEPVTVSWNSGALTSGV | |
| | HTFPAVLQSSGLYSLSSVVTVPSS | |
| | SLGTQTYICNVNHKPSNTKVDKR | |
| | VEPKSC (SEQ ID NO: 52) | |
| 95B06 | AMHVAQPAVVLASSRGIASFVCE | DMQMTQSPSSLSASVGDRVTITC |
| | YASPGKATEVRVTVLRQADSQV | RASQGIRNDLDWYQQKPGKVPK |
| | TEVCAATYMMGNELTFLDDSICT | RLIHAASSLQSGVPSRFSGSGSGT |
| | GTSSGNQVNLTIQGLRAMDTGL | EFTLTISSLQPEDFVTYYCLQHNT |
| | YICKVELMYPPPYYLGIGNGTQI | YPWTFGQGTKLEIKRTVAAPSVFI |
| | YVIDPEPCPDSDQEPKSSDKTHT | FPPSDEQLKSGTASVVCLLNNFYP |
| | CPPCPAPPVAGPSVFLFPPKPKDT | REAKVQWKVDNALQSGNSQESV |
| | LMISRTPEVTCVVVDVSHEDPEV | TEQDSKDSTYSLSSTLTLSKADYE |
| | KFNWYVDGVEVHNAKTKPREE | KHKVYACEVTHQGLSSPVTKSFN |
| | QYNSTYRVVSVLTVLHQDWLNG | RGEC (SEQ ID NO: 53) |
| | KEYKCAVSNKALPSSIEKTISKAK | |
| | GQPREPQVYTLPPSREEMTKNQV | |
| | SLTCLVKGFYPSDIAVEWESNGQ | |
| | PENNYKTTPPVLDSDGSFFLYSK | |
| | LTVDKSRWQQGNVFSCSVMHEA | |
| | LHNHYTQKSLSLSPGAGGGGSG | |
| | GGGSGGGGSEVQLLESGGGLVQ | |
| | PGGSLRLSCAASGFTFSSYAMTW | |
| | VRQAPGKGLEWVSDISGSGGSTK | |
| | YADSVKGRFTISRDNPKNTLYLQ | |
| | MNSLRAEDTAVYYCAKDLGFYS | |
| | TWDTDYWGQGTLVTVSSASTKG | |
| | PSVFPLAPSSKSTSGGTAALGCL | |
| | VKDYFPEPVTVSWNSGALTSGV | |
| | HTFPAVLQSSGLYSLSSVVTVPSS | |
| | SLGTQTYICNVNHKPSNTKVDKR | |
| | VEPKSC (SEQ ID NO: 54) | |
| 30F02 | AMHVAQPAVVLASSRGIASFVCE | SYVLTQPLSVSVALGQTARITCGG |
| | YASPGKATEVRVTVLRQADSQV | NNIGDKNVHWYQQKPGQAPVLV |
| | TEVCAATYMMGNELTFLDDSICT | ISRDSNRPSGIPERFSGSNSGNTAT |
| | GTSSGNQVNLTIQGLRAMDTGL | LTISRAQAGDESDYYCQVWDSST |
| | YICKVELMYPPPYYLGIGNGTQI | VVFGGGTKVTVLGQPKAAPSVTL |
| | YVIDPEPCPDSDQEPKSSDKTHT | FPPSSEELQANKATLVCLISDFYP |
| | CPPCPAPPVAGPSVFLFPPKPKDT | GAVTVAWKADSSPVKAGVETTT |
| | LMISRTPEVTCVVVDVSHEDPEV | PSKQSNNKYAASSYLSLTPEQWK |
| | KFNWYVDGVEVHNAKTKPREE | SHKSYSCQVTHEGSTVEKTVAPT |
| | QYNSTYRVVSVLTVLHQDWLNG | ECS (SEQ ID NO: 55) |
| | KEYKCAVSNKALPSSIEKTISKAK | |
| | GQPREPQVYTLPPSREEMTKNQV | |
| | SLTCLVKGFYPSDIAVEWESNGQ | |
| | PENNYKTTPPVLDSDGSFFLYSK | |
| | LTVDKSRWQQGNVFSCSVMHEA | |
| | LHNHYTQKSLSLSPGAGGGGSG | |
| | GGGSGGGGSQLQLQESGPGLVK | |
| | PSETLSLTCTVSGASISSSNHYWG | |
| | WGWIRQPPGKGLEWIGSIYYSGN | |
| | TYYNPSLKSRVTISVDTSKNQFSL | |
| | KLSSVTAADTAVYYCARHRGSY | |
| | FFDHWGQGTLVTVSSASTKGPSV | |
| | FPLAPSSKSTSGGTAALGCLVKD | |
| | YFPEPVTVSWNSGALTSGVHTFP | |
| | AVLQSSGLYSLSSVVTVPSSSLGT | |
| | QTYICNVNHKPSNTKVDKRVEP | |
| | KSC (SEQ ID NO: 56) | |
| 85F12 | AMHVAQPAVVLASSRGIASFVCE | SSELTQPPSVSVSPGQTASITCSGD |
| | YASPGKATEVRVTVLRQADSQV | KLGDKYACWYQQKAGQSPVVVI |
| | TEVCAATYMMGNELTFLDDSICT | YQDRKRPSGIPERFSGSNSGNTAT |
| | GTSSGNQVNLTIQGLRAMDTGL | LTISGTQAMDEADYYCQTWDRR |
| | YICKVELMYPPPYYLGIGNGTQI | TAVFGGGTKLTVLGQPKAAPSVT |
| | YVIDPEPCPDSDQEPKSSDKTHT | LFPPSSEELQANKATLVCLISDFYP |
| | CPPCPAPPVAGPSVFLFPPKPKDT | GAVTVAWKADSSPVKAGVETTT |
| | LMISRTPEVTCVVVDVSHEDPEV | PSKQSNNKYAASSYLSLTPEQWK |
| | KFNWYVDGVEVHNAKTKPREE | SHKSYSCQVTHEGSTVEKTVAPT |
| | QYNSTYRVVSVLTVLHQDWLNG | ECS (SEQ ID NO: 57) |
| | KEYKCAVSNKALPSSIEKTISKAK | |
| | GQPREPQVYTLPPSREEMTKNQV | |
| | SLTCLVKGFYPSDIAVEWESNGQ | |
| | PENNYKTTPPVLDSDGSFFLYSK | |
| | LTVDKSRWQQGNVFSCSVMHEA | |
| | LHNHYTQKSLSLSPGAGGGGSG | |
| | GGGSGGGGSQVQLQQSGPGLVK | |
| | PSQTLSLTCAISGDSVSSNSAVW | |
| | NWIRQSPSRGLEWLGRTDYRSK | |
| | WNNDYAVSVKSRITINPDTSKNQ | |
| | FSLQLNSVTPEDTAVYYCARGD | |
| | AGMASFDYWGQGTLVTVSSAST | |
| | KGPSVFPLAPSSKSTSGGTAALG | |
| | CLVKDYFPEPVTVSWNSGALTSG | |
| | VHTFPAVLQSSGLYSLSSVVTVP | |
| | SSSLGTQTYICNVNHKPSNTKVD | |
| | KRVEPKSC (SEQ ID NO: 58) | |
| 85F10 | AMHVAQPAVVLASSRGIASFVCE | SYELTQPLSVSVALGQTARITCGG |
| | YASPGKATEVRVTVLRQADSQV | NNIENKNVHWYQQKPGQAPVLVI |
| | TEVCAATYMMGNELTFLDDSICT | KRDSNRPSGIPERLSGSNSGNTAT |
| | GTSSGNQVNLTIQGLRAMDTGL | LTISRAQAGDEADYYCQVWDSST |
| | YICKVELMYPPPYYLGIGNGTQI | VVFGGGTKLTVLGQPKAAPSVTL |
| | YVIDPEPCPDSDQEPKSSDKTHT | FPPSSEELQANKATLVCLISDFYP |
| | CPPCPAPPVAGPSVFLFPPKPKDT | GAVTVAWKADSSPVKAGVETTT |
| | LMISRTPEVTCVVVDVSHEDPEV | PSKQSNNKYAASSYLSLTPEQWK |
| | KFNWYVDGVEVHNAKTKPREE | SHKSYSCQVTHEGSTVEKTVAPT |
| | QYNSTYRVVSVLTVLHQDWLNG | ECS (SEQ ID NO: 59) |
| | KEYKCAVSNKALPSSIEKTISKAK | |
| | GQPREPQVYTLPPSREEMTKNQV | |
| | SLTCLVKGFYPSDIAVEWESNGQ | |
| | PENNYKTTPPVLDSDGSFFLYSK | |
| | LTVDKSRWQQGNVFSCSVMHEA | |
| | LHNHYTQKSLSLSPGAGGGGSG | |
| | GGGSGGGGSEVQLLESGAGLLK | |
| | PSETLSLTCAVYGGSFSDYYWS | |
| | WIRQPPGKGLEWIGEINHSGSTN | |
| | YNPSLKSRVTISVDTSKNQFSLKL | |
| | SSVTAADTAVYYCARKRGANFF | |
| | DDWGQGTLVTVSSASTKGPSVFP | |
| | LAPSSKSTSGGTAALGCLVKDYF | |
| | PEPVTVSWNSGALTSGVHTFPAV | |
| | LQSSGLYSLSSVVTVPSSSLGTQT | |
| | YICNVNHKPSNTKVDKRVEPKSC | |
| | (SEQ ID NO: 60) | |
| CTLA4_anti | AMHVAQPAVVLASSRGIASFVCE | DIQMTQSPSSLSASVGDRVTITCR |
| OX40L | YASPGKATEVRVTVLRQADSQV | ASQGISSWLAWYQQKPEKAPKSL |
| fusion 4 | TEVCAATYMMGNELTFLDDSICT | IYAASSLQSGVPSRFSGSGSGTDF |
| | GTSSGNQVNLTIQGLRAMDTGL | TLTISSLQPEDFATYYCQQYNSYP |
| | YICKVELMYPPPYYLGIGNGTQI | YTFGQGTKLEIKRTVAAPSVFIFPP |
| | YVIDPEPCPDSDQEPKSSDKTHTS | SDEQLKSGTASVVCLLNNFYPRE |
| | PPSPAPELLGGSSVFLFPPKPKDT | AKVQWKVDNALQSGNSQESVTE |
| | LMISRTPEVTCVVVDVSHEDPEV | QDSKDSTYSLSSTLTLSKADYEKH |
| | KFNWYVDGVEVHNAKTKPREE | KVYACEVTHQGLSSPVTKSFNRG |
| | QYNSTYRVVSVLTVLHQDWLNG | EC (SEQ ID NO: 35) |
| | KEYKCKVSNKALPAPIEKTISKA | |
| | KGQPREPQVYTLPPSREEMTKNQ | |
| | VSLTCLVKGFYPSDIAVEWESNG | |
| | QPENNYKTTPPVLDSDGSFFLYS | |
| | KLTVDKSRWQQGNVFSCSVMHE | |
| | ALHNHYTQKSLSLSPGAGGGGS | |
| | GGGGSGGGGSEVQLLESGGGLV | |
| | QPGGSLRLSCAASGFTFNSYAMS | |
| | WVRQAPGKGLEWVSIISGSGGFT | |
| | YYADSVKGRFTISRDNSRTTLYL | |
| | QMNSLRAEDTAVYYCAKDRLV | |
| | APGTFDYWGQGALVTVSSASTK | |
| | GPSVFPLAPSSKSTSGGTAALGC | |
| | LVKDYFPEPVTVSWNSGALTSG | |
| | VHTFPAVLQSSGLYSLSSVVTVP | |
| | SSSLGTQTYICNVNHKPSNTKVD | |
| | KRVEPKSC (SEQ ID NO: 37) | |
| Larsen | MHVAQPAVVLASSRGIASFVCEY | SSELTQPLSVSVALGQTARMTCG |
| Variant_67B06 | ASPGKYTEVRVTVLRQADSQVT | GSDIENKNVHWYQQKPGQAPVL |
| | EVCAATYMMGNELTFLDDSICT | VIHRDNNRPSGIPERFSGSNSGNT |
| | GTSSGNQVNLTIQGLRAMDTGL | ATLTISRAQAGDEADYYCQVRDS |
| | YICKVELMYPPPYYEGIGNGTQI | NIVVFGGGTKLTVLGQPKAAPSV |
| | YVIDPEPCPDSDQEPKSSDKTHT | TLFPPSSEELQANKATLVCLISDFY |
| | CPPCPAPPVAGPSVFLFPPKPKDT | PGAVTVAWKADSSPVKAGVETT |
| | LMISRTPEVTCVVVDVSHEDPEV | TPSKQSNNKYAASSYLSLTPEQW |
| | KFNWYVDGVEVHNAKTKPREE | KSHKSYSCQVTHEGSTVEKTVAP |
| | QYNSTYRVVSVLTVLHQDWLNG | TECS (SEQ ID NO: 63) |
| | KEYKCAVSNKALAAPIEKTISKA | |
| | KGQPREPQVYTLPPSREEMTKNQ | |
| | VSLTCLVKGFYPSDIAVEWESNG | |
| | QPENNYKTTPPVLDSDGSFFLYS | |
| | KLTVDKSRWQQGNVFSCSVMHE | |
| | ALHNHYTQKSLSLSPGAGGGGS | |
| | GGGGSGGGGSQLQLQESGGPGL | |
| | VKPSETLSLTCSVSGGSISTSSYY | |
| | WGWIRQPPGKGLEWSGSIYYSGS | |
| | TYYRPSLQGRATISVDTSKNQFS | |
| | LKLTSVTAADTAVYYCARHRGS | |
| | YFFDIWGLGTLVTVSSASTKGPS | |
| | VFPLAPSSKSTSGGTAALGCLVK | |
| | DYFPEPVTVSWNSGALTSGVHTF | |
| | PAVLQSSGLYSLSSVVTVPSSSLG | |
| | TQTYICNVNHKPSNTKVDKRVEP | |
| | KSC (SEQ ID NO: 256) | |
| Xu Variant | MHVAQPAVVLASSRGIASFVCEY | SSELTQPLSVSVALGQTARMTCG |
| 1_67B06 | ASPGKHTEVRVTVLRQADSQVT | GSDIENKNVHWYQQKPGQAPVL |
| | EVCAANYMMGNEGTFLDDSICT | VIHRDNNRPSGIPERFSGSNSGNT |
| | GTSSGNQVNLTIQGLRAMDTGL | ATLTISRAQAGDEADYYCQVRDS |
| | YICMVEKMYPPPYYLGIGNGTQI | NIVVFGGGTKLTVLGQPKAAPSV |
| | YVIDPEPCPDSDQEPKSSDKTHT | TLFPPSSEELQANKATLVCLISDFY |
| | CPPCPAPPVAGPSVFLFPPKPKDT | PGAVTVAWKADSSPVKAGVETT |
| | LMISRTPEVTCVVVDVSHEDPEV | TPSKQSNNKYAASSYLSLTPEQW |
| | KFNWYVDGVEVHNAKTKPREE | KSHKSYSCQVTHEGSTVEKTVAP |
| | QYNSTYRVVSVLTVLHQDWLNG | TECS (SEQ ID NO: 63) |
| | KEYKCAVSNKALAAPIEKTISKA | |
| | KGQPREPQVYTLPPSREEMTKNQ | |
| | VSLTCLVKGFYPSDIAVEWESNG | |
| | QPENNYKTTPPVLDSDGSFFLYS | |
| | KLTVDKSRWQQGNVFSCSVMHE | |
| | ALHNHYTQKSLSLSPGAGGGGS | |
| | GGGGSGGGGSQLQLQESGGPGL | |
| | VKPSETLSLTCSVSGGSISTSSYY | |
| | WGWIRQPPGKGLEWSGSIYYSGS | |
| | TYYRPSLQGRATISVDTSKNQFS | |
| | LKLTSVTAADTAVYYCARHRGS | |
| | YFFDIWGLGTLVTVSSASTKGPS | |
| | VFPLAPSSKSTSGGTAALGCLVK | |
| | DYFPEPVTVSWNSGALTSGVHTF | |
| | PAVLQSSGLYSLSSVVTVPSSSLG | |
| | TQTYICNVNHKPSNTKVDKRVEP | |
| | KSC (SEQ ID NO: 257) | |
| Xu Variant | MHVAQPAVVLASSRGIASFVCEY | SSELTQPLSVSVALGQTARMTCG |
| 2_67B06 | ASPGKKTEVRVTVLRQADSQVT | GSDIENKNVHWYQQKPGQAPVL |
| | EVCAANYMMGNEGTFLDDSICT | VIHRDNNRPSGIPERFSGSNSGNT |
| | GTSSGNQVNLTIQGLRAMDTGL | ATLTISRAQAGDEADYYCQVRDS |
| | YICMVEKMYPPPYYLGIGNGTQI | NIVVFGGGTKLTVLGQPKAAPSV |
| | YVIDPEPCPDSDQEPKSSDKTHT | TLFPPSSEELQANKATLVCLISDFY |
| | CPPCPAPPVAGPSVFLFPPKPKDT | PGAVTVAWKADSSPVKAGVETT |
| | LMISRTPEVTCVVVDVSHEDPEV | TPSKQSNNKYAASSYLSLTPEQW |
| | KFNWYVDGVEVHNAKTKPREE | KSHKSYSCQVTHEGSTVEKTVAP |
| | QYNSTYRVVSVLTVLHQDWLNG | TECS (SEQ ID NO: 63) |
| | KEYKCAVSNKALAAPIEKTISKA | |
| | KGQPREPQVYTLPPSREEMTKNQ | |
| | VSLTCLVKGFYPSDIAVEWESNG | |
| | QPENNYKTTPPVLDSDGSFFLYS | |
| | KLTVDKSRWQQGNVFSCSVMHE | |
| | ALHNHYTQKSLSLSPGAGGGGS | |
| | GGGGSGGGGSQLQLQESGGPGL | |
| | VKPSETLSLTCSVSGGSISTSSYY | |
| | WGWIRQPPGKGLEWSGSIYYSGS | |
| | TYYRPSLQGRATISVDTSKNQFS | |
| | LKLTSVTAADTAVYYCARHRGS | |
| | YFFDIWGLGTLVTVSSASTKGPS | |
| | VFPLAPSSKSTSGGTAALGCLVK | |
| | DYFPEPVTVSWNSGALTSGVHTF | |
| | PAVLQSSGLYSLSSVVTVPSSSLG | |
| | TQTYICNVNHKPSNTKVDKRVEP | |
| | KSC (SEQ ID NO: 258) | |
| Xu Variant | MHVAQPAVVLASSRGIASFVCEY | SSELTQPLSVSVALGQTARMTCG |
| 3_67B06 | ASPGKTTEVRVTVLRQADSQVTE | GSDIENKNVHWYQQKPGQAPVL |
| | VCAANYMMGNEGTFLDDSICTG | VIHRDNNRPSGIPERFSGSNSGNT |
| | TSSGNQVNLTIQGLRAMDTGLYI | ATLTISRAQAGDEADYYCQVRDS |
| | CMVEKMYPPPYYLGIGNGTQIY | NIVVFGGGTKLTVLGQPKAAPSV |
| | VIDPEPCPDSDQEPKSSDKTHTCP | TLFPPSSEELQANKATLVCLISDFY |
| | PCPAPPVAGPSVFLFPPKPKDTL | PGAVTVAWKADSSPVKAGVETT |
| | MISRTPEVTCVVVDVSHEDPEVK | TPSKQSNNKYAASSYLSLTPEQW |
| | FNWYVDGVEVHNAKTKPREEQ | KSHKSYSCQVTHEGSTVEKTVAP |
| | YNSTYRVVSVLTVLHQDWLNGK | TECS (SEQ ID NO: 63) |
| | EYKCAVSNKALAAPIEKTISKAK | |
| | GQPREPQVYTLPPSREEMTKNQV | |
| | SLTCLVKGFYPSDIAVEWESNGQ | |
| | PENNYKTTPPVLDSDGSFFLYSK | |
| | LTVDKSRWQQGNVFSCSVMHEA | |
| | LHNHYTQKSLSLSPGAGGGGSG | |
| | GGGSGGGGSQLQLQESGGPGLV | |
| | KPSETLSLTCSVSGGSISTSSYYW | |
| | GWIRQPPGKGLEWSGSIYYSGST | |
| | YYRPSLQGRATISVDTSKNQFSL | |
| | KLTSVTAADTAVYYCARHRGSY | |
| | FFDIWGLGTLVTVSSASTKGPSV | |
| | FPLAPSSKSTSGGTAALGCLVKD | |
| | YFPEPVTVSWNSGALTSGVHTFP | |
| | AVLQSSGLYSLSSVVTVPSSSLGT | |
| | QTYICNVNHKPSNTKVDKRVEP | |
| | KSC (SEQ ID NO: 259) | |
| Xu Variant | MHVAQPAVVLASSRGIASFVCEY | SSELTQPLSVSVALGQTARMTCG |
| 4_67B06 | ASPGKWTEVRVTVLRQADSQVT | GSDIENKNVHWYQQKPGQAPVL |
| | EVCAANYMMGNEGTFLDDSICT | VIHRDNNRPSGIPERFSGSNSGNT |
| | GTSSGNQVNLTIQGLRAMDTGL | ATLTISRAQAGDEADYYCQVRDS |
| | YICMVEKMYPPPYYLGIGNGTQI | NIVVFGGGTKLTVLGQPKAAPSV |
| | YVIDPEPCPDSDQEPKSSDKTHT | TLFPPSSEELQANKATLVCLISDFY |
| | CPPCPAPPVAGPSVFLFPPKPKDT | PGAVTVAWKADSSPVKAGVETT |
| | LMISRTPEVTCVVVDVSHEDPEV | TPSKQSNNKYAASSYLSLTPEQW |
| | KFNWYVDGVEVHNAKTKPREE | KSHKSYSCQVTHEGSTVEKTVAP |
| | QYNSTYRVVSVLTVLHQDWLNG | TECS (SEQ ID NO: 63) |
| | KEYKCAVSNKALAAPIEKTISKA | |
| | KGQPREPQVYTLPPSREEMTKNQ | |
| | VSLTCLVKGFYPSDIAVEWESNG | |
| | QPENNYKTTPPVLDSDGSFFLYS | |
| | KLTVDKSRWQQGNVFSCSVMHE | |
| | ALHNHYTQKSLSLSPGAGGGGS | |
| | GGGGSGGGGSQLQLQESGGPGL | |
| | VKPSETLSLTCSVSGGSISTSSYY | |
| | WGWIRQPPGKGLEWSGSIYYSGS | |
| | TYYRPSLQGRATISVDTSKNQFS | |
| | LKLTSVTAADTAVYYCARHRGS | |
| | YFFDIWGLGTLVTVSSASTKGPS | |
| | VFPLAPSSKSTSGGTAALGCLVK | |
| | DYFPEPVTVSWNSGALTSGVHTF | |
| | PAVLQSSGLYSLSSVVTVPSSSLG | |
| | TQTYICNVNHKPSNTKVDKRVEP | |
| | KSC (SEQ ID NO: 260) | |
| Xu Variant | MHVAQPAVVLASSRGIASFVCEY | SSELTQPLSVSVALGQTARMTCG |
| 5_67B06 | ASPGKYTEVRVTVLRQADSQVT | GSDIENKNVHWYQQKPGQAPVL |
| | EVCAANYMMGNEGTFLDDSICT | VIHRDNNRPSGIPERFSGSNSGNT |
| | GTSSGNQVNLTIQGLRAMDTGL | ATLTISRAQAGDEADYYCQVRDS |
| | YICMVEKMYPPPYYLGIGNGTQI | NIVVFGGGTKLTVLGQPKAAPSV |
| | YVIDPEPCPDSDQEPKSSDKTHT | TLFPPSSEELQANKATLVCLISDFY |
| | CPPCPAPPVAGPSVFLFPPKPKDT | PGAVTVAWKADSSPVKAGVETT |
| | LMISRTPEVTCVVVDVSHEDPEV | TPSKQSNNKYAASSYLSLTPEQW |
| | KFNWYVDGVEVHNAKTKPREE | KSHKSYSCQVTHEGSTVEKTVAP |
| | QYNSTYRVVSVLTVLHQDWLNG | TECS (SEQ ID NO: 63) |
| | KEYKCAVSNKALAAPIEKTISKA | |
| | KGQPREPQVYTLPPSREEMTKNQ | |
| | VSLTCLVKGFYPSDIAVEWESNG | |
| | QPENNYKTTPPVLDSDGSFFLYS | |
| | KLTVDKSRWQQGNVFSCSVMHE | |
| | ALHNHYTQKSLSLSPGAGGGGS | |
| | GGGGSGGGGSQLQLQESGGPGL | |
| | VKPSETLSLTCSVSGGSISTSSYY | |
| | WGWIRQPPGKGLEWSGSIYYSGS | |
| | TYYRPSLQGRATISVDTSKNQFS | |
| | LKLTSVTAADTAVYYCARHRGS | |
| | YFFDIWGLGTLVTVSSASTKGPS | |
| | VFPLAPSSKSTSGGTAALGCLVK | |
| | DYFPEPVTVSWNSGALTSGVHTF | |
| | PAVLQSSGLYSLSSVVTVPSSSLG | |
| | TQTYICNVNHKPSNTKVDKRVEP | |
| | KSC (SEQ ID NO: 261) | |
| Xu Variant | MHVAQPAVVLASSRGIASFVCEY | SSELTQPLSVSVALGQTARMTCG |
| 6_67B06 | ASPGKHTEVRVTVLRQADSQVT | GSDIENKNVHWYQQKPGQAPVL |
| | EVCAAYYMMGNEGTFLDDSICT | VIHRDNNRPSGIPERFSGSNSGNT |
| | GTSSGNQVNLTIQGLRAMDTGL | ATLTISRAQAGDEADYYCQVRDS |
| | YICQVEKMYPPPYYLGIGNGTQI | NIVVFGGGTKLTVLGQPKAAPSV |
| | YVIDPEPCPDSDQEPKSSDKTHT | TLFPPSSEELQANKATLVCLISDFY |
| | CPPCPAPPVAGPSVFLFPPKPKDT | PGAVTVAWKADSSPVKAGVETT |
| | LMISRTPEVTCVVVDVSHEDPEV | TPSKQSNNKYAASSYLSLTPEQW |
| | KFNWYVDGVEVHNAKTKPREE | KSHKSYSCQVTHEGSTVEKTVAP |
| | QYNSTYRVVSVLTVLHQDWLNG | TECS (SEQ ID NO: 63) |
| | KEYKCAVSNKALAAPIEKTISKA | |
| | KGQPREPQVYTLPPSREEMTKNQ | |
| | VSLTCLVKGFYPSDIAVEWESNG | |
| | QPENNYKTTPPVLDSDGSFFLYS | |
| | KLTVDKSRWQQGNVFSCSVMHE | |
| | ALHNHYTQKSLSLSPGAGGGGS | |
| | GGGGSGGGGSQLQLQESGGPGL | |
| | VKPSETLSLTCSVSGGSISTSSYY | |
| | WGWIRQPPGKGLEWSGSIYYSGS | |
| | TYYRPSLQGRATISVDTSKNQFS | |
| | LKLTSVTAADTAVYYCARHRGS | |
| | YFFDIWGLGTLVTVSSASTKGPS | |
| | VFPLAPSSKSTSGGTAALGCLVK | |
| | DYFPEPVTVSWNSGALTSGVHTF | |
| | PAVLQSSGLYSLSSVVTVPSSSLG | |
| | TQTYICNVNHKPSNTKVDKRVEP | |
| | KSC (SEQ ID NO: 262) | |
| Xu Variant | MHVAQPAVVLASSRGIASFVCEY | SSELTQPLSVSVALGQTARMTCG |
| 7_67B06 | ASPGKKTEVRVTVLRQADSQVT | GSDIENKNVHWYQQKPGQAPVL |
| | EVCAAYYMMGNEGTFLDDSICT | VIHRDNNRPSGIPERFSGSNSGNT |
| | GTSSGNQVNLTIQGLRAMDTGL | ATLTISRAQAGDEADYYCQVRDS |
| | YICQVEKMYPPPYYLGIGNGTQI | NIVVFGGGTKLTVLGQPKAAPSV |
| | YVIDPEPCPDSDQEPKSSDKTHT | TLFPPSSEELQANKATLVCLISDFY |
| | CPPCPAPPVAGPSVFLFPPKPKDT | PGAVTVAWKADSSPVKAGVETT |
| | LMISRTPEVTCVVVDVSHEDPEV | TPSKQSNNKYAASSYLSLTPEQW |
| | KFNWYVDGVEVHNAKTKPREE | KSHKSYSCQVTHEGSTVEKTVAP |
| | QYNSTYRVVSVLTVLHQDWLNG | TECS (SEQ ID NO: 63) |
| | KEYKCAVSNKALAAPIEKTISKA | |
| | KGQPREPQVYTLPPSREEMTKNQ | |
| | VSLTCLVKGFYPSDIAVEWESNG | |
| | QPENNYKTTPPVLDSDGSFFLYS | |
| | KLTVDKSRWQQGNVFSCSVMHE | |
| | ALHNHYTQKSLSLSPGAGGGGS | |
| | GGGGSGGGGSQLQLQESGGPGL | |
| | VKPSETLSLTCSVSGGSISTSSYY | |
| | WGWIRQPPGKGLEWSGSIYYSGS | |
| | TYYRPSLQGRATISVDTSKNQFS | |
| | LKLTSVTAADTAVYYCARHRGS | |
| | YFFDIWGLGTLVTVSSASTKGPS | |
| | VFPLAPSSKSTSGGTAALGCLVK | |
| | DYFPEPVTVSWNSGALTSGVHTF | |
| | PAVLQSSGLYSLSSVVTVPSSSLG | |
| | TQTYICNVNHKPSNTKVDKRVEP | |
| | KSC (SEQ ID NO: 263) | |
| Xu Variant | MHVAQPAVVLASSRGIASFVCEY | SSELTQPLSVSVALGQTARMTCG |
| 8_67B06 | ASPGKTTEVRVTVLRQADSQVTE | GSDIENKNVHWYQQKPGQAPVL |
| | VCAAYYMMGNEGTFLDDSICTG | VIHRDNNRPSGIPERFSGSNSGNT |
| | TSSGNQVNLTIQGLRAMDTGLYI | ATLTISRAQAGDEADYYCQVRDS |
| | CQVEKMYPPPYYLGIGNGTQIYV | NIVVFGGGTKLTVLGQPKAAPSV |
| | IDPEPCPDSDQEPKSSDKTHTCPP | TLFPPSSEELQANKATLVCLISDFY |
| | CPAPPVAGPSVFLFPPKPKDTLMI | PGAVTVAWKADSSPVKAGVETT |
| | SRTPEVTCVVVDVSHEDPEVKFN | TPSKQSNNKYAASSYLSLTPEQW |
| | WYVDGVEVHNAKTKPREEQYN | KSHKSYSCQVTHEGSTVEKTVAP |
| | STYRVVSVLTVLHQDWLNGKEY | TECS (SEQ ID NO: 63) |
| | KCAVSNKALAAPIEKTISKAKGQ | |
| | PREPQVYTLPPSREEMTKNQVSL | |
| | TCLVKGFYPSDIAVEWESNGQPE | |
| | NNYKTTPPVLDSDGSFFLYSKLT | |
| | VDKSRWQQGNVFSCSVMHEAL | |
| | HNHYTQKSLSLSPGAGGGGSGG | |
| | GGSGGGGSQLQLQESGGPGLVK | |
| | PSETLSLTCSVSGGSISTSSYYWG | |
| | WIRQPPGKGLEWSGSIYYSGSTY | |
| | YRPSLQGRATISVDTSKNQFSLK | |
| | LTSVTAADTAVYYCARHRGSYF | |
| | FDIWGLGTLVTVSSASTKGPSVF | |
| | PLAPSSKSTSGGTAALGCLVKDY | |
| | FPEPVTVSWNSGALTSGVHTFPA | |
| | VLQSSGLYSLSSVVTVPSSSLGTQ | |
| | TYICNVNHKPSNTKVDKRVEPKS | |
| | C (SEQ ID NO: 264) | |
| Xu Variant | MHVAQPAVVLASSRGIASFVCEY | SSELTQPLSVSVALGQTARMTCG |
| 9_67B06 | ASPGKWTEVRVTVLRQADSQVT | GSDIENKNVHWYQQKPGQAPVL |
| | EVCAAYYMMGNEGTFLDDSICT | VIHRDNNRPSGIPERFSGSNSGNT |
| | GTSSGNQVNLTIQGLRAMDTGL | ATLTISRAQAGDEADYYCQVRDS |
| | YICQVEKMYPPPYYLGIGNGTQI | NIVVFGGGTKLTVLGQPKAAPSV |
| | YVIDPEPCPDSDQEPKSSDKTHT | TLFPPSSEELQANKATLVCLISDFY |
| | CPPCPAPPVAGPSVFLFPPKPKDT | PGAVTVAWKADSSPVKAGVETT |
| | LMISRTPEVTCVVVDVSHEDPEV | TPSKQSNNKYAASSYLSLTPEQW |
| | KFNWYVDGVEVHNAKTKPREE | KSHKSYSCQVTHEGSTVEKTVAP |
| | QYNSTYRVVSVLTVLHQDWLNG | TECS (SEQ ID NO: 63) |
| | KEYKCAVSNKALAAPIEKTISKA | |
| | KGQPREPQVYTLPPSREEMTKNQ | |
| | VSLTCLVKGFYPSDIAVEWESNG | |
| | QPENNYKTTPPVLDSDGSFFLYS | |
| | KLTVDKSRWQQGNVFSCSVMHE | |
| | ALHNHYTQKSLSLSPGAGGGGS | |
| | GGGGSGGGGSQLQLQESGGPGL | |
| | VKPSETLSLTCSVSGGSISTSSYY | |
| | WGWIRQPPGKGLEWSGSIYYSGS | |
| | TYYRPSLQGRATISVDTSKNQFS | |
| | LKLTSVTAADTAVYYCARHRGS | |
| | YFFDIWGLGTLVTVSSASTKGPS | |
| | VFPLAPSSKSTSGGTAALGCLVK | |
| | DYFPEPVTVSWNSGALTSGVHTF | |
| | PAVLQSSGLYSLSSVVTVPSSSLG | |
| | TQTYICNVNHKPSNTKVDKRVEP | |
| | KSC (SEQ ID NO: 265) | |
| Xu Variant | MHVAQPAVVLASSRGIASFVCEY | SSELTQPLSVSVALGQTARMTCG |
| 10_67B06 | ASPGKYTEVRVTVLRQADSQVT | GSDIENKNVHWYQQKPGQAPVL |
| | EVCAAYYMMGNEGTFLDDSICT | VIHRDNNRPSGIPERFSGSNSGNT |
| | GTSSGNQVNLTIQGLRAMDTGL | ATLTISRAQAGDEADYYCQVRDS |
| | YICQVEKMYPPPYYLGIGNGTQI | NIVVFGGGTKLTVLGQPKAAPSV |
| | YVIDPEPCPDSDQEPKSSDKTHT | TLFPPSSEELQANKATLVCLISDFY |
| | CPPCPAPPVAGPSVFLFPPKPKDT | PGAVTVAWKADSSPVKAGVETT |
| | LMISRTPEVTCVVVDVSHEDPEV | TPSKQSNNKYAASSYLSLTPEQW |
| | KFNWYVDGVEVHNAKTKPREE | KSHKSYSCQVTHEGSTVEKTVAP |
| | QYNSTYRVVSVLTVLHQDWLNG | TECS (SEQ ID NO: 63) |
| | KEYKCAVSNKALAAPIEKTISKA | |
| | KGQPREPQVYTLPPSREEMTKNQ | |
| | VSLTCLVKGFYPSDIAVEWESNG | |
| | QPENNYKTTPPVLDSDGSFFLYS | |
| | KLTVDKSRWQQGNVFSCSVMHE | |
| | ALHNHYTQKSLSLSPGAGGGGS | |
| | GGGGSGGGGSQLQLQESGGPGL | |
| | VKPSETLSLTCSVSGGSISTSSYY | |
| | WGWIRQPPGKGLEWSGSIYYSGS | |
| | TYYRPSLQGRATISVDTSKNQFS | |
| | LKLTSVTAADTAVYYCARHRGS | |
| | YFFDIWGLGTLVTVSSASTKGPS | |
| | VFPLAPSSKSTSGGTAALGCLVK | |
| | DYFPEPVTVSWNSGALTSGVHTF | |
| | PAVLQSSGLYSLSSVVTVPSSSLG | |
| | TQTYICNVNHKPSNTKVDKRVEP | |
| | KSC (SEQ ID NO: 266) | |
| Xu Variant | MHVAQPAVVLASSRGIASFVCEY | SSELTQPLSVSVALGQTARMTCG |
| 11_67B06 | ASPGKHTEVRVTVLRQADSQVT | GSDIENKNVHWYQQKPGQAPVL |
| | EVCAAYYMMGNEGTFLDDSICT | VIHRDNNRPSGIPERFSGSNSGNT |
| | GTSSGNQVNLTIQGLRAMDTGL | ATLTISRAQAGDEADYYCQVRDS |
| | YICQVEKMYPPPYYLGIGNGTQI | NIVVFGGGTKLTVLGQPKAAPSV |
| | YVIDPEPCPDSDQEPKSSDKTHT | TLFPPSSEELQANKATLVCLISDFY |
| | CPPCPAPPVAGPSVFLFPPKPKDT | PGAVTVAWKADSSPVKAGVETT |
| | LMISRTPEVTCVVVDVSHEDPEV | TPSKQSNNKYAASSYLSLTPEQW |
| | KFNWYVDGVEVHNAKTKPREE | KSHKSYSCQVTHEGSTVEKTVAP |
| | QYNSTYRVVSVLTVLHQDWLNG | TECS (SEQ ID NO: 63) |
| | KEYKCAVSNKALAAPIEKTISKA | |
| | KGQPREPQVYTLPPSREEMTKNQ | |
| | VSLTCLVKGFYPSDIAVEWESNG | |
| | QPENNYKTTPPVLDSDGSFFLYS | |
| | KLTVDKSRWQQGNVFSCSVMHE | |
| | ALHNHYTQKSLSLSPGAGGGGS | |
| | GGGGSGGGGSQLQLQESGGPGL | |
| | VKPSETLSLTCSVSGGSISTSSYY | |
| | WGWIRQPPGKGLEWSGSIYYSGS | |
| | TYYRPSLQGRATISVDTSKNQFS | |
| | LKLTSVTAADTAVYYCARHRGS | |
| | YFFDIWGLGTLVTVSSASTKGPS | |
| | VFPLAPSSKSTSGGTAALGCLVK | |
| | DYFPEPVTVSWNSGALTSGVHTF | |
| | PAVLQSSGLYSLSSVVTVPSSSLG | |
| | TQTYICNVNHKPSNTKVDKRVEP | |
| | KSC (SEQ ID NO: 267) | |
| Xu Variant | MHVAQPAVVLASSRGIASFVCEY | SSELTQPLSVSVALGQTARMTCG |
| 12_67B06 | ASPGKKTEVRVTVLRQADSQVT | GSDIENKNVHWYQQKPGQAPVL |
| | EVCAAYYMMGNEGTFLDDSICT | VIHRDNNRPSGIPERFSGSNSGNT |
| | GTSSGNQVNLTIQGLRAMDTGL | ATLTISRAQAGDEADYYCQVRDS |
| | YICQVEKMYPPPYYLGIGNGTQI | NIVVFGGGTKLTVLGQPKAAPSV |
| | YVIDPEPCPDSDQEPKSSDKTHT | TLFPPSSEELQANKATLVCLISDFY |
| | CPPCPAPPVAGPSVFLFPPKPKDT | PGAVTVAWKADSSPVKAGVETT |
| | LMISRTPEVTCVVVDVSHEDPEV | TPSKQSNNKYAASSYLSLTPEQW |
| | KFNWYVDGVEVHNAKTKPREE | KSHKSYSCQVTHEGSTVEKTVAP |
| | QYNSTYRVVSVLTVLHQDWLNG | TECS (SEQ ID NO: 63) |
| | KEYKCAVSNKALAAPIEKTISKA | |
| | KGQPREPQVYTLPPSREEMTKNQ | |
| | VSLTCLVKGFYPSDIAVEWESNG | |
| | QPENNYKTTPPVLDSDGSFFLYS | |
| | KLTVDKSRWQQGNVFSCSVMHE | |
| | ALHNHYTQKSLSLSPGAGGGGS | |
| | GGGGSGGGGSQLQLQESGGPGL | |
| | VKPSETLSLTCSVSGGSISTSSYY | |
| | WGWIRQPPGKGLEWSGSIYYSGS | |
| | TYYRPSLQGRATISVDTSKNQFS | |
| | LKLTSVTAADTAVYYCARHRGS | |
| | YFFDIWGLGTLVTVSSASTKGPS | |
| | VFPLAPSSKSTSGGTAALGCLVK | |
| | DYFPEPVTVSWNSGALTSGVHTF | |
| | PAVLQSSGLYSLSSVVTVPSSSLG | |
| | TQTYICNVNHKPSNTKVDKRVEP | |
| | KSC (SEQ ID NO: 268) | |
| Xu Variant | MHVAQPAVVLASSRGIASFVCEY | SSELTQPLSVSVALGQTARMTCG |
| 13_67B06 | ASPGKTTEVRVTVLRQADSQVTE | GSDIENKNVHWYQQKPGQAPVL |
| | VCAAYYMMGNEGTFLDDSICTG | VIHRDNNRPSGIPERFSGSNSGNT |
| | TSSGNQVNLTIQGLRAMDTGLYI | ATLTISRAQAGDEADYYCQVRDS |
| | CQVEKMYPPPYYLGIGNGTQIYV | NIVVFGGGTKLTVLGQPKAAPSV |
| | IDPEPCPDSDQEPKSSDKTHTCPP | TLFPPSSEELQANKATLVCLISDFY |
| | CPAPPVAGPSVFLFPPKPKDTLMI | PGAVTVAWKADSSPVKAGVETT |
| | SRTPEVTCVVVDVSHEDPEVKFN | TPSKQSNNKYAASSYLSLTPEQW |
| | WYVDGVEVHNAKTKPREEQYN | KSHKSYSCQVTHEGSTVEKTVAP |
| | STYRVVSVLTVLHQDWLNGKEY | TECS (SEQ ID NO: 63) |
| | KCAVSNKALAAPIEKTISKAKGQ | |
| | PREPQVYTLPPSREEMTKNQVSL | |
| | TCLVKGFYPSDIAVEWESNGQPE | |
| | NNYKTTPPVLDSDGSFFLYSKLT | |
| | VDKSRWQQGNVFSCSVMHEAL | |
| | HNHYTQKSLSLSPGAGGGGSGG | |
| | GGSGGGGSQLQLQESGGPGLVK | |
| | PSETLSLTCSVSGGSISTSSYYWG | |
| | WIRQPPGKGLEWSGSIYYSGSTY | |
| | YRPSLQGRATISVDTSKNQFSLK | |
| | LTSVTAADTAVYYCARHRGSYF | |
| | FDIWGLGTLVTVSSASTKGPSVF | |
| | PLAPSSKSTSGGTAALGCLVKDY | |
| | FPEPVTVSWNSGALTSGVHTFPA | |
| | VLQSSGLYSLSSVVTVPSSSLGTQ | |
| | TYICNVNHKPSNTKVDKRVEPKS | |
| | C (SEQ ID NO: 269) | |
| Xu Variant | MHVAQPAVVLASSRGIASFVCEY | SSELTQPLSVSVALGQTARMTCG |
| 14_67B06 | ASPGKWTEVRVTVLRQADSQVT | GSDIENKNVHWYQQKPGQAPVL |
| | EVCAAYYMMGNEGTFLDDSICT | VIHRDNNRPSGIPERFSGSNSGNT |
| | GTSSGNQVNLTIQGLRAMDTGL | ATLTISRAQAGDEADYYCQVRDS |
| | YICQVEKMYPPPYYLGIGNGTQI | NIVVFGGGTKLTVLGQPKAAPSV |
| | YVIDPEPCPDSDQEPKSSDKTHT | TLFPPSSEELQANKATLVCLISDFY |
| | CPPCPAPPVAGPSVFLFPPKPKDT | PGAVTVAWKADSSPVKAGVETT |
| | LMISRTPEVTCVVVDVSHEDPEV | TPSKQSNNKYAASSYLSLTPEQW |
| | KFNWYVDGVEVHNAKTKPREE | KSHKSYSCQVTHEGSTVEKTVAP |
| | QYNSTYRVVSVLTVLHQDWLNG | TECS (SEQ ID NO: 63) |
| | KEYKCAVSNKALAAPIEKTISKA | |
| | KGQPREPQVYTLPPSREEMTKNQ | |
| | VSLTCLVKGFYPSDIAVEWESNG | |
| | QPENNYKTTPPVLDSDGSFFLYS | |
| | KLTVDKSRWQQGNVFSCSVMHE | |
| | ALHNHYTQKSLSLSPGAGGGGS | |
| | GGGGSGGGGSQLQLQESGGPGL | |
| | VKPSETLSLTCSVSGGSISTSSYY | |
| | WGWIRQPPGKGLEWSGSIYYSGS | |
| | TYYRPSLQGRATISVDTSKNQFS | |
| | LKLTSVTAADTAVYYCARHRGS | |
| | YFFDIWGLGTLVTVSSASTKGPS | |
| | VFPLAPSSKSTSGGTAALGCLVK | |
| | DYFPEPVTVSWNSGALTSGVHTF | |
| | PAVLQSSGLYSLSSVVTVPSSSLG | |
| | TQTYICNVNHKPSNTKVDKRVEP | |
| | KSC (SEQ ID NO: 270) | |
| Xu Variant | MHVAQPAVVLASSRGIASFVCEY | SSELTQPLSVSVALGQTARMTCG |
| 15_67B06 | ASPGKYTEVRVTVLRQADSQVT | GSDIENKNVHWYQQKPGQAPVL |
| | EVCAAYYMMGNEGTFLDDSICT | VIHRDNNRPSGIPERFSGSNSGNT |
| | GTSSGNQVNLTIQGLRAMDTGL | ATLTISRAQAGDEADYYCQVRDS |
| | YICQVEKMYPPPYYLGIGNGTQI | NIVVFGGGTKLTVLGQPKAAPSV |
| | YVIDPEPCPDSDQEPKSSDKTHT | TLFPPSSEELQANKATLVCLISDFY |
| | CPPCPAPPVAGPSVFLFPPKPKDT | PGAVTVAWKADSSPVKAGVETT |
| | LMISRTPEVTCVVVDVSHEDPEV | TPSKQSNNKYAASSYLSLTPEQW |
| | KFNWYVDGVEVHNAKTKPREE | KSHKSYSCQVTHEGSTVEKTVAP |
| | QYNSTYRVVSVLTVLHQDWLNG | TECS (SEQ ID NO: 63) |
| | KEYKCAVSNKALAAPIEKTISKA | |
| | KGQPREPQVYTLPPSREEMTKNQ | |
| | VSLTCLVKGFYPSDIAVEWESNG | |
| | QPENNYKTTPPVLDSDGSFFLYS | |
| | KLTVDKSRWQQGNVFSCSVMHE | |
| | ALHNHYTQKSLSLSPGAGGGGS | |
| | GGGGSGGGGSQLQLQESGGPGL | |
| | VKPSETLSLTCSVSGGSISTSSYY | |
| | WGWIRQPPGKGLEWSGSIYYSGS | |
| | TYYRPSLQGRATISVDTSKNQFS | |
| | LKLTSVTAADTAVYYCARHRGS | |
| | YFFDIWGLGTLVTVSSASTKGPS | |
| | VFPLAPSSKSTSGGTAALGCLVK | |
| | DYFPEPVTVSWNSGALTSGVHTF | |
| | PAVLQSSGLYSLSSVVTVPSSSLG | |
| | TQTYICNVNHKPSNTKVDKRVEP | |
| | KSC (SEQ ID NO: 271) | |
| Xu Variant | MHVAQPAVVLASSRGIASFVCEY | SSELTQPLSVSVALGQTARMTCG |
| 16_67B06 | ASPGKHTEVRVTVLRQADSQVT | GSDIENKNVHWYQQKPGQAPVL |
| | EVCAANYMMGNEGTFLDDSICT | VIHRDNNRPSGIPERFSGSNSGNT |
| | GTSSGNQVNLTIQGLRAMDTGL | ATLTISRAQAGDEADYYCQVRDS |
| | YICQVEKMYPPPYYLGIGNGTQI | NIVVFGGGTKLTVLGQPKAAPSV |
| | YVIDPEPCPDSDQEPKSSDKTHT | TLFPPSSEELQANKATLVCLISDFY |
| | CPPCPAPPVAGPSVFLFPPKPKDT | PGAVTVAWKADSSPVKAGVETT |
| | LMISRTPEVTCVVVDVSHEDPEV | TPSKQSNNKYAASSYLSLTPEQW |
| | KFNWYVDGVEVHNAKTKPREE | KSHKSYSCQVTHEGSTVEKTVAP |
| | QYNSTYRVVSVLTVLHQDWLNG | TECS (SEQ ID NO: 63) |
| | KEYKCAVSNKALAAPIEKTISKA | |
| | KGQPREPQVYTLPPSREEMTKNQ | |
| | VSLTCLVKGFYPSDIAVEWESNG | |
| | QPENNYKTTPPVLDSDGSFFLYS | |
| | KLTVDKSRWQQGNVFSCSVMHE | |
| | ALHNHYTQKSLSLSPGAGGGGS | |
| | GGGGSGGGGSQLQLQESGGPGL | |
| | VKPSETLSLTCSVSGGSISTSSYY | |
| | WGWIRQPPGKGLEWSGSIYYSGS | |
| | TYYRPSLQGRATISVDTSKNQFS | |
| | LKLTSVTAADTAVYYCARHRGS | |
| | YFFDIWGLGTLVTVSSASTKGPS | |
| | VFPLAPSSKSTSGGTAALGCLVK | |
| | DYFPEPVTVSWNSGALTSGVHTF | |
| | PAVLQSSGLYSLSSVVTVPSSSLG | |
| | TQTYICNVNHKPSNTKVDKRVEP | |
| | KSC (SEQ ID NO: 272) | |
| Xu Variant | MHVAQPAVVLASSRGIASFVCEY | SSELTQPLSVSVALGQTARMTCG |
| 17_67B06 | ASPGKKTEVRVTVLRQADSQVT | GSDIENKNVHWYQQKPGQAPVL |
| | EVCAANYMMGNEGTFLDDSICT | VIHRDNNRPSGIPERFSGSNSGNT |
| | GTSSGNQVNLTIQGLRAMDTGL | ATLTISRAQAGDEADYYCQVRDS |
| | YICQVEKMYPPPYYLGIGNGTQI | NIVVFGGGTKLTVLGQPKAAPSV |
| | YVIDPEPCPDSDQEPKSSDKTHT | TLFPPSSEELQANKATLVCLISDFY |
| | CPPCPAPPVAGPSVFLFPPKPKDT | PGAVTVAWKADSSPVKAGVETT |
| | LMISRTPEVTCVVVDVSHEDPEV | TPSKQSNNKYAASSYLSLTPEQW |
| | KFNWYVDGVEVHNAKTKPREE | KSHKSYSCQVTHEGSTVEKTVAP |
| | QYNSTYRVVSVLTVLHQDWLNG | TECS (SEQ ID NO: 63) |
| | KEYKCAVSNKALAAPIEKTISKA | |
| | KGQPREPQVYTLPPSREEMTKNQ | |
| | VSLTCLVKGFYPSDIAVEWESNG | |
| | QPENNYKTTPPVLDSDGSFFLYS | |
| | KLTVDKSRWQQGNVFSCSVMHE | |
| | ALHNHYTQKSLSLSPGAGGGGS | |
| | GGGGSGGGGSQLQLQESGGPGL | |
| | VKPSETLSLTCSVSGGSISTSSYY | |
| | WGWIRQPPGKGLEWSGSIYYSGS | |
| | TYYRPSLQGRATISVDTSKNQFS | |
| | LKLTSVTAADTAVYYCARHRGS | |
| | YFFDIWGLGTLVTVSSASTKGPS | |
| | VFPLAPSSKSTSGGTAALGCLVK | |
| | DYFPEPVTVSWNSGALTSGVHTF | |
| | PAVLQSSGLYSLSSVVTVPSSSLG | |
| | TQTYICNVNHKPSNTKVDKRVEP | |
| | KSC (SEQ ID NO: 273) | |
| Xu Variant | MHVAQPAVVLASSRGIASFVCEY | SSELTQPLSVSVALGQTARMTCG |
| 18_67B06 | ASPGKTTEVRVTVLRQADSQVTE | GSDIENKNVHWYQQKPGQAPVL |
| | VCAANYMMGNEGTFLDDSICTG | VIHRDNNRPSGIPERFSGSNSGNT |
| | TSSGNQVNLTIQGLRAMDTGLYI | ATLTISRAQAGDEADYYCQVRDS |
| | CQVEKMYPPPYYLGIGNGTQIYV | NIVVFGGGTKLTVLGQPKAAPSV |
| | IDPEPCPDSDQEPKSSDKTHTCPP | TLFPPSSEELQANKATLVCLISDFY |
| | CPAPPVAGPSVFLFPPKPKDTLMI | PGAVTVAWKADSSPVKAGVETT |
| | SRTPEVTCVVVDVSHEDPEVKFN | TPSKQSNNKYAASSYLSLTPEQW |
| | WYVDGVEVHNAKTKPREEQYN | KSHKSYSCQVTHEGSTVEKTVAP |
| | STYRVVSVLTVLHQDWLNGKEY | TECS (SEQ ID NO: 63) |
| | KCAVSNKALAAPIEKTISKAKGQ | |
| | PREPQVYTLPPSREEMTKNQVSL | |
| | TCLVKGFYPSDIAVEWESNGQPE | |
| | NNYKTTPPVLDSDGSFFLYSKLT | |
| | VDKSRWQQGNVFSCSVMHEAL | |
| | HNHYTQKSLSLSPGAGGGGSGG | |
| | GGSGGGGSQLQLQESGGPGLVK | |
| | PSETLSLTCSVSGGSISTSSYYWG | |
| | WIRQPPGKGLEWSGSIYYSGSTY | |
| | YRPSLQGRATISVDTSKNQFSLK | |
| | LTSVTAADTAVYYCARHRGSYF | |
| | FDIWGLGTLVTVSSASTKGPSVF | |
| | PLAPSSKSTSGGTAALGCLVKDY | |
| | FPEPVTVSWNSGALTSGVHTFPA | |
| | VLQSSGLYSLSSVVTVPSSSLGTQ | |
| | TYICNVNHKPSNTKVDKRVEPKS | |
| | C (SEQ ID NO: 274) | |
| Xu Variant | MHVAQPAVVLASSRGIASFVCEY | SSELTQPLSVSVALGQTARMTCG |
| 19_67B06 | ASPGKWTEVRVTVLRQADSQVT | GSDIENKNVHWYQQKPGQAPVL |
| | EVCAANYMMGNEGTFLDDSICT | VIHRDNNRPSGIPERFSGSNSGNT |
| | GTSSGNQVNLTIQGLRAMDTGL | ATLTISRAQAGDEADYYCQVRDS |
| | YICQVEKMYPPPYYLGIGNGTQI | NIVVFGGGTKLTVLGQPKAAPSV |
| | YVIDPEPCPDSDQEPKSSDKTHT | TLFPPSSEELQANKATLVCLISDFY |
| | CPPCPAPPVAGPSVFLFPPKPKDT | PGAVTVAWKADSSPVKAGVETT |
| | LMISRTPEVTCVVVDVSHEDPEV | TPSKQSNNKYAASSYLSLTPEQW |
| | KFNWYVDGVEVHNAKTKPREE | KSHKSYSCQVTHEGSTVEKTVAP |
| | QYNSTYRVVSVLTVLHQDWLNG | TECS (SEQ ID NO: 63) |
| | KEYKCAVSNKALAAPIEKTISKA | |
| | KGQPREPQVYTLPPSREEMTKNQ | |
| | VSLTCLVKGFYPSDIAVEWESNG | |
| | QPENNYKTTPPVLDSDGSFFLYS | |
| | KLTVDKSRWQQGNVFSCSVMHE | |
| | ALHNHYTQKSLSLSPGAGGGGS | |
| | GGGGSGGGGSQLQLQESGGPGL | |
| | VKPSETLSLTCSVSGGSISTSSYY | |
| | WGWIRQPPGKGLEWSGSIYYSGS | |
| | TYYRPSLQGRATISVDTSKNQFS | |
| | LKLTSVTAADTAVYYCARHRGS | |
| | YFFDIWGLGTLVTVSSASTKGPS | |
| | VFPLAPSSKSTSGGTAALGCLVK | |
| | DYFPEPVTVSWNSGALTSGVHTF | |
| | PAVLQSSGLYSLSSVVTVPSSSLG | |
| | TQTYICNVNHKPSNTKVDKRVEP | |
| | KSC (SEQ ID NO: 275) | |
| Xu Variant | MHVAQPAVVLASSRGIASFVCEY | SSELTQPLSVSVALGQTARMTCG |
| 20_67B06 | ASPGKYTEVRVTVLRQADSQVT | GSDIENKNVHWYQQKPGQAPVL |
| | EVCAANYMMGNEGTFLDDSICT | VIHRDNNRPSGIPERFSGSNSGNT |
| | GTSSGNQVNLTIQGLRAMDTGL | ATLTISRAQAGDEADYYCQVRDS |
| | YICQVEKMYPPPYYLGIGNGTQI | NIVVFGGGTKLTVLGQPKAAPSV |
| | YVIDPEPCPDSDQEPKSSDKTHT | TLFPPSSEELQANKATLVCLISDFY |
| | CPPCPAPPVAGPSVFLFPPKPKDT | PGAVTVAWKADSSPVKAGVETT |
| | LMISRTPEVTCVVVDVSHEDPEV | TPSKQSNNKYAASSYLSLTPEQW |
| | KFNWYVDGVEVHNAKTKPREE | KSHKSYSCQVTHEGSTVEKTVAP |
| | QYNSTYRVVSVLTVLHQDWLNG | TECS (SEQ ID NO: 63) |
| | KEYKCAVSNKALAAPIEKTISKA | |
| | KGQPREPQVYTLPPSREEMTKNQ | |
| | VSLTCLVKGFYPSDIAVEWESNG | |
| | QPENNYKTTPPVLDSDGSFFLYS | |
| | KLTVDKSRWQQGNVFSCSVMHE | |
| | ALHNHYTQKSLSLSPGAGGGGS | |
| | GGGGSGGGGSQLQLQESGGPGL | |
| | VKPSETLSLTCSVSGGSISTSSYY | |
| | WGWIRQPPGKGLEWSGSIYYSGS | |
| | TYYRPSLQGRATISVDTSKNQFS | |
| | LKLTSVTAADTAVYYCARHRGS | |
| | YFFDIWGLGTLVTVSSASTKGPS | |
| | VFPLAPSSKSTSGGTAALGCLVK | |
| | DYFPEPVTVSWNSGALTSGVHTF | |
| | PAVLQSSGLYSLSSVVTVPSSSLG | |
| | TQTYICNVNHKPSNTKVDKRVEP | |
| | KSC (SEQ ID NO: 276) | |
| Bernett | MHVAQPAVVLASSRGIASFVCEY | SSELTQPLSVSVALGQTARMTCG |
| Variant | ASPGKHTEVRVTVLRQADSQVT | GSDIENKNVHWYQQKPGQAPVL |
| 1_67B06 | EVCFANYYMGNELTFDDDSICTG | VIHRDNNRPSGIPERFSGSNSGNT |
| | TSSGNQVNLTIQGLRAMDTGLYI | ATLTISRAQAGDEADYYCQVRDS |
| | CQVELMYPPPYYEGIGNGTQIYV | NIVVFGGGTKLTVLGQPKAAPSV |
| | IDPEPCPDSD | TLFPPSSEELQANKATLVCLISDFY |
| | QEPKSSDKTHTCPPCPAPPVAGP | PGAVTVAWKADSSPVKAGVETT |
| | SVFLFPPKPKDTLMISRTPEVTCV | TPSKQSNNKYAASSYLSLTPEQW |
| | VVDVSHEDPEVKFNWYVDGVE | KSHKSYSCQVTHEGSTVEKTVAP |
| | VHNAKTKPREEQYNSTYRVVSV | TECS (SEQ ID NO: 63) |
| | LTVLHQDWLNGKEYKCAVSNK | |
| | ALAAPIEKTISKAKGQPREPQVY | |
| | TLPPSREEMTKNQVSLTCLVKGF | |
| | YPSDIAVEWESNGQPENNYKTTP | |
| | PVLDSDGSFFLYSKLTVDKSRWQ | |
| | QGNVFSCSVMHEALHNHYTQKS | |
| | LSLSPGAGGGGSGGGGSGGGGS | |
| | QLQLQESGGPGLVKPSETLSLTC | |
| | SVSGGSISTSSYYWGWIRQPPGK | |
| | GLEWSGSIYYSGSTYYRPSLQGR | |
| | ATISVDTSKNQFSLKLTSVTAAD | |
| | TAVYYCARHRGSYFFDIWGLGT | |
| | LVTVSSASTKGPSVFPLAPSSKST | |
| | SGGTAALGCLVKDYFPEPVTVS | |
| | WNSGALTSGVHTFPAVLQSSGL | |
| | YSLSSVVTVPSSSLGTQTYICNVN | |
| | HKPSNTKVDKRVEPKSC (SEQ ID | |
| | NO: 277) | |
| Bernett Variant | MHVAQPAVVLASSRGIASFVCEY | SSELTQPLSVSVALGQTARMTCG |
| 2_67B06 | ASPGKKTEVRVTVLRQADSQVT | GSDIENKNVHWYQQKPGQAPVL |
| | EVCFANYYMGNELTFDDDSICTG | VIHRDNNRPSGIPERFSGSNSGNT |
| | TSSGNQVNLTIQGLRAMDTGLYI | ATLTISRAQAGDEADYYCQVRDS |
| | CQVELMYPPPYYEGIGNGTQIYV | NIVVFGGGTKLTVLGQPKAAPSV |
| | IDPEPCPDSD | TLFPPSSEELQANKATLVCLISDFY |
| | QEPKSSDKTHTCPPCPAPPVAGP | PGAVTVAWKADSSPVKAGVETT |
| | SVFLFPPKPKDTLMISRTPEVTCV | TPSKQSNNKYAASSYLSLTPEQW |
| | VVDVSHEDPEVKFNWYVDGVE | KSHKSYSCQVTHEGSTVEKTVAP |
| | VHNAKTKPREEQYNSTYRVVSV | TECS (SEQ ID NO: 63) |
| | LTVLHQDWLNGKEYKCAVSNK | |
| | ALAAPIEKTISKAKGQPREPQVY | |
| | TLPPSREEMTKNQVSLTCLVKGF | |
| | YPSDIAVEWESNGQPENNYKTTP | |
| | PVLDSDGSFFLYSKLTVDKSRWQ | |
| | QGNVFSCSVMHEALHNHYTQKS | |
| | LSLSPGAGGGGSGGGGSGGGGS | |
| | QLQLQESGGPGLVKPSETLSLTC | |
| | SVSGGSISTSSYYWGWIRQPPGK | |
| | GLEWSGSIYYSGSTYYRPSLQGR | |
| | ATISVDTSKNQFSLKLTSVTAAD | |
| | TAVYYCARHRGSYFFDIWGLGT | |
| | LVTVSSASTKGPSVFPLAPSSKST | |
| | SGGTAALGCLVKDYFPEPVTVS | |
| | WNSGALTSGVHTFPA VLQSSGL | |
| | YSLSSVVTVPSSSLGTQTYICNVN | |
| | HKPSNTKVDKRVEPKSC (SEQ ID | |
| | NO: 278) | |
| Bernett Variant | MHVAQPAVVLASSRGIASFVCEY | SSELTQPLSVSVALGQTARMTCG |
| 3_67B06 | ASPGKWTEVRVTVLRQADSQVT | GSDIENKNVHWYQQKPGQAPVL |
| | EVCFANYYMGNELTFDDDSICTG | VIHRDNNRPSGIPERFSGSNSGNT |
| | TSSGNQVNLTIQGLRAMDTGLYI | ATLTISRAQAGDEADYYCQVRDS |
| | CQVELMYPPPYYEGIGNGTQIYV | NIVVFGGGTKLTVLGQPKAAPSV |
| | IDPEPCPDSD | TLFPPSSEELQANKATLVCLISDFY |
| | QEPKSSDKTHTCPPCPAPPVAGP | PGAVTVAWKADSSPVKAGVETT |
| | SVFLFPPKPKDTLMISRTPEVTCV | TPSKQSNNKYAASSYLSLTPEQW |
| | VVDVSHEDPEVKFNWYVDGVE | KSHKSYSCQVTHEGSTVEKTVAP |
| | VHNAKTKPREEQYNSTYRVVSV | TECS (SEQ ID NO: 63) |
| | LTVLHQDWLNGKEYKCAVSNK | |
| | ALAAPIEKTISKAKGQPREPQVY | |
| | TLPPSREEMTKNQVSLTCLVKGF | |
| | YPSDIAVEWESNGQPENNYKTTP | |
| | PVLDSDGSFFLYSKLTVDKSRWQ | |
| | QGNVFSCSVMHEALHNHYTQKS | |
| | LSLSPGAGGGGSGGGGSGGGGS | |
| | QLQLQESGGPGLVKPSETLSLTC | |
| | SVSGGSISTSSYYWGWIRQPPGK | |
| | GLEWSGSIYYSGSTYYRPSLQGR | |
| | ATISVDTSKNQFSLKLTSVTAAD | |
| | TAVYYCARHRGSYFFDIWGLGT | |
| | LVTVSSASTKGPSVFPLAPSSKST | |
| | SGGTAALGCLVKDYFPEPVTVS | |
| | WNSGALTSGVHTFPAVLQSSGL | |
| | YSLSSVVTVPSSSLGTQTYICNVN | |
| | HKPSNTKVDKRVEPKSC (SEQ ID | |
| | NO: 279) | |
| Bernett Variant | MHVAQPAVVLASSRGIASFVCEY | SSELTQPLSVSVALGQTARMTCG |
| 4_67B06 | ASPGKYTEVRVTVLRQADSQVT | GSDIENKNVHWYQQKPGQAPVL |
| | EVCFANYYMGNELTFDDDSICTG | VIHRDNNRPSGIPERFSGSNSGNT |
| | TSSGNQVNLTIQGLRAMDTGLYI | ATLTISRAQAGDEADYYCQVRDS |
| | CQVELMYPPPYYEGIGNGTQIYV | NIVVFGGGTKLTVLGQPKAAPSV |
| | IDPEPCPDSD | TLFPPSSEELQANKATLVCLISDFY |
| | QEPKSSDKTHTCPPCPAPPVAGP | PGAVTVAWKADSSPVKAGVETT |
| | SVFLFPPKPKDTLMISRTPEVTCV | TPSKQSNNKYAASSYLSLTPEQW |
| | VVDVSHEDPEVKFNWYVDGVE | KSHKSYSCQVTHEGSTVEKTVAP |
| | VHNAKTKPREEQYNSTYRVVSV | TECS (SEQ ID NO: 63) |
| | LTVLHQDWLNGKEYKCAVSNK | |
| | ALAAPIEKTISKAKGQPREPQVY | |
| | TLPPSREEMTKNQVSLTCLVKGF | |
| | YPSDIAVEWESNGQPENNYKTTP | |
| | PVLDSDGSFFLYSKLTVDKSRWQ | |
| | QGNVFSCSVMHEALHNHYTQKS | |
| | LSLSPGAGGGGSGGGGSGGGGS | |
| | QLQLQESGGPGLVKPSETLSLTC | |
| | SVSGGSISTSSYYWGWIRQPPGK | |
| | GLEWSGSIYYSGSTYYRPSLQGR | |
| | ATISVDTSKNQFSLKLTSVTAAD | |
| | TAVYYCARHRGSYFFDIWGLGT | |
| | LVTVSSASTKGPSVFPLAPSSKST | |
| | SGGTAALGCLVKDYFPEPVTVS | |
| | WNSGALTSGVHTFPAVLQSSGL | |
| | YSLSSVVTVPSSSLGTQTYICNVN | |
| | HKPSNTKVDKRVEPKSC (SEQ ID | |
| | NO: 280) | |
| Bernett Variant | MHVAQPAVVLASSRGIASFVCEY | SSELTQPLSVSVALGQTARMTCG |
| 5_67B06 | ASPGKHTEVRVTVLRQADSQVT | GSDIENKNVHWYQQKPGQAPVL |
| | EVCFANYYMGNELTFEDDSICTG | VIHRDNNRPSGIPERFSGSNSGNT |
| | TSSGNQVNLTIQGLRAMDTGLYI | ATLTISRAQAGDEADYYCQVRDS |
| | CQVELMYPPPYYEGIGNGTQIYV | NIVVFGGGTKLTVLGQPKAAPSV |
| | IDPEPCPDSDQEPKSSDKTHTCPP | TLFPPSSEELQANKATLVCLISDFY |
| | CPAPPVAGPSVFLFPPKPKDTLMI | PGAVTVAWKADSSPVKAGVETT |
| | SRTPEVTCVVVDVSHEDPEVKFN | TPSKQSNNKYAASSYLSLTPEQW |
| | WYVDGVEVHNAKTKPREEQYN | KSHKSYSCQVTHEGSTVEKTVAP |
| | STYRVVSVLTVLHQDWLNGKEY | TECS (SEQ ID NO: 63) |
| | KCAVSNKALAAPIEKTISKAKGQ | |
| | PREPQVYTLPPSREEMTKNQVSL | |
| | TCLVKGFYPSDIAVEWESNGQPE | |
| | NNYKTTPPVLDSDGSFFLYSKLT | |
| | VDKSRWQQGNVFSCSVMHEAL | |
| | HNHYTQKSLSLSPGAGGGGSGG | |
| | GGSGGGGSQLQLQESGGPGLVK | |
| | PSETLSLTCSVSGGSISTSSYYWG | |
| | WIRQPPGKGLEWSGSIYYSGSTY | |
| | YRPSLQGRATISVDTSKNQFSLK | |
| | LTSVTAADTAVYYCARHRGSYF | |
| | FDIWGLGTLVTVSSASTKGPSVF | |
| | PLAPSSKSTSGGTAALGCLVKDY | |
| | FPEPVTVSWNSGALTSGVHTFPA | |
| | VLQSSGLYSLSSVVTVPSSSLGTQ | |
| | TYICNVNHKPSNTKVDKRVEPKS | |
| | C (SEQ ID NO: 281) | |
| Bernett Variant | MHVAQPAVVLASSRGIASFVCEY | SSELTQPLSVSVALGQTARMTCG |
| 6_67B06 | ASPGKKTEVRVTVLRQADSQVT | GSDIENKNVHWYQQKPGQAPVL |
| | EVCFANYYMGNELTFEDDSICTG | VIHRDNNRPSGIPERFSGSNSGNT |
| | TSSGNQVNLTIQGLRAMDTGLYI | ATLTISRAQAGDEADYYCQVRDS |
| | CQVELMYPPPYYEGIGNGTQIYV | NIVVFGGGTKLTVLGQPKAAPSV |
| | IDPEPCPDSDQEPKSSDKTHTCPP | TLFPPSSEELQANKATLVCLISDFY |
| | CPAPPVAGPSVFLFPPKPKDTLMI | PGAVTVAWKADSSPVKAGVETT |
| | SRTPEVTCVVVDVSHEDPEVKFN | TPSKQSNNKYAASSYLSLTPEQW |
| | WYVDGVEVHNAKTKPREEQYN | KSHKSYSCQVTHEGSTVEKTVAP |
| | STYRVVSVLTVLHQDWLNGKEY | TECS (SEQ ID NO: 63) |
| | KCAVSNKALAAPIEKTISKAKGQ | |
| | PREPQVYTLPPSREEMTKNQVSL | |
| | TCLVKGFYPSDIAVEWESNGQPE | |
| | NNYKTTPPVLDSDGSFFLYSKLT | |
| | VDKSRWQQGNVFSCSVMHEAL | |
| | HNHYTQKSLSLSPGAGGGGSGG | |
| | GGSGGGGSQLQLQESGGPGLVK | |
| | PSETLSLTCSVSGGSISTSSYYWG | |
| | WIRQPPGKGLEWSGSIYYSGSTY | |
| | YRPSLQGRATISVDTSKNQFSLK | |
| | LTSVTAADTAVYYCARHRGSYF | |
| | FDIWGLGTLVTVSSASTKGPSVF | |
| | PLAPSSKSTSGGTAALGCLVKDY | |
| | FPEPVTVSWNSGALTSGVHTFPA | |
| | VLQSSGLYSLSSVVTVPSSSLGTQ | |
| | TYICNVNHKPSNTKVDKRVEPKS | |
| | C (SEQ ID NO: 282) | |
| Bernett Variant | MHVAQPAVVLASSRGIASFVCEY | SSELTQPLSVSVALGQTARMTCG |
| 7_67B06 | ASPGKWTEVRVTVLRQADSQVT | GSDIENKNVHWYQQKPGQAPVL |
| | EVCFANYYMGNELTFEDDSICTG | VIHRDNNRPSGIPERFSGSNSGNT |
| | TSSGNQVNLTIQGLRAMDTGLYI | ATLTISRAQAGDEADYYCQVRDS |
| | CQVELMYPPPYYEGIGNGTQIYV | NIVVFGGGTKLTVLGQPKAAPSV |
| | IDPEPCPDSDQEPKSSDKTHTCPP | TLFPPSSEELQANKATLVCLISDFY |
| | CPAPPVAGPSVFLFPPKPKDTLMI | PGAVTVAWKADSSPVKAGVETT |
| | SRTPEVTCVVVDVSHEDPEVKFN | TPSKQSNNKYAASSYLSLTPEQW |
| | WYVDGVEVHNAKTKPREEQYN | KSHKSYSCQVTHEGSTVEKTVAP |
| | STYRVVSVLTVLHQDWLNGKEY | TECS (SEQ ID NO: 63) |
| | KCAVSNKALAAPIEKTISKAKGQ | |
| | PREPQVYTLPPSREEMTKNQVSL | |
| | TCLVKGFYPSDIAVEWESNGQPE | |
| | NNYKTTPPVLDSDGSFFLYSKLT | |
| | VDKSRWQQGNVFSCSVMHEAL | |
| | HNHYTQKSLSLSPGAGGGGSGG | |
| | GGSGGGGSQLQLQESGGPGLVK | |
| | PSETLSLTCSVSGGSISTSSYYWG | |
| | WIRQPPGKGLEWSGSIYYSGSTY | |
| | YRPSLQGRATISVDTSKNQFSLK | |
| | LTSVTAADTAVYYCARHRGSYF | |
| | FDIWGLGTLVTVSSASTKGPSVF | |
| | PLAPSSKSTSGGTAALGCLVKDY | |
| | FPEPVTVSWNSGALTSGVHTFPA | |
| | VLQSSGLYSLSSVVTVPSSSLGTQ | |
| | TYICNVNHKPSNTKVDKRVEPKS | |
| | C (SEQ ID NO: 283) | |
| Bernett Variant | MHVAQPAVVLASSRGIASFVCEY | SSELTQPLSVSVALGQTARMTCG |
| 8_67B06 | ASPGKYTEVRVTVLRQADSQVT | GSDIENKNVHWYQQKPGQAPVL |
| | EVCFANYYMGNELTFEDDSICTG | VIHRDNNRPSGIPERFSGSNSGNT |
| | TSSGNQVNLTIQGLRAMDTGLYI | ATLTISRAQAGDEADYYCQVRDS |
| | CQVELMYPPPYYEGIGNGTQIYV | NIVVFGGGTKLTVLGQPKAAPSV |
| | IDPEPCPDSDQEPKSSDKTHTCPP | TLFPPSSEELQANKATLVCLISDFY |
| | CPAPPVAGPSVFLFPPKPKDTLMI | PGAVTVAWKADSSPVKAGVETT |
| | SRTPEVTCVVVDVSHEDPEVKFN | TPSKQSNNKYAASSYLSLTPEQW |
| | WYVDGVEVHNAKTKPREEQYN | KSHKSYSCQVTHEGSTVEKTVAP |
| | STYRVVSVLTVLHQDWLNGKEY | TECS (SEQ ID NO: 63) |
| | KCAVSNKALAAPIEKTISKAKGQ | |
| | PREPQVYTLPPSREEMTKNQVSL | |
| | TCLVKGFYPSDIAVEWESNGQPE | |
| | NNYKTTPPVLDSDGSFFLYSKLT | |
| | VDKSRWQQGNVFSCSVMHEAL | |
| | HNHYTQKSLSLSPGAGGGGSGG | |
| | GGSGGGGSQLQLQESGGPGLVK | |
| | PSETLSLTCSVSGGSISTSSYYWG | |
| | WIRQPPGKGLEWSGSIYYSGSTY | |
| | YRPSLQGRATISVDTSKNQFSLK | |
| | LTSVTAADTAVYYCARHRGSYF | |
| | FDIWGLGTLVTVSSASTKGPSVF | |
| | PLAPSSKSTSGGTAALGCLVKDY | |
| | FPEPVTVSWNSGALTSGVHTFPA | |
| | VLQSSGLYSLSSVVTVPSSSLGTQ | |
| | TYICNVNHKPSNTKVDKRVEPKS | |
| | C (SEQ ID NO: 284) | |
| Oshima | MHVAQPAVVLASSRGIASFVCEY | SSELTQPLSVSVALGQTARMTCG |
| Variant_67B06 | ESPGKANEIRVTVLRQADSQVTE | GSDIENKNVHWYQQKPGQAPVL |
| | VCAMTYMKEDELTFLDDPSCTG | VIHRDNNRPSGIPERFSGSNSGNT |
| | TFSGNQVNLTIQGLRAMDTGLYI | ATLTISRAQAGDEADYYCQVRDS |
| | CKVELMYPPPYYEGIGNGTQIYV | NIVVFGGGTKLTVLGQPKAAPSV |
| | IDPEPCPDSDQEPKSSDKTHTCPP | TLFPPSSEELQANKATLVCLISDFY |
| | CPAPPVAGPSVFLFPPKPKDTLMI | PGAVTVAWKADSSPVKAGVETT |
| | SRTPEVTCVVVDVSHEDPEVKFN | TPSKQSNNKYAASSYLSLTPEQW |
| | WYVDGVEVHNAKTKPREEQYN | KSHKSYSCQVTHEGSTVEKTVAP |
| | STYRVVSVLTVLHQDWLNGKEY | TECS (SEQ ID NO: 63) |
| | KCAVSNKALAAPIEKTISKAKGQ | |
| | PREPQVYTLPPSREEMTKNQVSL | |
| | TCLVKGFYPSDIAVEWESNGQPE | |
| | NNYKTTPPVLDSDGSFFLYSKLT | |
| | VDKSRWQQGNVFSCSVMHEAL | |
| | HNHYTQKSLSLSPGAGGGGSGG | |
| | GGSGGGGSQLQLQESGGPGLVK | |
| | PSETLSLTCSVSGGSISTSSYYWG | |
| | WIRQPPGKGLEWSGSIYYSGSTY | |
| | YRPSLQGRATISVDTSKNQFSLK | |
| | LTSVTAADTAVYYCARHRGSYF | |
| | FDIWGLGTLVTVSSASTKGPSVF | |
| | PLAPSSKSTSGGTAALGCLVKDY | |
| | FPEPVTVSWNSGALTSGVHTFPA | |
| | VLQSSGLYSLSSVVTVPSSSLGTQ | |
| | TYICNVNHKPSNTKVDKRVEPKS | |
| | C (SEQ ID NO: 285) | |
| Douthwaite | MHVAQPAVVLASSRGRASFVCE | SSELTQPLSVSVALGQTARMTCG |
| Variant | YTNPSKATEVRVTVLRQADSQV | GSDIENKNVHWYQQKPGQAPVL |
| 1_67B06 | TEVCAATYMKGNEATFLDDSICT | VIHRDNNRPSGIPERFSGSNSGNT |
| | GTASGNQVNLTIRGLRAQDTGL | ATLTISRAQAGDEADYYCQVRDS |
| | YICQVELMYPPPYYLGIGNGTQI | NIVVFGGGTKLTVLGQPKAAPSV |
| | YVIDPEPCPDSDQEPKSSDKTHT | TLFPPSSEELQANKATLVCLISDFY |
| | CPPCPAPPVAGPSVFLFPPKPKDT | PGAVTVAWKADSSPVKAGVETT |
| | LMISRTPEVTCVVVDVSHEDPEV | TPSKQSNNKYAASSYLSLTPEQW |
| | KFNWYVDGVEVHNAKTKPREE | KSHKSYSCQVTHEGSTVEKTVAP |
| | QYNSTYRVVSVLTVLHQDWLNG | TECS (SEQ ID NO: 63) |
| | KEYKCAVSNKALAAPIEKTISKA | |
| | KGQPREPQVYTLPPSREEMTKNQ | |
| | VSLTCLVKGFYPSDIAVEWESNG | |
| | QPENNYKTTPPVLDSDGSFFLYS | |
| | KLTVDKSRWQQGNVFSCSVMHE | |
| | ALHNHYTQKSLSLSPGAGGGGS | |
| | GGGGSGGGGSQLQLQESGGPGL | |
| | VKPSETLSLTCSVSGGSISTSSYY | |
| | WGWIRQPPGKGLEWSGSIYYSGS | |
| | TYYRPSLQGRATISVDTSKNQFS | |
| | LKLTSVTAADTAVYYCARHRGS | |
| | YFFDIWGLGTLVTVSSASTKGPS | |
| | VFPLAPSSKSTSGGTAALGCLVK | |
| | DYFPEPVTVSWNSGALTSGVHTF | |
| | PAVLQSSGLYSLSSVVTVPSSSLG | |
| | TQTYICNVNHKPSNTKVDKRVEP | |
| | KSC (SEQ ID NO: 286) | |
| Douthwaite | MHVAQPAVVLASSVGRASFVCE | SSELTQPLSVSVALGQTARMTCG |
| Variant | YTNPSKATEVRVTVLRQADSQV | GSDIENKNVHWYQQKPGQAPVL |
| 2_67B06 | TEVCAATYMKGNEATFLDDSICT | VIHRDNNRPSGIPERFSGSNSGNT |
| | GTASGNQVNLTIRGLRAQDTGL | ATLTISRAQAGDEADYYCQVRDS |
| | YICQVELMYPPPYYLGIGNGTQI | NIVVFGGGTKLTVLGQPKAAPSV |
| | YVIDPEPCPDSDQEPKSSDKTHT | TLFPPSSEELQANKATLVCLISDFY |
| | CPPCPAPPVAGPSVFLFPPKPKDT | PGAVTVAWKADSSPVKAGVETT |
| | LMISRTPEVTCVVVDVSHEDPEV | TPSKQSNNKYAASSYLSLTPEQW |
| | KFNWYVDGVEVHNAKTKPREE | KSHKSYSCQVTHEGSTVEKTVAP |
| | QYNSTYRVVSVLTVLHQDWLNG | TECS (SEQ ID NO: 63) |
| | KEYKCAVSNKALAAPIEKTISKA | |
| | KGQPREPQVYTLPPSREEMTKNQ | |
| | VSLTCLVKGFYPSDIAVEWESNG | |
| | QPENNYKTTPPVLDSDGSFFLYS | |
| | KLTVDKSRWQQGNVFSCSVMHE | |
| | ALHNHYTQKSLSLSPGAGGGGS | |
| | GGGGSGGGGSQLQLQESGGPGL | |
| | VKPSETLSLTCSVSGGSISTSSYY | |
| | WGWIRQPPGKGLEWSGSIYYSGS | |
| | TYYRPSLQGRATISVDTSKNQFS | |
| | LKLTSVTAADTAVYYCARHRGS | |
| | YFFDIWGLGTLVTVSSASTKGPS | |
| | VFPLAPSSKSTSGGTAALGCLVK | |
| | DYFPEPVTVSWNSGALTSGVHTF | |
| | PAVLQSSGLYSLSSVVTVPSSSLG | |
| | TQTYICNVNHKPSNTKVDKRVEP | |
| | KSC (SEQ ID NO: 287) | |
| Douthwaite | MHVAQPAVVLASSRGRASFVCE | SSELTQPLSVSVALGQTARMTCG |
| Variant | YTNPSKATEVRVTVLRQADSQV | GSDIENKNVHWYQQKPGQAPVL |
| 3_67B06 | TEVCAATYMKGNEGTFLDDSICT | VIHRDNNRPSGIPERFSGSNSGNT |
| | GTASGNQVNLTIRGLRAQDTGL | ATLTISRAQAGDEADYYCQVRDS |
| | YICQVELMYPPPYYLGIGNGTQI | NIVVFGGGTKLTVLGQPKAAPSV |
| | YVIDPEPCPDSDQEPKSSDKTHT | TLFPPSSEELQANKATLVCLISDFY |
| | CPPCPAPPVAGPSVFLFPPKPKDT | PGAVTVAWKADSSPVKAGVETT |
| | LMISRTPEVTCVVVDVSHEDPEV | TPSKQSNNKYAASSYLSLTPEQW |
| | KFNWYVDGVEVHNAKTKPREE | KSHKSYSCQVTHEGSTVEKTVAP |
| | QYNSTYRVVSVLTVLHQDWLNG | TECS (SEQ ID NO: 63) |
| | KEYKCAVSNKALAAPIEKTISKA | |
| | KGQPREPQVYTLPPSREEMTKNQ | |
| | VSLTCLVKGFYPSDIAVEWESNG | |
| | QPENNYKTTPPVLDSDGSFFLYS | |
| | KLTVDKSRWQQGNVFSCSVMHE | |
| | ALHNHYTQKSLSLSPGAGGGGS | |
| | GGGGSGGGGSQLQLQESGGPGL | |
| | VKPSETLSLTCSVSGGSISTSSYY | |
| | WGWIRQPPGKGLEWSGSIYYSGS | |
| | TYYRPSLQGRATISVDTSKNQFS | |
| | LKLTSVTAADTAVYYCARHRGS | |
| | YFFDIWGLGTLVTVSSASTKGPS | |
| | VFPLAPSSKSTSGGTAALGCLVK | |
| | DYFPEPVTVSWNSGALTSGVHTF | |
| | PAVLQSSGLYSLSSVVTVPSSSLG | |
| | TQTYICNVNHKPSNTKVDKRVEP | |
| | KSC (SEQ ID NO: 288) | |
| Douthwaite | MHVAQPAVVLASSVGRASFVCE | SSELTQPLSVSVALGQTARMTCG |
| Variant | YTNPSKATEVRVTVLRQADSQV | GSDIENKNVHWYQQKPGQAPVL |
| 4_67B06 | TEVCAATYMKGNEGTFLDDSICT | VIHRDNNRPSGIPERFSGSNSGNT |
| | GTASGNQVNLTIRGLRAQDTGL | ATLTISRAQAGDEADYYCQVRDS |
| | YICQVELMYPPPYYLGIGNGTQI | NIVVFGGGTKLTVLGQPKAAPSV |
| | YVIDPEPCPDSDQEPKSSDKTHT | TLFPPSSEELQANKATLVCLISDFY |
| | CPPCPAPPVAGPSVFLFPPKPKDT | PGAVTVAWKADSSPVKAGVETT |
| | LMISRTPEVTCVVVDVSHEDPEV | TPSKQSNNKYAASSYLSLTPEQW |
| | KFNWYVDGVEVHNAKTKPREE | KSHKSYSCQVTHEGSTVEKTVAP |
| | QYNSTYRVVSVLTVLHQDWLNG | TECS (SEQ ID NO: 63) |
| | KEYKCAVSNKALAAPIEKTISKA | |
| | KGQPREPQVYTLPPSREEMTKNQ | |
| | VSLTCLVKGFYPSDIAVEWESNG | |
| | QPENNYKTTPPVLDSDGSFFLYS | |
| | KLTVDKSRWQQGNVFSCSVMHE | |
| | ALHNHYTQKSLSLSPGAGGGGS | |
| | GGGGSGGGGSQLQLQESGGPGL | |
| | VKPSETLSLTCSVSGGSISTSSYY | |
| | WGWIRQPPGKGLEWSGSIYYSGS | |
| | TYYRPSLQGRATISVDTSKNQFS | |
| | LKLTSVTAADTAVYYCARHRGS | |
| | YFFDIWGLGTLVTVSSASTKGPS | |
| | VFPLAPSSKSTSGGTAALGCLVK | |
| | DYFPEPVTVSWNSGALTSGVHTF | |
| | PAVLQSSGLYSLSSVVTVPSSSLG | |
| | TQTYICNVNHKPSNTKVDKRVEP | |
| | KSC (SEQ ID NO: 289) | |
| Douthwaite | MHVAQPAVVLASSRGRASFVCE | SSELTQPLSVSVALGQTARMTCG |
| Variant | YTNPSKATEVRVTVLRQADSQV | GSDIENKNVHWYQQKPGQAPVL |
| 5_67B06 | TEVCAATYMKGNEATFLDDSICT | VIHRDNNRPSGIPERFSGSNSGNT |
| | GTASGNQVNLTIRGLRASDTGLY | ATLTISRAQAGDEADYYCQVRDS |
| | ICQVELMYPPPYYLGIGNGTQIY | NIVVFGGGTKLTVLGQPKAAPSV |
| | VIDPEPCPDSDQEPKSSDKTHTCP | TLFPPSSEELQANKATLVCLISDFY |
| | PCPAPPVAGPSVFLFPPKPKDTL | PGAVTVAWKADSSPVKAGVETT |
| | MISRTPEVTCVVVDVSHEDPEVK | TPSKQSNNKYAASSYLSLTPEQW |
| | FNWYVDGVEVHNAKTKPREEQ | KSHKSYSCQVTHEGSTVEKTVAP |
| | YNSTYRVVSVLTVLHQDWLNGK | TECS (SEQ ID NO: 63) |
| | EYKCAVSNKALAAPIEKTISKAK | |
| | GQPREPQVYTLPPSREEMTKNQV | |
| | SLTCLVKGFYPSDIAVEWESNGQ | |
| | PENNYKTTPPVLDSDGSFFLYSK | |
| | LTVDKSRWQQGNVFSCSVMHEA | |
| | LHNHYTQKSLSLSPGAGGGGSG | |
| | GGGSGGGGSQLQLQESGGPGLV | |
| | KPSETLSLTCSVSGGSISTSSYYW | |
| | GWIRQPPGKGLEWSGSIYYSGST | |
| | YYRPSLQGRATISVDTSKNQFSL | |
| | KLTSVTAADTAVYYCARHRGSY | |
| | FFDIWGLGTLVTVSSASTKGPSV | |
| | FPLAPSSKSTSGGTAALGCLVKD | |
| | YFPEPVTVSWNSGALTSGVHTFP | |
| | AVLQSSGLYSLSSVVTVPSSSLGT | |
| | QTYICNVNHKPSNTKVDKRVEP | |
| | KSC (SEQ ID NO: 290) | |
| Douthwaite | MHVAQPAVVLASSVGRASFVCE | SSELTQPLSVSVALGQTARMTCG |
| Variant | YTNPSKATEVRVTVLRQADSQV | GSDIENKNVHWYQQKPGQAPVL |
| 6_67B06 | TEVCAATYMKGNEATFLDDSICT | VIHRDNNRPSGIPERFSGSNSGNT |
| | GTASGNQVNLTIRGLRASDTGLY | ATLTISRAQAGDEADYYCQVRDS |
| | ICQVELMYPPPYYLGIGNGTQIY | NIVVFGGGTKLTVLGQPKAAPSV |
| | VIDPEPCPDSDQEPKSSDKTHTCP | TLFPPSSEELQANKATLVCLISDFY |
| | PCPAPPVAGPSVFLFPPKPKDTL | PGAVTVAWKADSSPVKAGVETT |
| | MISRTPEVTCVVVDVSHEDPEVK | TPSKQSNNKYAASSYLSLTPEQW |
| | FNWYVDGVEVHNAKTKPREEQ | KSHKSYSCQVTHEGSTVEKTVAP |
| | YNSTYRVVSVLTVLHQDWLNGK | TECS (SEQ ID NO: 63) |
| | EYKCAVSNKALAAPIEKTISKAK | |
| | GQPREPQVYTLPPSREEMTKNQV | |
| | SLTCLVKGFYPSDIAVEWESNGQ | |
| | PENNYKTTPPVLDSDGSFFLYSK | |
| | LTVDKSRWQQGNVFSCSVMHEA | |
| | LHNHYTQKSLSLSPGAGGGGSG | |
| | GGGSGGGGSQLQLQESGGPGLV | |
| | KPSETLSLTCSVSGGSISTSSYYW | |
| | GWIRQPPGKGLEWSGSIYYSGST | |
| | YYRPSLQGRATISVDTSKNQFSL | |
| | KLTSVTAADTAVYYCARHRGSY | |
| | FFDIWGLGTLVTVSSASTKGPSV | |
| | FPLAPSSKSTSGGTAALGCLVKD | |
| | YFPEPVTVSWNSGALTSGVHTFP | |
| | AVLQSSGLYSLSSVVTVPSSSLGT | |
| | QTYICNVNHKPSNTKVDKRVEP | |
| | KSC (SEQ ID NO: 291) | |
| Douthwaite | MHVAQPAVVLASSRGRASFVCE | SSELTQPLSVSVALGQTARMTCG |
| Variant | YTNPSKATEVRVTVLRQADSQV | GSDIENKNVHWYQQKPGQAPVL |
| 7_67B06 | TEVCAATYMKGNEGTFLDDSICT | VIHRDNNRPSGIPERFSGSNSGNT |
| | GTASGNQVNLTIRGLRASDTGLY | ATLTISRAQAGDEADYYCQVRDS |
| | ICQVELMYPPPYYLGIGNGTQIY | NIVVFGGGTKLTVLGQPKAAPSV |
| | VIDPEPCPDSDQEPKSSDKTHTCP | TLFPPSSEELQANKATLVCLISDFY |
| | PCPAPPVAGPSVFLFPPKPKDTL | PGAVTVAWKADSSPVKAGVETT |
| | MISRTPEVTCVVVDVSHEDPEVK | TPSKQSNNKYAASSYLSLTPEQW |
| | FNWYVDGVEVHNAKTKPREEQ | KSHKSYSCQVTHEGSTVEKTVAP |
| | YNSTYRVVSVLTVLHQDWLNGK | TECS (SEQ ID NO: 63) |
| | EYKCAVSNKALAAPIEKTISKAK | |
| | GQPREPQVYTLPPSREEMTKNQV | |
| | SLTCLVKGFYPSDIAVEWESNGQ | |
| | PENNYKTTPPVLDSDGSFFLYSK | |
| | LTVDKSRWQQGNVFSCSVMHEA | |
| | LHNHYTQKSLSLSPGAGGGGSG | |
| | GGGSGGGGSQLQLQESGGPGLV | |
| | KPSETLSLTCSVSGGSISTSSYYW | |
| | GWIRQPPGKGLEWSGSIYYSGST | |
| | YYRPSLQGRATISVDTSKNQFSL | |
| | KLTSVTAADTAVYYCARHRGSY | |
| | FFDIWGLGTLVTVSSASTKGPSV | |
| | FPLAPSSKSTSGGTAALGCLVKD | |
| | YFPEPVTVSWNSGALTSGVHTFP | |
| | AVLQSSGLYSLSSVVTVPSSSLGT | |
| | QTYICNVNHKPSNTKVDKRVEP | |
| | KSC (SEQ ID NO: 292) | |
| Douthwaite | MHVAQPAVVLASSVGRASFVCE | SSELTQPLSVSVALGQTARMTCG |
| Variant | YTNPSKATEVRVTVLRQADSQV | GSDIENKNVHWYQQKPGQAPVL |
| 8_67B06 | TEVCAATYMKGNEGTFLDDSICT | VIHRDNNRPSGIPERFSGSNSGNT |
| | GTASGNQVNLTIRGLRASDTGLY | ATLTISRAQAGDEADYYCQVRDS |
| | ICQVELMYPPPYYLGIGNGTQIY | NIVVFGGGTKLTVLGQPKAAPSV |
| | VIDPEPCPDSDQEPKSSDKTHTCP | TLFPPSSEELQANKATLVCLISDFY |
| | PCPAPPVAGPSVFLFPPKPKDTL | PGAVTVAWKADSSPVKAGVETT |
| | MISRTPEVTCVVVDVSHEDPEVK | TPSKQSNNKYAASSYLSLTPEQW |
| | FNWYVDGVEVHNAKTKPREEQ | KSHKSYSCQVTHEGSTVEKTVAP |
| | YNSTYRVVSVLTVLHQDWLNGK | TECS (SEQ ID NO: 63) |
| | EYKCAVSNKALAAPIEKTISKAK | |
| | GQPREPQVYTLPPSREEMTKNQV | |
| | SLTCLVKGFYPSDIAVEWESNGQ | |
| | PENNYKTTPPVLDSDGSFFLYSK | |
| | LTVDKSRWQQGNVFSCSVMHEA | |
| | LHNHYTQKSLSLSPGAGGGGSG | |
| | GGGSGGGGSQLQLQESGGPGLV | |
| | KPSETLSLTCSVSGGSISTSSYYW | |
| | GWIRQPPGKGLEWSGSIYYSGST | |
| | YYRPSLQGRATISVDTSKNQFSL | |
| | KLTSVTAADTAVYYCARHRGSY | |
| | FFDIWGLGTLVTVSSASTKGPSV | |
| | FPLAPSSKSTSGGTAALGCLVKD | |
| | YFPEPVTVSWNSGALTSGVHTFP | |
| | AVLQSSGLYSLSSVVTVPSSSLGT | |
| | QTYICNVNHKPSNTKVDKRVEP | |
| | KSC (SEQ ID NO: 293) | |
| Larsen | MHVAQPAVVLASSRGIASFVCEY | SSELTQDPAVSVALGQTVRITCQG |
| Variant_89B09 | ASPGKYTEVRVTVLRQADSQVT | DSVRRFFAHWYQQKPGQAPVLVI |
| | EVCAATYMMGNELTFLDDSICT | SGKDTRPSGIPDRFSGSSSGNTASL |
| | GTSSGNQVNLTIQGLRAMDTGL | TITGAQAEDEADYYCNSRDSSGY |
| | YICKVELMYPPPYYEGIGNGTQI | LVLFGGGTKLTVLGQPKAAPSVT |
| | YVIDPEPCPDSDAMHVAQPAVV | LFPPSSEELQANKATLVCLISDFYP |
| | LASSRGIASFVCEYASPGKATEV | GAVTVAWKADSSPVKAGVETTT |
| | RVTVLRQADSQVTEVCAATYM | PSKQSNNKYAASSYLSLTPEQWK |
| | MGNELTFLDDSICTGTSSGNQVN | SHKSYSCQVTHEGSTVEKTVAPT |
| | LTIQGLRAMDTGLYICKVELMYP | ECS (SEQ ID NO: 67)) |
| | PPYYLGIGNGTQIYVIDPEPCPDS | |
| | DQEPKSSDKTHTCPPCPAPPVAG | |
| | PSVFLFPPKPKDTLMISRTPEVTC | |
| | VVVDVSHEDPEVKFNWYVDGV | |
| | EVHNAKTKPREEQYNSTYRVVS | |
| | VLTVLHQDWLNGKEYKCAVSN | |
| | KALAAPIEKTISKAKGQPREGPG | |
| | LVKPSETLSLTCSVSGGSISTSSY | |
| | YWGWIRQPPGKGLEWSGSIYYS | |
| | GSTYYRPSLQGRATISVDTSKNQ | |
| | FSLKLTSVTAADTAVYYCARHR | |
| | GSYFFDIWGLGTLVTVSSASTKG | |
| | PSVFPLAPSSKSTSGGTAALGCL | |
| | VKDYFPEPVTVSWNSGALTSGV | |
| | HTFPAVLQSSGLYSLSSVVTVPSS | |
| | SLGTQTYICNVNHKPSNTKVDKR | |
| | VEPKSC (SEQ ID NO: 294) | |
| Xu Variant | MHVAQPAVVLASSRGIASFVCEY | SSELTQDPAVSVALGQTVRITCQG |
| 1_89B09 | ASPGKHTEVRVTVLRQADSQVT | DSVRRFFAHWYQQKPGQAPVLVI |
| | EVCAANYMMGNEGTFLDDSICT | SGKDTRPSGIPDRFSGSSSGNTASL |
| | GTSSGNQVNLTIQGLRAMDTGL | TITGAQAEDEADYYCNSRDSSGY |
| | YICMVEKMYPPPYYLGIGNGTQI | LVLFGGGTKLTVLGQPKAAPSVT |
| | YVIDPEPCPDSDAMHVAQPAVV | LFPPSSEELQANKATLVCLISDFYP |
| | LASSRGIASFVCEYASPGKATEV | GAVTVAWKADSSPVKAGVETTT |
| | RVTVLRQADSQVTEVCAATYM | PSKQSNNKYAASSYLSLTPEQWK |
| | MGNELTFLDDSICTGTSSGNQVN | SHKSYSCQVTHEGSTVEKTVAPT |
| | LTIQGLRAMDTGLYICKVELMYP | ECS (SEQ ID NO: 67)) |
| | PPYYLGIGNGTQIYVIDPEPCPDS | |
| | DQEPKSSDKTHTCPPCPAPPVAG | |
| | PSVFLFPPKPKDTLMISRTPEVTC | |
| | VVVDVSHEDPEVKFNWYVDGV | |
| | EVHNAKTKPREEQYNSTYRVVS | |
| | VLTVLHQDWLNGKEYKCAVSN | |
| | KALAAPIEKTISKAKGQPREGPG | |
| | LVKPSETLSLTCSVSGGSISTSSY | |
| | YWGWIRQPPGKGLEWSGSIYYS | |
| | GSTYYRPSLQGRATISVDTSKNQ | |
| | FSLKLTSVTAADTAVYYCARHR | |
| | GSYFFDIWGLGTLVTVSSASTKG | |
| | PSVFPLAPSSKSTSGGTAALGCL | |
| | VKDYFPEPVTVSWNSGALTSGV | |
| | HTFPAVLQSSGLYSLSSVVTVPSS | |
| | SLGTQTYICNVNHKPSNTKVDKR | |
| | VEPKSC (SEQ ID NO: 295) | |
| Xu Variant | MHVAQPAVVLASSRGIASFVCEY | SSELTQDPAVSVALGQTVRITCQG |
| 2_89B09 | ASPGKKTEVRVTVLRQADSQVT | DSVRRFFAHWYQQKPGQAPVLVI |
| | EVCAANYMMGNEGTFLDDSICT | SGKDTRPSGIPDRFSGSSSGNTASL |
| | GTSSGNQVNLTIQGLRAMDTGL | TITGAQAEDEADYYCNSRDSSGY |
| | YICMVEKMYPPPYYLGIGNGTQI | LVLFGGGTKLTVLGQPKAAPSVT |
| | YVIDPEPCPDSDAMHVAQPAVV | LFPPSSEELQANKATLVCLISDFYP |
| | LASSRGIASFVCEYASPGKATEV | GAVTVAWKADSSPVKAGVETTT |
| | RVTVLRQADSQVTEVCAATYM | PSKQSNNKYAASSYLSLTPEQWK |
| | MGNELTFLDDSICTGTSSGNQVN | SHKSYSCQVTHEGSTVEKTVAPT |
| | LTIQGLRAMDTGLYICKVELMYP | ECS (SEQ ID NO: 67)) |
| | PPYYLGIGNGTQIYVIDPEPCPDS | |
| | DQEPKSSDKTHTCPPCPAPPVAG | |
| | PSVFLFPPKPKDTLMISRTPEVTC | |
| | VVVDVSHEDPEVKFNWYVDGV | |
| | EVHNAKTKPREEQYNSTYRVVS | |
| | VLTVLHQDWLNGKEYKCAVSN | |
| | KALAAPIEKTISKAKGQPREGPG | |
| | LVKPSETLSLTCSVSGGSISTSSY | |
| | YWGWIRQPPGKGLEWSGSIYYS | |
| | GSTYYRPSLQGRATISVDTSKNQ | |
| | FSLKLTSVTAADTAVYYCARHR | |
| | GSYFFDIWGLGTLVTVSSASTKG | |
| | PSVFPLAPSSKSTSGGTAALGCL | |
| | VKDYFPEPVTVSWNSGALTSGV | |
| | HTFPAVLQSSGLYSLSSVVTVPSS | |
| | SLGTQTYICNVNHKPSNTKVDKR | |
| | VEPKSC (SEQ ID NO: 296) | |
| Xu Variant | MHVAQPAVVLASSRGIASFVCEY | SSELTQDPAVSVALGQTVRITCQG |
| 3_89B09 | ASPGKTTEVRVTVLRQADSQVTE | DSVRRFFAHWYQQKPGQAPVLVI |
| | VCAANYMMGNEGTFLDDSICTG | SGKDTRPSGIPDRFSGSSSGNTASL |
| | TSSGNQVNLTIQGLRAMDTGLYI | TITGAQAEDEADYYCNSRDSSGY |
| | CMVEKMYPPPYYLGIGNGTQIY | LVLFGGGTKLTVLGQPKAAPSVT |
| | VIDPEPCPDSDAMHVAQPAVVL | LFPPSSEELQANKATLVCLISDFYP |
| | ASSRGIASFVCEYASPGKATEVR | GAVTVAWKADSSPVKAGVETTT |
| | VTVLRQADSQVTEVCAATYMM | PSKQSNNKYAASSYLSLTPEQWK |
| | GNELTFLDDSICTGTSSGNQVNL | SHKSYSCQVTHEGSTVEKTVAPT |
| | TIQGLRAMDTGLYICKVELMYPP | ECS (SEQ ID NO: 67)) |
| | PYYLGIGNGTQIYVIDPEPCPDSD | |
| | QEPKSSDKTHTCPPCPAPPVAGP | |
| | SVFLFPPKPKDTLMISRTPEVTCV | |
| | VVDVSHEDPEVKFNWYVDGVE | |
| | VHNAKTKPREEQYNSTYRVVSV | |
| | LTVLHQDWLNGKEYKCAVSNK | |
| | ALAAPIEKTISKAKGQPREGPGL | |
| | VKPSETLSLTCSVSGGSISTSSYY | |
| | WGWIRQPPGKGLEWSGSIYYSGS | |
| | TYYRPSLQGRATISVDTSKNQFS | |
| | LKLTSVTAADTAVYYCARHRGS | |
| | YFFDIWGLGTLVTVSSASTKGPS | |
| | VFPLAPSSKSTSGGTAALGCLVK | |
| | DYFPEPVTVSWNSGALTSGVHTF | |
| | PAVLQSSGLYSLSSVVTVPSSSLG | |
| | TQTYICNVNHKPSNTKVDKRVEP | |
| | KSC (SEQ ID NO: 297) | |
| Xu Variant | MHVAQPAVVLASSRGIASFVCEY | SSELTQDPAVSVALGQTVRITCQG |
| 4_89B09 | ASPGKWTEVRVTVLRQADSQVT | DSVRRFFAHWYQQKPGQAPVLVI |
| | EVCAANYMMGNEGTFLDDSICT | SGKDTRPSGIPDRFSGSSSGNTASL |
| | GTSSGNQVNLTIQGLRAMDTGL | TITGAQAEDEADYYCNSRDSSGY |
| | YICMVEKMYPPPYYLGIGNGTQI | LVLFGGGTKLTVLGQPKAAPSVT |
| | YVIDPEPCPDSDAMHVAQPAVV | LFPPSSEELQANKATLVCLISDFYP |
| | LASSRGIASFVCEYASPGKATEV | GAVTVAWKADSSPVKAGVETTT |
| | RVTVLRQADSQVTEVCAATYM | PSKQSNNKYAASSYLSLTPEQWK |
| | MGNELTFLDDSICTGTSSGNQVN | SHKSYSCQVTHEGSTVEKTVAPT |
| | LTIQGLRAMDTGLYICKVELMYP | ECS (SEQ ID NO: 67)) |
| | PPYYLGIGNGTQIYVIDPEPCPDS | |
| | DQEPKSSDKTHTCPPCPAPPVAG | |
| | PSVFLFPPKPKDTLMISRTPEVTC | |
| | VVVDVSHEDPEVKFNWYVDGV | |
| | EVHNAKTKPREEQYNSTYRVVS | |
| | VLTVLHQDWLNGKEYKCAVSN | |
| | KALAAPIEKTISKAKGQPREGPG | |
| | LVKPSETLSLTCSVSGGSISTSSY | |
| | YWGWIRQPPGKGLEWSGSIYYS | |
| | GSTYYRPSLQGRATISVDTSKNQ | |
| | FSLKLTSVTAADTAVYYCARHR | |
| | GSYFFDIWGLGTLVTVSSASTKG | |
| | PSVFPLAPSSKSTSGGTAALGCL | |
| | VKDYFPEPVTVSWNSGALTSGV | |
| | HTFPAVLQSSGLYSLSSVVTVPSS | |
| | SLGTQTYICNVNHKPSNTKVDKR | |
| | VEPKSC (SEQ ID NO: 298) | |
| Xu Variant | MHVAQPAVVLASSRGIASFVCEY | SSELTQDPAVSVALGQTVRITCQG |
| 5_89B09 | ASPGKYTEVRVTVLRQADSQVT | DSVRRFFAHWYQQKPGQAPVLVI |
| | EVCAANYMMGNEGTFLDDSICT | SGKDTRPSGIPDRFSGSSSGNTASL |
| | GTSSGNQVNLTIQGLRAMDTGL | TITGAQAEDEADYYCNSRDSSGY |
| | YICMVEKMYPPPYYLGIGNGTQI | LVLFGGGTKLTVLGQPKAAPSVT |
| | YVIDPEPCPDSDAMHVAQPAVV | LFPPSSEELQANKATLVCLISDFYP |
| | LASSRGIASFVCEYASPGKATEV | GAVTVAWKADSSPVKAGVETTT |
| | RVTVLRQADSQVTEVCAATYM | PSKQSNNKYAASSYLSLTPEQWK |
| | MGNELTFLDDSICTGTSSGNQVN | SHKSYSCQVTHEGSTVEKTVAPT |
| | LTIQGLRAMDTGLYICKVELMYP | ECS (SEQ ID NO: 67)) |
| | PPYYLGIGNGTQIYVIDPEPCPDS | |
| | DQEPKSSDKTHTCPPCPAPPVAG | |
| | PSVFLFPPKPKDTLMISRTPEVTC | |
| | VVVDVSHEDPEVKFNWYVDGV | |
| | EVHNAKTKPREEQYNSTYRVVS | |
| | VLTVLHQDWLNGKEYKCAVSN | |
| | KALAAPIEKTISKAKGQPREGPG | |
| | LVKPSETLSLTCSVSGGSISTSSY | |
| | YWGWIRQPPGKGLEWSGSIYYS | |
| | GSTYYRPSLQGRATISVDTSKNQ | |
| | FSLKLTSVTAADTAVYYCARHR | |
| | GSYFFDIWGLGTLVTVSSASTKG | |
| | PSVFPLAPSSKSTSGGTAALGCL | |
| | VKDYFPEPVTVSWNSGALTSGV | |
| | HTFPAVLQSSGLYSLSSVVTVPSS | |
| | SLGTQTYICNVNHKPSNTKVDKR | |
| | VEPKSC (SEQ ID NO: 299) | |
| Xu Variant | MHVAQPAVVLASSRGIASFVCEY | SSELTQDPAVSVALGQTVRITCQG |
| 6_89B09 | ASPGKHTEVRVTVLRQADSQVT | DSVRRFFAHWYQQKPGQAPVLVI |
| | EVCAAYYMMGNEGTFLDDSICT | SGKDTRPSGIPDRFSGSSSGNTASL |
| | GTSSGNQVNLTIQGLRAMDTGL | TITGAQAEDEADYYCNSRDSSGY |
| | YICQVEKMYPPPYYLGIGNGTQI | LVLFGGGTKLTVLGQPKAAPSVT |
| | YVIDPEPCPDSDAMHVAQPAVV | LFPPSSEELQANKATLVCLISDFYP |
| | LASSRGIASFVCEYASPGKATEV | GAVTVAWKADSSPVKAGVETTT |
| | RVTVLRQADSQVTEVCAATYM | PSKQSNNKYAASSYLSLTPEQWK |
| | MGNELTFLDDSICTGTSSGNQVN | SHKSYSCQVTHEGSTVEKTVAPT |
| | LTIQGLRAMDTGLYICKVELMYP | ECS (SEQ ID NO: 67)) |
| | PPYYLGIGNGTQIYVIDPEPCPDS | |
| | DQEPKSSDKTHTCPPCPAPPVAG | |
| | PSVFLFPPKPKDTLMISRTPEVTC | |
| | VVVDVSHEDPEVKFNWYVDGV | |
| | EVHNAKTKPREEQYNSTYRVVS | |
| | VLTVLHQDWLNGKEYKCAVSN | |
| | KALAAPIEKTISKAKGQPREGPG | |
| | LVKPSETLSLTCSVSGGSISTSSY | |
| | YWGWIRQPPGKGLEWSGSIYYS | |
| | GSTYYRPSLQGRATISVDTSKNQ | |
| | FSLKLTSVTAADTAVYYCARHR | |
| | GSYFFDIWGLGTLVTVSSASTKG | |
| | PSVFPLAPSSKSTSGGTAALGCL | |
| | VKDYFPEPVTVSWNSGALTSGV | |
| | HTFPAVLQSSGLYSLSSVVTVPSS | |
| | SLGTQTYICNVNHKPSNTKVDKR | |
| | VEPKSC (SEQ ID NO: 300) | |
| Xu Variant | MHVAQPAVVLASSRGIASFVCEY | SSELTQDPAVSVALGQTVRITCQG |
| 7_89B09 | ASPGKKTEVRVTVLRQADSQVT | DSVRRFFAHWYQQKPGQAPVLVI |
| | EVCAAYYMMGNEGTFLDDSICT | SGKDTRPSGIPDRFSGSSSGNTASL |
| | GTSSGNQVNLTIQGLRAMDTGL | TITGAQAEDEADYYCNSRDSSGY |
| | YICQVEKMYPPPYYLGIGNGTQI | LVLFGGGTKLTVLGQPKAAPSVT |
| | YVIDPEPCPDSDAMHVAQPAVV | LFPPSSEELQANKATLVCLISDFYP |
| | LASSRGIASFVCEYASPGKATEV | GAVTVAWKADSSPVKAGVETTT |
| | RVTVLRQADSQVTEVCAATYM | PSKQSNNKYAASSYLSLTPEQWK |
| | MGNELTFLDDSICTGTSSGNQVN | SHKSYSCQVTHEGSTVEKTVAPT |
| | LTIQGLRAMDTGLYICKVELMYP | ECS (SEQ ID NO: 67)) |
| | PPYYLGIGNGTQIYVIDPEPCPDS | |
| | DQEPKSSDKTHTCPPCPAPPVAG | |
| | PSVFLFPPKPKDTLMISRTPEVTC | |
| | VVVDVSHEDPEVKFNWYVDGV | |
| | EVHNAKTKPREEQYNSTYRVVS | |
| | VLTVLHQDWLNGKEYKCAVSN | |
| | KALAAPIEKTISKAKGQPREGPG | |
| | LVKPSETLSLTCSVSGGSISTSSY | |
| | YWGWIRQPPGKGLEWSGSIYYS | |
| | GSTYYRPSLQGRATISVDTSKNQ | |
| | FSLKLTSVTAADTAVYYCARHR | |
| | GSYFFDIWGLGTLVTVSSASTKG | |
| | PSVFPLAPSSKSTSGGTAALGCL | |
| | VKDYFPEPVTVSWNSGALTSGV | |
| | HTFPAVLQSSGLYSLSSVVTVPSS | |
| | SLGTQTYICNVNHKPSNTKVDKR | |
| | VEPKSC (SEQ ID NO: 301) | |
| Xu Variant | MHVAQPAVVLASSRGIASFVCEY | SSELTQDPAVSVALGQTVRITCQG |
| 8_89B09 | ASPGKTTEVRVTVLRQADSQVTE | DSVRRFFAHWYQQKPGQAPVLVI |
| | VCAAYYMMGNEGTFLDDSICTG | SGKDTRPSGIPDRFSGSSSGNTASL |
| | TSSGNQVNLTIQGLRAMDTGLYI | TITGAQAEDEADYYCNSRDSSGY |
| | CQVEKMYPPPYYLGIGNGTQIYV | LVLFGGGTKLTVLGQPKAAPSVT |
| | IDPEPCPDSDAMHVAQPAVVLAS | LFPPSSEELQANKATLVCLISDFYP |
| | SRGIASFVCEYASPGKATEVRVT | GAVTVAWKADSSPVKAGVETTT |
| | VLRQADSQVTEVCAATYMMGN | PSKQSNNKYAASSYLSLTPEQWK |
| | ELTFLDDSICTGTSSGNQVNLTIQ | SHKSYSCQVTHEGSTVEKTVAPT |
| | GLRAMDTGLYICKVELMYPPPY | ECS (SEQ ID NO: 67)) |
| | YLGIGNGTQIYVIDPEPCPDSDQE | |
| | PKSSDKTHTCPPCPAPPVAGPSVF | |
| | LFPPKPKDTLMISRTPEVTCVVV | |
| | DVSHEDPEVKFNWYVDGVEVH | |
| | NAKTKPREEQYNSTYRVVSVLT | |
| | VLHQDWLNGKEYKCAVSNKAL | |
| | AAPIEKTISKAKGQPREGPGLVKP | |
| | SETLSLTCSVSGGSISTSSYYWG | |
| | WIRQPPGKGLEWSGSIYYSGSTY | |
| | YRPSLQGRATISVDTSKNQFSLK | |
| | LTSVTAADTAVYYCARHRGSYF | |
| | FDIWGLGTLVTVSSASTKGPSVF | |
| | PLAPSSKSTSGGTAALGCLVKDY | |
| | FPEPVTVSWNSGALTSGVHTFPA | |
| | VLQSSGLYSLSSVVTVPSSSLGTQ | |
| | TYICNVNHKPSNTKVDKRVEPKS | |
| | C (SEQ ID NO: 302) | |
| Xu Variant | MHVAQPAVVLASSRGIASFVCEY | SSELTQDPAVSVALGQTVRITCQG |
| 9_89B09 | ASPGKWTEVRVTVLRQADSQVT | DSVRRFFAHWYQQKPGQAPVLVI |
| | EVCAAYYMMGNEGTFLDDSICT | SGKDTRPSGIPDRFSGSSSGNTASL |
| | GTSSGNQVNLTIQGLRAMDTGL | TITGAQAEDEADYYCNSRDSSGY |
| | YICQVEKMYPPPYYLGIGNGTQI | LVLFGGGTKLTVLGQPKAAPSVT |
| | YVIDPEPCPDSDAMHVAQPAVV | LFPPSSEELQANKATLVCLISDFYP |
| | LASSRGIASFVCEYASPGKATEV | GAVTVAWKADSSPVKAGVETTT |
| | RVTVLRQADSQVTEVCAATYM | PSKQSNNKYAASSYLSLTPEQWK |
| | MGNELTFLDDSICTGTSSGNQVN | SHKSYSCQVTHEGSTVEKTVAPT |
| | LTIQGLRAMDTGLYICKVELMYP | ECS (SEQ ID NO: 67)) |
| | PPYYLGIGNGTQIYVIDPEPCPDS | |
| | DQEPKSSDKTHTCPPCPAPPVAG | |
| | PSVFLFPPKPKDTLMISRTPEVTC | |
| | VVVDVSHEDPEVKFNWYVDGV | |
| | EVHNAKTKPREEQYNSTYRVVS | |
| | VLTVLHQDWLNGKEYKCAVSN | |
| | KALAAPIEKTISKAKGQPREGPG | |
| | LVKPSETLSLTCSVSGGSISTSSY | |
| | YWGWIRQPPGKGLEWSGSIYYS | |
| | GSTYYRPSLQGRATISVDTSKNQ | |
| | FSLKLTSVTAADTAVYYCARHR | |
| | GSYFFDIWGLGTLVTVSSASTKG | |
| | PSVFPLAPSSKSTSGGTAALGCL | |
| | VKDYFPEPVTVSWNSGALTSGV | |
| | HTFPAVLQSSGLYSLSSVVTVPSS | |
| | SLGTQTYICNVNHKPSNTKVDKR | |
| | VEPKSC (SEQ ID NO: 303) | |
| Xu Variant | MHVAQPAVVLASSRGIASFVCEY | SSELTQDPAVSVALGQTVRITCQG |
| 10_89B09 | ASPGKYTEVRVTVLRQADSQVT | DSVRRFFAHWYQQKPGQAPVLVI |
| | EVCAAYYMMGNEGTFLDDSICT | SGKDTRPSGIPDRFSGSSSGNTASL |
| | GTSSGNQVNLTIQGLRAMDTGL | TITGAQAEDEADYYCNSRDSSGY |
| | YICQVEKMYPPPYYLGIGNGTQI | LVLFGGGTKLTVLGQPKAAPSVT |
| | YVIDPEPCPDSDAMHVAQPAVV | LFPPSSEELQANKATLVCLISDFYP |
| | LASSRGIASFVCEYASPGKATEV | GAVTVAWKADSSPVKAGVETTT |
| | RVTVLRQADSQVTEVCAATYM | PSKQSNNKYAASSYLSLTPEQWK |
| | MGNELTFLDDSICTGTSSGNQVN | SHKSYSCQVTHEGSTVEKTVAPT |
| | LTIQGLRAMDTGLYICKVELMYP | ECS (SEQ ID NO: 67)) |
| | PPYYLGIGNGTQIYVIDPEPCPDS | |
| | DQEPKSSDKTHTCPPCPAPPVAG | |
| | PSVFLFPPKPKDTLMISRTPEVTC | |
| | VVVDVSHEDPEVKFNWYVDGV | |
| | EVHNAKTKPREEQYNSTYRVVS | |
| | VLTVLHQDWLNGKEYKCAVSN | |
| | KALAAPIEKTISKAKGQPREGPG | |
| | LVKPSETLSLTCSVSGGSISTSSY | |
| | YWGWIRQPPGKGLEWSGSIYYS | |
| | GSTYYRPSLQGRATISVDTSKNQ | |
| | FSLKLTSVTAADTAVYYCARHR | |
| | GSYFFDIWGLGTLVTVSSASTKG | |
| | PSVFPLAPSSKSTSGGTAALGCL | |
| | VKDYFPEPVTVSWNSGALTSGV | |
| | HTFPAVLQSSGLYSLSSVVTVPSS | |
| | SLGTQTYICNVNHKPSNTKVDKR | |
| | VEPKSC (SEQ ID NO: 304) | |
| Xu Variant | MHVAQPAVVLASSRGIASFVCEY | SSELTQDPAVSVALGQTVRITCQG |
| 11_89B09 | ASPGKHTEVRVTVLRQADSQVT | DSVRRFFAHWYQQKPGQAPVLVI |
| | EVCAAYYMMGNEGTFLDDSICT | SGKDTRPSGIPDRFSGSSSGNTASL |
| | GTSSGNQVNLTIQGLRAMDTGL | TITGAQAEDEADYYCNSRDSSGY |
| | YICQVEKMYPPPYYLGIGNGTQI | LVLFGGGTKLTVLGQPKAAPSVT |
| | YVIDPEPCPDSDAMHVAQPAVV | LFPPSSEELQANKATLVCLISDFYP |
| | LASSRGIASFVCEYASPGKATEV | GAVTVAWKADSSPVKAGVETTT |
| | RVTVLRQADSQVTEVCAATYM | PSKQSNNKYAASSYLSLTPEQWK |
| | MGNELTFLDDSICTGTSSGNQVN | SHKSYSCQVTHEGSTVEKTVAPT |
| | LTIQGLRAMDTGLYICKVELMYP | ECS (SEQ ID NO: 67)) |
| | PPYYLGIGNGTQIYVIDPEPCPDS | |
| | DQEPKSSDKTHTCPPCPAPPVAG | |
| | PSVFLFPPKPKDTLMISRTPEVTC | |
| | VVVDVSHEDPEVKFNWYVDGV | |
| | EVHNAKTKPREEQYNSTYRVVS | |
| | VLTVLHQDWLNGKEYKCAVSN | |
| | KALAAPIEKTISKAKGQPREGPG | |
| | LVKPSETLSLTCSVSGGSISTSSY | |
| | YWGWIRQPPGKGLEWSGSIYYS | |
| | GSTYYRPSLQGRATISVDTSKNQ | |
| | FSLKLTSVTAADTAVYYCARHR | |
| | GSYFFDIWGLGTLVTVSSASTKG | |
| | PSVFPLAPSSKSTSGGTAALGCL | |
| | VKDYFPEPVTVSWNSGALTSGV | |
| | HTFPAVLQSSGLYSLSSVVTVPSS | |
| | SLGTQTYICNVNHKPSNTKVDKR | |
| | VEPKSC (SEQ ID NO: 305) | |
| Xu Variant | MHVAQPAVVLASSRGIASFVCEY | SSELTQDPAVSVALGQTVRITCQG |
| 12_89B09 | ASPGKKTEVRVTVLRQADSQVT | DSVRRFFAHWYQQKPGQAPVLVI |
| | EVCAAYYMMGNEGTFLDDSICT | SGKDTRPSGIPDRESGSSSGNTASL |
| | GTSSGNQVNLTIQGLRAMDTGL | TITGAQAEDEADYYCNSRDSSGY |
| | YICQVEKMYPPPYYLGIGNGTQI | LVLFGGGTKLTVLGQPKAAPSVT |
| | YVIDPEPCPDSDAMHVAQPAVV | LFPPSSEELQANKATLVCLISDFYP |
| | LASSRGIASFVCEYASPGKATEV | GAVTVAWKADSSPVKAGVETTT |
| | RVTVLRQADSQVTEVCAATYM | PSKQSNNKYAASSYLSLTPEQWK |
| | MGNELTFLDDSICTGTSSGNQVN | SHKSYSCQVTHEGSTVEKTVAPT |
| | LTIQGLRAMDTGLYICKVELMYP | ECS (SEQ ID NO: 67)) |
| | PPYYLGIGNGTQIYVIDPEPCPDS | |
| | DQEPKSSDKTHTCPPCPAPPVAG | |
| | PSVFLFPPKPKDTLMISRTPEVTC | |
| | VVVDVSHEDPEVKFNWYVDGV | |
| | EVHNAKTKPREEQYNSTYRVVS | |
| | VLTVLHQDWLNGKEYKCAVSN | |
| | KALAAPIEKTISKAKGQPREGPG | |
| | LVKPSETLSLTCSVSGGSISTSSY | |
| | YWGWIRQPPGKGLEWSGSIYYS | |
| | GSTYYRPSLQGRATISVDTSKNQ | |
| | FSLKLTSVTAADTAVYYCARHR | |
| | GSYFFDIWGLGTLVTVSSASTKG | |
| | PSVFPLAPSSKSTSGGTAALGCL | |
| | VKDYFPEPVTVSWNSGALTSGV | |
| | HTFPAVLQSSGLYSLSSVVTVPSS | |
| | SLGTQTYICNVNHKPSNTKVDKR | |
| | VEPKSC (SEQ ID NO: 306) | |
| Xu Variant | MHVAQPAVVLASSRGIASFVCEY | SSELTQDPAVSVALGQTVRITCQG |
| 13_89B09 | ASPGKTTEVRVTVLRQADSQVTE | DSVRRFFAHWYQQKPGQAPVLVI |
| | VCAAYYMMGNEGTFLDDSICTG | SGKDTRPSGIPDRFSGSSSGNTASL |
| | TSSGNQVNLTIQGLRAMDTGLYI | TITGAQAEDEADYYCNSRDSSGY |
| | CQVEKMYPPPYYLGIGNGTQIYV | LVLFGGGTKLTVLGQPKAAPSVT |
| | IDPEPCPDSDAMHVAQPAVVLAS | LFPPSSEELQANKATLVCLISDFYP |
| | SRGIASFVCEYASPGKATEVRVT | GAVTVAWKADSSPVKAGVETTT |
| | VLRQADSQVTEVCAATYMMGN | PSKQSNNKYAASSYLSLTPEQWK |
| | ELTFLDDSICTGTSSGNQVNLTIQ | SHKSYSCQVTHEGSTVEKTVAPT |
| | GLRAMDTGLYICKVELMYPPPY | ECS (SEQ ID NO: 67)) |
| | YLGIGNGTQIYVIDPEPCPDSDQE | |
| | PKSSDKTHTCPPCPAPPVAGPSVF | |
| | LFPPKPKDTLMISRTPEVTCVVV | |
| | DVSHEDPEVKFNWYVDGVEVH | |
| | NAKTKPREEQYNSTYRVVSVLT | |
| | VLHQDWLNGKEYKCAVSNKAL | |
| | AAPIEKTISKAKGQPREGPGLVKP | |
| | SETLSLTCSVSGGSISTSSYYWG | |
| | WIRQPPGKGLEWSGSIYYSGSTY | |
| | YRPSLQGRATISVDTSKNQFSLK | |
| | LTSVTAADTAVYYCARHRGSYF | |
| | FDIWGLGTLVTVSSASTKGPSVF | |
| | PLAPSSKSTSGGTAALGCLVKDY | |
| | FPEPVTVSWNSGALTSGVHTFPA | |
| | VLQSSGLYSLSSVVTVPSSSLGTQ | |
| | TYICNVNHKPSNTKVDKRVEPKS | |
| | C (SEQ ID NO: 307) | |
| Xu Variant | MHVAQPAVVLASSRGIASFVCEY | SSELTQDPAVSVALGQTVRITCQG |
| 14_89B09 | ASPGKWTEVRVTVLRQADSQVT | DSVRRFFAHWYQQKPGQAPVLVI |
| | EVCAAYYMMGNEGTFLDDSICT | SGKDTRPSGIPDRFSGSSSGNTASL |
| | GTSSGNQVNLTIQGLRAMDTGL | TITGAQAEDEADYYCNSRDSSGY |
| | YICQVEKMYPPPYYLGIGNGTQI | LVLFGGGTKLTVLGQPKAAPSVT |
| | YVIDPEPCPDSDAMHVAQPAVV | LFPPSSEELQANKATLVCLISDFYP |
| | LASSRGIASFVCEYASPGKATEV | GAVTVAWKADSSPVKAGVETTT |
| | RVTVLRQADSQVTEVCAATYM | PSKQSNNKYAASSYLSLTPEQWK |
| | MGNELTFLDDSICTGTSSGNQVN | SHKSYSCQVTHEGSTVEKTVAPT |
| | LTIQGLRAMDTGLYICKVELMYP | ECS (SEQ ID NO: 67)) |
| | PPYYLGIGNGTQIYVIDPEPCPDS | |
| | DQEPKSSDKTHTCPPCPAPPVAG | |
| | PSVFLFPPKPKDTLMISRTPEVTC | |
| | VVVDVSHEDPEVKFNWYVDGV | |
| | EVHNAKTKPREEQYNSTYRVVS | |
| | VLTVLHQDWLNGKEYKCAVSN | |
| | KALAAPIEKTISKAKGQPREGPG | |
| | LVKPSETLSLTCSVSGGSISTSSY | |
| | YWGWIRQPPGKGLEWSGSIYYS | |
| | GSTYYRPSLQGRATISVDTSKNQ | |
| | FSLKLTSVTAADTAVYYCARHR | |
| | GSYFFDIWGLGTLVTVSSASTKG | |
| | PSVFPLAPSSKSTSGGTAALGCL | |
| | VKDYFPEPVTVSWNSGALTSGV | |
| | HTFPAVLQSSGLYSLSSVVTVPSS | |
| | SLGTQTYICNVNHKPSNTKVDKR | |
| | VEPKSC (SEQ ID NO: 308) | |
| Xu Variant | MHVAQPAVVLASSRGIASFVCEY | SSELTQDPAVSVALGQTVRITCQG |
| 15_89B09 | ASPGKYTEVRVTVLRQADSQVT | DSVRRFFAHWYQQKPGQAPVLVI |
| | EVCAAYYMMGNEGTFLDDSICT | SGKDTRPSGIPDRFSGSSSGNTASL |
| | GTSSGNQVNLTIQGLRAMDTGL | TITGAQAEDEADYYCNSRDSSGY |
| | YICQVEKMYPPPYYLGIGNGTQI | LVLFGGGTKLTVLGQPKAAPSVT |
| | YVIDPEPCPDSDAMHVAQPAVV | LFPPSSEELQANKATLVCLISDFYP |
| | LASSRGIASFVCEYASPGKATEV | GAVTVAWKADSSPVKAGVETTT |
| | RVTVLRQADSQVTEVCAATYM | PSKQSNNKYAASSYLSLTPEQWK |
| | MGNELTFLDDSICTGTSSGNQVN | SHKSYSCQVTHEGSTVEKTVAPT |
| | LTIQGLRAMDTGLYICKVELMYP | ECS (SEQ ID NO: 67)) |
| | PPYYLGIGNGTQIYVIDPEPCPDS | |
| | DQEPKSSDKTHTCPPCPAPPVAG | |
| | PSVFLFPPKPKDTLMISRTPEVTC | |
| | VVVDVSHEDPEVKFNWYVDGV | |
| | EVHNAKTKPREEQYNSTYRVVS | |
| | VLTVLHQDWLNGKEYKCAVSN | |
| | KALAAPIEKTISKAKGQPREGPG | |
| | LVKPSETLSLTCSVSGGSISTSSY | |
| | YWGWIRQPPGKGLEWSGSIYYS | |
| | GSTYYRPSLQGRATISVDTSKNQ | |
| | FSLKLTSVTAADTAVYYCARHR | |
| | GSYFFDIWGLGTLVTVSSASTKG | |
| | PSVFPLAPSSKSTSGGTAALGCL | |
| | VKDYFPEPVTVSWNSGALTSGV | |
| | HTFPAVLQSSGLYSLSSVVTVPSS | |
| | SLGTQTYICNVNHKPSNTKVDKR | |
| | VEPKSC (SEQ ID NO: 309) | |
| Xu Variant | MHVAQPAVVLASSRGIASFVCEY | SSELTQDPAVSVALGQTVRITCQG |
| 16_89B09 | ASPGKHTEVRVTVLRQADSQVT | DSVRRFFAHWYQQKPGQAPVLVI |
| | EVCAANYMMGNEGTFLDDSICT | SGKDTRPSGIPDRFSGSSSGNTASL |
| | GTSSGNQVNLTIQGLRAMDTGL | TITGAQAEDEADYYCNSRDSSGY |
| | YICQVEKMYPPPYYLGIGNGTQI | LVLFGGGTKLTVLGQPKAAPSVT |
| | YVIDPEPCPDSDAMHVAQPAVV | LFPPSSEELQANKATLVCLISDFYP |
| | LASSRGIASFVCEYASPGKATEV | GAVTVAWKADSSPVKAGVETTT |
| | RVTVLRQADSQVTEVCAATYM | PSKQSNNKYAASSYLSLTPEQWK |
| | MGNELTFLDDSICTGTSSGNQVN | SHKSYSCQVTHEGSTVEKTVAPT |
| | LTIQGLRAMDTGLYICKVELMYP | ECS (SEQ ID NO: 67)) |
| | PPYYLGIGNGTQIYVIDPEPCPDS | |
| | DQEPKSSDKTHTCPPCPAPPVAG | |
| | PSVFLFPPKPKDTLMISRTPEVTC | |
| | VVVDVSHEDPEVKFNWYVDGV | |
| | EVHNAKTKPREEQYNSTYRVVS | |
| | VLTVLHQDWLNGKEYKCAVSN | |
| | KALAAPIEKTISKAKGQPREGPG | |
| | LVKPSETLSLTCSVSGGSISTSSY | |
| | YWGWIRQPPGKGLEWSGSIYYS | |
| | GSTYYRPSLQGRATISVDTSKNQ | |
| | FSLKLTSVTAADTAVYYCARHR | |
| | GSYFFDIWGLGTLVTVSSASTKG | |
| | PSVFPLAPSSKSTSGGTAALGCL | |
| | VKDYFPEPVTVSWNSGALTSGV | |
| | HTFPAVLQSSGLYSLSSVVTVPSS | |
| | SLGTQTYICNVNHKPSNTKVDKR | |
| | VEPKSC (SEQ ID NO: 310) | |
| Xu Variant | MHVAQPAVVLASSRGIASFVCEY | SSELTQDPAVSVALGQTVRITCQG |
| 17_89B09 | ASPGKKTEVRVTVLRQADSQVT | DSVRRFFAHWYQQKPGQAPVLVI |
| | EVCAANYMMGNEGTFLDDSICT | SGKDTRPSGIPDRFSGSSSGNTASL |
| | GTSSGNQVNLTIQGLRAMDTGL | TITGAQAEDEADYYCNSRDSSGY |
| | YICQVEKMYPPPYYLGIGNGTQI | LVLFGGGTKLTVLGQPKAAPSVT |
| | YVIDPEPCPDSDAMHVAQPAVV | LFPPSSEELQANKATLVCLISDFYP |
| | LASSRGIASFVCEYASPGKATEV | GAVTVAWKADSSPVKAGVETTT |
| | RVTVLRQADSQVTEVCAATYM | PSKQSNNKYAASSYLSLTPEQWK |
| | MGNELTFLDDSICTGTSSGNQVN | SHKSYSCQVTHEGSTVEKTVAPT |
| | LTIQGLRAMDTGLYICKVELMYP | ECS (SEQ ID NO: 67)) |
| | PPYYLGIGNGTQIYVIDPEPCPDS | |
| | DQEPKSSDKTHTCPPCPAPPVAG | |
| | PSVFLFPPKPKDTLMISRTPEVTC | |
| | VVVDVSHEDPEVKFNWYVDGV | |
| | EVHNAKTKPREEQYNSTYRVVS | |
| | VLTVLHQDWLNGKEYKCAVSN | |
| | KALAAPIEKTISKAKGQPREGPG | |
| | LVKPSETLSLTCSVSGGSISTSSY | |
| | YWGWIRQPPGKGLEWSGSIYYS | |
| | GSTYYRPSLQGRATISVDTSKNQ | |
| | FSLKLTSVTAADTAVYYCARHR | |
| | GSYFFDIWGLGTLVTVSSASTKG | |
| | PSVFPLAPSSKSTSGGTAALGCL | |
| | VKDYFPEPVTVSWNSGALTSGV | |
| | HTFPAVLQSSGLYSLSSVVTVPSS | |
| | SLGTQTYICNVNHKPSNTKVDKR | |
| | VEPKSC (SEQ ID NO: 311) | |
| Xu Variant | MHVAQPAVVLASSRGIASFVCEY | SSELTQDPAVSVALGQTVRITCQG |
| 18_89B09 | ASPGKTTEVRVTVLRQADSQVTE | DSVRRFFAHWYQQKPGQAPVLVI |
| | VCAANYMMGNEGTFLDDSICTG | SGKDTRPSGIPDRFSGSSSGNTASL |
| | TSSGNQVNLTIQGLRAMDTGLYI | TITGAQAEDEADYYCNSRDSSGY |
| | CQVEKMYPPPYYLGIGNGTQIYV | LVLFGGGTKLTVLGQPKAAPSVT |
| | IDPEPCPDSDAMHVAQPAVVLAS | LFPPSSEELQANKATLVCLISDFYP |
| | SRGIASFVCEYASPGKATEVRVT | GAVTVAWKADSSPVKAGVETTT |
| | VLRQADSQVTEVCAATYMMGN | PSKQSNNKYAASSYLSLTPEQWK |
| | ELTFLDDSICTGTSSGNQVNLTIQ | SHKSYSCQVTHEGSTVEKTVAPT |
| | GLRAMDTGLYICKVELMYPPPY | ECS (SEQ ID NO: 67)) |
| | YLGIGNGTQIYVIDPEPCPDSDQE | |
| | PKSSDKTHTCPPCPAPPVAGPSVF | |
| | LFPPKPKDTLMISRTPEVTCVVV | |
| | DVSHEDPEVKFNWYVDGVEVH | |
| | NAKTKPREEQYNSTYRVVSVLT | |
| | VLHQDWLNGKEYKCAVSNKAL | |
| | AAPIEKTISKAKGQPREGPGLVKP | |
| | SETLSLTCSVSGGSISTSSYYWG | |
| | WIRQPPGKGLEWSGSIYYSGSTY | |
| | YRPSLQGRATISVDTSKNQFSLK | |
| | LTSVTAADTAVYYCARHRGSYF | |
| | FDIWGLGTLVTVSSASTKGPSVF | |
| | PLAPSSKSTSGGTAALGCLVKDY | |
| | FPEPVTVSWNSGALTSGVHTFPA | |
| | VLQSSGLYSLSSVVTVPSSSLGTQ | |
| | TYICNVNHKPSNTKVDKRVEPKS | |
| | C (SEQ ID NO: 312) | |
| Xu Variant | MHVAQPAVVLASSRGIASFVCEY | SSELTQDPAVSVALGQTVRITCQG |
| 19_89B09 | ASPGKWTEVRVTVLRQADSQVT | DSVRRFFAHWYQQKPGQAPVLVI |
| | EVCAANYMMGNEGTFLDDSICT | SGKDTRPSGIPDRESGSSSGNTASL |
| | GTSSGNQVNLTIQGLRAMDTGL | TITGAQAEDEADYYCNSRDSSGY |
| | YICQVEKMYPPPYYLGIGNGTQI | LVLFGGGTKLTVLGQPKAAPSVT |
| | YVIDPEPCPDSDAMHVAQPAVV | LFPPSSEELQANKATLVCLISDFYP |
| | LASSRGIASFVCEYASPGKATEV | GAVTVAWKADSSPVKAGVETTT |
| | RVTVLRQADSQVTEVCAATYM | PSKQSNNKYAASSYLSLTPEQWK |
| | MGNELTFLDDSICTGTSSGNQVN | SHKSYSCQVTHEGSTVEKTVAPT |
| | LTIQGLRAMDTGLYICKVELMYP | ECS (SEQ ID NO: 67)) |
| | PPYYLGIGNGTQIYVIDPEPCPDS | |
| | DQEPKSSDKTHTCPPCPAPPVAG | |
| | PSVFLFPPKPKDTLMISRTPEVTC | |
| | VVVDVSHEDPEVKFNWYVDGV | |
| | EVHNAKTKPREEQYNSTYRVVS | |
| | VLTVLHQDWLNGKEYKCAVSN | |
| | KALAAPIEKTISKAKGQPREGPG | |
| | LVKPSETLSLTCSVSGGSISTSSY | |
| | YWGWIRQPPGKGLEWSGSIYYS | |
| | GSTYYRPSLQGRATISVDTSKNQ | |
| | FSLKLTSVTAADTAVYYCARHR | |
| | GSYFFDIWGLGTLVTVSSASTKG | |
| | PSVFPLAPSSKSTSGGTAALGCL | |
| | VKDYFPEPVTVSWNSGALTSGV | |
| | HTFPAVLQSSGLYSLSSVVTVPSS | |
| | SLGTQTYICNVNHKPSNTKVDKR | |
| | VEPKSC (SEQ ID NO: 313) | |
| Xu Variant | MHVAQPAVVLASSRGIASFVCEY | SSELTQDPAVSVALGQTVRITCQG |
| 20_89B09 | ASPGKYTEVRVTVLRQADSQVT | DSVRRFFAHWYQQKPGQAPVLVI |
| | EVCAANYMMGNEGTFLDDSICT | SGKDTRPSGIPDRFSGSSSGNTASL |
| | GTSSGNQVNLTIQGLRAMDTGL | TITGAQAEDEADYYCNSRDSSGY |
| | YICQVEKMYPPPYYLGIGNGTQI | LVLFGGGTKLTVLGQPKAAPSVT |
| | YVIDPEPCPDSDAMHVAQPAVV | LFPPSSEELQANKATLVCLISDFYP |
| | LASSRGIASFVCEYASPGKATEV | GAVTVAWKADSSPVKAGVETTT |
| | RVTVLRQADSQVTEVCAATYM | PSKQSNNKYAASSYLSLTPEQWK |
| | MGNELTFLDDSICTGTSSGNQVN | SHKSYSCQVTHEGSTVEKTVAPT |
| | LTIQGLRAMDTGLYICKVELMYP | ECS (SEQ ID NO: 67)) |
| | PPYYLGIGNGTQIYVIDPEPCPDS | |
| | DQEPKSSDKTHTCPPCPAPPVAG | |
| | PSVFLFPPKPKDTLMISRTPEVTC | |
| | VVVDVSHEDPEVKFNWYVDGV | |
| | EVHNAKTKPREEQYNSTYRVVS | |
| | VLTVLHQDWLNGKEYKCAVSN | |
| | KALAAPIEKTISKAKGQPREGPG | |
| | LVKPSETLSLTCSVSGGSISTSSY | |
| | YWGWIRQPPGKGLEWSGSIYYS | |
| | GSTYYRPSLQGRATISVDTSKNQ | |
| | FSLKLTSVTAADTAVYYCARHR | |
| | GSYFFDIWGLGTLVTVSSASTKG | |
| | PSVFPLAPSSKSTSGGTAALGCL | |
| | VKDYFPEPVTVSWNSGALTSGV | |
| | HTFPAVLQSSGLYSLSSVVTVPSS | |
| | SLGTQTYICNVNHKPSNTKVDKR | |
| | VEPKSC (SEQ ID NO: 314) | |
| Bernett Variant | MHVAQPAVVLASSRGIASFVCEY | SSELTQDPAVSVALGQTVRITCQG |
| 1_89B09 | ASPGKHTEVRVTVLRQADSQVT | DSVRRFFAHWYQQKPGQAPVLVI |
| | EVCFANYYMGNELTFDDDSICTG | SGKDTRPSGIPDRFSGSSSGNTASL |
| | TSSGNQVNLTIQGLRAMDTGLYI | TITGAQAEDEADYYCNSRDSSGY |
| | CQVELMYPPPYYEGIGNGTQIYV | LVLFGGGTKLTVLGQPKAAPSVT |
| | IDPEPCPDSD | LFPPSSEELQANKATLVCLISDFYP |
| | AMHVAQPAVVLASSRGIASFVCE | GAVTVAWKADSSPVKAGVETTT |
| | YASPGKATEVRVTVLRQADSQV | PSKQSNNKYAASSYLSLTPEQWK |
| | TEVCAATYMMGNELTFLDDSICT | SHKSYSCQVTHEGSTVEKTVAPT |
| | GTSSGNQVNLTIQGLRAMDTGL | ECS (SEQ ID NO: 67)) |
| | YICKVELMYPPPYYLGIGNGTQI | |
| | YVIDPEPCPDSDQEPKSSDKTHT | |
| | CPPCPAPPVAGPSVFLFPPKPKDT | |
| | LMISRTPEVTCVVVDVSHEDPEV | |
| | KFNWYVDGVEVHNAKTKPREE | |
| | QYNSTYRVVSVLTVLHQDWLNG | |
| | KEYKCAVSNKALAAPIEKTISKA | |
| | KGQPREGPGLVKPSETLSLTCSV | |
| | SGGSISTSSYYWGWIRQPPGKGL | |
| | EWSGSIYYSGSTYYRPSLQGRATI | |
| | SVDTSKNQFSLKLTSVTAADTAV | |
| | YYCARHRGSYFFDIWGLGTLVT | |
| | VSSASTKGPSVFPLAPSSKSTSGG | |
| | TAALGCLVKDYFPEPVTVSWNS | |
| | GALTSGVHTFPAVLQSSGLYSLS | |
| | SVVTVPSSSLGTQTYICNVNHKP | |
| | SNTKVDKRVEPKSC (SEQ ID NO: | |
| | 315) | |
| Bernett Variant | MHVAQPAVVLASSRGIASFVCEY | SSELTQDPAVSVALGQTVRITCQG |
| 2_89B09 | ASPGKKTEVRVTVLRQADSQVT | DSVRRFFAHWYQQKPGQAPVLVI |
| | EVCFANYYMGNELTFDDDSICTG | SGKDTRPSGIPDRFSGSSSGNTASL |
| | TSSGNQVNLTIQGLRAMDTGLYI | TITGAQAEDEADYYCNSRDSSGY |
| | CQVELMYPPPYYEGIGNGTQIYV | LVLFGGGTKLTVLGQPKAAPSVT |
| | IDPEPCPDSD | LFPPSSEELQANKATLVCLISDFYP |
| | AMHVAQPAVVLASSRGIASFVCE | GAVTVAWKADSSPVKAGVETTT |
| | YASPGKATEVRVTVLRQADSQV | PSKQSNNKYAASSYLSLTPEQWK |
| | TEVCAATYMMGNELTFLDDSICT | SHKSYSCQVTHEGSTVEKTVAPT |
| | GTSSGNQVNLTIQGLRAMDTGL | ECS (SEQ ID NO: 67)) |
| | YICKVELMYPPPYYLGIGNGTQI | |
| | YVIDPEPCPDSDQEPKSSDKTHT | |
| | CPPCPAPPVAGPSVFLFPPKPKDT | |
| | LMISRTPEVTCVVVDVSHEDPEV | |
| | KFNWYVDGVEVHNAKTKPREE | |
| | QYNSTYRVVSVLTVLHQDWLNG | |
| | KEYKCAVSNKALAAPIEKTISKA | |
| | KGQPREGPGLVKPSETLSLTCSV | |
| | SGGSISTSSYYWGWIRQPPGKGL | |
| | EWSGSIYYSGSTYYRPSLQGRATI | |
| | SVDTSKNQFSLKLTSVTAADTAV | |
| | YYCARHRGSYFFDIWGLGTLVT | |
| | VSSASTKGPSVFPLAPSSKSTSGG | |
| | TAALGCLVKDYFPEPVTVSWNS | |
| | GALTSGVHTFPAVLQSSGLYSLS | |
| | SVVTVPSSSLGTQTYICNVNHKP | |
| | SNTKVDKRVEPKSC (SEQ ID NO: | |
| | 316) | |
| Bernett Variant | MHVAQPAVVLASSRGIASFVCEY | SSELTQDPAVSVALGQTVRITCQG |
| 3_89B09 | ASPGKWTEVRVTVLRQADSQVT | DSVRRFFAHWYQQKPGQAPVLVI |
| | EVCFANYYMGNELTFDDDSICTG | SGKDTRPSGIPDRFSGSSSGNTASL |
| | TSSGNQVNLTIQGLRAMDTGLYI | TITGAQAEDEADYYCNSRDSSGY |
| | CQVELMYPPPYYEGIGNGTQIYV | LVLFGGGTKLTVLGQPKAAPSVT |
| | IDPEPCPDSD | LFPPSSEELQANKATLVCLISDFYP |
| | AMHVAQPAVVLASSRGIASFVCE | GAVTVAWKADSSPVKAGVETTT |
| | YASPGKATEVRVTVLRQADSQV | PSKQSNNKYAASSYLSLTPEQWK |
| | TEVCAATYMMGNELTFLDDSICT | SHKSYSCQVTHEGSTVEKTVAPT |
| | GTSSGNQVNLTIQGLRAMDTGL | ECS (SEQ ID NO: 67)) |
| | YICKVELMYPPPYYLGIGNGTQI | |
| | YVIDPEPCPDSDQEPKSSDKTHT | |
| | CPPCPAPPVAGPSVFLFPPKPKDT | |
| | LMISRTPEVTCVVVDVSHEDPEV | |
| | KFNWYVDGVEVHNAKTKPREE | |
| | QYNSTYRVVSVLTVLHQDWLNG | |
| | KEYKCAVSNKALAAPIEKTISKA | |
| | KGQPREGPGLVKPSETLSLTCSV | |
| | SGGSISTSSYYWGWIRQPPGKGL | |
| | EWSGSIYYSGSTYYRPSLQGRATI | |
| | SVDTSKNQFSLKLTSVTAADTAV | |
| | YYCARHRGSYFFDIWGLGTLVT | |
| | VSSASTKGPSVFPLAPSSKSTSGG | |
| | TAALGCLVKDYFPEPVTVSWNS | |
| | GALTSGVHTFPAVLQSSGLYSLS | |
| | SVVTVPSSSLGTQTYICNVNHKP | |
| | SNTKVDKRVEPKSC (SEQ ID NO: | |
| | 317) | |
| Bernett Variant | MHVAQPAVVLASSRGIASFVCEY | SSELTQDPAVSVALGQTVRITCQG |
| 4_89B09 | ASPGKYTEVRVTVLRQADSQVT | DSVRRFFAHWYQQKPGQAPVLVI |
| | EVCFANYYMGNELTFDDDSICTG | SGKDTRPSGIPDRFSGSSSGNTASL |
| | TSSGNQVNLTIQGLRAMDTGLYI | TITGAQAEDEADYYCNSRDSSGY |
| | CQVELMYPPPYYEGIGNGTQIYV | LVLFGGGTKLTVLGQPKAAPSVT |
| | IDPEPCPDSD | LFPPSSEELQANKATLVCLISDFYP |
| | AMHVAQPAVVLASSRGIASFVCE | GAVTVAWKADSSPVKAGVETTT |
| | YASPGKATEVRVTVLRQADSQV | PSKQSNNKYAASSYLSLTPEQWK |
| | TEVCAATYMMGNELTFLDDSICT | SHKSYSCQVTHEGSTVEKTVAPT |
| | GTSSGNQVNLTIQGLRAMDTGL | ECS (SEQ ID NO: 67)) |
| | YICKVELMYPPPYYLGIGNGTQI | |
| | YVIDPEPCPDSDQEPKSSDKTHT | |
| | CPPCPAPPVAGPSVFLFPPKPKDT | |
| | LMISRTPEVTCVVVDVSHEDPEV | |
| | KFNWYVDGVEVHNAKTKPREE | |
| | QYNSTYRVVSVLTVLHQDWLNG | |
| | KEYKCAVSNKALAAPIEKTISKA | |
| | KGQPREGPGLVKPSETLSLTCSV | |
| | SGGSISTSSYYWGWIRQPPGKGL | |
| | EWSGSIYYSGSTYYRPSLQGRATI | |
| | SVDTSKNQFSLKLTSVTAADTAV | |
| | YYCARHRGSYFFDIWGLGTLVT | |
| | VSSASTKGPSVFPLAPSSKSTSGG | |
| | TAALGCLVKDYFPEPVTVSWNS | |
| | GALTSGVHTFPAVLQSSGLYSLS | |
| | SVVTVPSSSLGTQTYICNVNHKP | |
| | SNTKVDKRVEPKSC (SEQ ID NO: | |
| | 318) | |
| Bernett Variant | MHVAQPAVVLASSRGIASFVCEY | SSELTQDPAVSVALGQTVRITCQG |
| 5_89B09 | ASPGKHTEVRVTVLRQADSQVT | DSVRRFFAHWYQQKPGQAPVLVI |
| | EVCFANYYMGNELTFEDDSICTG | SGKDTRPSGIPDRFSGSSSGNTASL |
| | TSSGNQVNLTIQGLRAMDTGLYI | TITGAQAEDEADYYCNSRDSSGY |
| | CQVELMYPPPYYEGIGNGTQIYV | LVLFGGGTKLTVLGQPKAAPSVT |
| | IDPEPCPDSDAMHVAQPAVVLAS | LFPPSSEELQANKATLVCLISDFYP |
| | SRGIASFVCEYASPGKATEVRVT | GAVTVAWKADSSPVKAGVETTT |
| | VLRQADSQVTEVCAATYMMGN | PSKQSNNKYAASSYLSLTPEQWK |
| | ELTFLDDSICTGTSSGNQVNLTIQ | SHKSYSCQVTHEGSTVEKTVAPT |
| | GLRAMDTGLYICKVELMYPPPY | ECS (SEQ ID NO: 67)) |
| | YLGIGNGTQIYVIDPEPCPDSDQE | |
| | PKSSDKTHTCPPCPAPPVAGPSVF | |
| | LFPPKPKDTLMISRTPEVTCVVV | |
| | DVSHEDPEVKFNWYVDGVEVH | |
| | NAKTKPREEQYNSTYRVVSVLT | |
| | VLHQDWLNGKEYKCAVSNKAL | |
| | AAPIEKTISKAKGQPREGPGLVKP | |
| | SETLSLTCSVSGGSISTSSYYWG | |
| | WIRQPPGKGLEWSGSIYYSGSTY | |
| | YRPSLQGRATISVDTSKNQFSLK | |
| | LTSVTAADTAVYYCARHRGSYF | |
| | FDIWGLGTLVTVSSASTKGPSVF | |
| | PLAPSSKSTSGGTAALGCLVKDY | |
| | FPEPVTVSWNSGALTSGVHTFPA | |
| | VLQSSGLYSLSSVVTVPSSSLGTQ | |
| | TYICNVNHKPSNTKVDKRVEPKS | |
| | C (SEQ ID NO: 319) | |
| Bernett Variant | MHVAQPAVVLASSRGIASFVCEY | SSELTQDPAVSVALGQTVRITCQG |
| 6_89B09 | ASPGKKTEVRVTVLRQADSQVT | DSVRRFFAHWYQQKPGQAPVLVI |
| | EVCFANYYMGNELTFEDDSICTG | SGKDTRPSGIPDRFSGSSSGNTASL |
| | TSSGNQVNLTIQGLRAMDTGLYI | TITGAQAEDEADYYCNSRDSSGY |
| | CQVELMYPPPYYEGIGNGTQIYV | LVLFGGGTKLTVLGQPKAAPSVT |
| | IDPEPCPDSDAMHVAQPAVVLAS | LFPPSSEELQANKATLVCLISDFYP |
| | SRGIASFVCEYASPGKATEVRVT | GAVTVAWKADSSPVKAGVETTT |
| | VLRQADSQVTEVCAATYMMGN | PSKQSNNKYAASSYLSLTPEQWK |
| | ELTFLDDSICTGTSSGNQVNLTIQ | SHKSYSCQVTHEGSTVEKTVAPT |
| | GLRAMDTGLYICKVELMYPPPY | ECS (SEQ ID NO: 67)) |
| | YLGIGNGTQIYVIDPEPCPDSDQE | |
| | PKSSDKTHTCPPCPAPPVAGPSVF | |
| | LFPPKPKDTLMISRTPEVTCVVV | |
| | DVSHEDPEVKFNWYVDGVEVH | |
| | NAKTKPREEQYNSTYRVVSVLT | |
| | VLHQDWLNGKEYKCAVSNKAL | |
| | AAPIEKTISKAKGQPREGPGLVKP | |
| | SETLSLTCSVSGGSISTSSYYWG | |
| | WIRQPPGKGLEWSGSIYYSGSTY | |
| | YRPSLQGRATISVDTSKNQFSLK | |
| | LTSVTAADTAVYYCARHRGSYF | |
| | FDIWGLGTLVTVSSASTKGPSVF | |
| | PLAPSSKSTSGGTAALGCLVKDY | |
| | FPEPVTVSWNSGALTSGVHTFPA | |
| | VLQSSGLYSLSSVVTVPSSSLGTQ | |
| | TYICNVNHKPSNTKVDKRVEPKS | |
| | C (SEQ ID NO: 320) | |
| Bernett Variant | MHVAQPAVVLASSRGIASFVCEY | SSELTQDPAVSVALGQTVRITCQG |
| 7_89B09 | ASPGKWTEVRVTVLRQADSQVT | DSVRRFFAHWYQQKPGQAPVLVI |
| | EVCFANYYMGNELTFEDDSICTG | SGKDTRPSGIPDRFSGSSSGNTASL |
| | TSSGNQVNLTIQGLRAMDTGLYI | TITGAQAEDEADYYCNSRDSSGY |
| | CQVELMYPPPYYEGIGNGTQIYV | LVLFGGGTKLTVLGQPKAAPSVT |
| | IDPEPCPDSDAMHVAQPAVVLAS | LFPPSSEELQANKATLVCLISDFYP |
| | SRGIASFVCEYASPGKATEVRVT | GAVTVAWKADSSPVKAGVETTT |
| | VLRQADSQVTEVCAATYMMGN | PSKQSNNKYAASSYLSLTPEQWK |
| | ELTFLDDSICTGTSSGNQVNLTIQ | SHKSYSCQVTHEGSTVEKTVAPT |
| | GLRAMDTGLYICKVELMYPPPY | ECS (SEQ ID NO: 67)) |
| | YLGIGNGTQIYVIDPEPCPDSDQE | |
| | PKSSDKTHTCPPCPAPPVAGPSVF | |
| | LFPPKPKDTLMISRTPEVTCVVV | |
| | DVSHEDPEVKFNWYVDGVEVH | |
| | NAKTKPREEQYNSTYRVVSVLT | |
| | VLHQDWLNGKEYKCAVSNKAL | |
| | AAPIEKTISKAKGQPREGPGLVKP | |
| | SETLSLTCSVSGGSISTSSYYWG | |
| | WIRQPPGKGLEWSGSIYYSGSTY | |
| | YRPSLQGRATISVDTSKNQFSLK | |
| | LTSVTAADTAVYYCARHRGSYF | |
| | FDIWGLGTLVTVSSASTKGPSVF | |
| | PLAPSSKSTSGGTAALGCLVKDY | |
| | FPEPVTVSWNSGALTSGVHTFPA | |
| | VLQSSGLYSLSSVVTVPSSSLGTQ | |
| | TYICNVNHKPSNTKVDKRVEPKS | |
| | C (SEQ ID NO: 321) | |
| Bernett Variant | MHVAQPAVVLASSRGIASFVCEY | SSELTQDPAVSVALGQTVRITCQG |
| 8_89B09 | ASPGKYTEVRVTVLRQADSQVT | DSVRRFFAHWYQQKPGQAPVLVI |
| | EVCFANYYMGNELTFEDDSICTG | SGKDTRPSGIPDRFSGSSSGNTASL |
| | TSSGNQVNLTIQGLRAMDTGLYI | TITGAQAEDEADYYCNSRDSSGY |
| | CQVELMYPPPYYEGIGNGTQIYV | LVLFGGGTKLTVLGQPKAAPSVT |
| | IDPEPCPDSDAMHVAQPAVVLAS | LFPPSSEELQANKATLVCLISDFYP |
| | SRGIASFVCEYASPGKATEVRVT | GAVTVAWKADSSPVKAGVETTT |
| | VLRQADSQVTEVCAATYMMGN | PSKQSNNKYAASSYLSLTPEQWK |
| | ELTFLDDSICTGTSSGNQVNLTIQ | SHKSYSCQVTHEGSTVEKTVAPT |
| | GLRAMDTGLYICKVELMYPPPY | ECS (SEQ ID NO: 67)) |
| | YLGIGNGTQIYVIDPEPCPDSDQE | |
| | PKSSDKTHTCPPCPAPPVAGPSVF | |
| | LFPPKPKDTLMISRTPEVTCVVV | |
| | DVSHEDPEVKFNWYVDGVEVH | |
| | NAKTKPREEQYNSTYRVVSVLT | |
| | VLHQDWLNGKEYKCAVSNKAL | |
| | AAPIEKTISKAKGQPREGPGLVKP | |
| | SETLSLTCSVSGGSISTSSYYWG | |
| | WIRQPPGKGLEWSGSIYYSGSTY | |
| | YRPSLQGRATISVDTSKNQFSLK | |
| | LTSVTAADTAVYYCARHRGSYF | |
| | FDIWGLGTLVTVSSASTKGPSVF | |
| | PLAPSSKSTSGGTAALGCLVKDY | |
| | FPEPVTVSWNSGALTSGVHTFPA | |
| | VLQSSGLYSLSSVVTVPSSSLGTQ | |
| | TYICNVNHKPSNTKVDKRVEPKS | |
| | C (SEQ ID NO: 322) | |
| Oshima | MHVAQPAVVLASSRGIASFVCEY | SSELTQDPAVSVALGQTVRITCQG |
| Variant_89B09 | ESPGKANEIRVTVLRQADSQVTE | DSVRRFFAHWYQQKPGQAPVLVI |
| | VCAMTYMKEDELTFLDDPSCTG | SGKDTRPSGIPDRFSGSSSGNTASL |
| | TFSGNQVNLTIQGLRAMDTGLYI | TITGAQAEDEADYYCNSRDSSGY |
| | CKVELMYPPPYYEGIGNGTQIYV | LVLFGGGTKLTVLGQPKAAPSVT |
| | IDPEPCPDSDAMHVAQPAVVLAS | LFPPSSEELQANKATLVCLISDFYP |
| | SRGIASFVCEYASPGKATEVRVT | GAVTVAWKADSSPVKAGVETTT |
| | VLRQADSQVTEVCAATYMMGN | PSKQSNNKYAASSYLSLTPEQWK |
| | ELTFLDDSICTGTSSGNQVNLTIQ | SHKSYSCQVTHEGSTVEKTVAPT |
| | GLRAMDTGLYICKVELMYPPPY | ECS (SEQ ID NO: 67)) |
| | YLGIGNGTQIYVIDPEPCPDSDQE | |
| | PKSSDKTHTCPPCPAPPVAGPSVF | |
| | LFPPKPKDTLMISRTPEVTCVVV | |
| | DVSHEDPEVKFNWYVDGVEVH | |
| | NAKTKPREEQYNSTYRVVSVLT | |
| | VLHQDWLNGKEYKCAVSNKAL | |
| | AAPIEKTISKAKGQPREGPGLVKP | |
| | SETLSLTCSVSGGSISTSSYYWG | |
| | WIRQPPGKGLEWSGSIYYSGSTY | |
| | YRPSLQGRATISVDTSKNQFSLK | |
| | LTSVTAADTAVYYCARHRGSYF | |
| | FDIWGLGTLVTVSSASTKGPSVF | |
| | PLAPSSKSTSGGTAALGCLVKDY | |
| | FPEPVTVSWNSGALTSGVHTFPA | |
| | VLQSSGLYSLSSVVTVPSSSLGTQ | |
| | TYICNVNHKPSNTKVDKRVEPKS | |
| | C (SEQ ID NO: 323) | |
| Douthwaite | MHVAQPAVVLASSRGRASFVCE | SSELTQDPAVSVALGQTVRITCQG |
| Variant | YTNPSKATEVRVTVLRQADSQV | DSVRRFFAHWYQQKPGQAPVLVI |
| 1_89B09 | TEVCAATYMKGNEATFLDDSICT | SGKDTRPSGIPDRFSGSSSGNTASL |
| | GTASGNQVNLTIRGLRAQDTGL | TITGAQAEDEADYYCNSRDSSGY |
| | YICQVELMYPPPYYLGIGNGTQI | LVLFGGGTKLTVLGQPKAAPSVT |
| | YVIDPEPCPDSDAMHVAQPAVV | LFPPSSEELQANKATLVCLISDFYP |
| | LASSRGIASFVCEYASPGKATEV | GAVTVAWKADSSPVKAGVETTT |
| | RVTVLRQADSQVTEVCAATYM | PSKQSNNKYAASSYLSLTPEQWK |
| | MGNELTFLDDSICTGTSSGNQVN | SHKSYSCQVTHEGSTVEKTVAPT |
| | LTIQGLRAMDTGLYICKVELMYP | ECS (SEQ ID NO: 67)) |
| | PPYYLGIGNGTQIYVIDPEPCPDS | |
| | DQEPKSSDKTHTCPPCPAPPVAG | |
| | PSVFLFPPKPKDTLMISRTPEVTC | |
| | VVVDVSHEDPEVKFNWYVDGV | |
| | EVHNAKTKPREEQYNSTYRVVS | |
| | VLTVLHQDWLNGKEYKCAVSN | |
| | KALAAPIEKTISKAKGQPREGPG | |
| | LVKPSETLSLTCSVSGGSISTSSY | |
| | YWGWIRQPPGKGLEWSGSIYYS | |
| | GSTYYRPSLQGRATISVDTSKNQ | |
| | FSLKLTSVTAADTAVYYCARHR | |
| | GSYFFDIWGLGTLVTVSSASTKG | |
| | PSVFPLAPSSKSTSGGTAALGCL | |
| | VKDYFPEPVTVSWNSGALTSGV | |
| | HTFPAVLQSSGLYSLSSVVTVPSS | |
| | SLGTQTYICNVNHKPSNTKVDKR | |
| | VEPKSC (SEQ ID NO: 324) | |
| Douthwaite | MHVAQPAVVLASSVGRASFVCE | SSELTQDPAVSVALGQTVRITCQG |
| Variant | YTNPSKATEVRVTVLRQADSQV | DSVRRFFAHWYQQKPGQAPVLVI |
| 2_89B09 | TEVCAATYMKGNEATFLDDSICT | SGKDTRPSGIPDRFSGSSSGNTASL |
| | GTASGNQVNLTIRGLRAQDTGL | TITGAQAEDEADYYCNSRDSSGY |
| | YICQVELMYPPPYYLGIGNGTQI | LVLFGGGTKLTVLGQPKAAPSVT |
| | YVIDPEPCPDSDAMHVAQPAVV | LFPPSSEELQANKATLVCLISDFYP |
| | LASSRGIASFVCEYASPGKATEV | GAVTVAWKADSSPVKAGVETTT |
| | RVTVLRQADSQVTEVCAATYM | PSKQSNNKYAASSYLSLTPEQWK |
| | MGNELTFLDDSICTGTSSGNQVN | SHKSYSCQVTHEGSTVEKTVAPT |
| | LTIQGLRAMDTGLYICKVELMYP | ECS (SEQ ID NO: 67)) |
| | PPYYLGIGNGTQIYVIDPEPCPDS | |
| | DQEPKSSDKTHTCPPCPAPPVAG | |
| | PSVFLFPPKPKDTLMISRTPEVTC | |
| | VVVDVSHEDPEVKFNWYVDGV | |
| | EVHNAKTKPREEQYNSTYRVVS | |
| | VLTVLHQDWLNGKEYKCAVSN | |
| | KALAAPIEKTISKAKGQPREGPG | |
| | LVKPSETLSLTCSVSGGSISTSSY | |
| | YWGWIRQPPGKGLEWSGSIYYS | |
| | GSTYYRPSLQGRATISVDTSKNQ | |
| | FSLKLTSVTAADTAVYYCARHR | |
| | GSYFFDIWGLGTLVTVSSASTKG | |
| | PSVFPLAPSSKSTSGGTAALGCL | |
| | VKDYFPEPVTVSWNSGALTSGV | |
| | HTFPAVLQSSGLYSLSSVVTVPSS | |
| | SLGTQTYICNVNHKPSNTKVDKR | |
| | VEPKSC (SEQ ID NO: 325) | |
| Douthwaite | MHVAQPAVVLASSRGRASFVCE | SSELTQDPAVSVALGQTVRITCQG |
| Variant | YTNPSKATEVRVTVLRQADSQV | DSVRRFFAHWYQQKPGQAPVLVI |
| 3_89B09 | TEVCAATYMKGNEGTFLDDSICT | SGKDTRPSGIPDRFSGSSSGNTASL |
| | GTASGNQVNLTIRGLRAQDTGL | TITGAQAEDEADYYCNSRDSSGY |
| | YICQVELMYPPPYYLGIGNGTQI | LVLFGGGTKLTVLGQPKAAPSVT |
| | YVIDPEPCPDSDAMHVAQPAVV | LFPPSSEELQANKATLVCLISDFYP |
| | LASSRGIASFVCEYASPGKATEV | GAVTVAWKADSSPVKAGVETTT |
| | RVTVLRQADSQVTEVCAATYM | PSKQSNNKYAASSYLSLTPEQWK |
| | MGNELTFLDDSICTGTSSGNQVN | SHKSYSCQVTHEGSTVEKTVAPT |
| | LTIQGLRAMDTGLYICKVELMYP | ECS (SEQ ID NO: 67)) |
| | PPYYLGIGNGTQIYVIDPEPCPDS | |
| | DQEPKSSDKTHTCPPCPAPPVAG | |
| | PSVFLFPPKPKDTLMISRTPEVTC | |
| | VVVDVSHEDPEVKFNWYVDGV | |
| | EVHNAKTKPREEQYNSTYRVVS | |
| | VLTVLHQDWLNGKEYKCAVSN | |
| | KALAAPIEKTISKAKGQPREGPG | |
| | LVKPSETLSLTCSVSGGSISTSSY | |
| | YWGWIRQPPGKGLEWSGSIYYS | |
| | GSTYYRPSLQGRATISVDTSKNQ | |
| | FSLKLTSVTAADTAVYYCARHR | |
| | GSYFFDIWGLGTLVTVSSASTKG | |
| | PSVFPLAPSSKSTSGGTAALGCL | |
| | VKDYFPEPVTVSWNSGALTSGV | |
| | HTFPAVLQSSGLYSLSSVVTVPSS | |
| | SLGTQTYICNVNHKPSNTKVDKR | |
| | VEPKSC (SEQ ID NO: 326) | |
| Douthwaite | MHVAQPAVVLASSVGRASFVCE | SSELTQDPAVSVALGQTVRITCQG |
| Variant | YTNPSKATEVRVTVLRQADSQV | DSVRRFFAHWYQQKPGQAPVLVI |
| 4_89B09 | TEVCAATYMKGNEGTFLDDSICT | SGKDTRPSGIPDRFSGSSSGNTASL |
| | GTASGNQVNLTIRGLRAQDTGL | TITGAQAEDEADYYCNSRDSSGY |
| | YICQVELMYPPPYYLGIGNGTQI | LVLFGGGTKLTVLGQPKAAPSVT |
| | YVIDPEPCPDSDAMHVAQPAVV | LFPPSSEELQANKATLVCLISDFYP |
| | LASSRGIASFVCEYASPGKATEV | GAVTVAWKADSSPVKAGVETTT |
| | RVTVLRQADSQVTEVCAATYM | PSKQSNNKYAASSYLSLTPEQWK |
| | MGNELTFLDDSICTGTSSGNQVN | SHKSYSCQVTHEGSTVEKTVAPT |
| | LTIQGLRAMDTGLYICKVELMYP | ECS (SEQ ID NO: 67)) |
| | PPYYLGIGNGTQIYVIDPEPCPDS | |
| | DQEPKSSDKTHTCPPCPAPPVAG | |
| | PSVFLFPPKPKDTLMISRTPEVTC | |
| | VVVDVSHEDPEVKFNWYVDGV | |
| | EVHNAKTKPREEQYNSTYRVVS | |
| | VLTVLHQDWLNGKEYKCAVSN | |
| | KALAAPIEKTISKAKGQPREGPG | |
| | LVKPSETLSLTCSVSGGSISTSSY | |
| | YWGWIRQPPGKGLEWSGSIYYS | |
| | GSTYYRPSLQGRATISVDTSKNQ | |
| | FSLKLTSVTAADTAVYYCARHR | |
| | GSYFFDIWGLGTLVTVSSASTKG | |
| | PSVFPLAPSSKSTSGGTAALGCL | |
| | VKDYFPEPVTVSWNSGALTSGV | |
| | HTFPAVLQSSGLYSLSSVVTVPSS | |
| | SLGTQTYICNVNHKPSNTKVDKR | |
| | VEPKSC (SEQ ID NO: 327) | |
| Douthwaite | MHVAQPAVVLASSRGRASFVCE | SSELTQDPAVSVALGQTVRITCQG |
| Variant | YTNPSKATEVRVTVLRQADSQV | DSVRRFFAHWYQQKPGQAPVLVI |
| 5_89B09 | TEVCAATYMKGNEATFLDDSICT | SGKDTRPSGIPDRFSGSSSGNTASL |
| | GTASGNQVNLTIRGLRASDTGLY | TITGAQAEDEADYYCNSRDSSGY |
| | ICQVELMYPPPYYLGIGNGTQIY | LVLFGGGTKLTVLGQPKAAPSVT |
| | VIDPEPCPDSDAMHVAQPAVVL | LFPPSSEELQANKATLVCLISDFYP |
| | ASSRGIASFVCEYASPGKATEVR | GAVTVAWKADSSPVKAGVETTT |
| | VTVLRQADSQVTEVCAATYMM | PSKQSNNKYAASSYLSLTPEQWK |
| | GNELTFLDDSICTGTSSGNQVNL | SHKSYSCQVTHEGSTVEKTVAPT |
| | TIQGLRAMDTGLYICKVELMYPP | ECS (SEQ ID NO: 67)) |
| | PYYLGIGNGTQIYVIDPEPCPDSD | |
| | QEPKSSDKTHTCPPCPAPPVAGP | |
| | SVFLFPPKPKDTLMISRTPEVTCV | |
| | VVDVSHEDPEVKFNWYVDGVE | |
| | VHNAKTKPREEQYNSTYRVVSV | |
| | LTVLHQDWLNGKEYKCAVSNK | |
| | ALAAPIEKTISKAKGQPREGPGL | |
| | VKPSETLSLTCSVSGGSISTSSYY | |
| | WGWIRQPPGKGLEWSGSIYYSGS | |
| | TYYRPSLQGRATISVDTSKNQFS | |
| | LKLTSVTAADTAVYYCARHRGS | |
| | YFFDIWGLGTLVTVSSASTKGPS | |
| | VFPLAPSSKSTSGGTAALGCLVK | |
| | DYFPEPVTVSWNSGALTSGVHTF | |
| | PAVLQSSGLYSLSSVVTVPSSSLG | |
| | TQTYICNVNHKPSNTKVDKRVEP | |
| | KSC (SEQ ID NO: 328) | |
| Douthwaite | MHVAQPAVVLASSVGRASFVCE | SSELTQDPAVSVALGQTVRITCQG |
| Variant | YTNPSKATEVRVTVLRQADSQV | DSVRRFFAHWYQQKPGQAPVLVI |
| 6_89B09 | TEVCAATYMKGNEATFLDDSICT | SGKDTRPSGIPDRFSGSSSGNTASL |
| | GTASGNQVNLTIRGLRASDTGLY | TITGAQAEDEADYYCNSRDSSGY |
| | ICQVELMYPPPYYLGIGNGTQIY | LVLFGGGTKLTVLGQPKAAPSVT |
| | VIDPEPCPDSDAMHVAQPAVVL | LFPPSSEELQANKATLVCLISDFYP |
| | ASSRGIASFVCEYASPGKATEVR | GAVTVAWKADSSPVKAGVETTT |
| | VTVLRQADSQVTEVCAATYMM | PSKQSNNKYAASSYLSLTPEQWK |
| | GNELTFLDDSICTGTSSGNQVNL | SHKSYSCQVTHEGSTVEKTVAPT |
| | TIQGLRAMDTGLYICKVELMYPP | ECS (SEQ ID NO: 67)) |
| | PYYLGIGNGTQIYVIDPEPCPDSD | |
| | QEPKSSDKTHTCPPCPAPPVAGP | |
| | SVFLFPPKPKDTLMISRTPEVTCV | |
| | VVDVSHEDPEVKFNWYVDGVE | |
| | VHNAKTKPREEQYNSTYRVVSV | |
| | LTVLHQDWLNGKEYKCAVSNK | |
| | ALAAPIEKTISKAKGQPREGPGL | |
| | VKPSETLSLTCSVSGGSISTSSYY | |
| | WGWIRQPPGKGLEWSGSIYYSGS | |
| | TYYRPSLQGRATISVDTSKNQFS | |
| | LKLTSVTAADTAVYYCARHRGS | |
| | YFFDIWGLGTLVTVSSASTKGPS | |
| | VFPLAPSSKSTSGGTAALGCLVK | |
| | DYFPEPVTVSWNSGALTSGVHTF | |
| | PAVLQSSGLYSLSSVVTVPSSSLG | |
| | TQTYICNVNHKPSNTKVDKRVEP | |
| | KSC (SEQ ID NO: 329) | |
| Douthwaite | MHVAQPAVVLASSRGRASFVCE | SSELTQDPAVSVALGQTVRITCQG |
| Variant | YTNPSKATEVRVTVLRQADSQV | DSVRRFFAHWYQQKPGQAPVLVI |
| 7_89B09 | TEVCAATYMKGNEGTFLDDSICT | SGKDTRPSGIPDRFSGSSSGNTASL |
| | GTASGNQVNLTIRGLRASDTGLY | TITGAQAEDEADYYCNSRDSSGY |
| | ICQVELMYPPPYYLGIGNGTQIY | LVLFGGGTKLTVLGQPKAAPSVT |
| | VIDPEPCPDSDAMHVAQPAVVL | LFPPSSEELQANKATLVCLISDFYP |
| | ASSRGIASFVCEYASPGKATEVR | GAVTVAWKADSSPVKAGVETTT |
| | VTVLRQADSQVTEVCAATYMM | PSKQSNNKYAASSYLSLTPEQWK |
| | GNELTFLDDSICTGTSSGNQVNL | SHKSYSCQVTHEGSTVEKTVAPT |
| | TIQGLRAMDTGLYICKVELMYPP | ECS (SEQ ID NO: 67)) |
| | PYYLGIGNGTQIYVIDPEPCPDSD | |
| | QEPKSSDKTHTCPPCPAPPVAGP | |
| | SVFLFPPKPKDTLMISRTPEVTCV | |
| | VVDVSHEDPEVKFNWYVDGVE | |
| | VHNAKTKPREEQYNSTYRVVSV | |
| | LTVLHQDWLNGKEYKCAVSNK | |
| | ALAAPIEKTISKAKGQPREGPGL | |
| | VKPSETLSLTCSVSGGSISTSSYY | |
| | WGWIRQPPGKGLEWSGSIYYSGS | |
| | TYYRPSLQGRATISVDTSKNQFS | |
| | LKLTSVTAADTAVYYCARHRGS | |
| | YFFDIWGLGTLVTVSSASTKGPS | |
| | VFPLAPSSKSTSGGTAALGCLVK | |
| | DYFPEPVTVSWNSGALTSGVHTF | |
| | PAVLQSSGLYSLSSVVTVPSSSLG | |
| | TQTYICNVNHKPSNTKVDKRVEP | |
| | KSC (SEQ ID NO: 330) | |
| Douthwaite | MHVAQPAVVLASSVGRASFVCE | SSELTQDPAVSVALGQTVRITCQG |
| Variant | YTNPSKATEVRVTVLRQADSQV | DSVRRFFAHWYQQKPGQAPVLVI |
| 8_89B09 | TEVCAATYMKGNEGTFLDDSICT | SGKDTRPSGIPDRESGSSSGNTASL |
| | GTASGNQVNLTIRGLRASDTGLY | TITGAQAEDEADYYCNSRDSSGY |
| | ICQVELMYPPPYYLGIGNGTQIY | LVLFGGGTKLTVLGQPKAAPSVT |
| | VIDPEPCPDSDAMHVAQPAVVL | LFPPSSEELQANKATLVCLISDFYP |
| | ASSRGIASFVCEYASPGKATEVR | GAVTVAWKADSSPVKAGVETTT |
| | VTVLRQADSQVTEVCAATYMM | PSKQSNNKYAASSYLSLTPEQWK |
| | GNELTFLDDSICTGTSSGNQVNL | SHKSYSCQVTHEGSTVEKTVAPT |
| | TIQGLRAMDTGLYICKVELMYPP | ECS (SEQ ID NO: 67)) |
| | PYYLGIGNGTQIYVIDPEPCPDSD | |
| | QEPKSSDKTHTCPPCPAPPVAGP | |
| | SVFLFPPKPKDTLMISRTPEVTCV | |
| | VVDVSHEDPEVKFNWYVDGVE | |
| | VHNAKTKPREEQYNSTYRVVSV | |
| | LTVLHQDWLNGKEYKCAVSNK | |
| | ALAAPIEKTISKAKGQPREGPGL | |
| | VKPSETLSLTCSVSGGSISTSSYY | |
| | WGWIRQPPGKGLEWSGSIYYSGS | |
| | TYYRPSLQGRATISVDTSKNQFS | |
| | LKLTSVTAADTAVYYCARHRGS | |
| | YFFDIWGLGTLVTVSSASTKGPS | |
| | VFPLAPSSKSTSGGTAALGCLVK | |
| | DYFPEPVTVSWNSGALTSGVHTF | |
| | PAVLQSSGLYSLSSVVTVPSSSLG | |
| | TQTYICNVNHKPSNTKVDKRVEP | |
| | KSC (SEQ ID NO: 331) | |
| US 9139653 | AMHVAQPAVVLASSRGIASFVCE | DIQMTQSPSSLSASVGDRVTITCR |
| Variant 1 | YASPGKATEVRVTVLRQADSQV | ASQSISSYLNWYQQKPGKAPNLLI |
| | TEVCAATYMMGNELTFLDDSICT | YAASSLQSGVPSRFSGSGSETDFT |
| | GTSSGNQVNLTIQGLRAMDTGL | LTISSLQPEDFATYYCQQSHSVSF |
| | YICKVELMYPPPYYLGIGNGTQI | TFGPGTKVDIKRTVAAPSVFIFPPS |
| | YVIDPEPCPDSDQEPKSSDKTHT | DEQLKSGTASVVCLLNNFYPREA |
| | CPPCPAPEAAGGPSVFLFPPKPKD | KVQWKVDNALQSGNSQESVTEQ |
| | TLMISRTPEVTCVVVDVSHEDPE | DSKDSTYSLSSTLTLSKADYEKHK |
| | VKFNWYVDGVEVHNAKTKPRE | VYACEVTHQGLSSPVTKSFNRGE |
| | EQYNSTYRVVSVLTVLHQDWLN | C (SEQ ID NO: 253) |
| | GKEYKCKVSNKALPAPIEKTISK | |
| | AKGQPREPQVYTLPPSREEMTKN | |
| | QVSLTCLVKGFYPSDIAVEWESN | |
| | GQPENNYKTTPPVLDSDGSFFLY | |
| | SKLTVDKSRWQQGNVFSCSVMH | |
| | EALHNHYTQKSLSLSPGAGGGGS | |
| | GGGGSGGGGSEVQLVESGGGLV | |
| | QPGGSLRLSCAASGFTFSNYAMN | |
| | WVRQAPGKGLEWVSTISGSGGA | |
| | TRYADSVKGRFTISRDNSRNTVY | |
| | LQMNSLRVEDTAVFYCTKDRLI | |
| | MATVRGPYYYGMDVWGQGTTV | |
| | TVSSASTKGPSVFPLAPCSRSTSE | |
| | STAALGCLVKDYFPEPVTVSWNS | |
| | GALTSGVHTFPAVLQSSGLYSLS | |
| | SVVTVPSSSLGTKTYTCNVDHKP | |
| | SNTKVDKRV (SEQ ID NO: 332) | |
| US 7501496 | AMHVAQPAVVLASSRGIASFVCE | DIQMTQSPSSLSASVGDRVTITCR |
| Variant 1 | YASPGKATEVRVTVLRQADSQV | ASQGISSWLAWYQQKPEKAPKSL |
| | TEVCAATYMMGNELTFLDDSICT | IYAASSLQSGVPSRFSGSGSGTDF |
| | GTSSGNQVNLTIQGLRAMDTGL | TLTISSLQPEDFATYYCQQYNSYP |
| | YICKVELMYPPPYYLGIGNGTQI | YTFGQGTKLEIKRTVAAPSVFIFPP |
| | YVIDPEPCPDSDQEPKSSDKTHT | SDEQLKSGTASVVCLLNNFYPRE |
| | CPPCPAPEAAGGPSVFLFPPKPKD | AKVQWKVDNALQSGNSQESVTE |
| | TLMISRTPEVTCVVVDVSHEDPE | QDSKDSTYSLSSTLTLSKADYEKH |
| | VKFNWYVDGVEVHNAKTKPRE | KVYACEVTHQGLSSPVTKSFNRG |
| | EQYNSTYRVVSVLTVLHQDWLN | EC (SEQ ID NO: 255) |
| | GKEYKCKVSNKALPAPIEKTISK | |
| | AKGQPREPQVYTLPPSREEMTKN | |
| | QVSLTCLVKGFYPSDIAVEWESN | |
| | GQPENNYKTTPPVLDSDGSFFLY | |
| | SKLTVDKSRWQQGNVFSCSVMH | |
| | EALHNHYTQKSLSLSPGAGGGGS | |
| | GGGGSGGGGSEVQLLESGGGLV | |
| | QPGGSLRLSCAASGFTFNSYAMS | |
| | WVRQAPGKGLEWVSIISGSGGFT | |
| | YYADSVKGRFTISRDNSRTTLYL | |
| | QMNSLRAEDTAVYYCAKDRLV | |
| | APGTFDYWGQGALVTVSSASTK | |
| | GPSVFPLAPSSKSTSGGTAALGC | |
| | LVKDYFPEPVTVSWNSGALTSG | |
| | VHTFPAVLQSSGLYSLSSVVTVP | |
| | SSSLGTQTYICNVNHKPSNTKVD | |
| | KKV (SEQ ID NO: 333) | |
| US 9139653 | AMHVAQPAVVLASSRGIASFVCE | DIQMTQSPSSLSASVGDRVTITCR |
| Variant 2 | YASPGKATEVRVTVLRQADSQV | ASQSISSYLNWYQQKPGKAPNLLI |
| | TEVCAATYMMGNELTFLDDSICT | YAASSLQSGVPSRFSGSGSETDFT |
| | GTSSGNQVNLTIQGLRAMDTGL | LTISSLQPEDFATYYCQQSHSVSF |
| | YICKVELMYPPPYYLGIGNGTQI | TFGPGTKVDIKRTVAAPSVFIFPPS |
| | YVIDPEPCPDSDQEPKSSDKTHT | DEQLKSGTASVVCLLNNFYPREA |
| | CPPCPAPPVAGPSVFLFPPKPKDT | KVQWKVDNALQSGNSQESVTEQ |
| | LMISRTPEVTCVVVDVSHEDPEV | DSKDSTYSLSSTLTLSKADYEKHK |
| | KFNWYVDGVEVHNAKTKPREE | VYACEVTHQGLSSPVTKSFNRGE |
| | QYNSTYRVVSVLTVLHQDWLNG | C (SEQ ID NO: 253) |
| | KEYKCAVSNKALAAPIEKTISKA | |
| | KGQPREPQVYTLPPSREEMTKNQ | |
| | VSLTCLVKGFYPSDIAVEWESNG | |
| | QPENNYKTTPPVLDSDGSFFLYS | |
| | KLTVDKSRWQQGNVFSCSVMHE | |
| | ALHNHYTQKSLSLSPGAGGGGS | |
| | GGGGSGGGGSEVQLVESGGGLV | |
| | QPGGSLRLSCAASGFTFSNYAMN | |
| | WVRQAPGKGLEWVSTISGSGGA | |
| | TRYADSVKGRFTISRDNSRNTVY | |
| | LQMNSLRVEDTAVFYCTKDRLI | |
| | MATVRGPYYYGMDVWGQGTTV | |
| | TVSSASTKGPSVFPLAPCSRSTSE | |
| | STAALGCLVKDYFPEPVTVSWNS | |
| | GALTSGVHTFPAVLQSSGLYSLS | |
| | SVVTVPSSSLGTKTYTCNVDHKP | |
| | SNTKVDKRV (SEQ ID NO: 334) | |
| US 7501496 | AMHVAQPAVVLASSRGIASFVCE | DIQMTQSPSSLSASVGDRVTITCR |
| Variant 2 | YASPGKATEVRVTVLRQADSQV | ASQGISSWLAWYQQKPEKAPKSL |
| | TEVCAATYMMGNELTFLDDSICT | IYAASSLQSGVPSRFSGSGSGTDF |
| | GTSSGNQVNLTIQGLRAMDTGL | TLTISSLQPEDFATYYCQQYNSYP |
| | YICKVELMYPPPYYLGIGNGTQI | YTFGQGTKLEIKRTVAAPSVFIFPP |
| | YVIDPEPCPDSDQEPKSSDKTHT | SDEQLKSGTASVVCLLNNFYPRE |
| | CPPCPAPPVAGPSVFLFPPKPKDT | AKVQWKVDNALQSGNSQESVTE |
| | LMISRTPEVTCVVVDVSHEDPEV | QDSKDSTYSLSSTLTLSKADYEKH |
| | KFNWYVDGVEVHNAKTKPREE | KVYACEVTHQGLSSPVTKSFNRG |
| | QYNSTYRVVSVLTVLHQDWLNG | EC (SEQ ID NO: 255) |
| | KEYKCAVSNKALAAPIEKTISKA | |
| | KGQPREPQVYTLPPSREEMTKNQ | |
| | VSLTCLVKGFYPSDIAVEWESNG | |
| | QPENNYKTTPPVLDSDGSFFLYS | |
| | KLTVDKSRWQQGNVFSCSVMHE | |
| | ALHNHYTQKSLSLSPGAGGGGS | |
| | GGGGSGGGGSEVQLLESGGGLV | |
| | QPGGSLRLSCAASGFTENSYAMS | |
| | WVRQAPGKGLEWVSIISGSGGFT | |
| | YYADSVKGRFTISRDNSRTTLYL | |
| | QMNSLRAEDTAVYYCAKDRLV | |
| | APGTFDYWGQGALVTVSSASTK | |
| | GPSVFPLAPSSKSTSGGTAALGC | |
| | LVKDYFPEPVTVSWNSGALTSG | |
| | VHTFPAVLQSSGLYSLSSVVTVP | |
| | SSSLGTQTYICNVNHKPSNTKVD | |
| | KKV (SEQ ID NO: 335) | |

Also provided herein is a use of a heavy chain selected from any of the amino acid sequences listed in TABLE 7 in the preparation of a protein wherein the heavy chain is associated (e.g., via a disulfide bond) with a light chain selected from any of the amino acid sequences listed in TABLE 7.

Also contemplated in the present disclosure are nucleic acids encoding the proteins of the present invention. Nucleic acids encoding the proteins disclosed herein may be codon optimized for optimal expression using standard bioinformatic methods. Cells comprising one or more nucleic acid encoding a protein of the present invention are also contemplated and can be produced by a standard transfection or transduction method (e.g., electroporation, calcium chloride transfection, lipofection, lentiviral deliver, or adeno-associated virus delivery).

### I.E Characteristics of the Proteins

Proteins of the present invention are capable of specifically binding CD80 and/or CD86 and OX40L. In some embodiments, proteins of the present invention bind to CD80 and/or CD86, and OX40L with high affinity as measured by any one of a variety of assays known in field. The binding and immunosuppressive activity of the proteins of the present invention can be compared to CTLA4-Ig and/or anti-OX40L reference molecules.

CTLA4-Ig (Ref3_CTLA4Ig)
(SEQ ID NO: 34)
AMHVAQPAVVLASSRGIASFVCEYASPGKATEVRVTVLRQADSQVTE
VCAATYMMGNELTFLDDSICTGTSSGNQVNLTIQGLRAMDTGLYICK
VELMYPPPYYLGIGNGTQIYVIDPEPCPDSDQEPKSSDKTHTSPPSPAP
ELLGGSSVFLFPPKPKDTLMISRTPEVTCVVVDVSHEDPEVKFNWYV
DGVEVHNAKTKPREEQYNSTYRVVSVLTVLHQDWLNGKEYKCKVS
NKALPAPIEKTISKAKGQPREPQVYTLPPSRDELTKNQVSLTCLVKGF
YPSDIAVEWESNGQPENNYKTTPPVLDSDGSFFLYSKLTVDKSRWQQ
GNVFSCSVMHEALHNHYTQKSLSLSPGK
CTLA4-IgG_Variant
(SEQ ID NO: 71)
AMHVAQPAVVLASSRGIASFVCEYASPGKATEVRVTVLRQADSQVTE
VCAATYMMGNELTFLDDSICTGTSSGNQVNLTIQGLRAMDTGLYICK
VELMYPPPYYLGIGNGTQIYVIDPEPCPDSDQEPKSSDKTHTSPPSPAP
ELLGGSSVFLFPPKPKDTLMISRTPEVTCVVVDVSHEDPEVKFNWYV
DGVEVHNAKTKPREEQYNSTYRVVSVLTVLHQDWLNGKEYKCKVS
NKALPAPIEKTISKAKGQPREPQVYTLPPSREEMTKNQVSLTCLVKGF
YPSDIAVEWESNGQPENNYKTTPPVLDSDGSFFLYSKLTVDKSRWQQ
GNVFSCSVMHEALHNHYTQKSLSLSPG
IgG1_Fc_variant-CTLA4
(SEQ ID NO: 72)
EPKSSDKTHTCPPCPAPEAEGAPSVFLFPPKPKDTLMISRTPEVTCVVV
DVSHEDPEVKFNWYVDGVEVHNAKTKPREEQYNSTYRVVSVLTVLH
QDWLNGKEYKCKVSNKALPSSIEKTISKAKGQPREPQVYTLPPSREE
MTKNQVSLTCLVKGFYPSDIAVEWESNGQPENNYKTTPPVLDSDGSF
FLYSKLTVDKSRWQQGNVFSCSVMHEALHNHYTQKSLSLSPGAGGG
GSGGGGSGGGGSAMHVAQPAVVLASSRGIASFVCEYASPGKATEVR
VTVLRQADSQVTEVCAATYMMGNELTFLDDSICTGTSSGNQVNLTIQ
GLRAMDTGLYICKVELMYPPPYYLGIGNGTQIYVIDPEPCPDS
Ref2_Anti_OX40L
Heavy Chain Sequence
(SEQ ID NO: 36)
EVQLLESGGGLVQPGGSLRLSCAASGFTFNSYAMSWVRQAPGKGLE
WVSIISGSGGFTYYADSVKGRFTISRDNSRTTLYLQMNSLRAEDTAVY
YCAKDRLVAPGTFDYWGQGALVTVSSASTKGPSVFPLAPSSKSTSGG
TAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPE
LLGGPSVFLFPPKPKDTLMISRTPEVTCVVVDVSHEDPEVKFNWYVD
GVEVHNAKTKPREEQYNSTYRVVSVLTVLHQDWLNGKEYKCKVSN
KALPAPIEKTISKAKGQPREPQVYTLPPSRDELTKNQVSLTCLVKGFYP
SDIAVEWESNGQPENNYKTTPPVLDSDGSFFLYSKLTVDKSRWQQGN
VFSCSVMHEALHNHYTQKSLSLSPG
Light Chain Sequence
(SEQ ID NO: 35)
DIQMTQSPSS LSASVGDRVT ITCRASQGIS
SWLAWYQQKPEKAPKSLIYA ASSLQSGVPSRFSGSGSGTD
FTLTISSLQPEDFATYYCQQ
YNSYPYTFGQGTKLEIKRTVAAPSVFIFPPSDEQLKSGTASVVCLLNNF
YPREAKVQWKV DNALQSGNSQ
ESVTEQDSKDSTYSLSSTLTLSKADYEKHKVYACEVTHQGLSSPVTKS
FNRGEC
US Pat. No. 9,139,653 (Ref1_Anti-OX40L)
Heavy Chain Variable Domain (VH)
(SEQ ID NO: 31)
EVQLVESGGGLVQPGGSLRLSCAASGFTFSNYAMNWVRQAPGKGLE
WVSTISGSGGATRYADSVKGRFTISRDNSRNTVYLQMNSLRVEDTAV
FYCTKDRLIMATVRGPYYYGMDVWGQGTTVTVSS
Light Chain Variable Domain (VL)
(SEQ ID NO: 30)
DIQMTQSPSSLSASVGDRVTITCRASQSISSYLNWYQQKPGKAPNLLIY
AASSLQSGVPSRFSGSGSETDFTLTISSLQPEDFATYYCQQSHSVSFTF
GPGTKVDIK

For example, proteins described herein can specifically bind and neutralize OX40L on the surface of APCs, thereby inhibiting T cell costimulatory signaling and activation. In some embodiments, proteins of the present invention have an IC₅₀ of less than 12 nM, as measured in an OX40L neutralization/OX40-HEK reporter assay as described in EXAMPLE 3 below. In some embodiments, proteins of the present invention have an IC50 of 5 nM to 12 nM, 5 nM to 11.5 nM, 5 nM to 11 nM, 5 nM to 11.5 nM, 5 nM to 10 nM, 5 nM to 10.5 nM, or 5 nM to 9 nM as measured in an OX40L neutralization/OX40-HEK reporter assay.

Inhibition of T cell activation can also be measured using a primary T cell activation assay as described in EXAMPLE 3 below. For example, in some embodiments, proteins of the present invention have an IC₅₀ of 2 nM to 12 nM, 2 nM to 11.5 nM, 2 nM to 11 nM, 2 nM to 10.5 nM, 2 nM to 10 nM, or 2 nM to 9.5 nM as measured in a primary T cell activation assay.

In some embodiments, proteins of the present invention bind to OX40L with a K_{D} if less than 75 nM as measured in a Biacore assay (described in EXAMPLE 3 below). For example, in some embodiments, OX40L binding has a K_{D} of 20 nM to 75 nM, 20 nM to 70 nM, 20 nM to 65 nM, 20 nM to 60 nM, 25 nM to 75 nM, 25 nM to 70 nM, 25 nM to 65 nM, 25 nM to 60 nM, 30 nM to 75 nM, 30 nM to 70 nM, 30 nM to 65 nM, and 30 nM to 60 nM as measured in a Biacore assay.

Blocking of T cell costimulatory signaling inhibits T cell activation and the production of pro-inflammatory mediators. In some embodiments, proteins described herein significantly inhibit the production of at least one or more pro-inflammatory cytokines including, but not limited to, IL-2, IFNγ, IL-6, or TNFα. Inhibition of pro-inflammatory cytokine production can be measured using an in vitro mixed lymphocyte reaction assay and comparing cells treated with protein described herein with cells treated under the same conditions but in the absence of protein, or in the presence of a CTLA4-Ig (extracellular domain of human CTLA4 fused at the N-terminus of human IgG1 Fc domain; SEQ ID NO: 34) and an anti-OX40L antibody (SEQ ID NO: 35 and SEQ ID NO: 36; SEQ ID NO: 30 and SEQ ID NO: 31), presented individually or in combination of the two proteins presented separately. For example, in some embodiments, proteins described herein inhibit production of at least one proinflammatory cytokine by cells in a mixed lymphocyte reaction assay by at least 10%, at least 20%, at least 30%, at least 40%, at least 50%, or at least 60% as compared to cells treated under the same conditions but in the absence of protein.

T cell costimulatory blockade also results in the inhibition of T cell proliferation. In some embodiments, proteins described herein significantly inhibit alloreactive CD4+ and CD8+ T cell proliferation in an in vitro mixed lymphocyte reaction assay as compared to alloreactive CD4+ and CD8+ T cells treated under the same conditions but in the absence of protein, or in the presence of a CTLA4-Ig (extracellular domain of human CTLA4 fused at the N-terminus of human IgG1 Fc domain; SEQ ID NO: 34) and an anti-OX40L antibody (SEQ ID NO: 35 and SEQ ID NO: 36; SEQ ID NO: 30 and SEQ ID NO: 31), presented individually or in combination of the two proteins presented separately. For example, in some embodiments, proteins described herein inhibit CD4+ and CD8+ T cell proliferation in a mixed lymphocyte reaction assay by at least 10%, at least 20%, at least 30%, at least 40%, or at least 50% as compared to cells treated under the same conditions but in the absence of protein.

In certain embodiments, proteins described herein preferentially inhibit alloreactive CD4+ and CD8+ T cell proliferation as compared to regulatory T cell (T_{reg} or Treg) proliferation in an in vitro mixed lymphocyte reaction assay as compared to alloreactive CD4+ and CD8+T cells treated under the same conditions but in the absence of protein, or in the presence of a CTLA4-Ig (extracellular domain of human CTLA4 fused at the N-terminus of human IgG1 Fc domain; SEQ ID NO: 34) and an anti-OX40L antibody (SEQ ID NO: 35 and SEQ ID NO: 36; SEQ ID NO: 30 and SEQ ID NO: 31), presented individually or in combination of the two proteins presented separately. For example, in some embodiments, mixed lymphyocyte reaction assays treated with proteins described herein causes an increase in the T_{reg}:T_{effector} cell ratio by at least 10%, at least 20%, at least 30%, at least 40%, at least 50%, or at least 60% as compared to cells treated under the same conditions but in the absence of protein.

Inhibition of costimulatory CD28 and OX40 activation can promote differentiation of T_{reg} cells. In some embodiments, proteins described herein significantly enhance T_{reg} suppressive function in an in vitro mixed lymphocyte reaction assay as compared to T_{reg} cells treated under the same conditions but in the absence of protein, or in the presence of a CTLA4-Ig (extracellular domain of human CTLA4 fused at the N-terminus of human IgG1 Fc domain; SEQ ID NO: 34) and an anti-OX40L antibody (SEQ ID NO: 35 and SEQ ID NO: 36; SEQ ID NO: 30 and SEQ ID NO: 31), presented individually or in combination of the two proteins presented separately. For example, in some embodiments, mixed lymphyocyte reaction assays treated with proteins described herein causes a decrease in the number of proliferating T_{effector} (Teff or T_{eff}) cells in co-culture with T_{reg}s at least 10%, at least 20%, at least 30%, at least 40%, at least 50%, or at least 60% as compared to cells treated under the same conditions but in the absence of protein.

In some embodiments, proteins of the present invention significantly inhibit alloreactive CD4+ and CD8+ T cell proliferation in an adoptive transfer assay in humanized mice as compared to alloreactive CD4+ and CD8+ T cells in mice treated under the same conditions but in the absence of protein, or in the presence of a CTLA4-Ig (extracellular domain of human CTLA4 fused at the N-terminus of human IgG1 Fc domain; SEQ ID NO: 34) and an anti-OX40L antibody (SEQ ID NO: 35 and SEQ ID NO: 36; SEQ ID NO: 30 and SEQ ID NO: 31), presented individually or in combination of the two proteins presented separately. For example, in some embodiments, mice treated with proteins described herein causes a decrease in the number of proliferating alloreactive CD4+ and CD8+ T cells in an adoptive transfer assay by at least 10%, at least 20%, at least 30%, at least 40%, at least 50%, or at least 60% as compared to mice treated under the same conditions but in the absence of protein.

In some embodiments, proteins described herein significantly inhibit serum IFNγ levels in an adoptive transfer assay in humanized mice as compared to mice treated under the same conditions but in the absence of protein, or in the presence of a CTLA4-Ig (extracellular domain of human CTLA4 fused at the N-terminus of human IgG1 Fc domain; SEQ ID NO: 34) and an anti-OX40L antibody (SEQ ID NO: 35 and SEQ ID NO: 36; SEQ ID NO: 30 and SEQ ID NO: 31), presented individually or in combination of the two proteins presented separately. For example, in some embodiments, mice treated with proteins described herein causes a decrease in serum IFNγ levels in an adoptive transfer assay by at least 10%, at least 20%, at least 30%, at least 40%, at least 50%, or at least 60% as compared to mice treated under the same conditions but in the absence of protein.

In some embodiments of the present invention, proteins described herein are not significantly internalized into myeloid-derived dendritic cells (MDDCs).

In some embodiments, proteins described herein are cross reactive with cynomolgus OX40L. For example, a protein of the present invention can bind cynomolgus OX40L with a K_{D} of 20 nM to 120 nM, 40 nM to 120 nM, 60 nM to 120 nM, 80 nM to 120 nM, 20 nM to 100 nM, 40 nM to 100 nM, 60 nM to 100 nM, 80 nM to 100 nM, 20 nM to 80 nM, 40 nM to 80 nM, or 60 nM to 80 nM as measured by a steady-state affinity assay by Surface Plasmon Resonance (SPR). In certain embodiments, proteins of the present invention are not cross-reactive with mouse, rabbit, or rat OX40L. For example, a protein of the present invention has a K_{D} of greater than 200 nM, greater that 250 nM, or greater than 500 nM when measured by a steady-state affinity assay by Surface Plasmon Resonance (SPR) for binding to mouse, rabbit, or rat OX40L. In some embodiments, proteins of the present invention are cross reactive with cynomolgus CD80 and CD86. For example, a protein of the present invention can bind cynomolgus CD80 and CD86 with a K_{D} of 20 nM to 120 nM, 40 nM to 120 nM, 60 nM to 120 nM, 80 nM to 120 nM, 20 nM to 100 nM, 40 nM to 100 nM, 60 nM to 100 nM, 80 nM to 100 nM, 20 nM to 80 nM, 40 nM to 80 nM, or 60 nM to 80 nM as measured by a steady-state affinity assay by Surface Plasmon Resonance (SPR).

### II Antibodies

Also provided herein are antibodies that specifically bind OX40L. In some embodiments, an antibody that specifically binds OX40L comprises a heavy chain variable domain (VH) and light chain variable domain (VL) selected from the sequences listed in TABLE 2.

In some embodiments, the heavy chain variable domain and the light chain variable domain of antibodies described herein comprise VH and VL CDR sequences selected from the consensus VH and VL sequences listed in TABLE 2.

Unless indicated otherwise, the CDR sequences provided in TABLE 2 are determined under the IMGT unique numbering scheme

In some embodiments, antibodies of the present invention may be IgG, IgM, IgA, IgD, or IgE. In certain embodiments, antibodies described herein are IgG1, IgG1, IgG3, or IgG4. In certain embodiments, the antibodies of the present invention are human IgG1 antibodies.

In certain embodiments, antibodies that specifically bind to OX40L comprise a VHCDR3 sequence selected from the consensus sequence of SEQ ID NO: 220 or SEQ ID NO: 221.

In some embodiments, antibodies that specifically binds to OX40L comprise: a heavy chain variable complementarity-determining region 1 (VHCDR1) comprising an amino acid sequence of SEQ ID NO: 222; a heavy chain variable complementarity-determining region 2 (VHCDR2) comprising an amino acid sequence of SEQ ID NO: 223, SEQ ID NO: 224, or SEQ ID NO: 225); and a heavy chain variable complementarity-determining region 3 (VHCDR3) comprising an amino acid sequence of: SEQ ID NO: 220 or SEQ ID NO: 221.

In some embodiments, antibodies that specifically bind to OX40L comprise: a light chain variable complementarity-determining region 1 (VLCDR1) comprising an amino acid sequence of SEQ ID NO: 226 or SX₁RX₂X₃X₄, wherein X₁ is V or L, X₂ is R or N, X₃ is F or Y, and X₄ is F or Y; a light chain variable complementarity-determining region 2 (VLCDR2) comprising an amino acid sequence of RDN, GKD or RDS; and a light chain variable complementarity-determining region 3 (VLCDR3) comprising an amino acid sequence of SEQ ID NO: 231 or SEQ ID NO: 232.

In some embodiments, antibodies that specifically bind to OX40L comprise: a VHCDR1 comprising an amino acid sequence of SEQ ID NO: 222; a VHCDR2 comprising an amino acid sequence of SEQ ID NO: 223; a VHCDR3 comprising an amino acid sequence of SEQ ID NO: 220; a VLCDR1 comprising an amino acid sequence of SEQ ID NO: 226; a VLCDR2 comprising an amino acid sequence of RDN; and a VLCDR3 comprising an amino acid sequence of SEQ ID NO: 231.

In some embodiments, antibodies that specifically bind OX40L comprise: a VHCDR1 comprising an amino acid sequence of SEQ ID NO: 222; a VHCDR2 comprising an amino acid sequence of SEQ ID NO: 224; a VHCDR3 comprising an amino acid sequence of SEQ ID NO: 221; a VLCDR1 comprising an amino acid sequence of SX₁RX₂X₃X₄, wherein X₁ is V or L, X₂ is R or N, X₃ is F or Y, and X₄ is F or Y; a VLCDR2 comprising an amino acid sequence of GKD; and a VLCDR3 comprising an amino acid sequence of SEQ ID NO: 232.

In some embodiments, antibodies that specifically bind to OX40L comprise, according to the IMGT unique numbering scheme, VHCDR1, VHCDR2, VHCDR3, VLCDR1, VLCDR2, and VLCDR3 each comprising an amino acid sequence corresponding to the VHCDR1, VHCDR2, VHCDR3, VLCDR1, VLCDR2, and VLCDR3 sequences of a VHCDR and VLCDR consensus sequences of TABLE 2, respectively.

In certain embodiments, the antibody that specifically binds OX40L comprises an antibody heavy chain (HC) that comprises an amino acid sequence at least 90% (e.g., at least 91%, at least 92%, at least 93%, at least 94%, at least 95%, at least 96%, at least 97%, at least 98%, at least 99%, or 100%) identical to the HC of an antibody disclosed in TABLE 3, and an antibody light chain (LC) that comprises an amino acid sequence at least 90% (e.g., at least 91%, at least 92%, at least 93%, at least 94%, at least 95%, at least 96%, at least 97%, at least 98%, at least 99%, or 100%) identical to the LC of the same antibody disclosed in TABLE 3. In certain embodiments, the antibody that specifically binds OX40L comprises an antibody heavy chain variable domain (VH) that comprises an amino acid sequence at least 90% (e.g., at least 91%, at least 92%, at least 93%, at least 94%, at least 95%, at least 96%, at least 97%, at least 98%, at least 99%, or 100%) identical to the VH of an antibody disclosed in TABLE 3, and an antibody light chain variable domain (VL) that comprises an amino acid sequence at least 90% (e.g., at least 91%, at least 92%, at least 93%, at least 94%, at least 95%, at least 96%, at least 97%, at least 98%, at least 99%, or 100%) identical to the VL of the same antibody disclosed in TABLE 3. In certain embodiments, the antibody comprises the heavy chain CDR1, CDR2, and CDR3 and the light chain CDR1, CDR2, and CDR3, determined under IMGT unique numbering scheme, Kabat (see Kabat et al., (1991) Sequences of Proteins of Immunological Interest, NIH Publication No. 91-3242, Bethesda), Chothia (see, e.g., Chothia C & Lesk A M, (1987), J. Mol. Biol. 196: 901-917), MacCallum (see MacCallum R M et al., (1996) J. Mol. Biol. 262: 732-745), or any other CDR determination method known in the art, of the VH and VL sequences disclosed in TABLE 3.

In certain embodiments, the antibody that specifically binds to OX40L comprises a heavy chain complementarity-determining region 1 (VHCDR1) comprising an amino acid sequence of GGSISTSSYY (SEQ ID NO: 77), a heavy chain complementarity-determining region 2 (VHCDR2) comprising an amino acid sequence of IYYSGST (SEQ ID NO: 78), a heavy chain complementarity-determining region 3 (VHCDR3) comprising an amino acid sequence of ARHRGSYFFDI (SEQ ID NO: 79), a light chain complementarity-determining region 1 (VLCDR1) comprising an amino acid sequence of DIENKN (SEQ ID NO: 81), a light chain complementarity-determining region 2 (VLCDR2) comprising an amino acid sequence of RDN, and a light chain complementarity-determining region 3 (VLCDR3) comprising an amino acid sequence of QVRDSNIVV (SEQ ID NO: 83).

In some embodiments, the antibody that specifically binds to OX40L comprises a VHCDR1 comprising an amino acid sequence of GVSIRSNGYY (SEQ ID NO: 93), a VHCDR2 comprising an amino acid sequence of MDYSGT (SEQ ID NO: 94), a VHCDR3 comprising an amino acid sequence of ARERSNNWYPIDY (SEQ ID NO: 95), a VLCDR1 comprising an amino acid sequence of SVRRFF (SEQ ID NO: 97), a VLCDR2 comprising an amino acid sequence of GKD, and a VLCDR3 comprising an amino acid sequence of NSRDSSGYLVL (SEQ ID NO: 99).

In some embodiments, the antibody that specifically binds to OX40L comprises a VHCDR1 comprising an amino acid sequence of GASVSSSSYY (SEQ ID NO: 85), a VHCDR2 comprising an amino acid sequence of INYGGST (SEQ ID NO: 86), a VHCDR3 comprising an amino acid sequence of ARHRGIYHFDY (SEQ ID NO: 87), a VLCDR1 comprising an amino acid sequence of NIENKN (SEQ ID NO: 89), a VLCDR2 comprising an amino acid sequence of RDS, and a VLCDR3 comprising an amino acid sequence of QVWDSNTVV (SEQ ID NO: 91).

In some embodiments, the antibody that specifically binds to OX40L comprises a VHCDR1 comprising an amino acid sequence of GGSISSSSYY (SEQ ID NO: 101), a VHCDR2 comprising an amino acid sequence of IGSVDYSGNT (SEQ ID NO: 102), a VHCDR3 comprising an amino acid sequence of ARHRGIYFFDY (SEQ ID NO: 103), a VLCDR1 comprising an amino acid sequence of NIENKN (SEQ ID NO: 89), a VLCDR2 comprising an amino acid sequence of RDS, and a VLCDR3 comprising an amino acid sequence of QVWDSNTVV (SEQ ID NO: 91).

In some embodiments, the antibody that specifically binds to OX40L comprises a VHCDR1 comprising an amino acid sequence of GFTFSNYA (SEQ ID NO: 133), a VHCDR2 comprising an amino acid sequence of ISGSGGAT (SEQ ID NO: 113), a VHCDR3 comprising an amino acid sequence of TKDRLIMATVRGPYYYGMDV (SEQ ID NO: 114), a VLCDR1 comprising an amino acid sequence of QSISSY (SEQ ID NO: 121), a VLCDR2 comprising an amino acid sequence of AAS, and a VLCDR3 comprising an amino acid sequence of QQSHSVSFT (SEQ ID NO: 154).

In some embodiments, the antibody that specifically binds to OX40L comprises a VHCDR1 comprising an amino acid sequence of SYAMS (SEQ ID NO: 105), a VHCDR2 comprising an amino acid sequence of IISGSGGFTYYADSVK (SEQ ID NO: 106), a VHCDR3 comprising an amino acid sequence of DRLVAPGTFDY (SEQ ID NO: 107), a VLCDR1 comprising an amino acid sequence of RASQGISSWLA (SEQ ID NO: 169), a VLCDR2 comprising an amino acid sequence of AASSLQS (SEQ ID NO: 170), and a VLCDR3 comprising an amino acid sequence of QQYNSYPYT (SEQ ID NO: 171).

In certain embodiments, the antibody that specifically binds OX40L comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 76, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 80.

In certain embodiments, the antibody that specifically binds OX40L comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 92, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 96.

In certain embodiments, the antibody that specifically binds OX40L comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 84, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 88.

In certain embodiments, the antibody that specifically binds OX40L comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 100, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 104.

In certain embodiments, the antibody that specifically binds OX40L comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 31, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 30.

In certain embodiments, the antibody that specifically binds OX40L comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 33, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 32.

In certain embodiments, the antibody that specifically binds OX40L comprises a HC that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 8, and a LC that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 7.

In certain embodiments, the antibody that specifically binds OX40L comprises a HC that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 16, and a LC that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 15.

In certain embodiments, the antibody that specifically binds OX40L comprises a HC that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 14, and a LC that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 13.

In certain embodiments, the antibody that specifically binds OX40L comprises a HC that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 18, and a LC that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 17.

### III Therapeutic Applications

The present disclosure also provides pharmaceutical formulations that contain a therapeutically effective amount of a protein disclosed herein. The pharmaceutical formulation comprises one or more excipients and is maintained at a certain pH. The term “excipient,” as used herein, means any non-therapeutic agent added to the formulation to provide a desired physical or chemical property, for example, pH, osmolarity, viscosity, clarity, color, isotonicity, odor, sterility, stability, rate of dissolution or release, adsorption, or penetration.

The present application provides methods for treating autoimmune disease using a protein described herein and/or a pharmaceutical formulation described herein. The methods may be used to treat a variety of autoimmune diseases including, but not limited to, rheumatoid arthritis, juvenile idiopathic arthritis, psoriatic arthritis, atopic dermatitis, Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Bal6 disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Cutaneous Lupus, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Thyroid eye disease (TED), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, and Vogt-Koyanagi-Harada Disease.

The present application additionally provides methods for treating graft versus host disease (GVHD) in a patient, the method comprising administering a protein or pharmaceutical formulation as described herein.

### EXAMPLES

The disclosure now being generally described, will be more readily understood by reference to the following examples, which are included merely for purposes of illustration of certain aspects and embodiments of the present disclosure, and are not intended to limit the scope of the disclosure in any way.

### Example 1: Reagent Preparation, Immunization Method, and Screening Assays

This example describes the reagents, immunization method, and screening assays that were used to generate data provided herein.

### 1.1 Reagent Preparation and Screening Assays

### 1.1.1 Generation of OX40-HEK Reporter Cells

The HEK293 EBNA-NFkb-Luc cell line was transfected with a plasmid vector for stable expression of full-length human OX40 (SEQ ID NO: 250). Stable mini-pools were selected with hygromycin and screened for induction of luciferase activity after incubation of cells with recombinant His-tag human OX40L protein (R&D systems).

Human OX40 (underlining denotes signal peptide)
(SEQ ID NO: 250)
MCVGARRLGRGPCAALLLLGLGLSTVTGLHCVGDTYPSNDRCCHEC
RPGNGMVSRCSRSQNTVCRPCGPGFYNDVVSSKPCKPCTWCNLRSGS
ERKQLCTATQDTVCRCRAGTQPLDSYKPGVDCAPCPPGHFSPGDNQA
CKPWTNCTLAGKHTLQPASNSSDAICEDRDPPATQPQETQGPPARPIT
VQPTEAWPRTSQGPSTRPVEVPGGRAVAAILGLGLVLGLLGPLAILLA
LYLLRRDQRLPPDAHKPPGGGSFRTPIQEEQADAHSTLAKI

### 1.1.2 Production of Multi-Valent Human OX40L Recombinant Protein

Human OX40L extracellular domain (ECD) (amino acids 51-183 of SEQ ID NO: 2) was expressed fused to Avi-GST-6His-tag domains (“6His” disclosed as SEQ ID NO: 338), purified via the GST domain, and further purified by size exclusion chromatography. The resulting OX40L ECD fusion protein “AVI-GST-His6-hOX40L” (“His6” disclosed as SEQ ID NO: 338) is a soluble protein reagent that is multi-valent for OX40L ECD.

### 1.1.3 Production of CHO-S Cells Expressing Human, Cynomolgus Monkey or Mouse OX40L

CHO-S cells were transfected with a plasmid vector for stable expression of full-length human OX40L (SEQ ID NO: 2), full-length cynomolgus monkey OX40L (SEQ ID NO: 337), or full-length mouse OX40L (SEQ ID NO: 251). Stable mini-pools were selected with hygromycin and cells were further screened and selected by flow cytometry for surface expression of the OX40L proteins.

Cynomolgus Monkey OX40L Amino Acid Sequence
(SEQ ID NO: 337)
MERVQPLEENVGNAARPRFERNKLLLVASVIQGLGLLLCFTYICLHFS
ALQVSHQYPRIQSIKVQFTEYKKEEGFILTSQKEDEIMKVQNNSVIINC
DGFYLISLKGYFSQEVNISLHYQKDEEPLFQLKKVRSVNSLMVASLTY
KDKVYLNVTTDNTSLDDFHVNGGELILIHQNPGEFCVL
Mouse OX40L Amino Acid Sequence
(SEQ ID NO: 251)
MEGEGVQPLDENLENGSRPRFKWKKTLRLVVSGIKGAGMLLCFIYVC
LQL
SSSPAKDPPIQRLRGAVTRCEDGQLFISSYKNEYQTMEVQNNSVVIKC
DGLYIIYLKGSFFQEVKIDLHFREDHNPISIPMLNDGRRIVFTVVASLAF
KDKVYLTVNAPDTLCEHLQINDGELIVVQLTPGYCAPEGSYHSTVNQ
VPL

### 1.1.4 Flow Cytometry Assay to Screen for Human, Cynomolgus Monkey, or Mouse OX40L Binding

CHO-S cells expressing surface OX40L (human, cynomolgus monkey or mouse) generated in 1.1.2 were stained with a dilution series prepared from immunized rat serum or with recombinant anti-OX40L antibodies. Antibody binding to the CHO-S-OX40L cells was detected with fluorescently labelled anti-rat-IgG or anti-human IgG Fc secondary antibody as appropriate and measured by flow cytometry.

### 1.1.5 ELISA Screen for Binding to Recombinant Human, Cynomolgus Monkey, or Mouse OX40L

ELISA assay plates were coated with recombinant human (Novoprotein cat. #CJ45), cynomolgus monkey (Novoprotein cat. #CP72) or mouse OX40L (R&D systems cat. #1236-OX) proteins followed by incubation with a dilution series prepared from immunized rat serum or with recombinant anti-OX40L antibodies. Antibody binding to the coated ELISA plates containing recombinant OX40L was detected with peroxidase conjugated anti-rat-IgG or IgM or anti-human IgG Fc secondary antibody as appropriate.

1.1.6 ELISA Screen for Anti-OX40L Antibodies that Block OX40L:OX40 Protein Binding Interaction

Recombinant OX40-Fc fusion protein (R&D Systems) was coated onto 384-well ELISA assay plates and then blocked with BSA. Soluble recombinant OX40L-His protein (R&D Systems or in-house produced) was added to OX40-Fc coated assay plates and after incubation and washing, binding of soluble OX40L-His to coated OX40-Fc was detected with peroxidase-conjugated anti-His antibody. This condition gives the maximum signal of OX40L binding to OX40 without inhibition. To test anti-OX40L antibodies for inhibition of OX40L:OX40 binding, soluble OX40L-His protein was mixed with anti-OX40L antibodies, incubated, added to OX40-Fc coated assay plates and binding of OX40L to OX40 detected as above.

### 1.1.7 Human and Cynomolgus Monkey OX40L Steady-State Affinity Assay by Surface Plasmon Resonance (SPR)

Recombinant OX40L proteins (human OX40L protein, R&D systems, cat. #1054-OX; cynomolgus monkey OX40L, Novoprotein cat. #CP72) were immobilized onto Biacore CM5 sensor chips and anti-OX40L antibodies were bound to this surface at a range of concentrations. The response at steady state was measured with a Biacore T200 instrument (GE Healthcare) and plotted against the concentration of antibody and fit to a 1:1 binding model. The steady state KD was determined as the antibody concentration at a response equal to half the maximal response (Rₘₐₓ).

### 1.1.8 Human and Cynomolgus Monkey CD80 and CD86 Steady-State Affinity Assay by Surface Plasmon Resonance (SPR) or Bio-Layer Interferometry (BLI)

Goat anti-human IgG Fc (Jackson ImmunoResearch Goat anti-human IgG Fc Antibody, cat. #109-005-098), immobilized onto Biacore CM5 sensor chips, was used to capture experimental CTLA4_anti-OX40L fusion proteins or CTLA4-Ig reference proteins via their Fc regions. Recombinant CD80 proteins (human CD80, Novoprotein cat. #CK61; cynomolgus monkey CD80, Novoprotein cat. #CP54) and CD86 proteins (human CD86, Novoprotein . . . #C404) were bound to this surface at a range of concentrations. The response at steady state was measured with a Biacore T200 instrument (GE Healthcare) and plotted against the concentration of antibody and fit to a 1:1 binding model. The steady state KD was determined as the antibody concentration at a response equal to half the maximal response (Rₘₐₓ).

Cynomolgus monkey CD86 (Novoprotein cat. #CP41) steady-state affinity was measured with an Octet RED96 instrument (ForteBio) in a BLI assay. The Fc region of experimental CTLA4_anti-OX40L fusion proteins or of CTLA4-Ig reference protein was captured using anti-human IgG Fc Capture (AHC) Biosensors (ForteBio, cat. #18-5060). Recombinant cynomolgus monkey CD86 was bound to this surface at a range of concentrations. The response at steady state was plotted against the concentration of recombinant protein and fit to a 1:1 binding model. The steady state K_{D} was determined as the recombinant protein concentration at a response equal to half the maximal response (Rₘₐₓ).

### 1.1.9 OX40L Neutralization Assay Screen

HEK293 NFkB-Luc hOX40 reporter cells (as described in 1.1.1) were plated at 0.5×10⁶ cells/mL in serum-free media (Life Technologies: DMEM, Hygromycin B, Blasticidin and L-Glutamine), 50,000 cells/60 μl/well in a tissue culture treated clear bottom 96 well plate (Falcon) and incubated overnight at 37° C., 5% CO₂. A dilution series from immunized rat serum, or anti-OX40L antibodies was prepared in culture media in a 96 well plate (NUNC). 30 μl of the titration series were transferred to a 96 well plate (NUNC) containing 30 μL of 10 μg/mL recombinant human OX40L (described in 1.1.2) in each well. The antibody and recombinant OX40L mixture was incubated for 30 min. at room temperature, 20 μL of the mixture/well was added to the reporter cells and incubated overnight at 37° C., 5% CO₂. Each condition was performed in duplicate. 100 μL of SteadyGlo substrate (Promega, #E2520) was added to each well, plates were covered, protected from light and incubated at room temperature for 30 min. Plates were read utilizing the ultra-sensitive luminescence program (Luminescence 0.1 sec) on the Envision plate reader (Perkin Elmer).

### 1.1.10 Human OX40L Induced IL-2 in Primary T Cell Activation Assay

CD4+ T cells were isolated using RosetteSep Human T Cell Enrichment Kit (StemCell Technologies, 15062). 96-well plates were coated with 5 μg/mL anti-CD3 (BD Biosciences, 555338) and 5 μg/mL Anti-CD28 (BD Biosciences, 555725) for 1 hr at room temperature. Plates were washed with 200 μL 1×PBS, twice. Antibodies were prepared in 1:3.5 serial titration from 100 nM and then incubated with 10 μg/mL recombinant human OX40L (R&D Systems, 1054-OX) for 30 min at 37° C., 5% CO₂. Cells were resuspended at 1×10⁶ cells/mL and 600 μL of cells were added to a 0.5 mL 96 well deep well plate with 120 μL of the antibody/rhOX40L mixture. 200 μL of the cells/antibody/rhOX40L mixture was added to each well of the anti-CD3/anti-CD28 coated plate. Plates were incubated for 48 hrs at 37° C., 5% CO₂. After incubation, 50 μL of supernatant was collected and tested with human IL-2 AlphaLISA (Perkin Elmer, AL221C). Plates were read utilizing the AlphaScreen protocol on the Envision plate reader (Perkin Elmer).

### Example 2: Identification, Generation, and Characterization of Anti-OX40L Antibodies

This example describes the identification, generation, and characterization of anti-OX40L antibodies. In order to generate the anti-OX40L antibodies, OmniRat® rodents (a transgenic animal, purchased from Open Monoclonal Technology, Inc. (Palo Alto, CA, subsidiary of Ligand Pharmaceuticals, Inc., San Diego, CA), that expresses immunoglobulins with fully human variable regions) were first immunized with human OX40L DNA expression vector, followed by immunization rounds with his-tagged recombinant human OX40L extracellular domain (ECD) protein. Serum from immunized OmniRats® was screened for binding to CHO-S cells that expressed cell surface OX40L proteins using flow cytometry as described in 1.1.4 of Example 1. Serum from immunized OmniRats® was also screened for binding to recombinant OX40L proteins using ELISA as described in 1.1.5 of Example 1. After multiple rounds of immunization with human OX40L antigen via DNA expression vector and recombinant protein immunization, OmniRats® were identified with serum immune response for binding to human and cynomolgus OX40L. Sera were subsequently screened for neutralizing activity against soluble recombinant OX40L in the blocking ELISA assay described in 1.1.6 of Example 1 and human OX40L neutralization assay described in 1.1.9 of Example 1. Rats whose sera demonstrated good neutralizing activity and high titer to bind cell surface OX40L and soluble recombinant OX40L were selected. Cells were harvested from the spleen and lymph nodes of selected rats for subsequent direct B cell cloning and for B cell culture and screening followed by cloning.

### 2.1 B Cell Sorting, B Cell Culture, and Cloning of Anti-OX40L Antibodies

Single B cell sorting was performed from lymphocytes collected from selected immunized rats with high serum immune response that demonstrated OX40L neutralization activity and binding to OX40L target. Briefly, cells collected from spleen and/or lymph nodes of selected rats were incubated at 4° C., first with anti-rat CD32 (clone D34-485, BD Biosciences) to block Fc receptors on these cells, followed by multi-valent human OX40L recombinant protein (produced as described in 1.1.2 of Example 1). Cells were then washed and incubated with a mixture of FITC-conjugated goat anti-rat IgM (SouthernBiotech cat. #3020-02), PE-Cy7-conjugated mouse anti-rat CD45R (clone HIS 24, eBioscience), APC-conjugated mouse anti-His (clone AD1.1.10R, R&D) and depending on the genetic background of the selected OmniRat® and the sorting strategy PE-conjugated mouse anti-human Ig Lambda (clone 1-155-2, BD Biosciences) or V450-conjugated mouse anti-human Ig Kappa (clone G20-193, BD Biosciences). Single OX40L-binding B cells were sorted with a FACS Aria III (BD Biosciences) into each well of a 96 well plate. For direct B cell cloning, single OX40L-binding B cells were sorted directly into wells containing lysis buffer (0.1M DTT, 40 U/ml RNAse Inhibitor, Invitrogen, Cat #10777-019) and plates were sealed and immediately frozen on dry ice before storage at −80° C. For B cell culture and screening, OX40L-binding B cells were sorted into wells with B cell culture medium containing cytokines and irradiated in-house generated feeder cells. These B cell culture plates were incubated at 37° C. in a 5% CO₂ incubator for 7 days and then supernatants from each well were screened by ELISA for binding to human and cynomolgus OX40L as in described 1.1.5 of Example 1. Selected OX40L-binding B cell culture clones were recovered from the wells and placed into lysis buffer.

Ig V-gene cloning from each single sorted B cell was performed using a protocol modified from Tiller et al., J Immunol Methods, 2008, 1; 329 (1-2). Briefly, total RNA from single sorted B cells was reverse transcribed in a final volume of 14 μl/well in the original 96-well sorting plate with nuclease-free water (Invitrogen, Cat #AM9935) using final amounts/concentrations of 150 ng random hexamer primer (pd(N)6, Applied Biosystems, P/N N808-0127) and 50U Superscript IV reverse transcriptase (Invitrogen, Cat #18090050) following the manufacturer's protocol. Primers were modified based on previous publications (Wardemann et al, Science, 2003, 301:1374-1377) and/or designed by examining published Ig gene segment nucleotide sequences from IMGT®, the international Immunogenetics information system (http://www.imgt.org; (Lefranc et al., 2009) and NCBI (http://www.ncbi.nlm.nih.gov/igblast/) databases. Human Igh, IgK, and IgL V gene transcripts were amplified independently by two rounds of nested (IgH, IgK and IgL) PCR starting from 5 μl of cDNA as template. All PCR reactions were performed in 96-well plates in a total volume of 50 μl per well by using Platinum Multiplex PCR Reaction (Invitrogen, Cat #4464269) for first round of PCR, then by AccuPrime Taq DNA Polymerase High Fidelity kit, (Invitrogen, Cat #. 12346-094) for nested PCR following the manufacturer's protocol. The first round of PCR was performed at 95° C. for 2 min followed by 30 cycles of 94° C. for 30 sec, 50° C. for 30 sec, 72° C. for 40 sec, and final incubation at 72° C. for 5 min.

Nested second round PCR was performed with 5 μl of unpurified first round PCR product at 95° C. for 2 min followed by 5 cycles of 94° C. for 30 sec, 42° C. for 30 sec, 72° C. for 45 sec, and then 35 cycles of 94° C. for 30 sec, 55° C. for 30 sec, 72° C. for 45 sec, and final incubation at 72° C. for 5 min.

Paired V_{H} and V_{L} antibody sequences were obtained from 360 OX40L-binding B cells from direct B cell sorting and cloning, and 54 paired VH and V_{L} antibody sequences were obtained from B cell culture, screening and cloning. These paired VH and V_{L} sequences were cloned into IgG expression vectors for recombinant antibody expression as standard format IgG1 antibodies for further screening, characterization, and DNA sequence confirmation and analysis. Among the selected B cell clones, diversity of antibody gene sequences was captured, falling in a number of clonal sequence clusters.

### 2.2 Anti-OX40L Antibody Clone Screening and Selection

IgG Format: Recombinant anti-OX40L antibodies were produced with the Ig expression vectors constructed with the paired VH and V_{L} sequences obtained through direct B cell cloning and B cell culture approaches as described in section 2.1 of this example. Correctly paired heavy and light chain vectors derived from the B cell clones were used for small-scale transient transfection of Expi293F cells and then cell culture supernatants were collected after 7 days incubation. Cell culture supernatants containing recombinant anti-OX40L antibody clones were screened for binding to CHO-S cells expressing cell surface OX40L proteins by flow cytometry as in described in 1.1.4 of Example 1, for binding to recombinant OX40L proteins by ELISA as described in 1.1.5 of Example 1, for blocking OX40L protein from binding to OX40 by ELISA competition assay as in described in 1.1.6 of Example 1, and for neutralization of OX40L by OX40 reporter cell assay as described in 1.1.9 of Example 1.

Based on screening data for recombinant anti-OX40L clones in cell culture supernatants and analysis of the anti-OX40L clone sequences, a total of 85 anti-OX40L antibodies were selected to be produced at 15 ml scale from transient transfection of ExpiCHO cells and affinity purified with protein A beads to allow for more detailed characterization and comparison among the clones and equivalently produced reference proteins. These purified anti-OX40L antibodies were compared based on binding to CHO-S cells expressing cell surface OX40L proteins by flow cytometry as described in 1.1.4 of Example 1, binding to recombinant OX40L proteins by ELISA as described in 1.1.5 of Example 1, blocking OX40L protein from binding to OX40 by ELISA competition assay as described in 1.1.6 of Example 1, neutralization of OX40L by OX40 reporter cell assay as described in 1.1.9 of Example 1, and neutralization of OX40L-induced induced IL-2 in Primary T cell activation assay as described in 1.1.10 of Example 1, then 24 candidates were compared with steady-state K_{D} measurements for binding to OX40L protein by Surface Plasmon Resonance (SPR) as described in 1.1.7 of Example 1. Purified candidate anti-OX40L antibodies were compared as described above and further selected, with criteria that included clone sequence analysis, neutralization assay IC₅₀<20 nM (reporter cell and primary cell assays) and steady-state SPR binding K_{D}<60 nM. All selected candidates bound to human and cynomolgus OX40L and none were cross-reactive for binding to mouse OX40L. 22 out of 85 candidates met the above criteria. Antibody sequences of these 22 candidates were analyzed and 12 candidate clones that represented the antibody clonal sequence diversity were selected for further studies. Among these 12 anti-OX40L antibodies, clones 97G07 (IgG), 67B06 (IgG), 68F03 (IgG), 98E10 (IgG), 89B09 (IgG), 98C01 (IgG), 88B06 (IgG), and 85F10 (IgG) bound to human OX40L with K_{D} between 15-31.9 nM, which was similar to the binding K_{D} of the reference anti-OX40L antibodies. The rest of the clones bound to human OX40L with steady state K_{D}>32 nM. Selected data for these 12 anti-OX40L antibody clones are summarized in TABLE 8.

### 2.3 Characterization of Selected Anti-OX40L Antibody Clones

### 2.3.1 OX40L Neutralization Assay

Anti-OX40L antibody clones were tested for OX40L blocking function with the OX40L neutralization assay. Briefly, test antibodies, isotype control and known anti-OX40L reference antibody (positive controls) were serially titrated in culture media, incubated with recombinant human OX40L, and added to OX40 receptor expressing HEK-luciferase reporter cells as described in 1.1.9 of Example 1. Data were plotted with GraphPad Prism 8 software. Normalization was performed using Unstimulated cells as 0% and Stimulation with OX40L in the absence of antibodies as 100% of Luciferase activity from each experimental plate. IC₅₀ values for anti-OX40L antibodies were determined with GraphPad Prism by curve fitting using the log (inhibitor) vs normalized response-variable slope equation. Eleven anti-OX40L IgG antibody clones neutralized OX40L with varying potency on the HEK reporter assay. Clones 97G07 (IgG), 67B06 (IgG), 68F03 (IgG), 98E10 (IgG), 89B09 (IgG), 98C01 (IgG), and 95B06 (IgG) demonstrated IC₅₀ values of 3.6-7.5 nM, which was comparable to reference anti-OX40L antibodies. Clones 30F02 (IgG), 88B06 (IgG), 85F10 (IgG) and 84E11 (IgG) had IC₅₀ values of 17.7 to 72.7 nM. TABLE 8 summarizes data from this reporter assay.

2.3.2 Primary T cell Activation Assay

10 (clones 85F12 and 85F10 were not tested in this assay) of the anti-OX40L antibody clones were additionally tested with the Primary T cell activation assay described in 1.1.10. Briefly, CD4+ T cells were stimulated with anti-CD3, anti-CD28, and recombinant human OX40L, and anti-OX40L antibody clones were tested in serial dilution in this assay. OX40L mediated T cell activation resulted in high levels of IL-2 cytokine that can be reduced with increasing concentration of anti-OX40L IgG antibodies, indicating neutralization activity. Data were plotted with GraphPad Prism 8 software. Normalization was performed using Unstimulated T cells as 0% and Stimulated with OX40L in the absence of antibodies as 100% of IL-2 response from each experimental plate. IC₅₀ values were determined with GraphPad Prism by curve fitting using the log(inhibitor) vs normalized response-variable slope equation. TABLE 8 summarizes IC₅₀ (in nM+/−SEM) from 2 experiments. Most of the tested anti-OX40L antibody clones demonstrated OX40L neutralizing activity with IC₅₀<20 nM.

### 2.3.3 Steady State Affinity Assay

Steady state affinity assay was performed as described in section 1.1.7 of Example 1. TABLE 8 summarizes data from this assay.

TABLE 8
Data from Twelve Anti-OX40L (IgG)

| | OX40L | Primary T cell | Steady State |
|---|---|---|---|
| | neutralization/OX40- | activation assay | affinity human |
| Agents | HEK Reporter IC₅₀ | IC₅₀ | OX40L K_{D} |
| (IgG) | nM (+/−SEM) | nM (+/−SEM) | (nM) |
| 97G07 | 7.5 ± 5.32 | 16.7 ± 11.77 | 16.6 |
| 67B06 | 5.2 ± 3.66 | 20.9 ± 14.78 | 12.7 |
| 68F03 | 5.7 ± 3.99 | 18.8 ± 13.29 | 25.8 |
| 84E11 | 17.7 ± 12.50 | 8.8 ± 6.22 | 39.5 |
| 98E10 | 5.4 ± 3.85 | 8.8 ± 6.19 | 32.8 |
| 89B09 | 4.4 ± 3.13 | 10.7 ± 7.57 | 31.6 |
| 98C01 | 3.6 ± 2.57 | 12.9 ± 9.12 | 17.7 |
| 88B06 | 18.9 | 28.5 | 23.2 |
| 95B06 | 3.7 ± 2.66 | 7.4 ± 5.20 | 42.4 |
| 30F02 | 72.7 | 12.4 ± 8.77 | 53.9 |
| 85F12 | Not Tested | Not Tested | 75.6 |
| 85F10 | 54.6 ± 38.59 | Not Tested | 28.2 |

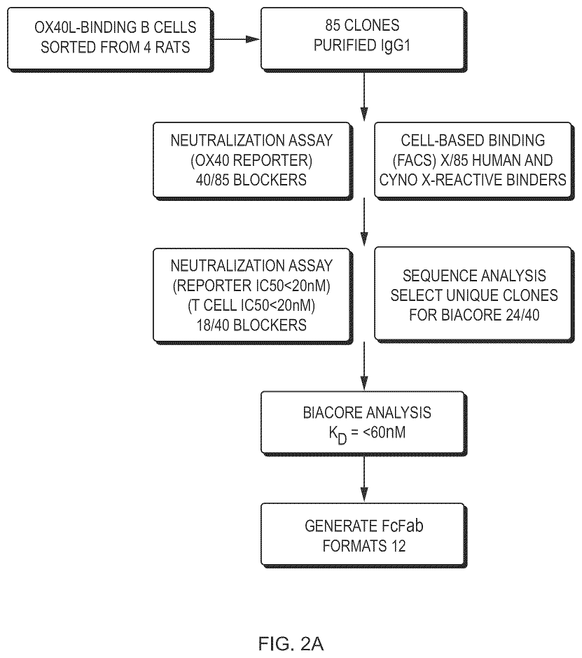

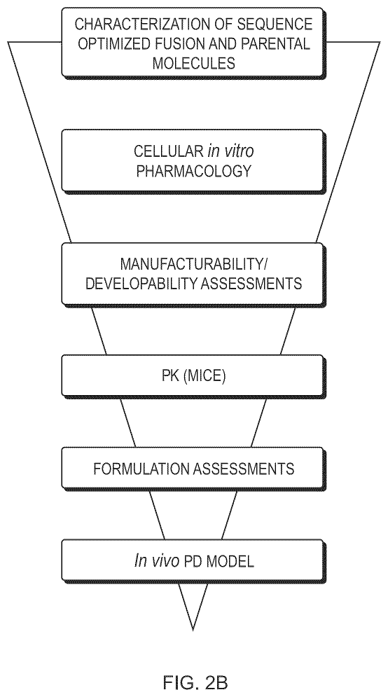

FIG. 2A is a flow chart providing the antibody screening cascade for anti-OX40L antibodies. The flow chart shows the assays that were used to screen and shortlist the anti-OX40L antibody clones to obtain twelve candidates. FIG. 2B is a flow chart providing the candidate characterization cascade.

### Example 3: Generation and Characterization of Anti-OX40L FcFab Antibodies and CTLA4_antiOX40L Fusion Proteins

FcFab Format and CTLA4_antiOX40L Fusion protein Format: This example describes how the twelve anti-OX40L clones were generated in FcFab and fusion with CTLA4 formats followed by characterization assays.

### 3.1 Generation of Anti-OX40L FcFAb and CTLA4_antiOX40L (“CTLA4-Fc-Anti-OX40L”) Fusion Proteins

The Fab domains of the twelve anti-OX40L clones (as described in section 2.2 of Example 2) were produced in two engineered protein formats: “FcFab” antibody and “CTLA4-Fc-anti-OX40L”. In both protein formats, the antibody Fab domain was attached at the C-terminus of a variant IgG1 Fc domain protein. In brief, the N-terminus of the heavy chain of the antibody clone, consisting of VH-CH1-and partial IgG1 hinge (EPKSC) sequence, was fused to the C-terminus of a 16 amino acid linker sequence that was fused to the C-terminus of the Fc polypeptide. The heavy chain polypeptides of these protein formats were co-expressed with the antibody clone light chain polypeptides, which associated with the heavy chain polypeptide of the Fab, and a disulfide bond was formed between the light chain and the C-terminal partial IgG1 hinge of the heavy chain.

The anti-OX40L FcFab antibodies heavy chain N-terminus began with the first amino acid of the IgG1 hinge element E216 (Kabat EU index numbering), as represented in sequence of SEQ ID NO: 73. The heavy chain and light chain amino acid sequences of the twelve CTLA4_anti OX40L fusion proteins are represented in SEQ ID NOs: 37-60.

Heavy chain DNA expression vectors for each of the anti-OX40L FcFab antibodies were produced by PCR amplification of the anti-OX40L antibody VH sequence and the PCR products were cloned into a FcFab heavy chain expression vector using 5′ BamHI and 3′NheI restriction enzyme sites that flanked the VH sequence and are part of the codon sequence of the 16-amino acid linker and the CH1 domain, respectively. The FcFab heavy chain expression vector was generated by gene synthesis (Quintara Biosciences) and then cloning the gene synthesis product into the transient expression vector pTT5 (Durocher et al., Nucleic Acids Res. 2002 Jan. 15; 30(2):E9.). The CTLA4_anti OX40L heavy chain expression vector was generated by gene synthesis (Genewiz) and then cloning the gene synthesis product into the transient expression vector pTT5. For each of the 12 CTLA4_anti OX40L heavy chain vectors, the clone specific VH sequence was cloned to be located between 5′ BamHI and 3′NheI restriction enzyme sites that flanked the VH sequence and are part of the codon sequence of the 16-amino acid linker and the CH1 domain, respectively. Exemplary DNA sequences constructed in these CTLA4_antiOX40L expression vectors are shown in SEQ ID NO: 74 (light chain sequence) and SEQ ID NO: 75 (heavy chain sequence).

97G07 heavy chain DNA sequence
(SEQ ID NO: 75)
gcaatgcacgtggcccagcctgctgtggtactggccagcagccgaggcatcgccagctttgtgtgtgagtacg
catctccaggcaaagccactgaggtccgggtgacagtgcttcggcaggctgacagccaggtgactgaagtct
gtgcggcaacctacatgatggggaatgagttgaccttcctagatgattccatctgcacgggcacctccagtgga
aatcaagtgaacctcactatccaaggactgagggctatggacacgggactctacatctgcaaggtggagctca
tgtacccaccgccatactacctgggcataggcaacggaacccagatttatgtaattgatccagaaccgtgccca
gattctgaccaggaacccaagagctccgacaagacccatacctgtcctccctgtcctgctccaccggtcgctg
ggccctccgtgttcctgttcccccccaagcccaaggacaccctgatgatcagcaggacccccgaggtgacct
gcgtggtggtggacgtgagccacgaggacccagaggtgaagttcaattggtatgtggacggcgtggaggtg
cacaacgccaagaccaagcccagagaggaacagtacaacagcacctacagggtggtgtccgtgctgaccgt
gctgcaccaggactggctgaacggcaaggaatacaagtgcgccgtatccaacaaggccctgccctccagca
tcgagaaaaccatcagcaaggccaagggccagccacgggagccccaggtgtacacactgcccccatctcg
ggaagaaatgaccaagaaccaggtgtccctgacctgtctggtgaagggcttttaccccagcgacatcgccgtg
gagtgggagagcaacggccagcccgagaacaactacaagaccaccccccctgtgctggacagcgacggc
agcttcttcctgtacagcaagctgaccgtggacaagtccaggtggcagcagggcaacgtgttcagctgcagc
gtgatgcacgaggccctgcacaaccactacacacagaagagcctgagcctgtccccgggtgccggaggtgg
cggaagtggtggcggtggctcaggtggcggtggatcccagctgcagctgcaggagtctggaccaggactg
gtgaagccctcggagaccctgtccctcacctgcactgtctctggtggctccatcagcagtagtagttactactgg
ggctggatccgccagcccccagggaaggggctggagtggattgggggtattgggagtgtcgattatagtgg
gaacacctactacaaaccgtccctcaagagtcgagtcaccatatccgtagacacgtccaagaaccagttttccc
tgaagctgagcactgtgaccgccgcagacacggctgtgtatcactgtgcgagacatcgtgggatatacttcttt
gactactggggccagggaaccctggtcaccgtctcttcagctagcaccaagggtccatcggtcttccccctgg
caccctcctccaagagcacctctgggggcacagcggccctgggctgcctggtcaaggactacttccccgaac
cagtgacggtgtcgtggaactcaggcgccctgaccagcggcgtgcacaccttcccggctgtcctacagtcctc
aggactctactccctcagcagcgtggtgaccgtgccctccagcagcttgggcacccagacctacatctgcaac
gtgaatcacaagcccagcaacaccaaggtggacaagagagttgagcccaaatcttgt
97G07 light chain DNA sequence
(SEQ ID NO: 74)
tactatgagctgactcagccactctcagtgtcagtggccctgggacagacggccaggattccctgtgggggaa
acaacattgaaaataaaaatgtgcactggtaccaacagaagccaggccaggcccctgtactggtcatctatag
ggatagcaaccggccctctgggatccctgagcgattctctggctccaacteggggaacacggccaccctgtc
catcaacagagcccaagccggggatgaggctgactattactgtcaggtgtgggacagcaacactgtgatattc
ggcggagggaccaaggtcaccgtcctaggacagcccaaggctgccccctcggtcactctgttcccgccctcc
tctgaggagcttcaagccaacaaggccacactggtgtgtctcataagtgacttctacccgggagccgtgacagt
ggcctggaaggcagatagcagccccgtcaaggcgggagtggagaccaccacaccctccaaacaaagcaa
caacaagtacgcggccagcagctacctgagcctgacgcctgagcagtggaagtcccacaaaagctacagct
gccaggtcacgcatgaagggagcaccgtggagaagacagtggcccctacagaatgttca

Anti-OX40L FcFab antibodies and the CTLA4_anti OX40L proteins were produced by transient transfection of the correctly paired light chain and heavy chain DNA expression vectors detailed above. The anti-OX40L FcFab antibodies were produced in a 15-ml scale transient transfection of ExpiCHO cells and affinity purified with protein A beads and characterized. The CTLA4_anti OX40L proteins were produced in a 1000-ml scale transient transfection of ExpiCHO cells and affinity purified with a protein A column (Hi-Trap MAbSelect SuRe, GE Healthcare) on an Akta Avant 25 chromatography system (GE Healthcare). Final protein concentrations were measured and proteins were characterized by analytical size exclusion chromatography (SEC), SDS-PAGE gels and tested for endotoxin before use in the planned studies.

Since it is not guaranteed that any anti-OX40L IgG when reformatted to anti-OX40L FcFab or CTLA4_anti OX40L fusion protein would retain binding and neutralizing characteristics to OX40L of the original anti-Ox40L IgG, the FcFab formats and fusion protein formats were tested according to the same screening assay cascade (FIG. 2A) as used for the IgG format. Positive reference control anti-OX40L IgG was used across all assays.

### 3.2 Characterization of Anti-OX40L FcFab Antibodies

### 3.2.1 OX40L Neutralization Assay

Anti-OX40L FcFAb antibody clones were tested for OX40L blocking function with the OX40L neutralization assay. Briefly, test antibodies, isotype control and known anti-OX40L reference antibody (positive controls) were serially titrated in culture media, incubated with recombinant human OX40L, and added to OX40 receptor expressing HEK-luciferase reporter cells as described in 1.1.9 of Example 1. Data were plotted with GraphPad Prism 8 software. Normalization was performed using unstimulated cells as 0% and stimulation with OX40L in the absence of antibodies as 100% of Luciferase activity from each experimental plate.

As can be seen by their IC₅₀ values, the OX40L blocking activity of FcFab antibody clones 97G07 (FcFab), 67B06 (FcFab), 68F03 (FcFab), 98E10 (FcFab), 89B09 (FcFab), 98C01 (FcFab) was similar to their IgG counterpart, suggesting that reformatting to FcFab did not considerable affect their neutralization potency. However, FcFab antibody clones 84E11 (FcFab), 88B06 (FcFab), 95B06 (FcFab), 30F02 (FcFab), 85F12 (FcFab) and 85F10 (FcFab) demonstrated a considerable decrease in potency, increasing their IC₅₀ by at least two-fold compared to their IgG counterpart. IC₅₀ values for anti-OX40L FcFab antibodies were determined with GraphPad Prism by curve fitting using the log (inhibitor) vs normalized response-variable slope equation. TABLE 9 summarizes data from duplicates from 1 experiment.

### 3.2.2 Primary T Cell Activation Assay

Eleven (clone 98C01 was not tested in this assay) of the anti-OX40L FcFab antibody clones were additionally tested with the Primary T cell activation assay described in 1.1.10. Briefly, CD4+ T cells were stimulated with anti-CD3, anti-CD28, and recombinant human OX40L, and anti-OX40L FcFAb antibody clones were tested in serial dilution in this assay. Data were plotted with GraphPad Prism 8 software. Normalization was performed using unstimulated T cells as 0% and stimulated with OX40L in the absence of antibodies as 100% of IL-2 response from each experimental plate. IC₅₀ values were determined with GraphPad Prism by curve fitting using the log(inhibitor) vs normalized response-variable slope equation. TABLE 9 summarizes IC₅₀ (in nM+/−SEM) from 2 experiments. 97G07 (FcFab), 67B06 (FcFab), 98E10 (FcFab), 89B09 (FcFab), 30F02 (FcFab), and 85F10 (FcFab) retained their potency similar to reference control anti-OX40L IC₅₀=5.7-9.3 nM) despite the reformatting.

### 3.2.3 Steady State Affinity Assay

As with the IgG clones, binding affinity to human OX40L was determined for all twelve FcFabs using steady state affinity assay as described in section 1.1.7 of Example 1. Most FcFabs retained binding to the target showing a range of affinity K_{D}=20.3-64.1 nM except for 84E11 (FcFab), which completely lost binding activity. TABLE 9 summarizes data from this assay.

TABLE 9
Data from 12 Anti-OX40L (FcFab)

| | OX40L | Primary T cell | Steady State |
|---|---|---|---|
| | neutralization/OX40- | activation assay | affinity human |
| Agents | HEK Reporter IC₅₀ | IC₅₀ | OX40L K_{D} |
| (FcFAb) | nM (+/−SEM) | nM (+/−SEM) | (nM) |
| 97G07 (FcFab) | 6.2 | 4.6 ± 3.22 | 27.9 |
| 67B06 (FcFab) | 5.5 | 7.2 ± 5.10 | 20.3 |
| 68F03 (FcFab) | 5.5 | 15.6 ± 11.00 | 33.1 |
| 84E11 (FcFab) | 57.2 | 15.2 ± 10.76 | No Binding |
| 98E10 (FcFab) | 5.7 | 5.3 ± 3.75 | 33.0 |
| 89B09 (FcFab) | 4.7 | 6.1 ± 4.28 | 44.8 |
| 98C01 (FcFab) | 5.4 | Not Tested | 27.8 |
| 88B06 (FcFab) | 101.9 | >161 | 35.5 |
| 95B06 (FcFab) | 30.79 | 38.0 ± 26.87 | 64.1 |
| 30F02 (FcFab) | 52.8 | 2.9 ± 2.02 | 56.0 |
| 85F12 (FcFab) | >161 | >161 | 34.1 |
| 85F10 (FcFab) | >161 | 6.3 ± 4.42 | 50.1 |

### 3.3 Characterization of CTLA4_Anti OX40L Fusion Proteins

### 3.3.1. OX40L Neutralization Assay

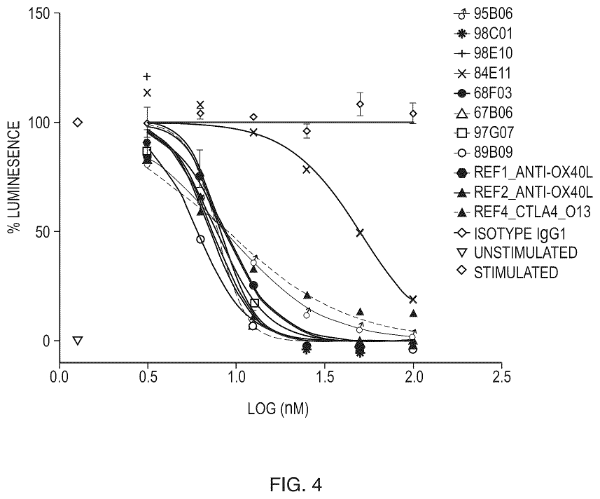

CTLA4_anti OX40L fusion proteins were tested for OX40L blocking function with the OX40L neutralization assay. Briefly, test fusion proteins, isotype control and known anti-OX40L reference antibody (positive controls) were serially titrated in culture media, incubated with recombinant human OX40L, and added to OX40 receptor expressing HEK-luciferase reporter cells as described in 1.1.9 of Example 1. Data were plotted with GraphPad Prism 8 software. Normalization was performed using unstimulated cells as 0% and stimulation with OX40L in the absence of fusion proteins as 100% of luciferase activity from each experimental plate. Data summarized in TABLE 10 from two experiments. FIG. 4 is a graph showing neutralization of OX40L for CTLA4_anti OX40L fusion proteins 95B06, 98C01, 98E10, 84E11, 68F03, 67B06, 97G07, 89B09, and Ref4_CTLA4_013, for the reference anti-OX40L monoclonal antibodies Ref1_Anti_OX40L and Ref2_Anti-OX40L, or for isotype control, using OX40 luciferase reporter assay. As shown in FIG. 4, seven CTLA4_anti OX40L fusion proteins (97G07, 68F03, 67B06, 98E10, 89B09, 98C01, 95B06) retained similar potency to a reference anti-OX40L monoclonal antibody. Five fusion proteins showed a significant drop in potency or lost their neutralization capacity compared to their IgG counterpart 84E11 (IgG) IC₅₀=17 nM→CTLA4_anti OX40L IC₅₀=50.2 nM; 88B06 (IgG), 30F02 (IgG), 85F12 (IgG), 85F10 (IgG) IC₅₀=19-72.7 nM 4 CTLA4_anti OX40L IC₅₀=>100 nM), data shown in TABLE 8.

### 3.3.2 Primary T Cell Activation Assay

Since 88B06, 30F02, 85F12, and 85F10 showed weak to no blocking, only eight fusion proteins were assessed for neutralizing human OX04L in the primary human T cell activation assay (described in 1.1.10 of Example 1). Data were plotted with GraphPad Prism 8 software. Normalization was performed using unstimulated T cells as 0% and stimulated with OX40L in the absence of fusion proteins as 100% of IL-2 response from each experimental plate. IC₅₀ values were determined with GraphPad Prism by curve fitting using the log(inhibitor) vs normalized response-variable slope equation. TABLE 10 summarizes IC₅₀ (in nM+/−SEM).

As shown in TABLE 10, fusion proteins 97G07, 67B06, 68F03, 84E11, 98E10, 89B09, and 98C01 retained potency similar to their IgG counterpart and reference control anti-OX40L IC₅₀=5.7-9.3 nM) despite the reformatting.

### 3.3.3 Steady State Affinity to OX40L, CD80, and CD86

Binding affinity to human OX40L was determined for all 12 CTLA4_anti OX40L fusion proteins using steady state affinity assay as described in section 1.1.7 of Example 1. As with the FcFab formats, most CTLA4_anti OX40L fusion proteins retained binding to the target showing a range of binding affinity K_{D}=27.6-106.1 nM. As expected, fusion protein 84E11, similar to its FcFab counterpart, lost binding affinity to human OX40L. TABLE 10 summarizes data from this assay.

Moreover, CTLA4_anti OX40L fusion proteins 89B09, 67B06 and 98C01 and a reference anti-OX40L antibody were evaluated for binding to human and cynomolgus monkey OX40L expressed on CHO-S cells by flow cytometry as described in section 1.1.4 of Example 1. The binding EC₅₀ for 89B09 (CHO-S-huOX40L EC₅₀=4.62 and CHO-S-cynoOX40L EC₅₀=4.37 nM), 67B06 (CHO-S-huOX40L EC₅₀=1.85 and CHO-S-cynoOX40L EC₅₀=2.77 nM), and 98C01 (CHO-S-huOX40L EC₅₀=2.79 and CHO-S-cynoOX40L EC₅₀=3.45 nM), were comparable to the binding EC₅₀ for the reference anti-OX40L (CHO-S-huOX40L EC₅₀=1.14 and CHO-S-cynoOX40L EC₅₀=0.95 nM) indicating excellent cross-reactivities between human and cynomolgus OX40L. Binding affinity to cynomolgus monkey OX40L was determined for fusion proteins 67B06 and 89B09 and compared to the reference anti-Ox40L antibody. The K_{D} of the fusion protein (85 nM) was found to be similar to that of the reference anti-OX40L antibody (57 nM).

Binding affinity to human and cynomolgus CD80 and CD86 was determined for CTLA4_anti OX40L fusion proteins and reference CTLA4-Ig proteins using steady state affinity assays as described in section 1.1.8 of Example 1. Steady state binding KD for 67B06 (human CD80/human CD86=538 nM/670 nM; cynomolgus CD80/cynomolgus CD86=486 nM/770 nM) and 89B09 (human CD80/human CD86=540 nM/654 nM; cynomolgus CD80/cynomolgus CD86=474 nM/750 nM) were comparable to the steady state binding KD determined for the CTLA4-Ig reference protein (human CD80/human CD86=450 nM/663 nM; cynomolgus CD80/cynomolgus CD86=433 nM/910 nM). Thus fusion of an anti-OX40L Fab domain to the C-terminus of an CTLA4-Ig protein to generate CTLA4_anti OX40L fusion proteins did not appreciably change the binding affinity of the N-terminal CTLA4 domain to CD80 or CD86. In conclusion, CTLA4_anti OX40L fusion proteins retained binding to and demonstrated excellent cross-reactivities between human and cynomolgus OX40L, CD80 and CD86.

The seven CTLA4_anti OX40L fusion proteins were rank ordered and four clonally diverse sequences were selected based on highly potent neutralization activity and affinity closest to the monoclonal anti-OX40L IgG reference antibodies.

TABLE 10
Data from twelve CTLA4_anti OX40L candidates

| | OX40L | Primary | Steady |
|---|---|---|---|
| | neutralization/ | T cell | State |
| | OX40-HEK | activation | affinity |
| | Reporter | assay | human |
| Agents (Fusion | IC₅₀ nM | IC₅₀ nM | OX40L K_{D} |
| proteins) | (+/−SEM) | (+/−SEM) | (nM) |
| 97G07 | 7.7 ± 0.95 | 7.1 | 57.4 |
| 67B06* | 7.3 ± 1.10 | 6.4 ± 3.71 | 27.6 |
| 68F03 | 10.1 ± 1.12 | 2.2 | 38.2 |
| 84E11 | 50.2 ± 0.86 | 8.1 | NB |
| 98E10 | 8.3 ± 0.86 | 3.5 ± 2.05 | 57.9 |
| 89B09** | 6.4 ± 0.88 | 5.4 ± 3.12 | 71.4 |
| 98C01 | 7.5 ± 1.22 | 5.1 ± 2.93 | 72.3 |
| 88B06 | >161 | Not tested | 63.8 |
| 95B06 | 12.7 ± 3.09 | 20.8 | 106.1 |
| 30F02 | >161 | Not tested | 68.5 |
| 85F12 | >161 | Not tested | 65.8 |
| 85F10 | 119.6 | Not tested | 51.1 |
| Ref1_anti-OX40L | 7.3 ± 0.66 | 5.7 ± 3.28 | 31.9 |
| Ref2_anti-OX40L | 6.6 ± 1.04 | 9.3 ± 5.39 | 15.0 |
| CTLA4_anti- | 8.8 | Not tested | Not tested |
| OX40L_O13 | | | |

*Variants of 67B06 comprising different bridging moieties all exhibited similar steady state binding to human OX40L (data not shown).
**Variants of 89B09 comprising different bridging moieties all exhibited similar steady state binding to human OX40L (data not shown).
Comparison of three formats

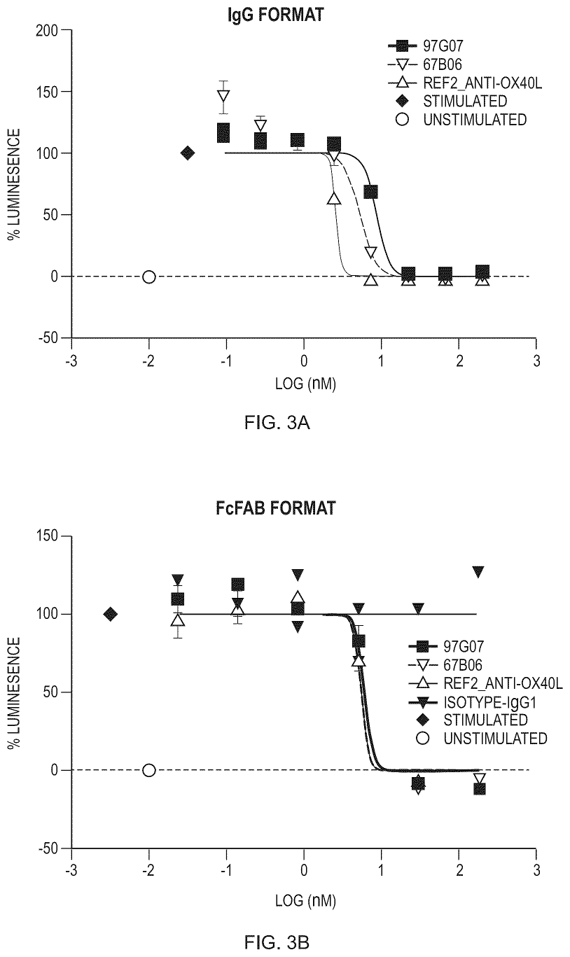

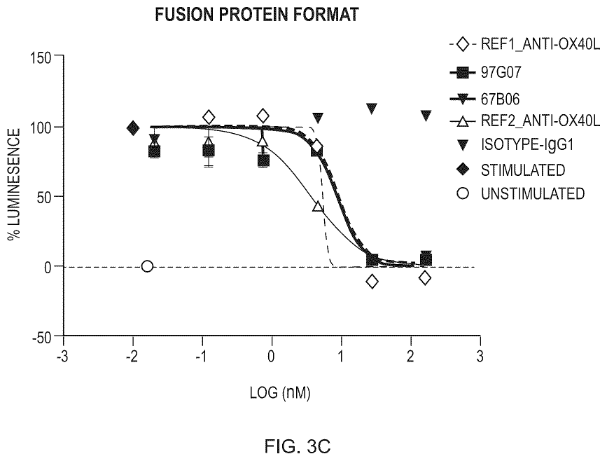

For direct comparison of data from three different formats (anti-OX40L (IgG), anti-OX40L (FcFab), and CTLA4_anti OX40L fusion protein), data was plotted from the neutralization assay. FIGS. 3A-3C are graphs showing neutralization of OX40L by anti-OX40L (97G07 and 67B06) in IgG (FIG. 3A), FcFab (FIG. 3B), and fusion protein (FIG. 3C) format using OX40 luciferase reporter assay. Monoclonal anti-OX40L antibodies (Ref1_Anti-OX40L and/or Ref2_Anti-OX40L) were used as reference controls. Anti-HEL IgG1 was used as isotype control (Isotype-IgG1).

### Example 4: Mechanism of Action of CTLA4_Anti OX40L Fusion Proteins Using In Vitro Assays

This example elucidates the mechanism of action of CTLA4_anti OX40L fusion proteins utilizing in vitro assays.

### 4.1 Allogenic MDDC:T Mixed Lymphocyte Reaction (MLR) Assay

The CTLA4_anti OX40L fusion proteins that retained good human OX40L neutralization and binding were tested for their bifunctional activities. To interrogate whether the fusion proteins can block inflammatory cytokines generated from T cells receiving co-stimulation signals through both the CD80/CD86-CD28 pathway and OX40L-OX40 pathway, a human allogenic MDDC: T mixed lymphocyte reaction (MLR) was set up. This assay utilized activated primary monocyte derived dendritic cells (MDDC) as antigen presenting cells that express all three targets CD80, CD86, and OX40L, allowing the assessment of both CD28 and OX40 pathways contributing to T cell proinflammatory cytokine release and T proliferation.

Briefly, freshly isolated monocytes were cultured in the presence of GM-CSF and IL-4 (in AIM-V media with 5% human AB serum, 50 U/ml Penicillin, 50 μg/ml Streptomycin) for 5 days to become immature monocyte-derived dendritic cells (iMDDC). Then iMMDCs were treated with 1 μg/ml of Prostaglandin E2 (PGE2), and 20 ng/ml TNFα, 10 ng/mL IL-1b, 20 ng/mL IL-6 for 2 days to generate matured MDDC. Cell surface expression of CD86, CD80, and OX40L on the matured MDDC were confirmed by flow cytometry. To set up mixed lymphocyte reactions, fresh PBMCs were isolated from buffy coat using a Ficoll gradient. Total T cells were isolated from the PBMCs using a pan-T cell isolation kit (Miltenyi Biotec, 130-096-535). In a flat bottom 96-well tissue culture plate, 20,000 MDDC and 80,000 pan T cells were plated in each well, mixed well, and treated with various concentrations of either media control, isotype control, or test agents. CTLA4_anti OX40L fusion proteins 89B09, 67B06, 98E10, and 98C01 were tested at three concentrations (0.8 nM, 6.25 nM, and 50 nM) compared to equimolar concentrations of single reference agents (Ref2_Anti-OX40L, Ref3_CTLA4Ig), and combination of monoclonal anti-OX40L antibody and CTLA4-Ig (combination). Then the cells were cultured at 37° C., 5% CO₂ for 4 days, followed by cytokine analysis released in the supernatant. The levels of IL-2, IFNγ, TNFα, and IL-6 were evaluated by AlphaLISA assay (Perkin Elmer).

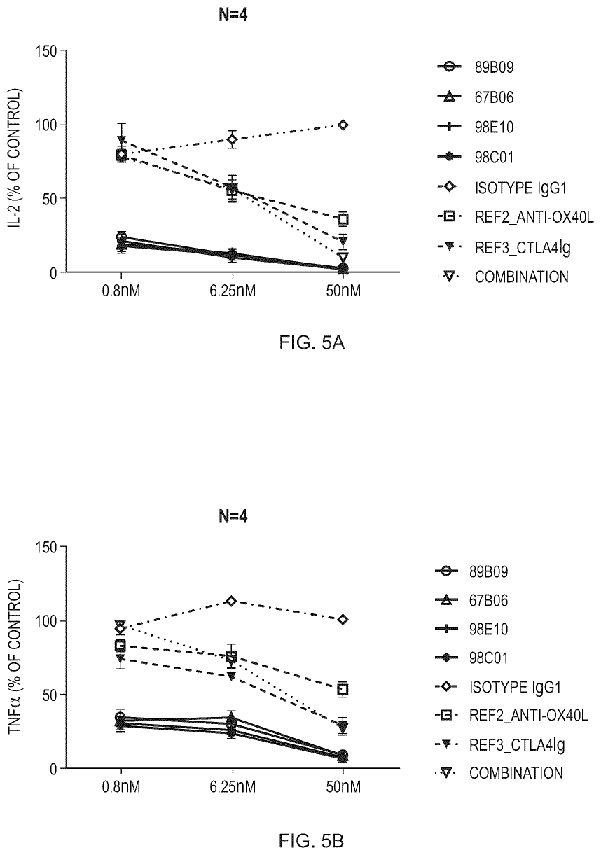

As shown in FIGS. 5A-5B, CTLA4-Ig (Ref3-CTLA4Ig) alone or anti-OX40L alone (Ref2_Anti-OX40L) showed concentration dependent reduction of cytokines, demonstrating that the assay can measure effects from either pathway blockades, albeit less potently at lower concentrations. In contrast, the fusion protein (67B06, 98E10, and 98C01) demonstrated superior suppression of IL-2 (FIG. 5A) and TNFα (FIG. 5B) cytokines at all concentrations tested compared to the single reference agents. Surprisingly, the fusion proteins potently suppressed the cytokines better than the combination of CTLA4-Ig and anti-OX40L, suggesting a synergistic effect by a bifunctional fusion protein compared to single agents in combination. Data shown was normalized from 4 donor MLR pairs. IC₅₀ values for all tested fusion proteins were determined by curve fitting using the log(inhibitor) vs normalized response-variable slope equation. TABLE 11 summarizes the IC₅₀ in nM+SEM from 4-6 donor pairs. Variants of 67B06 comprising different bridging moieties all exhibited similar inhibition of IL-2 and TNF_{α} (data not shown).

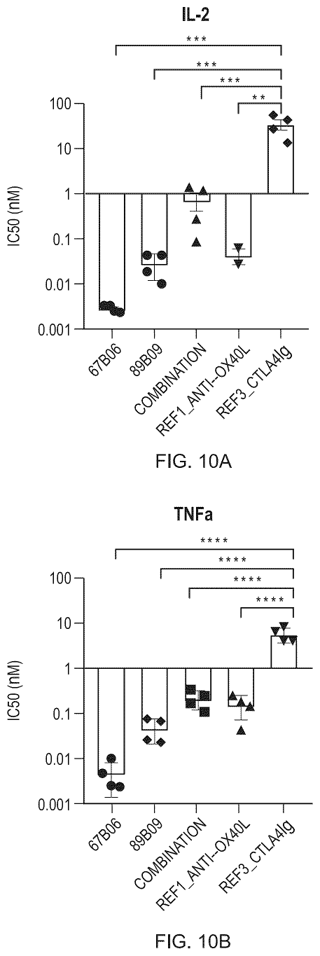

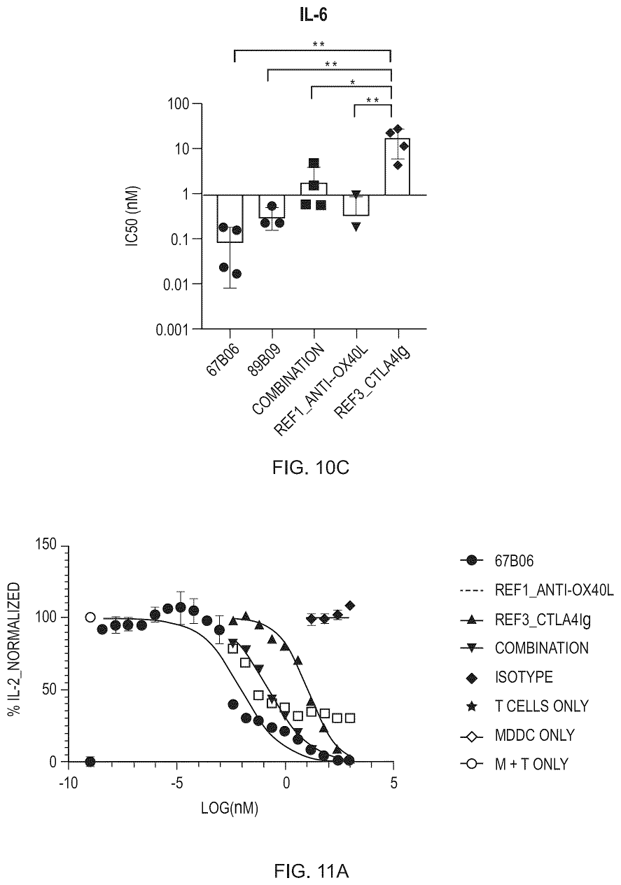

67B06, 89B09 fusion proteins were serially titrated in a subsequent assay to determine the IC₅₀ values for IL-2 (FIG. 10A), TNFα (FIG. 10B), and IL-6 (FIG. 10C) inhibition. FIGS. 10A-10C show that 89B09 and 67B06 were consistently the most potent agents in suppressing inflammatory cytokines IL-2, TNFα, and IL-6 compared to single agent controls or combination, with the IC₅₀ nM of each agent plotted as mean±SEM for each cytokine. Data shown is a summary of 4 different MDDC: T MLR pairs, statistical significance performed using one-way ANOVA, *p=<0.05, **p=<0.005, ***p=<0.0005, ****p=<00.00005.

The anti-OX40L treatment alone demonstrated measurable IC₅₀ for two out of the four MLR experiments, suggesting that the fusion proteins had a more robust and consistent effect across multiple experiments and multiple donors.

To test if the CTLA4_anti OX40L fusion protein modulated allo-reactive proliferation, isotype control fusion proteins, single agents and combination were tested in the MDDC: T MLR assay. For proliferation readouts, T cells were labelled with Cell Trace Violet dye (Invitrogen) and cultured with matured MDDCs as described above with the addition of 0.35 μL/mL anti-CD3 beads (StemCell, 10309). Fusion proteins (89B09, 67B06, 98E10, and 98C01), CTLA4-Ig (Ref3_CTLA4Ig), anti-OX40L (Ref2_Anti-OX40L), or combination of CTLA4-Ig and anti-OX40L (combination) were tested at equimolar concentrations of either 6.25 nM or 50 nM. The cells were subsequently cultured at 37° C. and 5% CO₂ for 4 days, stained with fluorescent labelled antibodies against the following markers CD3, CD4, and OX40 for FACS analysis on BD LSR Fortessa X-20 and analyzed for percent proliferating CD4+OX40+ and proliferating CD4-OX40+ or CD8+OX40+ cells by FlowJo.

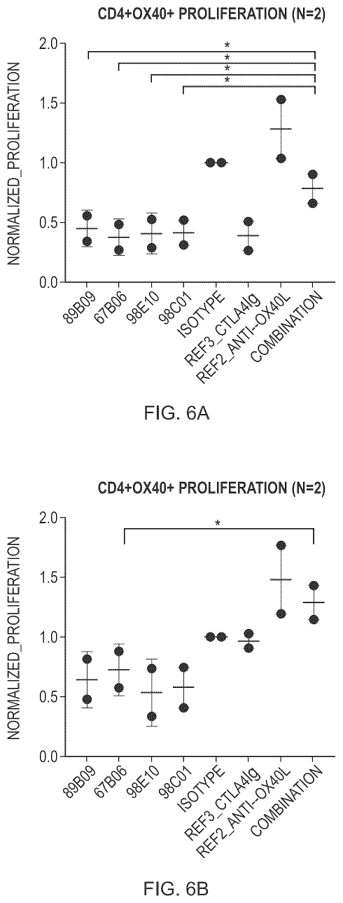

As shown in FIGS. 6A-6B, the fusion proteins (89B09, 67B06, 98E10, and 98C01) potently reduced proliferation of allo-reactive CD4+OX40+ and CD8+OX40+ cells better than the combination of CTLA4-Ig and anti-OX40L (combination) in a statistically significant manner. The results from two experiments were plotted as percentages on GraphPad PRISM8 and statistical significance performed using one-way ANOVA, *p=<0.05. This was significant because only treatment with fusion proteins but not the single agent reference controls or the combination were able to dampen CD8+OX40+ T cells which are often associated with memory T cells and disease pathology, suggesting that simultaneous dual blockade of both CD28 and OX40 pathways was necessary to achieve this effect.

### 4.2 Treg Induction Assay

Soluble OX40L has been shown to inhibit Treg induction in vitro. The fusion proteins (89B09, 67B06, and 98C01) were assessed for OX40L neutralizing function and their ability to restore Treg generation in a Treg inducing assay compared to reference anti-OX40L (Ref1_Anti-OX40L and Ref2_Anti-OX40L) and CTLA4-Ig (Ref3_CTLA4Ig) controls.

Naïve T cells were freshly isolated from human blood using human naïve CD4 T-cell Isolation Kit (Miltenyi). Complete RPMI1640 medium (supplemented with 10% heat-inactivated FBS, 2 mM L-glutamine, 50 IU Penicillin/50 μg/ml of Streptomycin, 1 mM Na Pyruvate, 55 μM β-mercaptoethanol, and 0.01M of HEPES) was used for the in vitro T cell culture. 100,000 naïve T cells were placed in each well of a 96-well tissue culture plate and stimulated with plate-bound anti-CD3 (5 ug/ml) and soluble anti-CD28 (1 ug/ml), plus 5 ng/ml of TGF-β and 50 U/ml of IL-2. The cells were cultured for 5 days at 37° C. in CO₂ incubator.

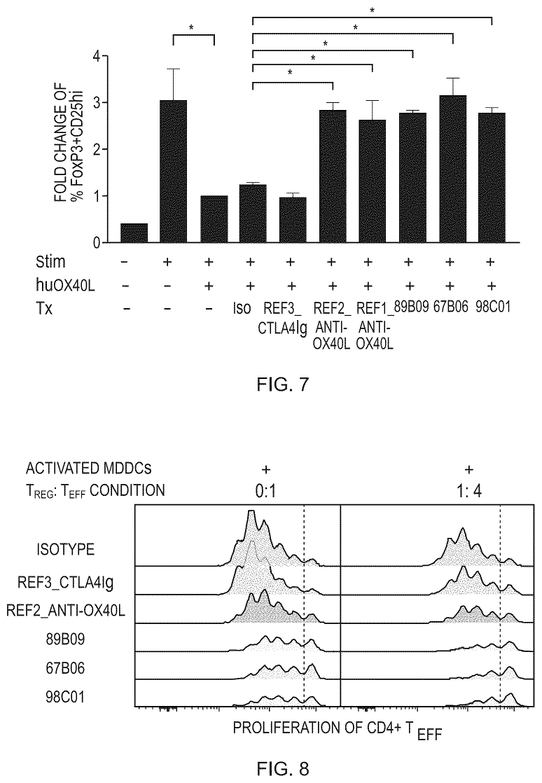

Recombinant soluble OX40L (1 μg/ml, R&D Systems) was added to most conditions except controls. Either isotype control IgG, fusion proteins or reference control treatments (50 nM) were added to respective wells on day 0. At the end of the experiment, cells were collected, and intracellular FoxP3 staining was performed using manufacturer's (eBioscience) instructions for ‘Staining Intracellular FoxP3”. FACS analysis was performed using the BD LSR Fortessa X-20. Data analysis was performed using FlowJo software. The frequency of Tregs were normalized to that of isotype treatment condition and statistical significance performed using one-way ANOVA, *p=<0.05. As shown in FIG. 7, in induction conditions containing soluble OX40L, iTreg generation decreased significantly by 3-fold. However, the presence of equimolar CTLA4_anti OX40L fusion proteins (89B09, 67B06, and 98C01) or the reference anti-OX40L antibodies (Ref1_Anti-OX40L and Ref2_Anti-OX40L), neutralized the effects of soluble OX40L and fully restored Treg induction in vitro. As expected, CTLA4-Ig (Ref3_CTLA4Ig), which did not bind OX40L, did not impact Treg restoration.

### 4.3 Three-Way MDDC: Treg: Teff MLR Assay

To demonstrate whether the fusion CTLA4_antiOX40L fusion proteins synergize with Tregs to further suppress Teff proliferation, a 3-way MDDC: Treg: Teff MLR assay was utilized.

Freshly isolated monocytes were cultured in the presence of 50 ng/ml GM-CSF and 50 ng/ml IL-4 (in AIM-V media with 5% human AB serum, 50 U/ml Penicillin, 50 μg/ml Streptomycin) for 5 days to become immature monocyte-derived dendritic cells and treated with 1 μg/ml of PGE2, 20 ng/ml TNFα, 20 ng/ml IL-6 and 10 ng/ml IL-10 for 2 days to become matured MDDC (MDDC). Cell surface expression of CD86, CD80, and OX40L was confirmed by FACS.

To set up mixed lymphocyte reactions, fresh PBMCs were isolated from buffy coat using a Ficoll gradient. CD4⁺ T cells were isolated from the PBMCs using a human CD4 T cell isolation kit (Miltenyi). Freshly isolated CD4⁺ T cells were then sorted into CD4⁺ CD25^{hi}CD127^{low} Treg and non-Treg CD4⁺ T (or Teff, T_{eff}, or T_{effector}) cells by flow cytometry. The sorted Tregs were labelled with 2 μM of CellTrace Violet, and Teff CD4⁺ T cells were labelled with 2 μM CellTrace CFSE. In a flat bottom 96-well tissue culture plate, 25,000 MDDC and 50,000 Teff CD4⁺ T cells were plated in each well and cultured in complete AIM-V medium ((supplemented with 5% human AB serum, soluble anti-CD3 (0.6 μl/ml) (StemCell), 50 IU Penicillin/50 μg/ml of Streptomycin)). In conditions with Treg, 12,500 Treg were added. The cultured cells were also treated with either media control, isotype control Ab, CTLA4-Ig, anti-OX40L reference antibody, CTLA4_anti OX40L fusion proteins (89B09, 67B06, and 98C01) at 10 nM.

In a separate experiment, fusion protein 89B09 and associated controls were tested at 10 nM in the presence of various Treg:Teff ratio conditions. Then the cells were cultured at 37° C., 5% CO₂ for 4 to 5 days before the cells were analyzed with BD LSR Fortessa X-20. Effector T cell proliferation was determined by CFSE dilution. The histograms were analyzed by FlowJo, the ratio of Treg to Teff, and absolute cell counts of proliferating Teff cells were plotted using Graphpad PRISM8.

As shown in FIG. 8, the fusion proteins (89B09, 67B06, 98C01) demonstrated an even greater suppression of Teff proliferation compared to the control agents in the presence of Tregs (1:4 Treg:Teff condition). This result indicated that the fusion proteins can more effectively dampen Teffs and can work together with Tregs to affect further Teff suppression.

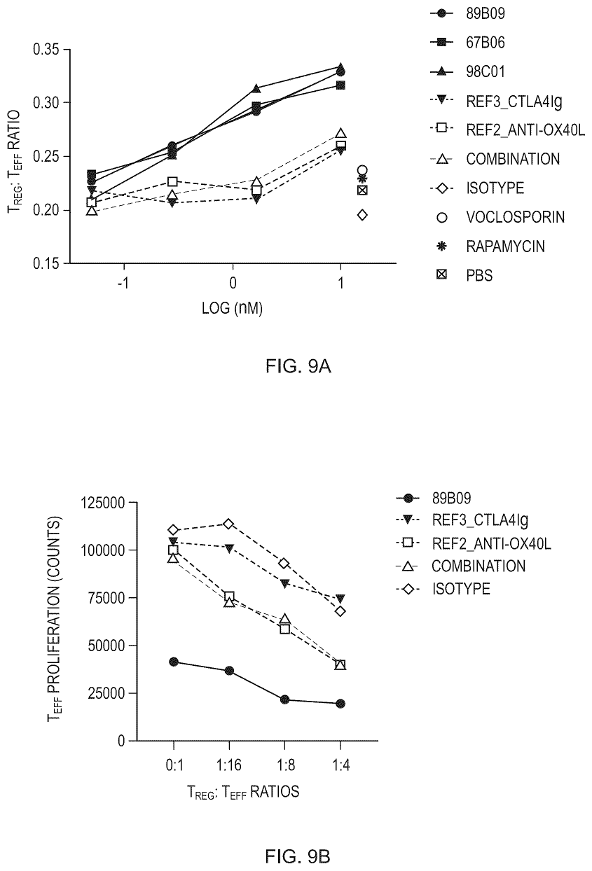

Rapamycin is an mTOR inhibitor and a general T cell immunosuppressant used in transplantation to effectively suppress Teff while increasing Tregs, thereby increasing the Treg:Teff ratio. Voclosporin, a second generation cyclosporin, is a calcineurin inhibitor and known T cell immunosuppressant that recently received FDA approval to treat lupus nephritis. The same MLR assay as described above was performed with varying concentrations of fusion proteins, single agents, combination, and 100 nM rapamycin or volcopsorin. As shown in FIG. 9A, CTLA4_anti OX40L fusion proteins (89B09, 67B06, 98C01) increased Treg: Teff ratio in a concentration dependent manner compared to single agents or the combination. Rapamycin or voclosporin did not increase Treg: Teff ratio in this in vitro assay although clinically efficacious concentrations were tested.

Since Tregs also require CD28 signaling for activation and proliferation, it was essential to address whether the fusion proteins may inhibit Tregs and negatively impact their suppressive function on Teff. By modulating the Treg:Teff ratios in the 3-way MLR cultures discussed above, the effect of fusion protein treatment on inhibiting T effector proliferation in the presence of increasing Tregs was also tested. As shown in FIG. 9B, cultures treated with isotype control, the increasing Treg: Teff ratios decreased Teff proliferation as expected. CTLA4-Ig treatment did not provide additional modulation of Teff suppression on top of the Tregs while anti-OX40L and combination provided modest modulation of Teff proliferation. Impressively, the fusion protein 891B09 suppressed the Teff proliferation better than the other treatments in the presence of increasing Tregs, indicating that the fusion protein did not negatively impact Treg function but worked synergistically with Tregs to more effectively dampen Teff proliferation. The observation that the fusion protein but not the combination of single agents worked synergistically with Tregs to preferentially decrease Teff proliferation was a surprising finding and uncovered potentially interesting mechanistic differences between treatment with CTLA4_anti OX40L fusion proteins with dual co-stimulation blocking properties compared to treatment with the combination of single agents.

TABLE 11

| | Invitro | Invitro | Cellular | Steady State | Steady State |
|---|---|---|---|---|---|
| | MLR | MLR | binding to | affinity | affinity |
| | IL-2 | TNFα | human | human | Human |
| | IC₅₀ nM | IC₅₀ nM | OX40L | OX40L | CD80/CD86 |
| Agents | (+/−SEM) | (+/−SEM) | EC₅₀ (nM) | K_{D} (nM) | K_{D} (nM) |
| 67B06 (1) | 0.02 ± 0.004 | 0.06 ± 0.018 | 1.5 | 27.6/35.2 | 538/670 |
| 89B09 (1) | 0.03 ± 0.01 | 0.12 ± 0.05 | 4.1 | 71.4/53.1 | 654/654 |
| 98C01 (1) | 0.04 | NT | 4.2 | 72.3/54.7 | |
| 98E10 (1) | NA | NT | 1.7 | 57.9/63.9 | |
| 30F02 (1) | NT | NT | | 68.5 | |
| Ref1 anti- | 0.69 ± 0.26 | 1.47 ± 0.52 | | 31.9 | |
| OX40L | | | | | |
| IgG mAb | | | | | |
| Ref2 anti- | NT | NT | | 15.0 | |
| OX40L | | | | | |
| IgG mAb | | | | | |
| Ref 3 | 12.4 ± 2.7 | 9.72 ± 1.4 | NB | NB | 450/663 |
| CTLA4- | | | | | |
| Ig | | | | | |

### Example 5: Characterization of the Potency of CTLA4_anti OX40L Fusion Proteins

This example describes the facts that contribute to the superior potency of CTLA4_anti OX40L fusion proteins. The unexpected aspect of the 67B06 fusion protein showing much greater (orders of magnitude) potency compared to combination treatment indicated that the superior potency is a distinct feature of a single bifunctional protein compared to the combination of two separate molecules. In order to investigate what factors could be contributing to the superior potency on dampening cytokines seen in the fusion protein, the receptor occupancy of fusion proteins, single agents, and combination were compared to the target ligands (CD80 and OX40L) and IL-2 production was measured in the MDDC: T MLR assay

### 5.1 CD80 and OX40L Receptor Occupancy Assay

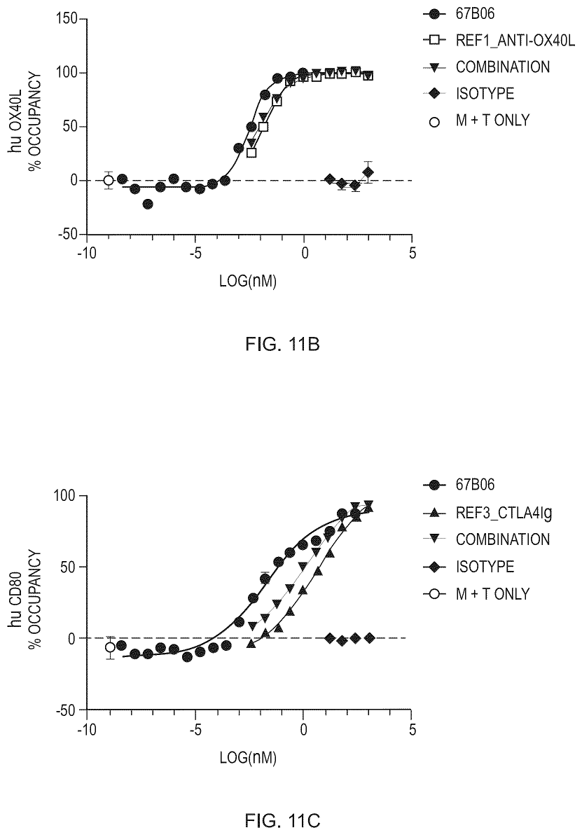

Percent receptor occupancy of CD80 and OX40L by CTLA4_anti OX40L fusion proteins or the single activity reference controls in the allogenic MDDC: T MLR assay was assessed by flow cytometry at day 4. Briefly, cells were washed with PBS and stained with anti-CD11c (clone 3.9, Biolegend), anti-CD80 (clone 2D10, Biolegend), anti-CD86 (clone FUN-1, BD), and anti-OX40L (clone ik-1, BD) and assessed by FACS on the BD Fortessa to determine CD80, CD86 and OX40L expression on the MDDCs. Mean fluorescent intensity was determined for CD80, CD86 and OX40L, dose-response curves were fitted using GraphPad PRISM 8. Transformation of the data to Percent Occupancy was performed using the following equation, where MFI_{NS} was set as 100% inhibition and MDDC:T cocultures in the absence of antibodies, designated as “M+T only” in FIG. 11 legend, (0CPDΔMFI) as minimum inhibition:

$$\% inhibition for sample A = 100 - ( A Δ MFI / 0 CPD Δ MFI ) * 100$$

$${MFI}_{T} = Total median fluorescent intensity of PE channel for population in question$$

$${MFI}_{NS} = Nonspecific median fluorescent intensity of PE in replicate samples incubated$$

$$Δ MFI = {MFI}_{T} - {MFI}_{NS} = background corrected MFI$$

$$0 CPD Δ MFI = Δ MFI of sample dosed with 0 mg / mL compound$$

As shown in FIGS. 11A-11C, the increased potency of the CTLA4_anti OX40L fusion proteins was due to increased receptor occupancy of CD80 and not OX40L. The 67B06 fusion protein potently inhibited the IL-2 production in this assay compared to single reference agents or their combination (FIG. 11A). This effect was demonstrated by 67B06 occupancy of the target CD80, with a 50% receptor occupancy (RO₅₀) at 0.019 nM compared to CTLA4-Ig (RO₅₀=3.5 nM) or combination (RO₅₀=1.03 nM). This result showed that 67B06 had a 184-fold increase in potency for CD80 binding over CTLA4-Ig and a 54-fold increase over the combination (FIG. 11B). 67B06 occupied OX40L with a potency of RO₅₀=0.003 nM compared to anti-OX40L (RO₅₀=0.02 nM) or the combination treatment (RO₅₀=0.01 nM), representing a 6-fold increase and a 3-fold increase in potency on OX40L, respectively (FIG. 11C). Therefore, it is likely that the strong effect of 67B06 to dampen cytokines was attributed more potent CD80 occupancy due to the avidity of the protein by anchoring to its other target OX40L.

### 5.2 Internalization Assay

To test if other mechanisms such as physical internalization or clearance of the test agents may affect their potency in the MDDC:T MLR assay, internalization assay using live-cell imaging technique in real-time was performed.

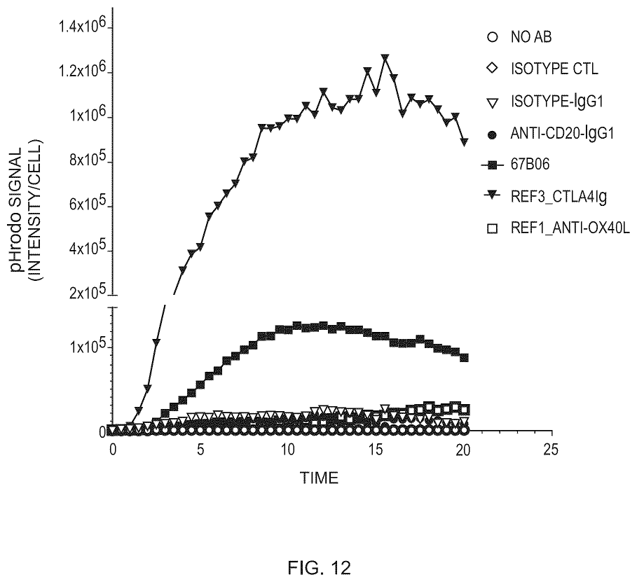

Real-time internalization of CTLA4-Ig, anti-OX40L and the CTLA4_anti OX40L 67B06 fusion proteins were compared on MDDCs over 20h on the live-cell imager, Celldiscoverer7 (Zeiss). Briefly, activated MDDCs were incubated with 100 nM pHrodo labeled antibodies. Internalization of 67B06, CTLA4-Ig, and anti-OX40L agents were tracked by leveraging pH rodo dyes which fluoresce at low pH when internalized into endocytic compartments of the cell. Negative controls for internalization such as pHrodo labelled isotype control and anti-CD20 antibody Rituxumab were tested in the same assay. Internalization of the test agents and the detected pHrodo signals were followed over time and recorded. CTLA4-Ig has been reported to be internalized upon binding to its target, while that may be an efficient way to block the targets CD80 and CD86 by removing them from the cell surface, the target mediated drug clearance may affect its potency. As shown in FIG. 12, CTLA4-Ig (Ref3_CTLA4Ig) was rapidly internalized within 6h and is continually internalized over 20h as indicated by increased pHrodo signal in the MDDCs (FIG. 12). On the other hand, anti-CD20 antibody Rituxumab (Anti-CD20-IgG1), Isotype controls (Isotype Ctl and Isotype-IgG1), or anti-OX40L reference control (Ref1_Anti-OX40L) did not internalize. Although CTLA4_anti OX40L fusion protein (67B06) bound the same CD80 and CD86 targets as CTLA4-Ig, it strikingly did not share the same internalization profile, and in fact, showed limited internalization into MDDCs over a 20 h period (FIG. 12). This may allow the fusion to block the targets CD80, CD86 and OX40L over a longer period of time, without being internalized or cleared, which could explain its enhanced potency compared to single agent comparators in the MDDC: T MLR assay.

### 5.3 MDDC: SLE PBMC MLR Assay

To evaluate the enhanced potency of the CTLA4_anti OX40L fusion proteins in suppressing additional proinflammatory cytokines, a MDDC: SLE PBMC assay was developed. A similar assay set up to the Allogenic MDDC: T MLR assay in Section 3.1 was performed to assess the effect of the fusion proteins in PBMCs derived from systemic erythematosus (SLE) patients. The assay conditions are similar to 3.1 except that patient PBMCs was utilized instead of T cells from healthy volunteers. At Day 4, supernatants from the co-culture were collected and analyzed for proinflammatory cytokines (GM-CSF, Granzyme B, IFN γ, IL-2, IL-4, IL-10, IL-13, IL-17A, TNFα, IL-9, IL-17/IL-25, IL-17F, IL-21, IL-22, MIP-3u) using the MSD Immuno-Oncology Group 1 and Biomarker Group 1 kit. Data from 3 donor pairs were plotted using GraphPad PRISM 8. For ELISPOT readouts, the MDDC: SLE PBMC assay required the addition of anti-CD3. Cell cultures were seeded directly on the IFNγ ELISPOT plate and assayed on Day 4 according to manufacturer's directions (Human IFNγ, R&D Systems). Spots were counted on the CTL ImmunoSpot S6 Analyzer and data analysis performed using GraphPad PRISM 8.

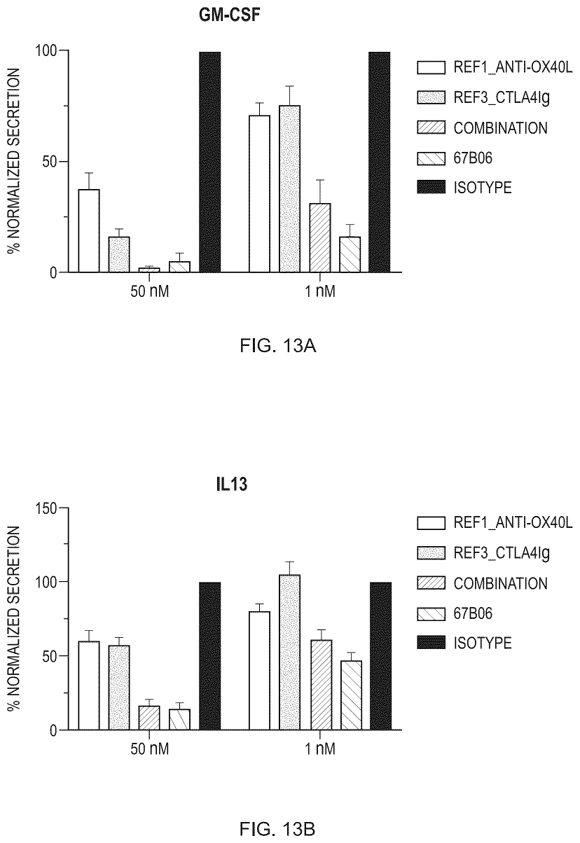

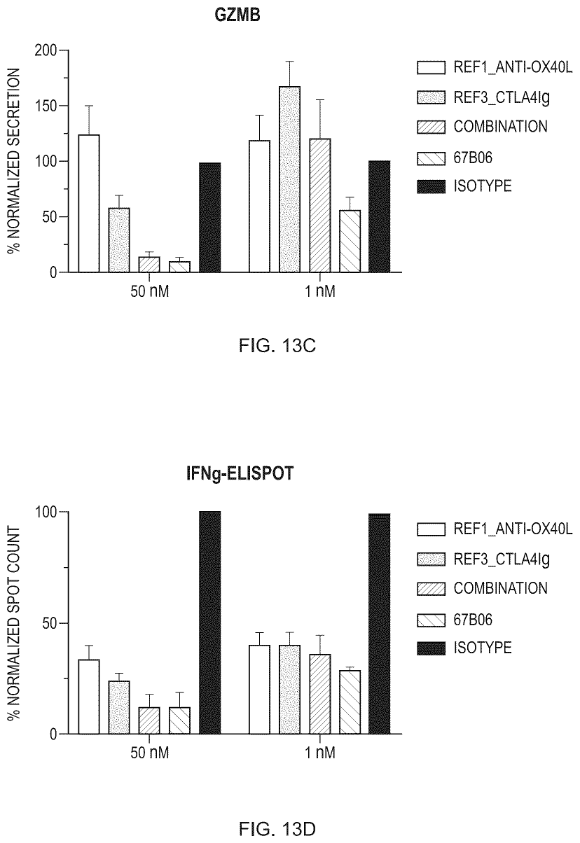

As shown in FIGS. 13A-13C, lupus PBMCs when co-cultured with MDDCs induced elevated levels of IL-2 (data not shown), TNFα (data not shown), GMCSF (FIG. 13A), IL-13 (FIG. 13B), and GZMB (FIG. 13C) in the culture supernatant and these cytokines can be potently suppressed by 1 nM of fusion protein (67B06) compared to the single agents or combination. At 50 nM both fusion and combination treatment are equally efficacious in dampening these cytokines. Allo-reactive IFNγ producing cells were greatly reduced by the fusion protein and combination treatment at 50 nM compared to single agents. FIGS. 13A-13C show normalized data from 3 donors.

### Example 6: In Vivo Pharmacodynamic Effects of CTLA4_Anti OX40L Fusion Protein

This example describes the in vivo pharmacodynamic effects of CTLA4_anti OX40L fusion proteins using humanized MLR PD model.

### 6.1 In Vivo Humanized MLR PD Model

Since none of the CTLA4_anti OX40L fusion proteins cross-react with mouse OX40L, a humanized in vivo pharmacodynamic (PD) model to evaluate the fusion proteins was developed. Briefly, monocyte derived dendritic cells were activated as previously described in the in vitro MDDC: T MLR assay in Section 3.1. On day −1, NOD.Cg-Prkd^{scid}Il2rg^{tmJwji}/SzJ or NSG mice (Jackson Labs) were treated i.v. with various concentrations of fusion proteins (67B06, 89B09), single arm controls or isotype control, followed by transfer of mixed activated MDDC (1×10⁶ cells) and freshly isolated allogenic T cells (1×10⁷ cells) via i.p. on Day 0. After 6 days, serum was collected for human cytokine measurements using MSD kit (human u-Plex proinflammatory combo kit, cat #K15049K-2). The levels of IFNγ and statistical analysis (one-way ANOVA) was performed using GraphPad PRISM 8.

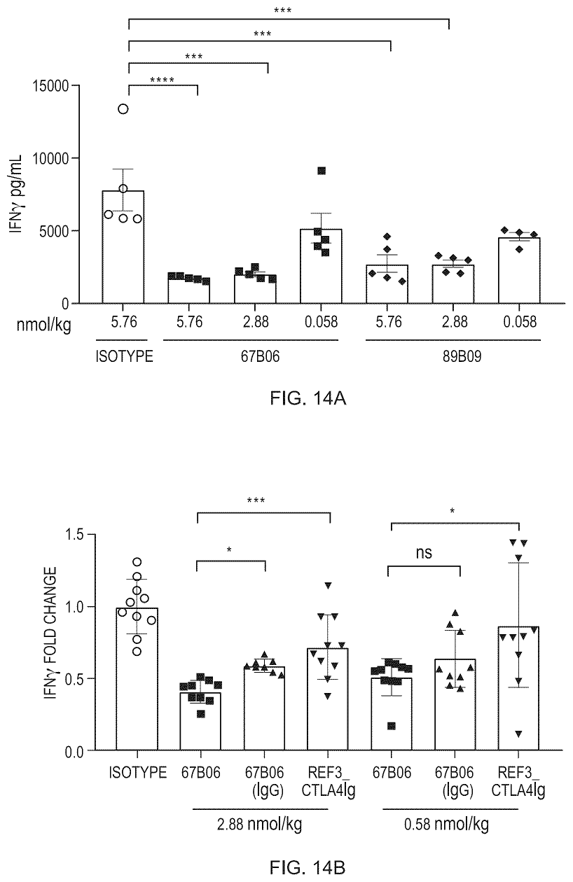

As shown in FIG. 14A, both 67B06 and 89B09 dose dependently decreased human IFNγ production compared to isotype control, demonstrating in vivo efficacy in the mice. In a separate study, mice were treated with a single equal molar (2.88 nmol/kg or 0.58 nmol/kg) dose of 67B06 fusion protein, each single agent control CTLA4-Ig, or anti-OX40L 67B06 (IgG) compared to isotype control to evaluate whether the enhanced potency of the fusion protein observed in vitro was reflected in vivo. As shown in FIG. 14B, fusion protein 67B06 at 2.88 nmol/kg (equivalent to 0.5 mg/kg) offered superior suppression of human IFNγ production compared to the comparators at the same equimolar concentration tested.

### Example 7: In Vivo Efficacy of CTLA4_Anti OX40L Fusion Proteins

This example describes the in vivo efficacy of CTLA4_anti OX40L fusion proteins using xenotransplant, acute, and chronic-graft vs host disease (xeno-GVHD) models.

The xeno-GVHD model is a well characterized model that is dependent on the infused human PBMCs generating a strong xeno-reactive response to the host mice that lack the murine immune system. The xeno-GVHD model has well known limitations in the immune subsets that populate the animal and induce disease. For example, human B cells and myeloid cells do not survive longer than 7-10 days in the mice due to lack of survival factors. Human T cells, however, survive and expand well in the mice such that at Day 7 most of the engrafted cells in the mice are T cells. The expanded T cells are xeno-reactive, infiltrating target organs such as the liver, lung and gut, causing tissue damage with increased inflammatory cytokines like IFNγ, leading to reduction in body weight. If untreated, the mice succumb to the GVHD. Therefore, this model has been useful to evaluate many T cell targeting biologics which can suppress the T cells and alleviate the GVHD, by assessing their effects on reducing GVHD-mediated body weight loss and IFNγ production.

In a first experiment, the CTLA4_anti OX40L fusion protein (67B06) and anti-OX40L (Ref1_Anti-OX40L) were evaluated. Briefly, female NSG mice (n=8 per group) were irradiated (200 rad) and administered 200 μg/mouse (equivalent to 10 mg/kg) of fusion protein, single agent reference comparator, or Isotype control IgG intraperitoneally 3 times a week (Q3W) starting on Day-1. On Day 0, mice received 10 million human peripheral blood mononuclear cells (PBMCs). On Day 7, engrafted human CD45+ cells were phenotyped by flow cytometry. The study was terminated on Day 25, and endpoint measurements of survival, body weight loss, serum IFNγ were measured. Statistical analysis (one-way ANOVA) was performed using Prism GraphPad 8.

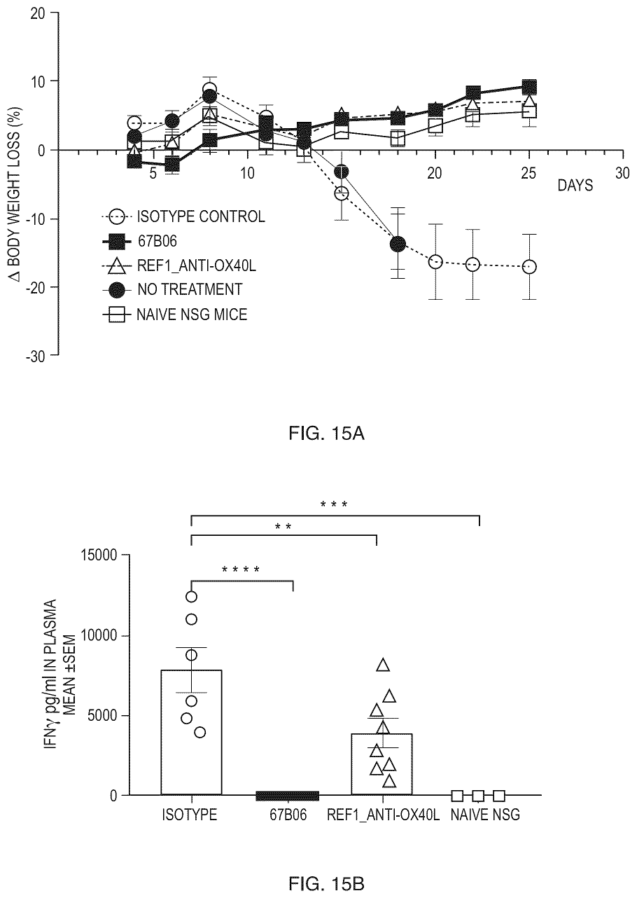

FIG. 15A is a graph showing the effect of CTLA4_anti OX40L fusion protein (67B06), reference monoclonal anti-OX40L antibody (Ref1_Anti_OX40L), or isotype control on weight loss in xeno-GVHD murine model. FIG. 15B is a graph showing the effect of CTLA4_anti OX40L fusion protein (67B06), reference monoclonal anti-OX40L antibody (Ref1_Anti_OX40L), or isotype control on IFNγ production in xeno-GVHD murine model. As shown in FIGS. 15A-15B, although both agents rescued the mice from body weight loss, only 67B06 fusion protein showed superior reduction of IFNγ compared to anti-OX40L treatment alone (FIG. 15B ANOVA: ****p=<0.0001, *p=0.0175, ***p=0.0002).

In a second experiment performed and evaluated essentially as described above, the CTLA4_anti OX40L fusion protein (67B06) and CTLA4-Ig were compared. CTLA4_anti OX40L fusion protein (67B06), CTLA4-Ig (Ref3_CTLA4Ig) or isotype control were administered at a dose of 20 μg/mouse or of 1 μg/mouse (equivalent to 1 mg/kg or 0.05 mg/kg respectively) intraperitoneally once a week (Q1W).

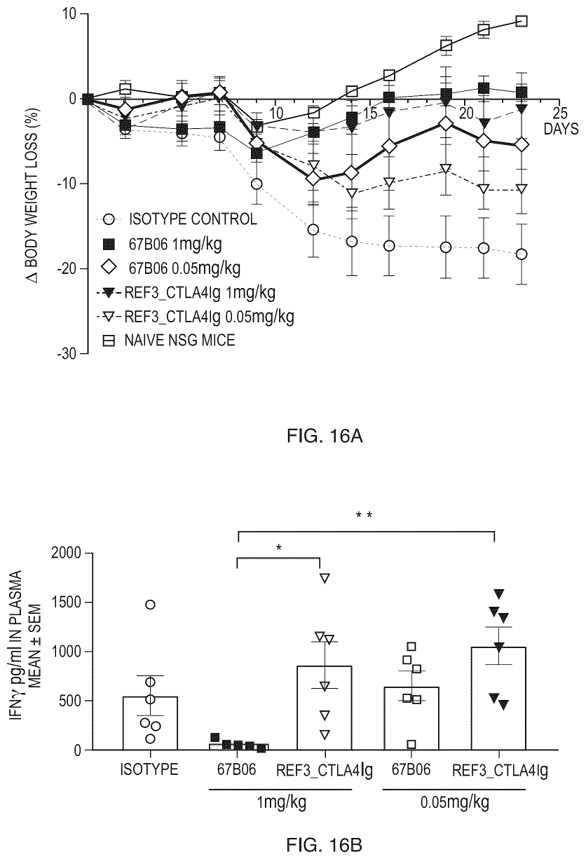

FIG. 16A is a graph showing the effect of CTLA4_anti OX40L fusion protein (67B06), reference CTLA4Ig (Ref3_CTLA4Ig), or isotype control administered i.p. 1 mg/kg or 0.05 mg/kg Q1W on weight loss in xeno-GVHD murine model. FIG. 16B is a graph showing the effect of CTLA4_anti OX40L fusion protein (67B06), reference CTLA4Ig (Ref3_CTLA4Ig), or isotype control on IFNγ production in xeno-GVHD murine model. As shown in FIGS. 16A-16B, although both agents rescued the mice from body weight loss, only 67B06 fusion protein showed superior reduction of IFNγ compared to CTLA4Ig treatment alone (FIG. 16B ANOVA: *p=0.0455, **p=0.0086).

In a further experiment using this model, the effects of CTLA4_anti OX40L fusion protein (67B06) were compared to the effects of CTLA4Ig and anti-Ox40L used in combination. CTLA4_anti-OX40L fusion proteins (67B06), Ref3_CTLA4Ig, Ref1_Anti-Ox40L, and the combination of Ref3_CTLA4Ig and Ref1_Anti-OX40L were administered at a dose of 20 μg/mouse (1 mg/kg) intraperitoneally once a week (Q1W). The study was terminated at day 41.

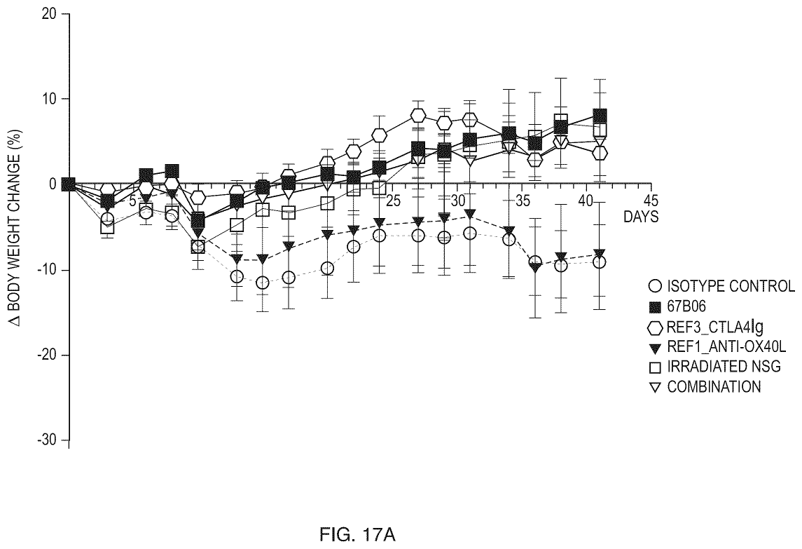

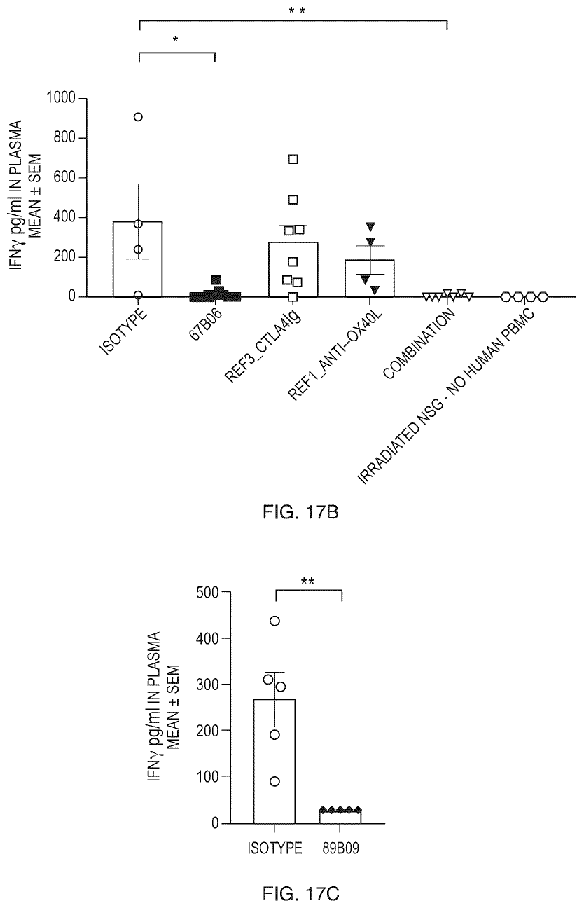

FIG. 17A is a graph showing the effect of CTLA4_anti OX40L fusion proteins (67B06) reference CTLA4Ig (Ref3_CTLA4Ig), anti-OX40L (Ref1_Anti-OX40L), combination (Ref3 and Ref1), or isotype control on weight loss in a xeno-GVHD murine model. FIG. 17B is a graph showing the effect of CTLA4_anti OX40L fusion protein (67B06) reference CTLA4Ig (Ref3_CTLA4Ig), anti-OX40L (Ref1_Anti-OX40L), combination (Ref3 and Ref1) or isotype control on IFNγ production in a xeno-GVHD murine model. As shown in FIGS. 17A-17B, although both fusion protein (67B06), reference CTLA4Ig (Ref3_CTLA4Ig) and combination (Ref3_CTLA4Ig and Ref1_Anti-OX40L) rescued the mice from body weight loss, only fusion protein 67B06 or combination showed superior reduction of IFNγ compared to CTLA4Ig or anti-OX40L treatment alone (FIG. 17B ANOVA: *p=0.0110, **p=0.0084).

In a separate experiment, a different CTLA4_anti-OX40L fusion protein (89B09) is tested and compared to isotype control. Briefly, the fusion protein (89B09) or isotype control are administered at a dose of 20 μg/mouse (1 mg/kg) intraperitoneally once a week (Q1W) starting at Day −1. Similar to the experiments for fusion protein 67B06, mouse body weight loss is monitored and IFNγ production is compared to isotype treatment alone on Day 41 of treatment.

FIG. 17C is a graph showing the effect of CTLA4_anti OX40L fusion protein (89B09) or isotype control on IFNγ production in a xeno-GVHD murine model on Day 12. The graph shows significant reduction in IFNγ production by the fusion protein (ANOVA: **p=0.0036).

In a further acute GVHD model, severe combined immunodeficient (SCID) mice (aged 5 to 10 weeks), are injected with 20 μg rat anti-mouse IL-2RD antibody to deplete endogenous NK cells. The next day, mice receive 2.5 Gy of irradiation using a cesium source. Four hours later, each mouse receives 10 million total human PBMCs by intraperitoneal (IP) injection followed immediately by intravenous (IV) injection of CTLA4_anti OX40L fusion protein, reference monoclonal anti-OX40L antibody or isotype control at various dosages. Alternatively, IV injection of CTLA4_anti OX40L fusion protein, reference monoclonal anti-OX40L antibody or isotype control at various dosages can be delayed until day 3 or day 6 post-IP injection of PBMCs. Mice are weighed every 3 to 4 days and received anti-IL-2RD antibody weekly. At day 12, mice are sacrificed for assessment of gross pathology, flow cytometry analysis of splenocytes, liver and intestine histology, and serum collection for cytokine and antibody analysis.

### INCORPORATION BY REFERENCE

The entire disclosure of each of the patent documents and scientific articles referred to herein is incorporated by reference for all purposes.

### EQUIVALENTS

The disclosure may be embodied in other specific forms without departing from the spirit or essential characteristics thereof. The foregoing embodiments are therefore to be considered in all respects illustrative rather than limiting the disclosure described herein. Various structural elements of the different embodiments and various disclosed method steps may be utilized in various combinations and permutations, and all such variants are to be considered forms of the disclosure. Scope of the disclosure is thus indicated by the appended claims rather than by the foregoing description, and all changes that come within the meaning and range of equivalency of the claims are intended to be embraced therein.

1-118. (canceled)

119. A protein comprising an extracellular domain of CTLA4 or functional fragment thereof and a polypeptide or complex of two or more polypeptides that specifically binds OX40L, wherein the polypeptide or complex of two or more polypeptides that specifically binds OX40L comprises an antigen-binding site.

120. The protein of claim 119, wherein the CTLA4 extracellular domain or functional fragment thereof comprises a sequence having at least 90% sequence identity to SEQ ID NO: 29, SEQ ID NO: 173, or SEQ ID NO: 174.

121. The protein of claim 120, wherein the antigen-binding site comprises a Fab or an antibody.

122. The protein of claim 121, wherein the Fab comprises a polypeptide comprising the sequence of SEQ ID NO: 336 connected to the C-terminus of the Fab heavy chain CH1 domain.

123. The protein of claim 119, further comprising a bridging moiety, wherein the bridging moiety comprises a polypeptide of a human IgG1 Fc domain; optionally wherein the polypeptide of a human IgG1 Fc domain comprises a sequence having at least 90% or 100% sequence identity to an amino acid sequence selected from a group consisting of SEQ ID NO: 178, SEQ ID NO: 237, SEQ ID NO: 238, SEQ ID NO: 214, SEQ ID NO: 216, SEQ ID NO: 179, SEQ ID NO: 180, SEQ ID NO: 239, and SEQ ID NO: 240.

124. The protein of claim 123, wherein the bridging moiety further comprises a hinge polypeptide comprising an amino acid sequence having at least 90% sequence identity to an amino acid sequence selected from a group consisting of SEQ ID NO: 175, SEQ ID NO: 242, SEQ ID NO: 176, SEQ ID NO: 249, SEQ ID NO: 177, SEQ ID NO: 243, SEQ ID NO: 212, SEQ ID NO: 244, SEQ ID NO: 213, SEQ ID NO: 245, SEQ ID NO: 247, and SEQ ID NO: 248.

125. The protein of claim 124, wherein the:
(a) extracellular domain of CTLA4 or functional fragment thereof is connected to the N-terminus of the bridging moiety; or
(b) polypeptide or complex of two or more polypeptides that specifically binds OX40L is connected to the C-terminus of the bridging moiety;
optionally wherein a linker polypeptide connects the polypeptide or complex of two or more polypeptides that specifically binds OX40L to the bridging moiety.

126. The protein of claim 125, further comprising a second polypeptide or complex of two or more polypeptides that specifically binds OX40L, wherein the bridging moiety is a polypeptide of a human IgG1 Fc domain and the polypeptide or complex of two or more polypeptides that specifically binds OX40L is connected to the C-terminus of the polypeptide of the human IgG1 Fc domain and the second polypeptide or complex of two or more polypeptides that specifically binds OX40L is connected to the C-terminus of a second polypeptide of a human IgG1 Fc domain.

127. The protein of claim 126, wherein the second polypeptide or complex of two or more polypeptides that specifically binds OX40L is connected to the bridging moiety by a linker polypeptide; optionally wherein the linker polypeptide comprises a (GGGGS)ₙ (SEQ ID NO: 181) sequence, wherein n is 1 to 12.

128. The protein of claim 121, wherein the antigen-binding site comprises:
(a) a heavy chain variable complementarity-determining region 1 (VHCDR1) comprising an amino acid sequence GX₁SX₂X₃X₄SX₅YY (SEQ ID NO: 222), wherein X₁ is A, G, or V; X₂ is V, or I; X₃ is S or R; X₄ is S, or T; and X₅ is S, or G;
(b) a heavy chain variable complementarity-determining region 2 (VHCDR2) comprising an amino acid sequence selected from the group consisting of:
(1) IX₁YX₂GST (SEQ ID NO: 223) wherein X₁ is Y, or N, and X₂ is S, or G,
(2) X₁DYSGT (SEQ ID NO: 224) wherein X₁ is I or M, and
(3) IGSVDYSGX₁T (SEQ ID NO: 225) wherein X₁ is N, A, or S;
(c) a heavy chain variable complementarity-determining region 3 (VHCDR3) comprising an amino acid sequence selected from the group consisting of:
(1) ARHRGX₁YX₂_{^{FD}}X₃ (SEQ ID NO: 220) wherein X₁ is S or I, X₂ is F or H, and X₃ is I or Y; and
(2) ARERSX₁X₂WYPX₃DY (SEQ ID NO: 221) wherein X₁ is N or S, X₂ is N, D, G or S, and X₃ is I or F;
(d) a light chain variable complementarity-determining region 1 (VLCDR1) comprising an amino acid sequence selected from the group consisting of:
(1) X₁IENKN (SEQ ID NO: 226) wherein X₁ is N or D, and
(2) SX₁RX₂X₃X₄, wherein X₁ is V or L, X₂ is R or N, X₃ is F or Y, and X₄ is F or Y;
(e) a light chain variable complementarity-determining region 2 (VLCDR2) comprising an amino acid sequence selected from the group consisting of RDN, GKD, and RDS; and
(f) a light chain variable complementarity-determining region 3 (VLCDR3) comprising an amino acid sequence selected from the group consisting of:
(1) QVX₁DSX₂X₃VV (SEQ ID NO: 231) wherein X₁ is R or W; X₂ is N, T, or A; and X₃ is I, T, or A, and
(2) NSRDSSGYX1VX2 (SEQ ID NO: 232) wherein X₁ is L or H, and X₂ is L or V.

129. The protein of claim 121, wherein the antigen-binding site comprises:
(i) (a) a VHCDR1 comprising an amino acid sequence of GGSISTSSYY (SEQ ID NO: 77), a VHCDR2 comprising an amino acid sequence of IYYSGST (SEQ ID NO: 78), a VHCDR3 comprising an amino acid sequence of ARHRGSYFFDI (SEQ ID NO: 79), a VLCDR1 comprising an amino acid sequence of DIENKN (SEQ ID NO: 81), a VLCDR2 comprising an amino acid sequence of RDN, and a VLCDR3 comprising an amino acid sequence of QVRDSNIVV (SEQ ID NO: 83);
(b) a VHCDR1 comprising an amino acid sequence of GVSIRSNGYY (SEQ ID NO: 93), a VHCDR2 comprising an amino acid sequence of MDYSGT (SEQ ID NO: 94), a VHCDR3 comprising an amino acid sequence of ARERSNNWYPIDY (SEQ ID NO: 95), a VLCDR1 comprising an amino acid sequence of SVRRFF (SEQ ID NO: 97), a VLCDR2 comprising an amino acid sequence of GKD, and a VLCDR3 comprising an amino acid sequence of NSRDSSGYLVL (SEQ ID NO: 99);
(c) a VHCDR1 comprising an amino acid sequence of GASVSSSSYY (SEQ ID NO: 85), a VHCDR2 comprising an amino acid sequence of INYGGST (SEQ ID NO: 86), a VHCDR3 comprising an amino acid sequence of ARHRGIYHFDY (SEQ ID NO: 87), a VLCDR1 comprising an amino acid sequence of NIENKN (SEQ ID NO: 89), a VLCDR2 comprising an amino acid sequence of RDS, and a VLCDR3 comprising an amino acid sequence of QVWDSNTVV (SEQ ID NO: 91);
(d) a VHCDR1 comprising an amino acid sequence of GGSISSSSYY (SEQ ID NO: 101), a VHCDR2 comprising an amino acid sequence of IGSVDYSGNT (SEQ ID NO: 102), a VHCDR3 comprising an amino acid sequence of ARHRGIYFFDY (SEQ ID NO: 103), a VLCDR1 comprising an amino acid sequence of NIENKN (SEQ ID NO: 89), a VLCDR2 comprising an amino acid sequence of RDS, and a VLCDR3 comprising an amino acid sequence of QVWDSNTVV (SEQ ID NO: 91); or
(e) according to the IMGT unique numbering scheme, VHCDR1, VHCDR2, VHCDR3, VLCDR1, VLCDR2, and VLCDR3 each comprising an amino acid sequence corresponding to the VHCDR1, VHCDR2, VHCDR3, VLCDR1, VLCDR2, and VLCDR3 sequences of a heavy chain variable domain and a light chain variable domain listed in TABLE 3, respectively; and/or
(ii) (a) an amino acid sequence having at least 90% sequence identity to SEQ ID NO: 76, SEQ ID NO: 92, SEQ ID NO: 84, or SEQ ID NO: 100, and an amino acid sequence having at least 90% sequence identity to SEQ ID NO: 80, SEQ ID NO: 96, SEQ ID NO: 88, or SEQ ID NO: 104;
(b) an amino acid sequence having at least 90% sequence identity to SEQ ID NO: 76, and an amino acid sequence having at least 90% sequence identity to SEQ ID NO: 80;
(c) an amino acid sequence having at least 90% sequence identity to SEQ ID NO: 92, and an amino acid sequence having at least 90% sequence identity to SEQ ID NO: 96; or
(d) according to the IMGT unique numbering scheme, a heavy chain variable domain and a light chain variable domain each comprising an amino acid sequence corresponding to the heavy chain variable domain and light chain variable domain sequences of a heavy chain variable domain and light chain variable domain listed in TABLE 3, respectively; and/or
(iii) (a) an amino acid sequence having at least 90% sequence identity to SEQ ID NO: 8, SEQ ID NO: 16, SEQ ID NO: 14, or SEQ ID NO: 18, and an amino acid sequence having at least 90% sequence identity to SEQ ID NO: 7, SEQ ID NO: 15, SEQ ID NO: 13, or SEQ ID NO: 17;
(b) an amino acid sequence having at least 90% sequence identity to SEQ ID NO: 8, and an amino acid sequence having at least 90% sequence identity to SEQ ID NO: 7;
(c) an amino acid sequence having at least 90% sequence identity to SEQ ID NO: 16, and an amino acid sequence having at least 90% sequence identity to SEQ ID NO: 15; or
(d) according to the IMGT unique numbering scheme, a heavy chain and a light chain each comprising an amino acid sequence corresponding to the heavy chain and light chain sequences of a heavy chain and light chain listed in TABLE 3, respectively.

130. The protein of claim 121, wherein the antigen-binding site binds at:
(i) position 60 and/or 83 of the extracellular domain of human OX40L;
(ii) position 17, 18, 19, 20, 21, 23, 26, 28, 60, 83, 110, 111, 112, 113, and 114 of the extracellular domain of human OX40L; or
(iii) position 58, 59, 60, 61, 62, 63, 81, 82, and 83 of the extracellular domain of human OX40L.

131. The protein of claim 121 comprising:
(i) (a) a polypeptide comprising the extracellular domain of CTLA4 comprising SEQ ID NO: 174;
(b) a bridging moiety comprising a sequence comprising of SEQ ID NO: 177 and a sequence comprising of SEQ ID NO: 179; and
(c) an antigen-binding site comprising a VHCDR1 comprising an amino acid sequence of GGSISTSSYY (SEQ ID NO: 77), a VHCDR2 comprising an amino acid sequence of IYYSGST (SEQ ID NO: 78), a VHCDR3 comprising an amino acid sequence of ARHRGSYFFDI (SEQ ID NO: 79), a VLCDR1 comprising an amino acid sequence of DIENKN (SEQ ID NO: 81), a VLCDR2 comprising an amino acid sequence of RDN, and a VLCDR3 comprising an amino acid sequence of QVRDSNIVV (SEQ ID NO: 83); or
(ii) a first arm and a second arm, the first arm comprising:
(a) a first polypeptide comprising the extracellular domain of CTLA4 comprising SEQ ID NO: 174;
(b) a first bridging moiety comprising a sequence comprising of SEQ ID NO: 177 and a sequence comprising of SEQ ID NO: 179; and
(c) a first antigen-binding site comprising, a VHCDR1 comprising an amino acid sequence of GGSISTSSYY (SEQ ID NO: 77), a VHCDR2 comprising an amino acid sequence of IYYSGST (SEQ ID NO: 78), a VHCDR3 comprising an amino acid sequence of ARHRGSYFFDI (SEQ ID NO: 79), a VLCDR1 comprising an amino acid sequence of DIENKN (SEQ ID NO: 81), a VLCDR2 comprising an amino acid sequence of RDN, and a VLCDR3 comprising an amino acid sequence of QVRDSNIVV (SEQ ID NO: 83), and
the second arm comprising:
(a) a second polypeptide comprising the extracellular domain of CTLA4 comprising SEQ ID NO: 174;
(b) a second bridging moiety comprising a sequence comprising of SEQ ID NO: 177 and a sequence comprising of SEQ ID NO: 179; and
(c) a second antigen-binding site comprising a VHCDR1 comprising an amino acid sequence of GGSISTSSYY (SEQ ID NO: 77), a VHCDR2 comprising an amino acid sequence of IYYSGST (SEQ ID NO: 78), a VHCDR3 comprising an amino acid sequence of ARHRGSYFFDI (SEQ ID NO: 79), a VLCDR1 comprising an amino acid sequence of DIENKN (SEQ ID NO: 81), a VLCDR2 comprising an amino acid sequence of RDN, and a VLCDR3 comprising an amino acid sequence of QVRDSNIVV (SEQ ID NO: 83),
wherein the first arm and the second arm dimerize with each other.

132. The protein of claim 121 comprising:
(i) (a) a polypeptide comprising the extracellular domain of CTLA4 comprising SEQ ID NO: 174;
(b) a bridging moiety comprising a sequence comprising of SEQ ID NO: 213 and a sequence comprising of SEQ ID NO: 216; and
(c) an antigen-binding site comprising a VHCDR1 comprising an amino acid sequence of GVSIRSNGYY (SEQ ID NO: 93), a VHCDR2 comprising an amino acid sequence of MDYSGT (SEQ ID NO: 94), a VHCDR3 comprising an amino acid sequence of ARERSNNWYPIDY (SEQ ID NO: 95), a VLCDR1 comprising an amino acid sequence of SVRRFF (SEQ ID NO: 97), a VLCDR2 comprising an amino acid sequence of GKD, and a VLCDR3 comprising an amino acid sequence of NSRDSSGYLVL (SEQ ID NO: 99);
(ii) a first arm and a second arm, the first arm comprising:
(a) a first polypeptide comprising the extracellular domain of CTLA4 comprising SEQ ID NO: 174;
(b) a first bridging moiety comprising a sequence comprising of SEQ ID NO: 213 and a sequence comprising of SEQ ID NO: 216; and
(c) a first antigen-binding site comprising a VHCDR1 comprising an amino acid sequence of GVSIRSNGYY (SEQ ID NO: 93), a VHCDR2 comprising an amino acid sequence of MDYSGT (SEQ ID NO: 94), a VHCDR3 comprising an amino acid sequence of ARERSNNWYPIDY (SEQ ID NO: 95), a VLCDR1 comprising an amino acid sequence of SVRRFF (SEQ ID NO: 97), a VLCDR2 comprising an amino acid sequence of GKD, and a VLCDR3 comprising an amino acid sequence of NSRDSSGYLVL (SEQ ID NO: 99); and
the second arm comprising:
(a) a second polypeptide comprising the extracellular domain of CTLA4 comprising SEQ ID NO: 174;
(b) a second bridging moiety comprising a sequence comprising of SEQ ID NO: 213 and a sequence comprising of SEQ ID NO: 216; and
(c) a second antigen-binding site comprising a VHCDR1 comprising an amino acid sequence of GVSIRSNGYY (SEQ ID NO: 93), a VHCDR2 comprising an amino acid sequence of MDYSGT (SEQ ID NO: 94), a VHCDR3 comprising an amino acid sequence of ARERSNNWYPIDY (SEQ ID NO: 95), a VLCDR1 comprising an amino acid sequence of SVRRFF (SEQ ID NO: 97), a VLCDR2 comprising an amino acid sequence of GKD, and a VLCDR3 comprising an amino acid sequence of NSRDSSGYLVL (SEQ ID NO: 99),
wherein the first arm and the second arm dimerize with each other;
(iii) (a) a polypeptide comprising the extracellular domain of CTLA4 comprising SEQ ID NO: 174;
(b) a bridging moiety comprising a sequence comprising of SEQ ID NO: 213 and a sequence comprising of SEQ ID NO: 214; and
(c) an antigen-binding site comprising a VHCDR1 comprising an amino acid sequence of GASVSSSSYY (SEQ ID NO: 85), a VHCDR2 comprising an amino acid sequence of INYGGST (SEQ ID NO: 86), a VHCDR3 comprising an amino acid sequence of ARHRGIYHFDY (SEQ ID NO: 87), a VLCDR1 comprising an amino acid sequence of NIENKN (SEQ ID NO: 89), a VLCDR2 comprising an amino acid sequence of RDS, and a VLCDR3 comprising an amino acid sequence of QVWDSNTVV (SEQ ID NO: 91);
(iv) a first arm and a second arm, the first arm comprising:
(a) a first polypeptide complex the extracellular domain of CTLA4 comprising SEQ ID NO: 174;
(b) a first bridging moiety comprising a sequence comprising of SEQ ID NO: 213 and a sequence comprising of SEQ ID NO: 214; and
(c) a first antigen-binding site comprising a VHCDR1 comprising an amino acid sequence of GASVSSSSYY (SEQ ID NO: 85), a VHCDR2 comprising an amino acid sequence of INYGGST (SEQ ID NO: 86), a VHCDR3 comprising an amino acid sequence of ARHRGIYHFDY (SEQ ID NO: 87), a VLCDR1 comprising an amino acid sequence of NIENKN (SEQ ID NO: 89), a VLCDR2 comprising an amino acid sequence of RDS, and a VLCDR3 comprising an amino acid sequence of QVWDSNTVV (SEQ ID NO: 91), and
the second arm comprising:
(a) a second polypeptide comprising the extracellular domain of CTLA4 comprising SEQ ID NO: 174;
(b) a second bridging moiety comprising a sequence comprising of SEQ ID NO: 213 and a sequence comprising of SEQ ID NO: 214; and
(c) a second antigen-binding site comprising a VHCDR1 comprising an amino acid sequence of GASVSSSSYY (SEQ ID NO: 85), a VHCDR2 comprising an amino acid sequence of INYGGST (SEQ ID NO: 86), a VHCDR3 comprising an amino acid sequence of ARHRGIYHFDY (SEQ ID NO: 87), a VLCDR1 comprising an amino acid sequence of NIENKN (SEQ ID NO: 89), a VLCDR2 comprising an amino acid sequence of RDS, and a VLCDR3 comprising an amino acid sequence of QVWDSNTVV (SEQ ID NO: 91),
wherein the first arm and the second arm dimerize with each other;
(v) (a) a polypeptide comprising the extracellular domain of CTLA4 comprising SEQ ID NO: 174;
(b) a bridging moiety comprising a sequence comprising of SEQ ID NO: 213 and a sequence comprising of SEQ ID NO: 216; and
(c) an antigen-binding site comprising a VHCDR1 comprising an amino acid sequence of GGSISSSSYY (SEQ ID NO: 101), a VHCDR2 comprising an amino acid sequence of IGSVDYSGNT (SEQ ID NO: 102), a VHCDR3 comprising an amino acid sequence of ARHRGIYFFDY (SEQ ID NO: 103), a VLCDR1 comprising an amino acid sequence of NIENKN (SEQ ID NO: 89), a VLCDR2 comprising an amino acid sequence of RDS, and a VLCDR3 comprising an amino acid sequence of QVWDSNTVV (SEQ ID NO: 91);
(vi) a first arm and a second arm, the first arm comprising:
(a) a first polypeptide comprising the extracellular domain of CTLA4 comprising SEQ ID NO: 174;
(b) a first bridging moiety comprising a sequence comprising of SEQ ID NO: 213 and a sequence comprising of SEQ ID NO: 216; and
(c) a first antigen-binding site comprising a VHCDR1 comprising an amino acid sequence of GGSISSSSYY (SEQ ID NO: 101), a VHCDR2 comprising an amino acid sequence of IGSVDYSGNT (SEQ ID NO: 102), a VHCDR3 comprising an amino acid sequence of ARHRGIYFFDY (SEQ ID NO: 103), a VLCDR1 comprising an amino acid sequence of NIENKN (SEQ ID NO: 89), a VLCDR2 comprising an amino acid sequence of RDS, and a VLCDR3 comprising an amino acid sequence of QVWDSNTVV (SEQ ID NO: 91), and
the second arm comprising:
(a) a second polypeptide comprising the extracellular domain of CTLA4 comprising SEQ ID NO: 174;
(b) a second bridging moiety comprising a sequence comprising of SEQ ID NO: 213 and a sequence comprising of SEQ ID NO: 216; and
(c) a second antigen-binding site comprising a VHCDR1 comprising an amino acid sequence of GGSISSSSYY (SEQ ID NO: 101), a VHCDR2 comprising an amino acid sequence of IGSVDYSGNT (SEQ ID NO: 102), a VHCDR3 comprising an amino acid sequence of ARHRGIYFFDY (SEQ ID NO: 103), a VLCDR1 comprising an amino acid sequence of NIENKN (SEQ ID NO: 89), a VLCDR2 comprising an amino acid sequence of RDS, and a VLCDR3 comprising an amino acid sequence of QVWDSNTVV (SEQ ID NO: 91),
wherein the first arm and the second arm dimerize with each other;
(vii) (a) a polypeptide comprising the extracellular domain of CTLA4 comprising SEQ ID NO: 174;
(b) a bridging moiety comprising a sequence comprising of SEQ ID NO: 213 and a sequence comprising of SEQ ID NO: 216; and
(c) an antigen-binding site comprising a VHCDR1 comprising an amino acid sequence of GGSISTSSYY (SEQ ID NO: 77), a VHCDR2 comprising an amino acid sequence of IYYSGST (SEQ ID NO: 78), a VHCDR3 comprising an amino acid sequence of ARHRGSYFFDI (SEQ ID NO: 79), a VLCDR1 comprising an amino acid sequence of DIENKN (SEQ ID NO: 81), a VLCDR2 comprising an amino acid sequence of RDN, and a VLCDR3 comprising an amino acid sequence of QVRDSNIVV (SEQ ID NO: 83);
(viii) a first arm and a second arm, the first arm comprising:
(a) a first polypeptide comprising the extracellular domain of CTLA4 comprising SEQ ID NO: 174;
(b) a first bridging moiety comprising a sequence comprising of SEQ ID NO: 213 and a sequence comprising of SEQ ID NO: 216; and
(c) a first antigen-binding site comprising a VHCDR1 comprising an amino acid sequence of GGSISTSSYY (SEQ ID NO: 77), a VHCDR2 comprising an amino acid sequence of IYYSGST (SEQ ID NO: 78), a VHCDR3 comprising an amino acid sequence of ARHRGSYFFDI (SEQ ID NO: 79), a VLCDR1 comprising an amino acid sequence of DIENKN (SEQ ID NO: 81), a VLCDR2 comprising an amino acid sequence of RDN, and a VLCDR3 comprising an amino acid sequence of QVRDSNIVV (SEQ ID NO: 83), and
the second arm comprising:
(a) a second polypeptide comprising the extracellular domain of CTLA4 comprising SEQ ID NO: 174;
(b) a second bridging moiety comprising a sequence comprising of SEQ ID NO: 213 and a sequence comprising of SEQ ID NO: 216; and
(c) a second antigen-binding site comprising a VHCDR1 comprising an amino acid sequence of GGSISTSSYY (SEQ ID NO: 77), a VHCDR2 comprising an amino acid sequence of IYYSGST (SEQ ID NO: 78), a VHCDR3 comprising an amino acid sequence of ARHRGSYFFDI (SEQ ID NO: 79), a VLCDR1 comprising an amino acid sequence of DIENKN (SEQ ID NO: 81), a VLCDR2 comprising an amino acid sequence of RDN, and a VLCDR3 comprising an amino acid sequence of QVRDSNIVV (SEQ ID NO: 83),
wherein the first arm and the second arm dimerize with each other;
(ix) (a) a polypeptide comprising the extracellular domain of CTLA4 comprising a sequence selected from a group consisting of SEQ ID NO: 174, SEQ ID NO: 119, SEQ ID NO: 215, SEQ ID NO: 233, SEQ ID NO: 234, and SEQ ID NO: 235;
(b) a bridging moiety comprising a sequence selected from a group consisting of SEQ ID NO: 175, SEQ ID NO: 176, SEQ ID NO: 177, SEQ ID NO: 212, SEQ ID NO: 213, and SEQ ID NO: 248, and a sequence selected from a group consisting of SEQ ID NO: 178, SEQ ID NO: 237, SEQ ID NO: 214, SEQ ID NO: 216, SEQ ID NO: 179, SEQ ID NO: 180, SEQ ID NO: 239, and SEQ ID NO: 240; and
(c) an antigen-binding site comprising, according to the IMGT unique numbering scheme, VHCDR1, VHCDR2, VHCDR3, VLCDR1, VLCDR2, and VLCDR3 each comprising an amino acid sequence corresponding to the VHCDR1, VHCDR2, VHCDR3, VLCDR1, VLCDR2, and VLCDR3 sequences of a heavy chain variable domain and a light chain variable domain listed in TABLE 3, respectively; or
(x) a first arm and a second arm, the first arm comprising:
(a) a first polypeptide comprising the extracellular domain of CTLA4 comprising a sequence selected from a group consisting of SEQ ID NO: 174, SEQ ID NO: 119, SEQ ID NO: 215, SEQ ID NO: 233, SEQ ID NO: 234, and SEQ ID NO: 235;
(b) a first bridging moiety comprising a sequence selected from a group consisting of SEQ ID NO: 175, SEQ ID NO: 176, SEQ ID NO: 177, SEQ ID NO: 212, SEQ ID NO: 213, and SEQ ID NO: 248, and a sequence selected from a group consisting of SEQ ID NO: 178, SEQ ID NO: 237, SEQ ID NO: 214, SEQ ID NO: 216, SEQ ID NO: 179, SEQ ID NO: 180, SEQ ID NO: 239, and SEQ ID NO: 240; and
(c) a first antigen-binding site comprising, according to the IMGT unique numbering scheme, VHCDR1, VHCDR2, VHCDR3, VLCDR1, VLCDR2, and VLCDR3 each comprising an amino acid sequence corresponding to the VHCDR1, VHCDR2, VHCDR3, VLCDR1, VLCDR2, and VLCDR3 sequences of a heavy chain variable domain and a light chain variable domain listed in TABLE 3, respectively, and
the second arm comprising:
(a) a second polypeptide comprising the extracellular domain of CTLA4 comprising a sequence selected from a group consisting of SEQ ID NO: 174, SEQ ID NO: 119, SEQ ID NO: 215, SEQ ID NO: 233, SEQ ID NO: 234, and SEQ ID NO: 235;
(b) a second bridging moiety comprising a sequence selected from a group consisting of SEQ ID NO: 175, SEQ ID NO: 176, SEQ ID NO: 177, SEQ ID NO: 212, SEQ ID NO: 213, and SEQ ID NO: 248, and a sequence selected from a group consisting of SEQ ID NO: 178, SEQ ID NO: 237, SEQ ID NO: 214, SEQ ID NO: 216, SEQ ID NO: 179, SEQ ID NO: 180, SEQ ID NO: 239, and SEQ ID NO: 240; and
(c) a second antigen-binding site comprising, according to the IMGT unique numbering scheme, VHCDR1, VHCDR2, VHCDR3, VLCDR1, VLCDR2, and VLCDR3 each comprising an amino acid sequence corresponding to the VHCDR1, VHCDR2, VHCDR3, VLCDR1, VLCDR2, and VLCDR3 sequences of a heavy chain variable domain and a light chain variable domain listed in TABLE 3, respectively,
wherein the first arm and the second arm dimerize with each other.

133. The protein of claim 132, wherein:
(i) the antigen-binding site further comprises a partial hinge polypeptide comprising the sequence of SEQ ID NO: 336; or
(ii) the first antigen-binding site and second antigen-binding site both further comprise a partial hinge polypeptide comprising the sequence of SEQ ID NO: 336.

134. The protein of claim 121 comprising:
(i) an amino acid sequence at least 90% identical to the amino acid sequence selected from the group consisting of SEQ ID NO: 40, SEQ ID NO: 62, SEQ ID NO: 64, SEQ ID NO: 182, SEQ ID NO: 183, SEQ ID NO: 184, SEQ ID NO: 185, SEQ ID NO: 186, SEQ ID NO: 48, SEQ ID NO: 68, SEQ ID NO: 187, SEQ ID NO: 188, SEQ ID NO: 189, SEQ ID NO: 190, SEQ ID NO: 191, SEQ ID NO: 46, SEQ ID NO: 66, SEQ ID NO: 192, SEQ ID NO: 193, SEQ ID NO: 194, SEQ ID NO: 195, SEQ ID NO: 196, SEQ ID NO: 50, SEQ ID NO: 70, SEQ ID NO: 197, SEQ ID NO: 198, SEQ ID NO: 199, SEQ ID NO: 200, SEQ ID NO: 201, SEQ ID NO: 38, SEQ ID NO: 73, SEQ ID NO: 42, SEQ ID NO: 44, SEQ ID NO: 52, SEQ ID NO: 54, SEQ ID NO: 56, SEQ ID NO: 58, SEQ ID NO: 60, SEQ ID NO: 37, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308, SEQ ID NO: 309, SEQ ID NO: 310, SEQ ID NO: 311, SEQ ID NO: 312, SEQ ID NO: 313, SEQ ID NO: 314, SEQ ID NO: 315, SEQ ID NO: 316, SEQ ID NO: 317, SEQ ID NO: 318, SEQ ID NO: 319, SEQ ID NO: 320, SEQ ID NO: 321, SEQ ID NO: 322, SEQ ID NO: 323, SEQ ID NO: 324, SEQ ID NO: 325, SEQ ID NO: 326, SEQ ID NO: 327, SEQ ID NO: 328, SEQ ID NO: 329, SEQ ID NO: 330, SEQ ID NO: 331, SEQ ID NO: 332, SEQ ID NO: 333, SEQ ID NO: 334, and SEQ ID NO: 335; or
(ii) polypeptides comprising amino acid sequences at least 90% identical to the amino acid sequences of:
SEQ ID NO: 40 and SEQ ID NO: 39;
SEQ ID NO: 62 and SEQ ID NO: 61;
SEQ ID NO: 64 and SEQ ID NO: 63;
SEQ ID NO: 182 and SEQ ID NO: 63;
SEQ ID NO: 183 and SEQ ID NO: 63;
SEQ ID NO: 184 and SEQ ID NO: 63;
SEQ ID NO: 185 and SEQ ID NO: 63;
SEQ ID NO: 186 and SEQ ID NO: 63;
SEQ ID NO: 48 and SEQ ID NO: 47;
SEQ ID NO: 68 and SEQ ID NO: 67;
SEQ ID NO: 187 and SEQ ID NO: 67;
SEQ ID NO: 188 and SEQ ID NO: 67;
SEQ ID NO: 189 and SEQ ID NO: 67;
SEQ ID NO: 190 and SEQ ID NO: 67;
SEQ ID NO: 191 and SEQ ID NO: 67;
SEQ ID NO: 46 and SEQ ID NO: 45;
SEQ ID NO: 66 and SEQ ID NO: 65;
SEQ ID NO: 192 and SEQ ID NO: 65;
SEQ ID NO: 193 and SEQ ID NO: 65;
SEQ ID NO: 194 and SEQ ID NO: 65;
SEQ ID NO: 195 and SEQ ID NO: 65;
SEQ ID NO: 196 and SEQ ID NO: 65;
SEQ ID NO: 50 and SEQ ID NO: 49;
SEQ ID NO: 70 and SEQ ID NO: 69;
SEQ ID NO: 197 and SEQ ID NO: 69;
SEQ ID NO: 198 and SEQ ID NO: 69;
SEQ ID NO: 199 and SEQ ID NO: 69;
SEQ ID NO: 200 and SEQ ID NO: 69;
SEQ ID NO: 201 and SEQ ID NO: 69;
SEQ ID NO: 38 and SEQ ID NO: 5;
SEQ ID NO: 73 and SEQ ID NO: 5;
SEQ ID NO: 42 and SEQ ID NO: 41;
SEQ ID NO: 44 and SEQ ID NO: 43;
SEQ ID NO: 52 and SEQ ID NO: 51;
SEQ ID NO: 54 and SEQ ID NO: 53;
SEQ ID NO: 56 and SEQ ID NO: 55;
SEQ ID NO: 58 and SEQ ID NO: 57;
SEQ ID NO: 60 and SEQ ID NO: 59;
SEQ ID NO: 37 and SEQ ID NO: 35;
SEQ ID NO: 256 and SEQ ID NO: 63;
SEQ ID NO: 257 and SEQ ID NO: 63;
SEQ ID NO: 258 and SEQ ID NO: 63;
SEQ ID NO: 259 and SEQ ID NO: 63;
SEQ ID NO: 260 and SEQ ID NO: 63;
SEQ ID NO: 261 and SEQ ID NO: 63;
SEQ ID NO: 262 and SEQ ID NO: 63;
SEQ ID NO: 263 and SEQ ID NO: 63;
SEQ ID NO: 264 and SEQ ID NO: 63;
SEQ ID NO: 265 and SEQ ID NO: 63;
SEQ ID NO: 266 and SEQ ID NO: 63;
SEQ ID NO: 267 and SEQ ID NO: 63;
SEQ ID NO: 268 and SEQ ID NO: 63;
SEQ ID NO: 269 and SEQ ID NO: 63;
SEQ ID NO: 270 and SEQ ID NO: 63;
SEQ ID NO: 271 and SEQ ID NO: 63;
SEQ ID NO: 272 and SEQ ID NO: 63;
SEQ ID NO: 273 and SEQ ID NO: 63;
SEQ ID NO: 274 and SEQ ID NO: 63;
SEQ ID NO: 275 and SEQ ID NO: 63;
SEQ ID NO: 276 and SEQ ID NO: 63;
SEQ ID NO: 277 and SEQ ID NO: 63;
SEQ ID NO: 278 and SEQ ID NO: 63;
SEQ ID NO: 279 and SEQ ID NO: 63;
SEQ ID NO: 280 and SEQ ID NO: 63;
SEQ ID NO: 281 and SEQ ID NO: 63;
SEQ ID NO: 282 and SEQ ID NO: 63;
SEQ ID NO: 283 and SEQ ID NO: 63;
SEQ ID NO: 284 and SEQ ID NO: 63;
SEQ ID NO: 285 and SEQ ID NO: 63;
SEQ ID NO: 286 and SEQ ID NO: 63;
SEQ ID NO: 287 and SEQ ID NO: 63;
SEQ ID NO: 288 and SEQ ID NO: 63;
SEQ ID NO: 289 and SEQ ID NO: 63;
SEQ ID NO: 290 and SEQ ID NO: 63;
SEQ ID NO: 291 and SEQ ID NO: 63;
SEQ ID NO: 292 and SEQ ID NO: 63;
SEQ ID NO: 293 and SEQ ID NO: 63;
SEQ ID NO: 294 and SEQ ID NO: 67;
SEQ ID NO: 295 and SEQ ID NO: 67;
SEQ ID NO: 296 and SEQ ID NO: 67;
SEQ ID NO: 297 and SEQ ID NO: 67;
SEQ ID NO: 298, and SEQ ID NO: 67;
SEQ ID NO: 299 and SEQ ID NO: 67;
SEQ ID NO: 300 and SEQ ID NO: 67;
SEQ ID NO: 301 and SEQ ID NO: 67;
SEQ ID NO: 302 and SEQ ID NO: 67;
SEQ ID NO: 303 and SEQ ID NO: 67;
SEQ ID NO: 304 and SEQ ID NO: 67;
SEQ ID NO: 305 and SEQ ID NO: 67;
SEQ ID NO: 306 and SEQ ID NO: 67;
SEQ ID NO: 307 and SEQ ID NO: 67;
SEQ ID NO: 308 and SEQ ID NO: 67;
SEQ ID NO: 309 and SEQ ID NO: 67;
SEQ ID NO: 310 and SEQ ID NO: 67;
SEQ ID NO: 311 and SEQ ID NO: 67;
SEQ ID NO: 312 and SEQ ID NO: 67;
SEQ ID NO: 313 and SEQ ID NO: 67;
SEQ ID NO: 314 and SEQ ID NO: 67;
SEQ ID NO: 315 and SEQ ID NO: 67;
SEQ ID NO: 316 and SEQ ID NO: 67;
SEQ ID NO: 317 and SEQ ID NO: 67;
SEQ ID NO: 318 and SEQ ID NO: 67;
SEQ ID NO: 319 and SEQ ID NO: 67;
SEQ ID NO: 320 and SEQ ID NO: 67;
SEQ ID NO: 321 and SEQ ID NO: 67;
SEQ ID NO: 322 and SEQ ID NO: 67;
SEQ ID NO: 323 and SEQ ID NO: 67;
SEQ ID NO: 324 and SEQ ID NO: 67;
SEQ ID NO: 325 and SEQ ID NO: 67;
SEQ ID NO: 326 and SEQ ID NO: 67;
SEQ ID NO: 327 and SEQ ID NO: 67;
SEQ ID NO: 328 and SEQ ID NO: 67;
SEQ ID NO: 329 and SEQ ID NO: 67;
SEQ ID NO: 330 and SEQ ID NO: 67;
SEQ ID NO: 331 and SEQ ID NO: 67;
SEQ ID NO: 332 and SEQ ID NO: 253;
SEQ ID NO: 333 and SEQ ID NO: 255;
SEQ ID NO: 334 and SEQ ID NO: 253; or
SEQ ID NO: 335 and SEQ ID NO: 255.

135. The protein of claim 121 comprising:
(i) polypeptides comprising amino acid sequences of SEQ ID NO: 62 and SEQ ID NO: 61;
(ii) two polypeptides comprising amino acid sequences of SEQ ID NO: 62 and two polypeptides comprising amino acid sequences of SEQ ID NO: 61;
(iii) polypeptides comprising amino acid sequences of SEQ ID NO: 63 and SEQ ID NO: 64;
(iv) two polypeptides comprising amino acid sequences of SEQ ID NO: 63 and two polypeptides comprising amino acid sequences of SEQ ID NO: 64;
(v) polypeptides comprising amino acid sequences of SEQ ID NO: 68 and SEQ ID NO: 67; or
(vi) two polypeptides comprising amino acid sequences of SEQ ID NO: 68 and two polypeptides comprising amino acid sequences of SEQ ID NO: 67.

136. The protein of claim 119, wherein:
(i) the protein has an IC₅₀ less than 12 nM as measured in an OX40L neutralization/OX40-HEK reporter assay; optionally wherein the protein has an IC₅₀ of 5 nM to 12 nM as measured in an OX40L neutralization/OX40-HEK reporter assay; further optionally wherein the protein has an IC₅₀ of 5 nM to 9 nM as measured in an OX40L neutralization/OX40-HEK reporter assay; and/or
(ii) the protein has an IC₅₀ of 2 nM to 12 nM as measured in a primary T cell activation assay; optionally wherein the protein has an IC₅₀ of 2 nM to 9.5 nM as measured in a primary T cell activation assay; and/or
(iii) the protein has a K_{D} for OX40L binding of less than 75 nM as measured in a Biacore assay; optionally the protein has a K_{D} for OX40L binding of 20 nM to 75 nM as measured in a Biacore assay; and/or
(iv) the protein significantly inhibits at least one of IL-2, IFNγ, IL-6, and TNFα production by cells in an in vitro mixed lymphocyte reaction assay as compared to cells treated under the same conditions but in the absence of protein, or in the presence of a human CTLA4 extracellular domain fused at the N-terminus of a human IgG1 Fc domain (CTLA4-Ig) and an anti-OX40L antibody, presented individually or in combination of the two proteins presented separately; and/or
(v) the protein significantly inhibits alloreactive CD4+ and CD8+ T cell proliferation in an in vitro mixed lymphocyte reaction assay as compared to alloreactive CD4+ and CD8+ T cells treated under the same conditions but in the absence of protein, or in the presence of a CTLA4-Ig and/or an anti-OX40L antibody; and/or
(vi) the protein preferentially inhibits alloreactive CD4+ and CD8+ T cell proliferation as compared to regulatory T cell (T_{reg}) proliferation in an in vitro mixed lymphocyte reaction assay as compared to alloreactive CD4+ and CD8+ T cells treated under the same conditions but in the absence of protein, or in the presence of a CTLA4-Ig and/or an anti-OX40L antibody; and/or
(vii) the protein significantly enhances T_{reg} suppressive function in an in vitro mixed lymphocyte reaction assay as compared to T_{reg} cells treated under the same conditions but in the absence of protein, or in the presence of a CTLA4-Ig and/or an anti-OX40L antibody; and/or
(viii) the protein significantly inhibits alloreactive CD4+ and CD8+ T cell proliferation in an adoptive transfer assay in humanized mice as compared to alloreactive CD4+ and CD8+ T cells in mice treated under the same conditions but in the absence of protein, or in the presence of a CTLA4-Ig and/or an anti-OX40L antibody; and/or
(ix) the protein significantly inhibits serum IFNγ levels in an adoptive transfer assay in humanized mice as compared to mice treated under the same conditions but in the absence of protein, or in the presence of a CTLA4-Ig and/or an anti-OX40L antibody; and/or
(x) the protein is not significantly internalized into myeloid-derived dendritic cells (MDDCs); and/or
(xi) the protein is cross-reactive with cynomolgus OX40L; and/or
(xii) the protein is not cross-reactive with mouse, rabbit, or rat OX40L; and/or
(xiii) the protein is cross reactive with cynomolgus CD80 and CD86.

137. A formulation comprising the protein of claim 119 and a pharmaceutically acceptable carrier.

138. A nucleic acid encoding the protein of claim 119.

139. A cell comprising one or more nucleic acids encoding the protein of claim 119.

140. A method of treating an autoimmune disease in a patient, the method comprising administering to the patient the protein of claim 119.

141. The method of claim 140, wherein the autoimmune disease is selected from the group consisting of rheumatoid arthritis, juvenile idiopathic arthritis, psoriatic arthritis, atopic dermatitis, Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Bal6 disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Cutaneous Lupus, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Thyroid eye disease (TED), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, and Vogt-Koyanagi-Harada Disease.

142. A method of treating graft versus host disease (GVHD) in a patient, the method comprising administering to a patient a protein of claim 119.

143. An antibody or functional fragment thereof comprising:
(i) a heavy chain variable complementarity-determining region 1 (VHCDR1) comprising an amino acid sequence of GGSISTSSYY (SEQ ID NO: 77), a heavy chain variable complementarity-determining region 2 (VHCDR2) comprising an amino acid sequence of IYYSGST (SEQ ID NO: 78), a heavy chain variable complementarity-determining region 3 (VHCDR3) comprising an amino acid sequence of ARHRGSYFFDI (SEQ ID NO: 79), a light chain variable complementarity-determining region 1 (VLCDR1) comprising an amino acid sequence of DIENKN (SEQ ID NO: 81), a light chain variable complementarity-determining region 2 (VLCDR2) comprising an amino acid sequence of RDN, and a light chain variable complementarity-determining region 3 (VLCDR3) comprising an amino acid sequence of QVRDSNIVV (SEQ ID NO: 83) (according to the IMGT unique numbering scheme); or
(ii) a VHCDR1 comprising an amino acid sequence of GVSIRSNGYY (SEQ ID NO: 93), a VHCDR2 comprising an amino acid sequence of MDYSGT (SEQ ID NO: 94), a VHCDR3 comprising an amino acid sequence of ARERSNNWYPIDY (SEQ ID NO: 95), a VLCDR1 comprising an amino acid sequence of SVRRFF (SEQ ID NO: 97), a VLCDR2 comprising an amino acid sequence of GKD, and a VLCDR3 comprising an amino acid sequence of NSRDSSGYLVL (SEQ ID NO: 99) (according to the IMGT unique numbering scheme); or
(iii) a VHCDR1 comprising an amino acid sequence of GASVSSSSYY (SEQ ID NO: 85), a VHCDR2 comprising an amino acid sequence of INYGGST (SEQ ID NO: 86), a VHCDR3 comprising an amino acid sequence of ARHRGIYHFDY (SEQ ID NO: 87), a VLCDR1 comprising an amino acid sequence of NIENKN (SEQ ID NO: 89), a VLCDR2 comprising an amino acid sequence of RDS, and a VLCDR3 comprising an amino acid sequence of QVWDSNTVV (SEQ ID NO: 91) (according to the IMGT unique numbering scheme); or
(iv) a VHCDR1 comprising an amino acid sequence of GGSISSSSYY (SEQ ID NO: 101), a VHCDR2 comprising an amino acid sequence of IGSVDYSGNT (SEQ ID NO: 102), a VHCDR3 comprising an amino acid sequence of ARHRGIYFFDY (SEQ ID NO: 103), a VLCDR1 comprising an amino acid sequence of NIENKN (SEQ ID NO: 89), a VLCDR2 comprising an amino acid sequence of RDS, and a VLCDR3 comprising an amino acid sequence of QVWDSNTVV (SEQ ID NO: 91) (according to the IMGT unique numbering scheme); or
(v) a VHCDR1 comprising an amino acid sequence of GGSISSSSYY (SEQ ID NO: 109), a VHCDR2 comprising an amino acid sequence of IGSVDYSGNT (SEQ ID NO: 110), a VHCDR3 comprising an amino acid sequence of ARHRGIYFFDY (SEQ ID NO: 103), a VLCDR1 comprising an amino acid sequence of NIENKN (SEQ ID NO: 89), a VLCDR2 comprising an amino acid sequence of RDS, and a VLCDR3 comprising an amino acid sequence of QVWDSNTVI (SEQ ID NO: 115) (according to the IMGT unique numbering scheme); or
(vi) a VHCDR1 comprising an amino acid sequence of GGSIDTSSQY (SEQ ID NO: 117), a VHCDR2 comprising an amino acid sequence of IYFSGST (SEQ ID NO: 118), a VHCDR3 comprising an amino acid sequence of ARHRGIYFFDY (SEQ ID NO: 103), a VLCDR1 comprising an amino acid sequence of NIENKN (SEQ ID NO: 89), a VLCDR2 comprising an amino acid sequence of RDT, and a VLCDR3 comprising an amino acid sequence of QVWDSNTVI (SEQ ID NO: 123) (according to the IMGT unique numbering scheme); or
(vii) a VHCDR1 comprising an amino acid sequence of GGSISSGSFY (SEQ ID NO: 125), a VHCDR2 comprising an amino acid sequence of IYYSGST (SEQ ID NO: 126), a VHCDR3 comprising an amino acid sequence of ARERSSSWYPFDY (SEQ ID NO: 127), a VLCDR1 comprising an amino acid sequence of SLRNYF (SEQ ID NO: 129), a VLCDR2 comprising an amino acid sequence of GKN, and a VLCDR3 comprising an amino acid sequence of NSRDSSGYHVV (SEQ ID NO: 131) (according to the IMGT unique numbering scheme); or
(viii) a VHCDR1 comprising an amino acid sequence of GFTFSNYA (SEQ ID NO: 133), a VHCDR2 comprising an amino acid sequence of ISGSGDST (SEQ ID NO: 134), a VHCDR3 comprising an amino acid sequence of AKDRTPVYGLDV (SEQ ID NO: 135), a VLCDR1 comprising an amino acid sequence of NIGRKN (SEQ ID NO: 137), a VLCDR2 comprising an amino acid sequence of GDS, and a VLCDR3 comprising an amino acid sequence of QVWDSSTV (SEQ ID NO: 139) (according to the IMGT unique numbering scheme); or
(ix) a VHCDR1 comprising an amino acid sequence of GFTFSSYA (SEQ ID NO: 141), a VHCDR2 comprising an amino acid sequence of ISGSGGST (SEQ ID NO: 142), a VHCDR3 comprising an amino acid sequence of AKDLGFYSTWDTDY (SEQ ID NO: 143), a VLCDR1 comprising an amino acid sequence of QGIRND (SEQ ID NO: 145), a VLCDR2 comprising an amino acid sequence of AAS, and a VLCDR3 comprising an amino acid sequence of LQHNTYPWT (SEQ ID NO: 147) (according to the IMGT unique numbering scheme); or
(x) a VHCDR1 comprising an amino acid sequence of GASISSSNHYWG (SEQ ID NO: 149), a VHCDR2 comprising an amino acid sequence of IYYSGNT (SEQ ID NO: 150), a VHCDR3 comprising an amino acid sequence of ARHRGSYFFDH (SEQ ID NO: 151), a VLCDR1 comprising an amino acid sequence of NIGDKN (SEQ ID NO: 153), a VLCDR2 comprising an amino acid sequence of RDS, and a VLCDR3 comprising an amino acid sequence of QVWDSSTVV (SEQ ID NO: 155) (according to the IMGT unique numbering scheme); or
(xi) a VHCDR1 comprising an amino acid sequence of GDSVSSNSAV (SEQ ID NO: 157), a VHCDR2 comprising an amino acid sequence of TDYRSKWNN (SEQ ID NO: 158), a VHCDR3 comprising an amino acid sequence of ARGDAGMASFDY (SEQ ID NO: 159), a VLCDR1 comprising an amino acid sequence of KLGDKY (SEQ ID NO: 161), a VLCDR2 comprising an amino acid sequence of QDR, and a VLCDR3 comprising an amino acid sequence of QTWDRRTAV (SEQ ID NO: 163) (according to the IMGT unique numbering scheme); or
(xii) a VHCDR1 comprising an amino acid sequence of GGSFSDYY (SEQ ID NO: 165), a VHCDR2 comprising an amino acid sequence of INHSGST (SEQ ID NO: 166), a VHCDR3 comprising an amino acid sequence of ARKRGANFFDD (SEQ ID NO: 167), a VLCDR1 comprising an amino acid sequence of NIENKN (SEQ ID NO: 89), a VLCDR2 comprising an amino acid sequence of RDS, and a VLCDR3 comprising an amino acid sequence of QVWDSSTVV (SEQ ID NO: 155) (according to the IMGT unique numbering scheme); and
optionally wherein the antibody is a human IgG1 antibody.

144. The antibody of claim 143, wherein:
(i) the heavy chain variable domain (VH) comprises an amino acid sequence at least 90% identical to SEQ ID NO: 76, and the light chain variable domain (VL) comprises an amino acid sequence at least 90% identical to SEQ ID NO: 80; or
(ii) the VH comprises an amino acid sequence at least 90% identical to SEQ ID NO: 92, and the VL comprises an amino acid sequence at least 90% identical to SEQ ID NO: 96; or
(iii) the VH comprises an amino acid sequence at least 90% identical to SEQ ID NO: 84, and the VL comprises an amino acid sequence at least 90% identical to SEQ ID NO: 88; or
(iv) the VH comprises an amino acid sequence at least 90% identical to SEQ ID NO: 100, and the VL comprises an amino acid sequence at least 90% identical to SEQ ID NO: 104; or
(v) the VH comprises an amino acid sequence at least 90% identical to SEQ ID NO: 108, and the VL comprises an amino acid sequence at least 90% identical to SEQ ID NO: 112; or
(vi) the VH comprises an amino acid sequence at least 90% identical to SEQ ID NO: 116, and the VL comprises an amino acid sequence at least 90% identical to SEQ ID NO: 120; or
(vii) the VH comprises an amino acid sequence at least 90% identical to SEQ ID NO: 124, and the VL comprises an amino acid sequence at least 90% identical to SEQ ID NO: 128; or
(viii) the VH comprises an amino acid sequence at least 90% identical to SEQ ID NO: 132, and the VL comprises an amino acid sequence at least 90% identical to SEQ ID NO: 136; or
(ix) the VH comprises an amino acid sequence at least 90% identical to SEQ ID NO: 140, and the VL comprises an amino acid sequence at least 90% identical to SEQ ID NO: 144; or
(x) the VH comprises an amino acid sequence at least 90% identical to SEQ ID NO: 148, and the VL comprises an amino acid sequence at least 90% identical to SEQ ID NO: 152; or
(xi) the VH comprises an amino acid sequence at least 90% identical to SEQ ID NO: 156, and the VL comprises an amino acid sequence at least 90% identical to SEQ ID NO: 160; or
(xii) the VH comprises an amino acid sequence at least 90% identical to SEQ ID NO: 164, and the VL comprises an amino acid sequence at least 90% identical to SEQ ID NO: 168;
optionally wherein the antibody is a human IgG1 antibody.


SEQUENCE LISTING (338 sequences)

SEQ ID NO: 1 - protein, 249 aa, Homo sapiens
LHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 2 - protein, 183 aa, Homo sapiens
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 3 - protein, 254 aa, Homo sapiens
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 4 - protein, 306 aa, Homo sapiens
APLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCF

SEQ ID NO: 5 - protein, 212 aa, synthetic construct
YYELTQPLSVSVALGQTARIPCGGNNIENKNVHWYQQKPGQAPVLVIYRDSNRPSGIPERFSGSNSGNTATLSINRAQAGDEADYYCQVWDSNTVIFGGGTKVTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 6 - protein, 452 aa, synthetic construct
QLQLQESGPGLVKPSETLSLTCTVSGGSISSSSYYWGWIRQPPGKGLEWIGGIGSVDYSGNTYYKPSLKSRVTISVDTSKNQFSLKLSTVTAADTAVYHCARHRGIYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 212 aa, synthetic construct
SYELTQPLSVSVALGQTARMTCGGSDIENKNVHWYQQKPGQAPVLVIHRDNNRPSGIPERFSGSNSGNTATLTISRAQAGDEADYYCQVRDSNIVVFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 8 - protein, 449 aa, synthetic construct
QLQLQESGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 212 aa, synthetic construct
SFELTQPLSVSVALGQTARITCGGINIENKNVHYYQQKPGQAPVLVIYRDTNRPSGIPERFSGSNSGNTATLTISRAQAGDEADYYCQVWDSNTVIFGGGTKVTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 10 - protein, 449 aa, synthetic construct
QLQLQESGPGLVKPSETLSLTCTVSGGSIDTSSQYWAWIRQPPGKGLEWIGGIYFSGSTYQNPSLKSRVTTSVDTSKNQFSLRLSSMTAADTAVYYCARHRGIYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 214 aa, synthetic construct
SSELTQDPAVSVALGQTVRITCQGDSLRNYFANWYQQKPGQAPVLVIYGKNNRPSGIPDRFSGSSSGNTASLTITGAQAEDEADYYCNSRDSSGYHVVFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 12 - protein, 451 aa, synthetic construct
QLQLQESGPGLVKPSETLSLTCTVSGGSISSGSFYWGWIRQPPGKGLEWIGTIYYSGSTYYNPSLKSRVAISVDTSKNQFYLNLNFVTAADTAVYHCARERSSSWYPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 212 aa, synthetic construct
SYELTQPLSVSVALGQTARITCGGNNIENKNVHWYQQMPGQAPVLVIYRDSNRPSGIPERFSGSNSGNTATLTINRAQAGDEADYYCQVWDSNTVVFGGGTKVTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 14 - protein, 449 aa, synthetic construct
QLQLQESGPGLVKPSETLSLTCTVAGASVSSSSYYWGWIRQSPGKGLEWIGTINYGGSTYHNPSLKRRVTVSVDTSKNQFSLKLTSVTAADTAVYYCARHRGIYHFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 214 aa, synthetic construct
SSELTQDPAVSVALGQTVRITCQGDSVRRFFAHWYQQKPGQAPILVISGKDTRPSGIPDRFSGSISGNTASLTITGAQAEDEADYYCNSRDSSGYLVLFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 16 - protein, 450 aa, synthetic construct
EVQLVESGPGLVKPSETLSLTCTVSGVSIRSNGYYWGWIRQSPGKGLEWIGNMDYSGTYQNPSLTSRVTISGDASKNQFSLKLRSVTAADTAVYFCARERSNNWYPIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 212 aa, synthetic construct
SYELTQPPSVSVALGQTARITCGGNNIENKNVHWYQQKPGQAPVLVIYRDSNRPSGIPERFSGSNSGNTATLSINRAQAGDEADYYCQVWDSNTVVFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 18 - protein, 452 aa, synthetic construct
QVQLQESGPGLVKPSETLSLTCTVSGGSISSSSYYWGWIRQPPGKGLEWIGGIGSVDYSGNTYYKPSLKSRVTISVDTSKNQFSLKLSSVTAADTAVYHCARHRGIYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 211 aa, synthetic construct
SYELTQPLSVSVALGQTARISCGGNNIGRKNVHWYQQKPGQAPVLVIYGDSNRPSGIPERFSGSNSGNTATLTIGRAQAGDEADYYCQVWDSSTVFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 20 - protein, 449 aa, synthetic construct
EVQLLESGGGLVQPGGSLRLSCAASGFTFSNYAMSWVRQAPGKGLEWVSVISGSGDSTYYAESVKGRFTISRDNSKNTLYLQMTSLRAEDTAEYYCAKDRTPVYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 214 aa, synthetic construct
DMQMTQSPSSLSASVGDRVTITCRASQGIRNDLDWYQQKPGKVPKRLIHAASSLQSGVPSRFSGSGSGTEFTLTISSLQPEDFVTYYCLQHNTYPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 451 aa, synthetic construct
EVQLLESGGGLVQPGGSLRLSCAASGFTFSSYAMTWVRQAPGKGLEWVSDISGSGGSTKYADSVKGRFTISRDNPKNTLYLQMNSLRAEDTAVYYCAKDLGFYSTWDTDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 212 aa, synthetic construct
SYVLTQPLSVSVALGQTARITCGGNNIGDKNVHWYQQKPGQAPVLVISRDSNRPSGIPERFSGSNSGNTATLTISRAQAGDESDYYCQVWDSSTVVFGGGTKVTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 24 - protein, 451 aa, synthetic construct
QLQLQESGPGLVKPSETLSLTCTVSGASISSSNHYWGWGWIRQPPGKGLEWIGSIYYSGNTYYNPSLKSRVTISVDTSKNQFSLKLSSVTAADTAVYYCARHRGSYFFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 212 aa, synthetic construct
SSELTQPPSVSVSPGQTASITCSGDKLGDKYACWYQQKAGQSPVVVIYQDRKRPSGIPERFSGSNSGNTATLTISGTQAMDEADYYCQTWDRRTAVFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 26 - protein, 452 aa, synthetic construct
QVQLQQSGPGLVKPSQTLSLTCAISGDSVSSNSAVWNWIRQSPSRGLEWLGRTDYRSKWNNDYAVSVKSRITINPDTSKNQFSLQLNSVTPEDTAVYYCARGDAGMASFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 212 aa, synthetic construct
SYELTQPLSVSVALGQTARITCGGNNIENKNVHWYQQKPGQAPVLVIKRDSNRPSGIPERLSGSNSGNTATLTISRAQAGDEADYYCQVWDSSTVVFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 28 - protein, 447 aa, synthetic construct
EVQLLESGAGLLKPSETLSLTCAVYGGSFSDYYWSWIRQPPGKGLEWIGEINHSGSTNYNPSLKSRVTISVDTSKNQFSLKLSSVTAADTAVYYCARKRGANFFDDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 126 aa, Homo sapiens [sequence omitted]

SEQ ID NO: 30 - protein, 107 aa, synthetic construct
DIQMTQSPSSLSASVGDRVTITCRASQSISSYLNWYQQKPGKAPNLLIYAASSLQSGVPSRFSGSGSETDFTLTISSLQPEDFATYYCQQSHSVSFTFGPGTKVDIK

SEQ ID NO: 31 - protein, 127 aa, synthetic construct
EVQLVESGGGLVQPGGSLRLSCAASGFTFSNYAMNWVRQAPGKGLEWVSTISGSGGATRYADSVKGRFTISRDNSRNTVYLQMNSLRVEDTAVFYCTKDRLIMATVRGPYYYGMDVWGQGTTVTVSS

SEQ ID NO: 32 - protein, 107 aa, synthetic construct
DIQMTQSPSSLSASVGDRVTITCRASQGISSWLAWYQQKPEKAPKSLIYAASSLQSGVPSRFSGSGSGTDFTLTISSLQPEDFATYYCQQYNSYPYTFGQGTKLEIK

SEQ ID NO: 33 - protein, 120 aa, synthetic construct
EVQLLESGGGLVQPGGSLRLSCAASGFTFNSYAMSWVRQAPGKGLEWVSIISGSGGFTYYADSVKGRFTISRDNSRTTLYLQMNSLRAEDTAVYYCAKDRLVAPGTFDYWGQGALVTVSS

SEQ ID NO: 34 - protein, 358 aa, synthetic construct
AMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 214 aa, synthetic construct
DIQMTQSPSSLSASVGDRVTITCRASQGISSWLAWYQQKPEKAPKSLIYAASSLQSGVPSRFSGSGSGTDFTLTISSLQPEDFATYYCQQYNSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 449 aa, synthetic construct
EVQLLESGGGLVQPGGSLRLSCAASGFTFNSYAMSWVRQAPGKGLEWVSIISGSGGFTYYADSVKGRFTISRDNSRTTLYLQMNSLRAEDTAVYYCAKDRLVAPGTFDYWGQGALVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 37 - protein, 596 aa, synthetic construct
AMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSEVQLLESGGGLVQPGGSLRLSCAASGFTFNSYAMSWVRQAPGKGLEWVSIISGSGGFTYYADSVKGRFTISRDNSRTTLYLQMNSLRAEDTAVYYCAKDRLVAPGTFDYWGQGALVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 38 - protein, 597 aa, synthetic construct
AMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQLQLQESGPGLVKPSETLSLTCTVSGGSISSSSYYWGWIRQPPGKGLEWIGGIGSVDYSGNTYYKPSLKSRVTISVDTSKNQFSLKLSTVTAADTAVYHCARHRGIYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 39 - protein, 212 aa, synthetic construct; identical to SEQ ID NO: 7

SEQ ID NO: 40 - protein, 594 aa, synthetic construct
AMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQLQLQESGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 41 - protein, 212 aa, synthetic construct; identical to SEQ ID NO: 9

SEQ ID NO: 42 - protein, 594 aa, synthetic construct
AMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQLQLQESGPGLVKPSETLSLTCTVSGGSIDTSSQYWAWIRQPPGKGLEWIGGIYFSGSTYQNPSLKSRVTTSVDTSKNQFSLRLSSMTAADTAVYYCARHRGIYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 43 - protein, 214 aa, synthetic construct; identical to SEQ ID NO: 11

SEQ ID NO: 44 - protein, 596 aa, synthetic construct
AMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQLQLQESGPGLVKPSETLSLTCTVSGGSISSGSFYWGWIRQPPGKGLEWIGTIYYSGSTYYNPSLKSRVAISVDTSKNQFYLNLNFVTAADTAVYHCARERSSSWYPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 45 - protein, 212 aa, synthetic construct; identical to SEQ ID NO: 13

SEQ ID NO: 46 - protein, 594 aa, synthetic construct
AMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQLQLQESGPGLVKPSETLSLTCTVAGASVSSSSYYWGWIRQSPGKGLEWIGTINYGGSTYHNPSLKRRVTVSVDTSKNQFSLKLTSVTAADTAVYYCARHRGIYHFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 47 - protein, 214 aa, synthetic construct; identical to SEQ ID NO: 15

SEQ ID NO: 48 - protein, 595 aa, synthetic construct
AMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSEVQLVESGPGLVKPSETLSLTCTVSGVSIRSNGYYWGWIRQSPGKGLEWIGNMDYSGTYQNPSLTSRVTISGDASKNQFSLKLRSVTAADTAVYFCARERSNNWYPIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 49 - protein, 212 aa, synthetic construct; identical to SEQ ID NO: 17

SEQ ID NO: 50 - protein, 597 aa, synthetic construct
AMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQVQLQESGPGLVKPSETLSLTCTVSGGSISSSSYYWGWIRQPPGKGLEWIGGIGSVDYSGNTYYKPSLKSRVTISVDTSKNQFSLKLSSVTAADTAVYHCARHRGIYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 51 - protein, 211 aa, synthetic construct; identical to SEQ ID NO: 19

SEQ ID NO: 52 - protein, 594 aa, synthetic construct
AMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSEVQLLESGGGLVQPGGSLRLSCAASGFTFSNYAMSWVRQAPGKGLEWVSVISGSGDSTYYAESVKGRFTISRDNSKNTLYLQMTSLRAEDTAEYYCAKDRTPVYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 53 - protein, 214 aa, synthetic construct; identical to SEQ ID NO: 21

SEQ ID NO: 54 - protein, 596 aa, synthetic construct
AMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSEVQLLESGGGLVQPGGSLRLSCAASGFTFSSYAMTWVRQAPGKGLEWVSDISGSGGSTKYADSVKGRFTISRDNPKNTLYLQMNSLRAEDTAVYYCAKDLGFYSTWDTDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 55 - protein, 212 aa, synthetic construct; identical to SEQ ID NO: 23

SEQ ID NO: 56 - protein, 596 aa, synthetic construct
AMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQLQLQESGPGLVKPSETLSLTCTVSGASISSSNHYWGWGWIRQPPGKGLEWIGSIYYSGNTYYNPSLKSRVTISVDTSKNQFSLKLSSVTAADTAVYYCARHRGSYFFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 57 - protein, 212 aa, synthetic construct; identical to SEQ ID NO: 25

SEQ ID NO: 58 - protein, 597 aa, synthetic construct
AMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQVQLQQSGPGLVKPSQTLSLTCAISGDSVSSNSAVWNWIRQSPSRGLEWLGRTDYRSKWNNDYAVSVKSRITINPDTSKNQFSLQLNSVTPEDTAVYYCARGDAGMASFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 59 - protein, 212 aa, synthetic construct; identical to SEQ ID NO: 27

SEQ ID NO: 60 - protein, 592 aa, synthetic construct
AMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSEVQLLESGAGLLKPSETLSLTCAVYGGSFSDYYWSWIRQPPGKGLEWIGEINHSGSTNYNPSLKSRVTISVDTSKNQFSLKLSSVTAADTAVYYCARKRGANFFDDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 61 - protein, 212 aa, synthetic construct
SYELTQPLSVSVALGQTARITCGGSDIENKNVHWYQQKPGQAPVLVIHRDNNRPSGIPERFSGSNSGNTATLTISRAQAGDEADYYCQVRDSNIVVFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 62 - protein, 594 aa, synthetic construct
AMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQLQLQESGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 63 - protein, 212 aa, synthetic construct
SSELTQPLSVSVALGQTARMTCGGSDIENKNVHWYQQKPGQAPVLVIHRDNNRPSGIPERFSGSNSGNTATLTISRAQAGDEADYYCQVRDSNIVVFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 64 - protein, 595 aa, synthetic construct
AMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQLQLQESGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 65 - protein, 212 aa, synthetic construct
SYELTQPLSVSVALGQTARITCGGNNIENKNVHWYQQKPGQAPVLVIYRDSNRPSGIPERFSGSNSGNTATLTISRAQAGDEADYYCQVWDSTTVVFGGGTKVTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 66 - protein, 594 aa, synthetic construct
AMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQLQLQESGPGLVKPSETLSLTCTVSGASVSSSSYYWGWIRQPPGKGLEWIGTINYGGSTYHNPSLKRRVTISVDTSKNQFSLKLSSVTAADTAVYYCARHRGIYHFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 67 - protein, 214 aa, synthetic construct
SSELTQDPAVSVALGQTVRITCQGDSVRRFFAHWYQQKPGQAPVLVISGKDTRPSGIPDRFSGSSSGNTASLTITGAQAEDEADYYCNSRDSSGYLVLFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 68 - protein, 595 aa, synthetic construct
AMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQLQLQESGPGLVKPSETLSLTCTVSGVSIRSSGYYWGWIRQPPGKGLEWIGNIDYSGTYQNPSLTSRVTISVDTSKNQFSLKLSSVTAADTAVYYCARERSNNWYPIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 69 - protein, 212 aa, synthetic construct
SYELTQPPSMSVAPGQTARITCGGSNIEDKNVHWYQQKPGQAPVLVIKRDSDRPSGIPERFSGSNSGNTATLTISRAQAGDEAEYYCQVWDSSTVVFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 70 - protein, 597 aa, synthetic construct
AMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQVQLQESGPGLVKPSETLSLTCTVSGGSISSSSYYWGWIRQPPGKGLEWIGGIGSVDYSGNTYYKPSLKSRVTISVDTSKNQFSLKLSSVTAADTAVYHCARHRGIYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 71 - protein, 357 aa, synthetic construct
AMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 72 - protein, 371 aa, synthetic construct
EPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDS

SEQ ID NO: 73 - protein, 471 aa, synthetic construct
EPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQLQLQESGPGLVKPSETLSLTCTVSGGSISSSSYYWGWIRQPPGKGLEWIGGIGSVDYSGNTYYKPSLKSRVTISVDTSKNQFSLKLSTVTAADTAVYHCARHRGIYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 74 - DNA, 636 bp, synthetic construct
  source 1..636: mol_type = other DNA
tactatgagctgactcagccactctcagtgtcagtggccctgggacagacggccaggattccctgtgggggaaacaacattgaaaataaaaatgtgcactggtaccaacagaagccaggccaggcccctgtactggtcatctatagggatagcaaccggccctctgggatccctgagcgattctctggctccaactcggggaacacggccaccctgtccatcaacagagcccaagccggggatgaggctgactattactgtcaggtgtgggacagcaacactgtgatattcggcggagggaccaaggtcaccgtcctaggacagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacaaaagctacagctgccaggtcacgcatgaagggagcaccgtggagaagacagtggcccctacagaatgttca

SEQ ID NO: 75 - DNA, 1791 bp, synthetic construct
  source 1..1791: mol_type = other DNA
gcaatgcacgtggcccagcctgctgtggtactggccagcagccgaggcatcgccagctttgtgtgtgagtacgcatctccaggcaaagccactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggctatggacacgggactctacatctgcaaggtggagctcatgtacccaccgccatactacctgggcataggcaacggaacccagatttatgtaattgatccagaaccgtgcccagattctgaccaggaacccaagagctccgacaagacccatacctgtcctccctgtcctgctccaccggtcgctgggccctccgtgttcctgttcccccccaagcccaaggacaccctgatgatcagcaggacccccgaggtgacctgcgtggtggtggacgtgagccacgaggacccagaggtgaagttcaattggtatgtggacggcgtggaggtgcacaacgccaagaccaagcccagagaggaacagtacaacagcacctacagggtggtgtccgtgctgaccgtgctgcaccaggactggctgaacggcaaggaatacaagtgcgccgtatccaacaaggccctgccctccagcatcgagaaaaccatcagcaaggccaagggccagccacgggagccccaggtgtacacactgcccccatctcgggaagaaatgaccaagaaccaggtgtccctgacctgtctggtgaagggcttttaccccagcgacatcgccgtggagtgggagagcaacggccagcccgagaacaactacaagaccaccccccctgtgctggacagcgacggcagcttcttcctgtacagcaagctgaccgtggacaagtccaggtggcagcagggcaacgtgttcagctgcagcgtgatgcacgaggccctgcacaaccactacacacagaagagcctgagcctgtccccgggtgccggaggtggcggaagtggtggcggtggctcaggtggcggtggatcccagctgcagctgcaggagtctggaccaggactggtgaagccctcggagaccctgtccctcacctgcactgtctctggtggctccatcagcagtagtagttactactggggctggatccgccagcccccagggaaggggctggagtggattgggggtattgggagtgtcgattatagtgggaacacctactacaaaccgtccctcaagagtcgagtcaccatatccgtagacacgtccaagaaccagttttccctgaagctgagcactgtgaccgccgcagacacggctgtgtatcactgtgcgagacatcgtgggatatacttctttgactactggggccagggaaccctggtcaccgtctcttcagctagcaccaagggtccatcggtcttccccctggcaccctcctccaagagcacctctgggggcacagcggccctgggctgcctggtcaaggactacttccccgaaccagtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgt

SEQ ID NO: 76 - protein, 119 aa, synthetic construct
QLQLQESGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSS

SEQ ID NO: 77 - protein, 10 aa, synthetic construct
GGSISTSSYY

SEQ ID NO: 78 - protein, 7 aa, synthetic construct
IYYSGST

SEQ ID NO: 79 - protein, 11 aa, synthetic construct
ARHRGSYFFDI

SEQ ID NO: 80 - protein, 106 aa, synthetic construct
SYELTQPLSVSVALGQTARMTCGGSDIENKNVHWYQQKPGQAPVLVIHRDNNRPSGIPERFSGSNSGNTATLTISRAQAGDEADYYCQVRDSNIVVFGGGTKLTVL

SEQ ID NO: 81 - protein, 6 aa, synthetic construct
DIENKN

SEQ ID NO: 82 - skipped

SEQ ID NO: 83 - protein, 9 aa, synthetic construct
QVRDSNIVV

SEQ ID NO: 84 - protein, 119 aa, synthetic construct
QLQLQESGPGLVKPSETLSLTCTVAGASVSSSSYYWGWIRQSPGKGLEWIGTINYGGSTYHNPSLKRRVTVSVDTSKNQFSLKLTSVTAADTAVYYCARHRGIYHFDYWGQGTLVTVSS

SEQ ID NO: 85 - protein, 10 aa, synthetic construct
GASVSSSSYY

SEQ ID NO: 86 - protein, 7 aa, synthetic construct
INYGGST

SEQ ID NO: 87 - protein, 11 aa, synthetic construct
ARHRGIYHFDY

SEQ ID NO: 88 - protein, 106 aa, synthetic construct
SYELTQPLSVSVALGQTARITCGGNNIENKNVHWYQQMPGQAPVLVIYRDSNRPSGIPERFSGSNSGNTATLTINRAQAGDEADYYCQVWDSNTVVFGGGTKVTVL

SEQ ID NO: 89 - protein, 6 aa, synthetic construct
NIENKN

SEQ ID NO: 90 - skipped

SEQ ID NO: 91 - protein, 9 aa, synthetic construct
QVWDSNTVV

SEQ ID NO: 92 - protein, 120 aa, synthetic construct
EVQLVESGPGLVKPSETLSLTCTVSGVSIRSNGYYWGWIRQSPGKGLEWIGNMDYSGTYQNPSLTSRVTISGDASKNQFSLKLRSVTAADTAVYFCARERSNNWYPIDYWGQGTLVTVSS

SEQ ID NO: 93 - protein, 10 aa, synthetic construct
GVSIRSNGYY

SEQ ID NO: 94 - protein, 6 aa, synthetic construct
MDYSGT

SEQ ID NO: 95 - protein, 13 aa, synthetic construct
ARERSNNWYPIDY

SEQ ID NO: 96 - protein, 108 aa, synthetic construct
SSELTQDPAVSVALGQTVRITCQGDSVRRFFAHWYQQKPGQAPILVISGKDTRPSGIPDRFSGSISGNTASLTITGAQAEDEADYYCNSRDSSGYLVLFGGGTKLTVL

SEQ ID NO: 97 - protein, 6 aa, synthetic construct
SVRRFF

SEQ ID NO: 98 - skipped

SEQ ID NO: 99 - protein, 11 aa, synthetic construct
NSRDSSGYLVL

SEQ ID NO: 100 - protein, 122 aa, synthetic construct
QVQLQESGPGLVKPSETLSLTCTVSGGSISSSSYYWGWIRQPPGKGLEWIGGIGSVDYSGNTYYKPSLKSRVTISVDTSKNQFSLKLSSVTAADTAVYHCARHRGIYFFDYWGQGTLVTVSS

SEQ ID NO: 101 - protein, 10 aa, synthetic construct
GGSISSSSYY

SEQ ID NO: 102 - protein, 10 aa, synthetic construct
IGSVDYSGNT

SEQ ID NO: 103 - protein, 11 aa, synthetic construct
ARHRGIYFFDY

SEQ ID NO: 104 - protein, 106 aa, synthetic construct
SYELTQPPSVSVALGQTARITCGGNNIENKNVHWYQQKPGQAPVLVIYRDSNRPSGIPERFSGSNSGNTATLSINRAQAGDEADYYCQVWDSNTVVFGGGTKLTVL

SEQ ID NO: 105 - protein, 5 aa, synthetic construct
SYAMS

SEQ ID NO: 106 - protein, 16 aa, synthetic construct
IISGSGGFTYYADSVK

SEQ ID NO: 107 - protein, 11 aa, synthetic construct
DRLVAPGTFDY

SEQ ID NO: 108 - protein, 122 aa, synthetic construct
QLQLQESGPGLVKPSETLSLTCTVSGGSISSSSYYWGWIRQPPGKGLEWIGGIGSVDYSGNTYYKPSLKSRVTISVDTSKNQFSLKLSTVTAADTAVYHCARHRGIYFFDYWGQGTLVTVSS

SEQ ID NO: 109 - protein, 10 aa, synthetic construct
GGSISSSSYY

SEQ ID NO: 110 - protein, 10 aa, synthetic construct
IGSVDYSGNT

SEQ ID NO: 111 - protein, 20 aa, synthetic construct
GGGGSGGGGSGGGGSGGGGS

SEQ ID NO: 112 - protein, 106 aa, synthetic construct
YYELTQPLSVSVALGQTARIPCGGNNIENKNVHWYQQKPGQAPVLVIYRDSNRPSGIPERFSGSNSGNTATLSINRAQAGDEADYYCQVWDSNTVIFGGGTKVTVL

SEQ ID NO: 113 - protein, 8 aa, synthetic construct
ISGSGGAT

SEQ ID NO: 114 - protein, 20 aa, synthetic construct
TKDRLIMATVRGPYYYGMDV

SEQ ID NO: 115 - protein, 9 aa, synthetic construct
QVWDSNTVI

SEQ ID NO: 116 - protein, 119 aa, synthetic construct
QLQLQESGPGLVKPSETLSLTCTVSGGSIDTSSQYWAWIRQPPGKGLEWIGGIYFSGSTYQNPSLKSRVTTSVDTSKNQFSLRLSSMTAADTAVYYCARHRGIYFFDYWGQGTLVTVSS

SEQ ID NO: 117 - protein, 10 aa, synthetic construct
GGSIDTSSQY

SEQ ID NO: 118 - protein, 7 aa, synthetic construct
IYFSGST

SEQ ID NO: 119 - protein, 124 aa, synthetic construct
MHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVIDPEPCPDSD

SEQ ID NO: 120 - protein, 106 aa, synthetic construct
SFELTQPLSVSVALGQTARITCGGINIENKNVHYYQQKPGQAPVLVIYRDTNRPSGIPERFSGSNSGNTATLTISRAQAGDEADYYCQVWDSNTVIFGGGTKVTVL

SEQ ID NO: 121 - protein, 6 aa, synthetic construct
QSISSY

SEQ ID NO: 122 - skipped

SEQ ID NO: 123 - protein, 9 aa, synthetic construct
QVWDSNTVI

SEQ ID NO: 124 - protein, 121 aa, synthetic construct
QLQLQESGPGLVKPSETLSLTCTVSGGSISSGSFYWGWIRQPPGKGLEWIGTIYYSGSTYYNPSLKSRVAISVDTSKNQFYLNLNFVTAADTAVYHCARERSSSWYPFDYWGQGTLVTVSS

SEQ ID NO: 125 - protein, 10 aa, synthetic construct
GGSISSGSFY

SEQ ID NO: 126 - protein, 7 aa, synthetic construct
IYYSGST

SEQ ID NO: 127 - protein, 13 aa, synthetic construct
ARERSSSWYPFDY

SEQ ID NO: 128 - protein, 108 aa, synthetic construct
SSELTQDPAVSVALGQTVRITCQGDSLRNYFANWYQQKPGQAPVLVIYGKNNRPSGIPDRFSGSSSGNTASLTITGAQAEDEADYYCNSRDSSGYHVVFGGGTKLTVL

SEQ ID NO: 129 - protein, 6 aa, synthetic construct
SLRNYF

SEQ ID NO: 130 - skipped

SEQ ID NO: 131 - protein, 11 aa, synthetic construct
NSRDSSGYHVV

SEQ ID NO: 132 - protein, 119 aa, synthetic construct
EVQLLESGGGLVQPGGSLRLSCAASGFTFSNYAMSWVRQAPGKGLEWVSVISGSGDSTYYAESVKGRFTISRDNSKNTLYLQMTSLRAEDTAEYYCAKDRTPVYGLDVWGQGTTVTVSS

SEQ ID NO: 133 - protein, 8 aa, synthetic construct
GFTFSNYA

SEQ ID NO: 134 - protein, 8 aa, synthetic construct
ISGSGDST

SEQ ID NO: 135 - protein, 12 aa, synthetic construct
AKDRTPVYGLDV

SEQ ID NO: 136 - protein, 105 aa, synthetic construct
SYELTQPLSVSVALGQTARISCGGNNIGRKNVHWYQQKPGQAPVLVIYGDSNRPSGIPERFSGSNSGNTATLTIGRAQAGDEADYYCQVWDSSTVFGGGTKLTVL

SEQ ID NO: 137 - protein, 6 aa, synthetic construct
NIGRKN

SEQ ID NO: 138 - skipped

SEQ ID NO: 139 - protein, 8 aa, synthetic construct
QVWDSSTV

SEQ ID NO: 140 - protein, 121 aa, synthetic construct
EVQLLESGGGLVQPGGSLRLSCAASGFTFSSYAMTWVRQAPGKGLEWVSDISGSGGSTKYADSVKGRFTISRDNPKNTLYLQMNSLRAEDTAVYYCAKDLGFYSTWDTDYWGQGTLVTVSS

SEQ ID NO: 141 - protein, 8 aa, synthetic construct
GFTFSSYA

SEQ ID NO: 142 - protein, 8 aa, synthetic construct
ISGSGGST

SEQ ID NO: 143 - protein, 14 aa, synthetic construct
AKDLGFYSTWDTDY

SEQ ID NO: 144 - protein, 107 aa, synthetic construct
DMQMTQSPSSLSASVGDRVTITCRASQGIRNDLDWYQQKPGKVPKRLIHAASSLQSGVPSRFSGSGSGTEFTLTISSLQPEDFVTYYCLQHNTYPWTFGQGTKLEIK

SEQ ID NO: 145 - protein, 6 aa, synthetic construct
QGIRND

SEQ ID NO: 146 - skipped

SEQ ID NO: 147 - protein, 9 aa, synthetic construct
LQHNTYPWT

SEQ ID NO: 148 - protein, 121 aa, synthetic construct
QLQLQESGPGLVKPSETLSLTCTVSGASISSSNHYWGWGWIRQPPGKGLEWIGSIYYSGNTYYNPSLKSRVTISVDTSKNQFSLKLSSVTAADTAVYYCARHRGSYFFDHWGQGTLVTVSS

SEQ ID NO: 149 - protein, 12 aa, synthetic construct
GASISSSNHYWG

SEQ ID NO: 150 - protein, 7 aa, synthetic construct
IYYSGNT

SEQ ID NO: 151 - protein, 11 aa, synthetic construct
ARHRGSYFFDH

SEQ ID NO: 152 - protein, 106 aa, synthetic construct
SYVLTQPLSVSVALGQTARITCGGNNIGDKNVHWYQQKPGQAPVLVISRDSNRPSGIPERFSGSNSGNTATLTISRAQAGDESDYYCQVWDSSTVVFGGGTKVTVL

SEQ ID NO: 153 - protein, 6 aa, synthetic construct
NIGDKN

SEQ ID NO: 154 - protein, 9 aa, synthetic construct
QQSHSVSFT

SEQ ID NO: 155 - protein, 9 aa, synthetic construct
QVWDSSTVV

SEQ ID NO: 156 - protein, 122 aa, synthetic construct
QVQLQQSGPGLVKPSQTLSLTCAISGDSVSSNSAVWNWIRQSPSRGLEWLGRTDYRSKWNNDYAVSVKSRITINPDTSKNQFSLQLNSVTPEDTAVYYCARGDAGMASFDYWGQGTLVTVSS

SEQ ID NO: 157 - protein, 10 aa, synthetic construct
GDSVSSNSAV

SEQ ID NO: 158 - protein, 9 aa, synthetic construct
TDYRSKWNN

SEQ ID NO: 159 - protein, 12 aa, synthetic construct
ARGDAGMASFDY

SEQ ID NO: 160 - protein, 106 aa, synthetic construct
SSELTQPPSVSVSPGQTASITCSGDKLGDKYACWYQQKAGQSPVVVIYQDRKRPSGIPERFSGSNSGNTATLTISGTQAMDEADYYCQTWDRRTAVFGGGTKLTVL

SEQ ID NO: 161 - protein, 6 aa, synthetic construct
KLGDKY

SEQ ID NO: 162 - skipped

SEQ ID NO: 163 - protein, 9 aa, synthetic construct
QTWDRRTAV

SEQ ID NO: 164 - protein, 117 aa, synthetic construct
EVQLLESGAGLLKPSETLSLTCAVYGGSFSDYYWSWIRQPPGKGLEWIGEINHSGSTNYNPSLKSRVTISVDTSKNQFSLKLSSVTAADTAVYYCARKRGANFFDDWGQGTLVTVSS

SEQ ID NO: 165 - protein, 8 aa, synthetic construct
GGSFSDYY

SEQ ID NO: 166 - protein, 7 aa, synthetic construct
INHSGST

SEQ ID NO: 167 - protein, 11 aa, synthetic construct
ARKRGANFFDD

SEQ ID NO: 168 - protein, 106 aa, synthetic construct
SYELTQPLSVSVALGQTARITCGGNNIENKNVHWYQQKPGQAPVLVIKRDSNRPSGIPERLSGSNSGNTATLTISRAQAGDEADYYCQVWDSSTVVFGGGTKLTVL

SEQ ID NO: 169 - protein, 11 aa, synthetic construct
RASQGISSWLA

SEQ ID NO: 170 - protein, 7 aa, synthetic construct
AASSLQS

SEQ ID NO: 171 - protein, 9 aa, synthetic construct
QQYNSYPYT

SEQ ID NO: 172 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 173 - protein, 125 aa, Homo sapiens
AMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSD

SEQ ID NO: 174 - protein, 124 aa, Homo sapiens [sequence omitted]

SEQ ID NO: 175 - protein, 22 aa, Homo sapiens
EPKSCDKTHTCPPCPAPELLGG

SEQ ID NO: 176 - protein, 22 aa, synthetic construct
EPKSSDKTHTCPPCPAPELLGG

SEQ ID NO: 177 - protein, 22 aa, synthetic construct
EPKSSDKTHTCPPCPAPEAAGG

SEQ ID NO: 178 - protein, 210 aa, Homo sapiens
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 179 - protein, 210 aa, Homo sapiens
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGA

SEQ ID NO: 180 - protein, 209 aa, Homo sapiens
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 181 - protein, 60 aa, synthetic construct
  VARIANT 6..60: note = GGGGS repeats may be deleted
GGGGSGGGGSGGGGSGGGGSGGGGSGGGGSGGGGSGGGGSGGGGSGGGGSGGGGSGGGGS

SEQ ID NO: 182 - protein, 596 aa, synthetic construct
KAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQLQLQESGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 183 - protein, 594 aa, synthetic construct
MHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQLQLQESGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 184 - protein, 595 aa, synthetic construct
KAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQLQLQESGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 185 - protein, 593 aa, synthetic construct
MHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQLQLQESGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 186 - protein, 594 aa, synthetic construct
AMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQLQLQESGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 187 - protein, 597 aa, synthetic construct
KAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSEVQLVESGPGLVKPSETLSLTCTVSGVSIRSNGYYWGWIRQSPGKGLEWIGNMDYSGTYQNPSLTSRVTISGDASKNQFSLKLRSVTAADTAVYFCARERSNNWYPIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 188 - protein, 595 aa, synthetic construct
MHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSEVQLVESGPGLVKPSETLSLTCTVSGVSIRSNGYYWGWIRQSPGKGLEWIGNMDYSGTYQNPSLTSRVTISGDASKNQFSLKLRSVTAADTAVYFCARERSNNWYPIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 189 - protein, 596 aa, synthetic construct
KAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSEVQLVESGPGLVKPSETLSLTCTVSGVSIRSNGYYWGWIRQSPGKGLEWIGNMDYSGTYQNPSLTSRVTISGDASKNQFSLKLRSVTAADTAVYFCARERSNNWYPIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 190 - protein, 594 aa, synthetic construct
MHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSEVQLVESGPGLVKPSETLSLTCTVSGVSIRSNGYYWGWIRQSPGKGLEWIGNMDYSGTYQNPSLTSRVTISGDASKNQFSLKLRSVTAADTAVYFCARERSNNWYPIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 191 - protein, 595 aa, synthetic construct
AMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSEVQLVESGPGLVKPSETLSLTCTVSGVSIRSNGYYWGWIRQSPGKGLEWIGNMDYSGTYQNPSLTSRVTISGDASKNQFSLKLRSVTAADTAVYFCARERSNNWYPIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 192 - protein, 596 aa, synthetic construct
KAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQLQLQESGPGLVKPSETLSLTCTVAGASVSSSSYYWGWIRQSPGKGLEWIGTINYGGSTYHNPSLKRRVTVSVDTSKNQFSLKLTSVTAADTAVYYCARHRGIYHFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 193 - protein, 594 aa, synthetic construct
MHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQLQLQESGPGLVKPSETLSLTCTVAGASVSSSSYYWGWIRQSPGKGLEWIGTINYGGSTYHNPSLKRRVTVSVDTSKNQFSLKLTSVTAADTAVYYCARHRGIYHFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 194 - protein, 595 aa, synthetic construct
KAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQLQLQESGPGLVKPSETLSLTCTVAGASVSSSSYYWGWIRQSPGKGLEWIGTINYGGSTYHNPSLKRRVTVSVDTSKNQFSLKLTSVTAADTAVYYCARHRGIYHFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 195 - protein, 593 aa, synthetic construct
MHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQLQLQESGPGLVKPSETLSLTCTVAGASVSSSSYYWGWIRQSPGKGLEWIGTINYGGSTYHNPSLKRRVTVSVDTSKNQFSLKLTSVTAADTAVYYCARHRGIYHFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 196 - protein, 594 aa, synthetic construct
AMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQLQLQESGPGLVKPSETLSLTCTVAGASVSSSSYYWGWIRQSPGKGLEWIGTINYGGSTYHNPSLKRRVTVSVDTSKNQFSLKLTSVTAADTAVYYCARHRGIYHFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 197 - protein, 599 aa, synthetic construct
KAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQVQLQESGPGLVKPSETLSLTCTVSGGSISSSSYYWGWIRQPPGKGLEWIGGIGSVDYSGNTYYKPSLKSRVTISVDTSKNQFSLKLSSVTAADTAVYHCARHRGIYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 198 - protein, 597 aa, synthetic construct
MHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQVQLQESGPGLVKPSETLSLTCTVSGGSISSSSYYWGWIRQPPGKGLEWIGGIGSVDYSGNTYYKPSLKSRVTISVDTSKNQFSLKLSSVTAADTAVYHCARHRGIYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 199 - protein, 598 aa, synthetic construct
KAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQVQLQESGPGLVKPSETLSLTCTVSGGSISSSSYYWGWIRQPPGKGLEWIGGIGSVDYSGNTYYKPSLKSRVTISVDTSKNQFSLKLSSVTAADTAVYHCARHRGIYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 200 - protein, 596 aa, synthetic construct
MHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQVQLQESGPGLVKPSETLSLTCTVSGGSISSSSYYWGWIRQPPGKGLEWIGGIGSVDYSGNTYYKPSLKSRVTISVDTSKNQFSLKLSSVTAADTAVYHCARHRGIYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 201 - protein, 597 aa, synthetic construct
AMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQVQLQESGPGLVKPSETLSLTCTVSGGSISSSSYYWGWIRQPPGKGLEWIGGIGSVDYSGNTYYKPSLKSRVTISVDTSKNQFSLKLSSVTAADTAVYHCARHRGIYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 202 - protein, 21 aa, synthetic construct
  VARIANT 19: note = L or E
RVESKYGPPCPPCPAPEFXGG

SEQ ID NO: 203 - protein, 21 aa, synthetic construct
RVESKYGPPCPPCPAPEFLGG

SEQ ID NO: 204 - protein, 21 aa, synthetic construct
RVESKYGPPCPPCPAPEFEGG

SEQ ID NO: 205 - protein, 208 aa, Homo sapiens
PSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCVKGFYPSDIAVEWESNGQPENNYKTTPPVLDSDGSFFLYSKLTVDKSRWQEGNVFSCSVMHEALHNHYTQKSLSLSLG

SEQ ID NO: 206 - protein, 20 aa, synthetic construct
GSGSGSGSGSGSGSGSGSGS

SEQ ID NO: 207 - protein, 30 aa, synthetic construct
GGSGGSGGSGGSGGSGGSGGSGGSGGSGGS

SEQ ID NO: 208 - protein, 40 aa, synthetic construct
GGGSGGGSGGGSGGGSGGGSGGGSGGGSGGGSGGGSGGGS

SEQ ID NO: 209 - protein, 40 aa, synthetic construct
GGSGGGSGGGSGGGSGGGSGGGSGGGSGGGSGGGSGGGSG

SEQ ID NO: 210 - protein, 50 aa, synthetic construct
GGSGGGGSGGGGSGGGGSGGGGSGGGGSGGGGSGGGGSGGGGSGGGGSGG

SEQ ID NO: 211 - protein, 100 aa, synthetic construct
GGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGG

SEQ ID NO: 212 - protein, 22 aa, synthetic construct
EPKSCDKTHTCPPCPAPEAAGG

SEQ ID NO: 213 - protein, 21 aa, synthetic construct
EPKSSDKTHTCPPCPAPPVAG

SEQ ID NO: 214 - protein, 210 aa, Homo sapiens
PSVFLFPPKPKDTLMISRTPEVTCVVVDVSHEDPEVKFNWYVDGVEVHNAKTKPREEQYNSTYRVVSVLTVLHQDWLNGKEYKCAVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGA

SEQ ID NO: 215 - protein, 124 aa, synthetic construct
  VARIANT 29: note = H, K, T, W or Y
  VARIANT 51: note = N or Y
  VARIANT 93: note = M or Q
MHVAQPAVVLASSRGIASFVCEYASPGKXTEVRVTVLRQADSQVTEVCAAXYMMGNEGTFLDDSICTGTSSGNQVNLTIQGLRAMDTGLYICXVEKMYPPPYYLGIGNGTQIYVIDPEPCPDSD

SEQ ID NO: 216 - protein, 210 aa, Homo sapiens
PSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGA

SEQ ID NO: 217 - protein, 210 aa, Homo sapiens
PSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 218 - protein, 19 aa, Homo sapiens
ESKYGPPCPSCPAPEFLGG

SEQ ID NO: 219 - protein, 585 aa, Homo sapiens [sequence omitted]

SEQ ID NO: 220 - protein, 11 aa, synthetic construct
  VARIANT 6: note = S or I
  VARIANT 8: note = F or H
  VARIANT 11: note = I or Y
ARHRGXYXFDX

SEQ ID NO: 221 - protein, 13 aa, synthetic construct
  VARIANT 6: note = N or S
  VARIANT 7: note = N, D, G or S
  VARIANT 11: note = I or F
ARERSXXWYPXDY

SEQ ID NO: 222 - protein, 10 aa, synthetic construct
  VARIANT 2: note = A, G or V
  VARIANT 4: note = V or I
  VARIANT 5: note = S or R
  VARIANT 6: note = S or T
  VARIANT 8: note = S or G
GXSXXXSXYY

SEQ ID NO: 223 - protein, 7 aa, synthetic construct
  VARIANT 2: note = Y or N
  VARIANT 4: note = S or G
IXYXGST

SEQ ID NO: 224 - protein, 6 aa, synthetic construct
  VARIANT 1: note = I or M
XDYSGT

SEQ ID NO: 225 - protein, 10 aa, synthetic construct
  VARIANT 9: note = N, A or S
IGSVDYSGXT

SEQ ID NO: 226 - protein, 6 aa, synthetic construct
  VARIANT 1: note = N or D
XIENKN

SEQ ID NO: 227-229 - skipped (3 entries)

SEQ ID NO: 230 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 231 - protein, 9 aa, synthetic construct
  VARIANT 3: note = R or W
  VARIANT 6: note = N, T or A
  VARIANT 7: note = I, T or A
QVXDSXXVV

SEQ ID NO: 232 - protein, 11 aa, synthetic construct
  VARIANT 9: note = L or H
  VARIANT 11: note = L or V
NSRDSSGYXVX

SEQ ID NO: 233 - protein, 124 aa, synthetic construct
  VARIANT 29: note = H, K, W or Y
  VARIANT 61: note = D or E
MHVAQPAVVLASSRGIASFVCEYASPGKXTEVRVTVLRQADSQVTEVCFANYYMGNELTFXDDSICTGTSSGNQVNLTIQGLRAMDTGLYICQVELMYPPPYYEGIGNGTQIYVIDPEPCPDSD

SEQ ID NO: 234 - protein, 124 aa, synthetic construct
MHVAQPAVVLASSRGIASFVCEYESPGKANEIRVTVLRQADSQVTEVCAMTYMKEDELTFLDDPSCTGTFSGNQVNLTIQGLRAMDTGLYICKVELMYPPPYYEGIGNGTQIYVIDPEPCPDSD

SEQ ID NO: 235 - protein, 124 aa, synthetic construct
  VARIANT 16: note = R or V
  VARIANT 58: note = A or G
  VARIANT 85: note = Q or S
MHVAQPAVVLASSRGXASFVCEYTNPSKATEVRVTVLRQADSQVTEVCAATYMKGNEXTFLDDSICTGTASGNQVNLTIRGLRAXDTGLYICQVELMYPPPYYLGIGNGTQIYVIDPEPCPDSD

SEQ ID NO: 236 - protein, 123 aa, synthetic construct
MLRLLLALNLFPSIQVTGNKILVKQSPMLVAYDNAVNLSWKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 237 - protein, 210 aa, Homo sapiens
PSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 238 - protein, 210 aa, Homo sapiens
PSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 239 - protein, 220 aa, Homo sapiens
PSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 240 - protein, 219 aa, Homo sapiens
PSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 241 - protein, 209 aa, Homo sapiens
PSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPG

SEQ ID NO: 242 - protein, 23 aa, Homo sapiens
QEPKSCDKTHTCPPCPAPELLGG

SEQ ID NO: 243 - protein, 23 aa, synthetic construct
QEPKSSDKTHTCPPCPAPEAAGG

SEQ ID NO: 244 - protein, 23 aa, synthetic construct
QEPKSCDKTHTCPPCPAPEAAGG

SEQ ID NO: 245 - protein, 22 aa, synthetic construct
QEPKSSDKTHTCPPCPAPPVAG

SEQ ID NO: 246 - protein, 18 aa, Homo sapiens
ERKCCVECPPCPAPPVAG

SEQ ID NO: 247 - protein, 13 aa, synthetic construct
QEPKSSDKTHTSP

SEQ ID NO: 248 - protein, 12 aa, synthetic construct
EPKSSDKTHTSP

SEQ ID NO: 249 - protein, 23 aa, synthetic construct
QEPKSSDKTHTCPPCPAPELLGG

SEQ ID NO: 250 - protein, 277 aa, Homo sapiens
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 251 - protein, 198 aa, Mus musculus
MEGEGVQPLDENLENGSRPRFKWKKTLRLVVSGIKGAGMLLCFIYVCLQLSSSPAKDPPIQRLRGAVTRCEDGQLFISSYKNEYQTMEVQNNSVVIKCDGLYIIYLKGSFFQEVKIDLHFREDHNPISIPMLNDGRRIVFTVVASLAFKDKVYLTVNAPDTLCEHLQINDGELIVVQLTPGYCAPEGSYHSTVNQVPL

SEQ ID NO: 252 - protein, 454 aa, synthetic construct
EVQLVESGGGLVQPGGSLRLSCAASGFTFSNYAMNWVRQAPGKGLEWVSTISGSGGATRYADSVKGRFTISRDNSRNTVYLQMNSLRVEDTAVFYCTKDRLIMATVRGPYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 253 - protein, 214 aa, synthetic construct
DIQMTQSPSSLSASVGDRVTITCRASQSISSYLNWYQQKPGKAPNLLIYAASSLQSGVPSRFSGSGSETDFTLTISSLQPEDFATYYCQQSHSVSFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 254 - protein, 449 aa, synthetic construct; identical to SEQ ID NO: 36

SEQ ID NO: 255 - protein, 214 aa, synthetic construct; identical to SEQ ID NO: 35

SEQ ID NO: 256 - protein, 594 aa, synthetic construct
MHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQLQLQESGGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 257 - protein, 594 aa, synthetic construct
MHVAQPAVVLASSRGIASFVCEYASPGKHTEVRVTVLRQADSQVTEVCAANYMMGNEGTFLDDSICTGTSSGNQVNLTIQGLRAMDTGLYICMVEKMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQLQLQESGGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 258 - protein, 594 aa, synthetic construct
MHVAQPAVVLASSRGIASFVCEYASPGKKTEVRVTVLRQADSQVTEVCAANYMMGNEGTFLDDSICTGTSSGNQVNLTIQGLRAMDTGLYICMVEKMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQLQLQESGGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 259 - protein, 594 aa, synthetic construct
MHVAQPAVVLASSRGIASFVCEYASPGKTTEVRVTVLRQADSQVTEVCAANYMMGNEGTFLDDSICTGTSSGNQVNLTIQGLRAMDTGLYICMVEKMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQLQLQESGGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 260 - protein, 594 aa, synthetic construct
MHVAQPAVVLASSRGIASFVCEYASPGKWTEVRVTVLRQADSQVTEVCAANYMMGNEGTFLDDSICTGTSSGNQVNLTIQGLRAMDTGLYICMVEKMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQLQLQESGGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 261 - protein, 594 aa, synthetic construct
MHVAQPAVVLASSRGIASFVCEYASPGKYTEVRVTVLRQADSQVTEVCAANYMMGNEGTFLDDSICTGTSSGNQVNLTIQGLRAMDTGLYICMVEKMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQLQLQESGGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 262 - protein, 594 aa, synthetic construct
MHVAQPAVVLASSRGIASFVCEYASPGKHTEVRVTVLRQADSQVTEVCAAYYMMGNEGTFLDDSICTGTSSGNQVNLTIQGLRAMDTGLYICQVEKMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQLQLQESGGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 263 - protein, 594 aa, synthetic construct
MHVAQPAVVLASSRGIASFVCEYASPGKKTEVRVTVLRQADSQVTEVCAAYYMMGNEGTFLDDSICTGTSSGNQVNLTIQGLRAMDTGLYICQVEKMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQLQLQESGGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 264 - protein, 594 aa, synthetic construct
MHVAQPAVVLASSRGIASFVCEYASPGKTTEVRVTVLRQADSQVTEVCAAYYMMGNEGTFLDDSICTGTSSGNQVNLTIQGLRAMDTGLYICQVEKMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQLQLQESGGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 265 - protein, 594 aa, synthetic construct
MHVAQPAVVLASSRGIASFVCEYASPGKWTEVRVTVLRQADSQVTEVCAAYYMMGNEGTFLDDSICTGTSSGNQVNLTIQGLRAMDTGLYICQVEKMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQLQLQESGGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 266 - protein, 594 aa, synthetic construct
MHVAQPAVVLASSRGIASFVCEYASPGKYTEVRVTVLRQADSQVTEVCAAYYMMGNEGTFLDDSICTGTSSGNQVNLTIQGLRAMDTGLYICQVEKMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQLQLQESGGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 267 - protein, 594 aa, synthetic construct; identical to SEQ ID NO: 262

SEQ ID NO: 268 - protein, 594 aa, synthetic construct; identical to SEQ ID NO: 263

SEQ ID NO: 269 - protein, 594 aa, synthetic construct; identical to SEQ ID NO: 264

SEQ ID NO: 270 - protein, 594 aa, synthetic construct; identical to SEQ ID NO: 265

SEQ ID NO: 271 - protein, 594 aa, synthetic construct; identical to SEQ ID NO: 266

SEQ ID NO: 272 - protein, 594 aa, synthetic construct
MHVAQPAVVLASSRGIASFVCEYASPGKHTEVRVTVLRQADSQVTEVCAANYMMGNEGTFLDDSICTGTSSGNQVNLTIQGLRAMDTGLYICQVEKMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQLQLQESGGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 273 - protein, 594 aa, synthetic construct
MHVAQPAVVLASSRGIASFVCEYASPGKKTEVRVTVLRQADSQVTEVCAANYMMGNEGTFLDDSICTGTSSGNQVNLTIQGLRAMDTGLYICQVEKMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQLQLQESGGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 274 - protein, 594 aa, synthetic construct
MHVAQPAVVLASSRGIASFVCEYASPGKTTEVRVTVLRQADSQVTEVCAANYMMGNEGTFLDDSICTGTSSGNQVNLTIQGLRAMDTGLYICQVEKMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQLQLQESGGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 275 - protein, 594 aa, synthetic construct
MHVAQPAVVLASSRGIASFVCEYASPGKWTEVRVTVLRQADSQVTEVCAANYMMGNEGTFLDDSICTGTSSGNQVNLTIQGLRAMDTGLYICQVEKMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQLQLQESGGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 276 - protein, 594 aa, synthetic construct
MHVAQPAVVLASSRGIASFVCEYASPGKYTEVRVTVLRQADSQVTEVCAANYMMGNEGTFLDDSICTGTSSGNQVNLTIQGLRAMDTGLYICQVEKMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQLQLQESGGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 277 - protein, 594 aa, synthetic construct
MHVAQPAVVLASSRGIASFVCEYASPGKHTEVRVTVLRQADSQVTEVCFANYYMGNELTFDDDSICTGTSSGNQVNLTIQGLRAMDTGLYICQVELMYPPPYYEGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQLQLQESGGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 278 - protein, 594 aa, synthetic construct
MHVAQPAVVLASSRGIASFVCEYASPGKKTEVRVTVLRQADSQVTEVCFANYYMGNELTFDDDSICTGTSSGNQVNLTIQGLRAMDTGLYICQVELMYPPPYYEGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQLQLQESGGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 279 - protein, 594 aa, synthetic construct
MHVAQPAVVLASSRGIASFVCEYASPGKWTEVRVTVLRQADSQVTEVCFANYYMGNELTFDDDSICTGTSSGNQVNLTIQGLRAMDTGLYICQVELMYPPPYYEGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQLQLQESGGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 280 - protein, 594 aa, synthetic construct
MHVAQPAVVLASSRGIASFVCEYASPGKYTEVRVTVLRQADSQVTEVCFANYYMGNELTFDDDSICTGTSSGNQVNLTIQGLRAMDTGLYICQVELMYPPPYYEGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQLQLQESGGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 281 - protein, 594 aa, synthetic construct
MHVAQPAVVLASSRGIASFVCEYASPGKHTEVRVTVLRQADSQVTEVCFANYYMGNELTFEDDSICTGTSSGNQVNLTIQGLRAMDTGLYICQVELMYPPPYYEGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQLQLQESGGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 282 - protein, 594 aa, synthetic construct
MHVAQPAVVLASSRGIASFVCEYASPGKKTEVRVTVLRQADSQVTEVCFANYYMGNELTFEDDSICTGTSSGNQVNLTIQGLRAMDTGLYICQVELMYPPPYYEGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQLQLQESGGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 283 - protein, 594 aa, synthetic construct
MHVAQPAVVLASSRGIASFVCEYASPGKWTEVRVTVLRQADSQVTEVCFANYYMGNELTFEDDSICTGTSSGNQVNLTIQGLRAMDTGLYICQVELMYPPPYYEGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQLQLQESGGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 284 - protein, 594 aa, synthetic construct
MHVAQPAVVLASSRGIASFVCEYASPGKYTEVRVTVLRQADSQVTEVCFANYYMGNELTFEDDSICTGTSSGNQVNLTIQGLRAMDTGLYICQVELMYPPPYYEGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQLQLQESGGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 285 - protein, 594 aa, synthetic construct
MHVAQPAVVLASSRGIASFVCEYESPGKANEIRVTVLRQADSQVTEVCAMTYMKEDELTFLDDPSCTGTFSGNQVNLTIQGLRAMDTGLYICKVELMYPPPYYEGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQLQLQESGGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 286 - protein, 594 aa, synthetic construct
MHVAQPAVVLASSRGRASFVCEYTNPSKATEVRVTVLRQADSQVTEVCAATYMKGNEATFLDDSICTGTASGNQVNLTIRGLRAQDTGLYICQVELMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQLQLQESGGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 287 - protein, 594 aa, synthetic construct
MHVAQPAVVLASSVGRASFVCEYTNPSKATEVRVTVLRQADSQVTEVCAATYMKGNEATFLDDSICTGTASGNQVNLTIRGLRAQDTGLYICQVELMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQLQLQESGGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 288 - protein, 594 aa, synthetic construct
MHVAQPAVVLASSRGRASFVCEYTNPSKATEVRVTVLRQADSQVTEVCAATYMKGNEGTFLDDSICTGTASGNQVNLTIRGLRAQDTGLYICQVELMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQLQLQESGGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 289 - protein, 594 aa, synthetic construct
MHVAQPAVVLASSVGRASFVCEYTNPSKATEVRVTVLRQADSQVTEVCAATYMKGNEGTFLDDSICTGTASGNQVNLTIRGLRAQDTGLYICQVELMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQLQLQESGGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 290 - protein, 594 aa, synthetic construct
MHVAQPAVVLASSRGRASFVCEYTNPSKATEVRVTVLRQADSQVTEVCAATYMKGNEATFLDDSICTGTASGNQVNLTIRGLRASDTGLYICQVELMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQLQLQESGGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 291 - protein, 594 aa, synthetic construct
MHVAQPAVVLASSVGRASFVCEYTNPSKATEVRVTVLRQADSQVTEVCAATYMKGNEATFLDDSICTGTASGNQVNLTIRGLRASDTGLYICQVELMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQLQLQESGGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 292 - protein, 594 aa, synthetic construct
MHVAQPAVVLASSRGRASFVCEYTNPSKATEVRVTVLRQADSQVTEVCAATYMKGNEGTFLDDSICTGTASGNQVNLTIRGLRASDTGLYICQVELMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQLQLQESGGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 293 - protein, 594 aa, synthetic construct
MHVAQPAVVLASSVGRASFVCEYTNPSKATEVRVTVLRQADSQVTEVCAATYMKGNEGTFLDDSICTGTASGNQVNLTIRGLRASDTGLYICQVELMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQLQLQESGGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 294 - protein, 594 aa, synthetic construct
MHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVIDPEPCPDSDAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 295 - protein, 594 aa, synthetic construct
MHVAQPAVVLASSRGIASFVCEYASPGKHTEVRVTVLRQADSQVTEVCAANYMMGNEGTFLDDSICTGTSSGNQVNLTIQGLRAMDTGLYICMVEKMYPPPYYLGIGNGTQIYVIDPEPCPDSDAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 296 - protein, 594 aa, synthetic construct
MHVAQPAVVLASSRGIASFVCEYASPGKKTEVRVTVLRQADSQVTEVCAANYMMGNEGTFLDDSICTGTSSGNQVNLTIQGLRAMDTGLYICMVEKMYPPPYYLGIGNGTQIYVIDPEPCPDSDAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 297 - protein, 594 aa, synthetic construct
MHVAQPAVVLASSRGIASFVCEYASPGKTTEVRVTVLRQADSQVTEVCAANYMMGNEGTFLDDSICTGTSSGNQVNLTIQGLRAMDTGLYICMVEKMYPPPYYLGIGNGTQIYVIDPEPCPDSDAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 298 - protein, 594 aa, synthetic construct
MHVAQPAVVLASSRGIASFVCEYASPGKWTEVRVTVLRQADSQVTEVCAANYMMGNEGTFLDDSICTGTSSGNQVNLTIQGLRAMDTGLYICMVEKMYPPPYYLGIGNGTQIYVIDPEPCPDSDAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 299 - protein, 594 aa, synthetic construct
MHVAQPAVVLASSRGIASFVCEYASPGKYTEVRVTVLRQADSQVTEVCAANYMMGNEGTFLDDSICTGTSSGNQVNLTIQGLRAMDTGLYICMVEKMYPPPYYLGIGNGTQIYVIDPEPCPDSDAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 300 - protein, 594 aa, synthetic construct
MHVAQPAVVLASSRGIASFVCEYASPGKHTEVRVTVLRQADSQVTEVCAAYYMMGNEGTFLDDSICTGTSSGNQVNLTIQGLRAMDTGLYICQVEKMYPPPYYLGIGNGTQIYVIDPEPCPDSDAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 301 - protein, 594 aa, synthetic construct
MHVAQPAVVLASSRGIASFVCEYASPGKKTEVRVTVLRQADSQVTEVCAAYYMMGNEGTFLDDSICTGTSSGNQVNLTIQGLRAMDTGLYICQVEKMYPPPYYLGIGNGTQIYVIDPEPCPDSDAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 302 - protein, 594 aa, synthetic construct
MHVAQPAVVLASSRGIASFVCEYASPGKTTEVRVTVLRQADSQVTEVCAAYYMMGNEGTFLDDSICTGTSSGNQVNLTIQGLRAMDTGLYICQVEKMYPPPYYLGIGNGTQIYVIDPEPCPDSDAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 303 - protein, 594 aa, synthetic construct
MHVAQPAVVLASSRGIASFVCEYASPGKWTEVRVTVLRQADSQVTEVCAAYYMMGNEGTFLDDSICTGTSSGNQVNLTIQGLRAMDTGLYICQVEKMYPPPYYLGIGNGTQIYVIDPEPCPDSDAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 304 - protein, 594 aa, synthetic construct
MHVAQPAVVLASSRGIASFVCEYASPGKYTEVRVTVLRQADSQVTEVCAAYYMMGNEGTFLDDSICTGTSSGNQVNLTIQGLRAMDTGLYICQVEKMYPPPYYLGIGNGTQIYVIDPEPCPDSDAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 305 - protein, 594 aa, synthetic construct; identical to SEQ ID NO: 300

SEQ ID NO: 306 - protein, 594 aa, synthetic construct; identical to SEQ ID NO: 301

SEQ ID NO: 307 - protein, 594 aa, synthetic construct; identical to SEQ ID NO: 302

SEQ ID NO: 308 - protein, 594 aa, synthetic construct; identical to SEQ ID NO: 303

SEQ ID NO: 309 - protein, 594 aa, synthetic construct; identical to SEQ ID NO: 304

SEQ ID NO: 310 - protein, 594 aa, synthetic construct
MHVAQPAVVLASSRGIASFVCEYASPGKHTEVRVTVLRQADSQVTEVCAANYMMGNEGTFLDDSICTGTSSGNQVNLTIQGLRAMDTGLYICQVEKMYPPPYYLGIGNGTQIYVIDPEPCPDSDAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 311 - protein, 594 aa, synthetic construct
MHVAQPAVVLASSRGIASFVCEYASPGKKTEVRVTVLRQADSQVTEVCAANYMMGNEGTFLDDSICTGTSSGNQVNLTIQGLRAMDTGLYICQVEKMYPPPYYLGIGNGTQIYVIDPEPCPDSDAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 312 - protein, 594 aa, synthetic construct
MHVAQPAVVLASSRGIASFVCEYASPGKTTEVRVTVLRQADSQVTEVCAANYMMGNEGTFLDDSICTGTSSGNQVNLTIQGLRAMDTGLYICQVEKMYPPPYYLGIGNGTQIYVIDPEPCPDSDAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 313 - protein, 594 aa, synthetic construct
MHVAQPAVVLASSRGIASFVCEYASPGKWTEVRVTVLRQADSQVTEVCAANYMMGNEGTFLDDSICTGTSSGNQVNLTIQGLRAMDTGLYICQVEKMYPPPYYLGIGNGTQIYVIDPEPCPDSDAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 314 - protein, 594 aa, synthetic construct
MHVAQPAVVLASSRGIASFVCEYASPGKYTEVRVTVLRQADSQVTEVCAANYMMGNEGTFLDDSICTGTSSGNQVNLTIQGLRAMDTGLYICQVEKMYPPPYYLGIGNGTQIYVIDPEPCPDSDAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 315 - protein, 594 aa, synthetic construct
MHVAQPAVVLASSRGIASFVCEYASPGKHTEVRVTVLRQADSQVTEVCFANYYMGNELTFDDDSICTGTSSGNQVNLTIQGLRAMDTGLYICQVELMYPPPYYEGIGNGTQIYVIDPEPCPDSDAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 316 - protein, 594 aa, synthetic construct
MHVAQPAVVLASSRGIASFVCEYASPGKKTEVRVTVLRQADSQVTEVCFANYYMGNELTFDDDSICTGTSSGNQVNLTIQGLRAMDTGLYICQVELMYPPPYYEGIGNGTQIYVIDPEPCPDSDAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 317 - protein, 594 aa, synthetic construct
MHVAQPAVVLASSRGIASFVCEYASPGKWTEVRVTVLRQADSQVTEVCFANYYMGNELTFDDDSICTGTSSGNQVNLTIQGLRAMDTGLYICQVELMYPPPYYEGIGNGTQIYVIDPEPCPDSDAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 318 - protein, 594 aa, synthetic construct
MHVAQPAVVLASSRGIASFVCEYASPGKYTEVRVTVLRQADSQVTEVCFANYYMGNELTFDDDSICTGTSSGNQVNLTIQGLRAMDTGLYICQVELMYPPPYYEGIGNGTQIYVIDPEPCPDSDAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 319 - protein, 594 aa, synthetic construct
MHVAQPAVVLASSRGIASFVCEYASPGKHTEVRVTVLRQADSQVTEVCFANYYMGNELTFEDDSICTGTSSGNQVNLTIQGLRAMDTGLYICQVELMYPPPYYEGIGNGTQIYVIDPEPCPDSDAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 320 - protein, 594 aa, synthetic construct
MHVAQPAVVLASSRGIASFVCEYASPGKKTEVRVTVLRQADSQVTEVCFANYYMGNELTFEDDSICTGTSSGNQVNLTIQGLRAMDTGLYICQVELMYPPPYYEGIGNGTQIYVIDPEPCPDSDAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 321 - protein, 594 aa, synthetic construct
MHVAQPAVVLASSRGIASFVCEYASPGKWTEVRVTVLRQADSQVTEVCFANYYMGNELTFEDDSICTGTSSGNQVNLTIQGLRAMDTGLYICQVELMYPPPYYEGIGNGTQIYVIDPEPCPDSDAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 322 - protein, 594 aa, synthetic construct
MHVAQPAVVLASSRGIASFVCEYASPGKYTEVRVTVLRQADSQVTEVCFANYYMGNELTFEDDSICTGTSSGNQVNLTIQGLRAMDTGLYICQVELMYPPPYYEGIGNGTQIYVIDPEPCPDSDAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 323 - protein, 594 aa, synthetic construct
MHVAQPAVVLASSRGIASFVCEYESPGKANEIRVTVLRQADSQVTEVCAMTYMKEDELTFLDDPSCTGTFSGNQVNLTIQGLRAMDTGLYICKVELMYPPPYYEGIGNGTQIYVIDPEPCPDSDAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 324 - protein, 594 aa, synthetic construct
MHVAQPAVVLASSRGRASFVCEYTNPSKATEVRVTVLRQADSQVTEVCAATYMKGNEATFLDDSICTGTASGNQVNLTIRGLRAQDTGLYICQVELMYPPPYYLGIGNGTQIYVIDPEPCPDSDAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 325 - protein, 594 aa, synthetic construct
MHVAQPAVVLASSVGRASFVCEYTNPSKATEVRVTVLRQADSQVTEVCAATYMKGNEATFLDDSICTGTASGNQVNLTIRGLRAQDTGLYICQVELMYPPPYYLGIGNGTQIYVIDPEPCPDSDAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 326 - protein, 594 aa, synthetic construct
MHVAQPAVVLASSRGRASFVCEYTNPSKATEVRVTVLRQADSQVTEVCAATYMKGNEGTFLDDSICTGTASGNQVNLTIRGLRAQDTGLYICQVELMYPPPYYLGIGNGTQIYVIDPEPCPDSDAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 327 - protein, 594 aa, synthetic construct
MHVAQPAVVLASSVGRASFVCEYTNPSKATEVRVTVLRQADSQVTEVCAATYMKGNEGTFLDDSICTGTASGNQVNLTIRGLRAQDTGLYICQVELMYPPPYYLGIGNGTQIYVIDPEPCPDSDAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 328 - protein, 594 aa, synthetic construct
MHVAQPAVVLASSRGRASFVCEYTNPSKATEVRVTVLRQADSQVTEVCAATYMKGNEATFLDDSICTGTASGNQVNLTIRGLRASDTGLYICQVELMYPPPYYLGIGNGTQIYVIDPEPCPDSDAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 329 - protein, 594 aa, synthetic construct
MHVAQPAVVLASSVGRASFVCEYTNPSKATEVRVTVLRQADSQVTEVCAATYMKGNEATFLDDSICTGTASGNQVNLTIRGLRASDTGLYICQVELMYPPPYYLGIGNGTQIYVIDPEPCPDSDAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 330 - protein, 594 aa, synthetic construct
MHVAQPAVVLASSRGRASFVCEYTNPSKATEVRVTVLRQADSQVTEVCAATYMKGNEGTFLDDSICTGTASGNQVNLTIRGLRASDTGLYICQVELMYPPPYYLGIGNGTQIYVIDPEPCPDSDAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 331 - protein, 594 aa, synthetic construct
MHVAQPAVVLASSVGRASFVCEYTNPSKATEVRVTVLRQADSQVTEVCAATYMKGNEGTFLDDSICTGTASGNQVNLTIRGLRASDTGLYICQVELMYPPPYYLGIGNGTQIYVIDPEPCPDSDAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREGPGLVKPSETLSLTCSVSGGSISTSSYYWGWIRQPPGKGLEWSGSIYYSGSTYYRPSLQGRATISVDTSKNQFSLKLTSVTAADTAVYYCARHRGSYFFDIWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 332 - protein, 598 aa, synthetic construct
AMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSEVQLVESGGGLVQPGGSLRLSCAASGFTFSNYAMNWVRQAPGKGLEWVSTISGSGGATRYADSVKGRFTISRDNSRNTVYLQMNSLRVEDTAVFYCTKDRLIMATVRGPYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 333 - protein, 591 aa, synthetic construct
AMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSEVQLLESGGGLVQPGGSLRLSCAASGFTFNSYAMSWVRQAPGKGLEWVSIISGSGGFTYYADSVKGRFTISRDNSRTTLYLQMNSLRAEDTAVYYCAKDRLVAPGTFDYWGQGALVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 334 - protein, 597 aa, synthetic construct
AMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSEVQLVESGGGLVQPGGSLRLSCAASGFTFSNYAMNWVRQAPGKGLEWVSTISGSGGATRYADSVKGRFTISRDNSRNTVYLQMNSLRVEDTAVFYCTKDRLIMATVRGPYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 335 - protein, 590 aa, synthetic construct
AMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSEVQLLESGGGLVQPGGSLRLSCAASGFTFNSYAMSWVRQAPGKGLEWVSIISGSGGFTYYADSVKGRFTISRDNSRTTLYLQMNSLRAEDTAVYYCAKDRLVAPGTFDYWGQGALVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 336 - protein, 5 aa, Homo sapiens
EPKSC

SEQ ID NO: 337 - protein, 183 aa, Macaca fascicularis
MERVQPLEENVGNAARPRFERNKLLLVASVIQGLGLLLCFTYICLHFSALQVSHQYPRIQSIKVQFTEYKKEEGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 338 - protein, 6 aa, synthetic construct
HHHHHH